(19) United States
(12) Patent Application Publication
(10) Pub. No.: US 2026/0234108 A1
(43) Pub. Date: Aug. 13, 2026

(54) PLURALITY OF HOST MATERIALS AND ORGANIC ELECTROLUMINESCENT DEVICE COMPRISING THE SAME

(71) Applicant: DuPont Specialty Materials Korea Ltd., Chungcheongnam-do (KR)

(72) Inventors: Bitnari KIM, Gyeonggi-do (KR); Hee-Ryong KANG, Gyeonggi-do (KR); Tae-Jin LEE, Gyeonggi-do (KR); Sang-Hee CHO, Gyeonggi-do (KR)

(21) Appl. No.: 19/637,068

(22) Filed: Apr. 2, 2026

Related U.S. Application Data
(63) Continuation of application No. 16/092,826, filed on Apr. 14, 2017, now abandoned.

(30) Foreign Application Priority Data
Apr. 18, 2016 (KR) 10-2016-0046863
Apr. 12, 2017 (KR) 10-2017-0047529

(51) Int. Cl. C07D 209/80; C07D 209/86; C07D 401/04; C07D 401/14; C07D 403/04; C07D 403/10; C07D 403/14; C09K 11/06; H10K 50/11; H10K 50/15; H10K 50/16; H10K 50/17; H10K 71/16; H10K 85/30; H10K 85/60; H10K 101/00; H10K 101/10

(52) U.S. Cl. CPC C07D 209/80; C09K 11/06; H10K 85/615; H10K 85/631; H10K 85/633; H10K 85/636; H10K 85/654; H10K 85/6572; H10K 85/6576; C07D 209/86; C07D 401/04; C07D 401/14; C07D 403/04; C07D 403/10; C07D 403/14; C09K 2211/185; H10K 50/11; H10K 50/156; H10K 50/16; H10K 50/171; H10K 71/164; H10K 85/342; H10K 2101/10; H10K 2101/90

(57) ABSTRACT

The present disclosure relates to a plurality of host materials and an organic electroluminescent device comprising the same. The organic electroluminescent device of the present disclosure can have improved lifespan properties, by comprising a plurality of host compounds in a specific combination.

### CLAIM OF BENEFIT OF PRIOR APPLICATION

This application is a continuation of U.S. patent application Ser. No. 16/092,826, filed Oct. 11, 2018, which is the National Stage Entry of PCT/KR2017/004034, filed Apr. 14, 2017, both of which are incorporated by reference herein in their entirety.

### TECHNICAL FIELD

The present disclosure relates to a plurality of host materials and an organic electroluminescent device comprising the same.

### BACKGROUND ART

An electroluminescent device (EL device) is a self-light-emitting display device which has advantages in that it provides a wider viewing angle, a greater contrast ratio, and a faster response time. The first organic EL device was developed by Eastman Kodak in 1987, by using small aromatic diamine molecules and aluminum complexes as materials for forming a light-emitting layer [Appl. Phys. Lett. 51, 913, 1987].

An organic EL device (OLED) changes electric energy into light by applying electricity to an organic light-emitting material, and commonly comprises an anode, a cathode, and an organic layer formed between the two electrodes. The organic layer of the organic EL device may comprise a hole injection layer, a hole transport layer, a hole auxiliary layer, a light-emitting auxiliary layer, an electron blocking layer, a light-emitting layer (containing host and dopant materials), an electron buffer layer, a hole blocking layer, an electron transport layer, an electron injection layer, etc. The materials used in the organic layer can be classified into a hole injection material, a hole transport material, a hole auxiliary material, a light-emitting auxiliary material, an electron blocking material, a light-emitting material, an electron buffer material, a hole blocking material, an electron transport material, an electron injection material, etc., depending on functions. In the organic EL device, holes from an anode and electrons from a cathode are injected into a light-emitting layer by the application of electric voltage, and an exciton having high energy is produced by the recombination of the holes and electrons. The organic light-emitting compound moves into an excited state by the energy and emits light from energy when the organic light-emitting compound returns to the ground state from the excited state.

The most important factor determining luminous efficiency in an organic EL device is light-emitting materials. The light-emitting materials are required to have the following features: high quantum efficiency, high movement degree of an electron and a hole, and uniformality and stability of the formed light-emitting material layer. The light-emitting material is classified into blue, green, and red light-emitting materials according to the light-emitting color, and further includes yellow or orange light-emitting materials. Furthermore, the light-emitting material is classified into a host material and a dopant material in a functional aspect. Recently, an urgent task is the development of an organic EL device having high efficiency and long lifespan. In particular, the development of highly excellent light-emitting material over conventional materials is urgently required, considering the EL properties necessary for medium- and large-sized OLED panels. For this, preferably, as a solvent in a solid state and an energy transmitter, a host material should have high purity and a suitable molecular weight in order to be deposited under vacuum. Furthermore, a host material is required to have high glass transition temperature and pyrolysis temperature to achieve thermal stability, high electrochemical stability to achieve a long lifespan, easy formability of an amorphous thin film, good adhesion with adjacent layers, and no movement between layers.

A light-emitting material can be used as a combination of a host and a dopant to improve color purity, luminous efficiency, and stability. Generally, an EL device having excellent characteristics has a structure comprising a light-emitting layer formed by doping a dopant to a host. Since host materials greatly influence the efficiency and lifespan of the EL device when using a dopant/host material system as a light-emitting material, their selection is important.

Korean Patent Application Laid-Open No. 2013-0106255 discloses an organic electroluminescent device using an arylamine-based compound containing a carbazole as a hole transport material. However, said reference does not specifically disclose that the carbazole-amine-based compound is used as a co-host material or a premixed host material.

Korean Patent Application Laid-Open No. 2015-0129928 discloses an organic light-emitting device comprising an indolocarbazole derivative compound and a triphenylene-based compound as a light-emitting material. However, the light-emitting material in said reference must comprise a triphenylene-based compound, and said reference does not specifically disclose an organic electroluminescent device comprising an indolocarbazole derivative compound and a carbazole-amine-based compound as a plurality of host materials.

### DISCLOSURE OF THE INVENTION

### Problems to be Solved

The object of the present disclosure is to provide an organic electroluminescent device having long lifespan.

### Solution to Problems

As a result of intensive studies to solve the technical problem above, the present inventors found that the above objective can be achieved by a plurality of host materials comprising at least one first host compound and at least one second host compound, wherein the first host compound is represented by the following formula 1:

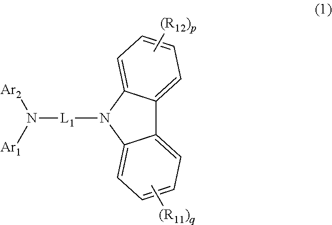

wherein
Ar₁ and Ar₂, each independently, represent a substituted or unsubstituted (C6-C30)aryl, or a substituted or unsubstituted (3- to 30-membered)heteroaryl;
L₁ represents a substituted or unsubstituted (C6-C30)arylene;
R₁₁ and R₁₂, each independently, represent hydrogen, deuterium, a substituted or unsubstituted (C1-C30)alkyl, a substituted or unsubstituted (C6-C30)aryl(C1-C30)alkyl, a substituted or unsubstituted (C6-C30)aryl, a substituted or unsubstituted (C1-C30)alkyl(C6-C30)aryl, a substituted or unsubstituted (3- to 30-membered)heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to adjacent R₁₁ and R₁₂ to form an unsubstituted benzene ring; and
p and q, each independently, represent an integer of 1 to 4, where if p and q, each independently, are an integer of 2 or more, each of R₁₁ and R₁₂ may be the same or different;
and the second host compound is represented by the following formula 2:

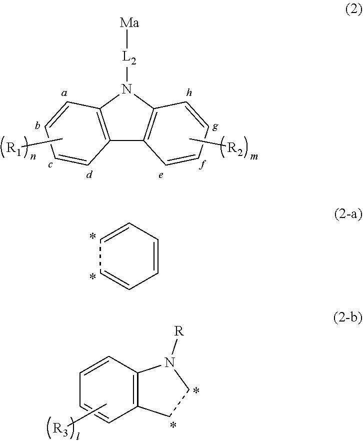

wherein
Ma represents a substituted or unsubstituted nitrogen-containing (3- to 30-membered)heteroaryl;
L₂ represents a single bond, a substituted or unsubstituted (C6-C30)arylene, or a substituted or unsubstituted nitrogen-containing (3- to 30-membered)heteroarylene;
formula 2 and formula 2-a are fused at the positions of a and b, b and c, c and d, e and f, f and g, or g and h of formula 2 and at the positions of * of formula 2-a to form at least one ring; or formula 2 and formula 2-b are fused at the positions of e and f, f and g, or g and h of formula 2 and at the positions of * of formula 2-b to form a ring;
R₁ to R₃, each independently, represent hydrogen, deuterium, a substituted or unsubstituted (C1-C30)alkyl, a substituted or unsubstituted (C6-C30)aryl(C1-C30)alkyl, a substituted or unsubstituted (C6-C30)aryl, a substituted or unsubstituted (C1-C30)alkyl(C6-C30)aryl, a substituted or unsubstituted (3- to 30-membered)heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to adjacent R₁ to R₃ to form a substituted or unsubstituted, mono- or polycyclic, (C3-C30) alicyclic or aromatic ring, or the combination thereof, whose carbon atom(s) may be replaced with at least one heteroatom selected from nitrogen, oxygen, and sulfur;
R represents hydrogen, deuterium, a substituted or unsubstituted (C1-C30)alkyl, a substituted or unsubstituted (C6-C30)aryl(C1-C30)alkyl, a substituted or unsubstituted (C6-C30)aryl, a substituted or unsubstituted (C1-C30)alkyl(C6-C30)aryl, a substituted or unsubstituted (3- to 30-membered)heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
n, m, and l, each independently, represent an integer of 1 to 4, where if n, m, and l, each independently, are an integer of 2 or more, each of R₁ to R₃ may be the same or different; and
the heteroaryl(ene) contains at least one heteroatom selected from B, N, O, S, Si, and P.

### Effects of the Invention

By using the plurality of host materials according to the present disclosure, it is possible to provide an organic electroluminescent device having long lifespan, and a display system or a lighting system using the same.

### EMBODIMENTS OF THE INVENTION

Hereinafter, the present disclosure will be described in detail. However, the following description is intended to explain the disclosure, and is not meant in any way to restrict the scope of the disclosure.

The benzocarbazole-amine-based compound, which is the first host compound according to the present disclosure, is not generally used as a light-emitting material due to its very high LUMO (lowest unoccupied molecular orbital) energy level. The present inventors found that the organic electroluminescent device of the present disclosure can achieve improved lifespan properties compared to the conventional organic electroluminescent device by comprising the first host compound, which is a benzocarbazole-amine-based compound, as a light-emitting material and comprising a plurality of host materials in a specific combination.

Hereinafter, the organic electroluminescent device comprising the host compounds represented by formulas 1 and 2 will be described in more detail.

In formula 1, Ar₁ and Ar₂, each independently, represent a substituted or unsubstituted (C6-C30)aryl, or a substituted or unsubstituted (3- to 30-membered)heteroaryl, preferably, a substituted or unsubstituted (C6-C25)aryl, or a substituted or unsubstituted (5- to 30-membered)heteroaryl, and more preferably, a substituted or unsubstituted (C6-C20)aryl, or a substituted or unsubstituted (5- to 25-membered)heteroaryl. Specifically, Ar₁ and Ar₂, each independently, may be a phenyl unsubstituted or substituted with at least one deuterium, an unsubstituted naphthylphenyl, an unsubstituted biphenyl, an unsubstituted naphthyl, an unsubstituted phenylnaphthyl, an unsubstituted binaphthyl, an unsubstituted terphenyl, a fluorenyl substituted with at least one methyl, a carbazolyl substituted with a phenyl, or an unsubstituted dibenzothiophenyl.

In formula 1, L₁ represents a substituted or unsubstituted (C6-C30)arylene, preferably, a substituted or unsubstituted (C6-C25)arylene, and more preferably, a substituted or unsubstituted (C6-C18)arylene. Specifically, L₁ may be a phenylene unsubstituted or substituted with a diphenylamino, an unsubstituted biphenylene, an unsubstituted naphthylene, or a fluorenylene substituted with at least one methyl.

In formula 1, R₁₁ and R₁₂, each independently, represent hydrogen, deuterium, a substituted or unsubstituted (C1-C30)alkyl, a substituted or unsubstituted (C6-C30)aryl(C1-C30)alkyl, a substituted or unsubstituted (C6-C30)aryl, a substituted or unsubstituted (C1-C30)alkyl(C6-C30)aryl, a substituted or unsubstituted (3- to 30-membered)heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to adjacent R₁₁ and R₁₂ to form a substituted or unsubstituted, mono- or polycyclic, (C3-C30) alicyclic or aromatic ring, or the combination thereof, whose carbon atom(s) may be replaced with at least one heteroatom selected from nitrogen, oxygen, and sulfur. Preferably, R₁₁ and R₁₂, each independently, represent hydrogen, or a substituted or unsubstituted (C6-C25)aryl, or are linked to adjacent R₁₁ and R₁₂ to form a substituted or unsubstituted, mono- or polycyclic, (C3-C25) aromatic ring; more preferably, represent hydrogen, or a substituted or unsubstituted (C6-C18)aryl, or are linked to adjacent R₁₁ and R₁₂ to form at least one unsubstituted benzene ring; and, for example, represent hydrogen or an unsubstituted phenyl, or are linked to adjacent R₁₁ and R₁₂ to form an unsubstituted benzene ring.

Formula 2 is fused with formula 2-a or formula 2-b to form an aromatic ring, in which formula 2 and formula 2-a may be fused at the positions of a and b, b and c, c and d, e and f, f and g, or g and h of formula 2 and at the positions of * of formula 2-a to form at least one ring; or formula 2 and formula 2-b may be fused at the positions of e and f, f and g, or g and h of formula 2 and at the positions of * of formula 2-b to form a ring.

In formula 2, Ma represents a substituted or unsubstituted nitrogen-containing (3- to 30-membered)heteroaryl, preferably, a substituted or unsubstituted nitrogen-containing (5- to 25-membered)heteroaryl, and more preferably, a substituted nitrogen-containing (5- to 18-membered)heteroaryl. According to one embodiment of the present disclosure, Ma is a monocyclic ring-type heteroaryl selected from the group consisting of a substituted or unsubstituted pyrrolyl, a substituted or unsubstituted imidazolyl, a substituted or unsubstituted pyrazolyl, a substituted or unsubstituted triazinyl, a substituted or unsubstituted tetrazinyl, a substituted or unsubstituted triazolyl, a substituted or unsubstituted tetrazolyl, a substituted or unsubstituted pyridyl, a substituted or unsubstituted pyrazinyl, a substituted or unsubstituted pyrimidinyl, and a substituted or unsubstituted pyridazinyl, or a fused ring-type heteroaryl selected from the group consisting of a substituted or unsubstituted benzimidazolyl, a substituted or unsubstituted isoindolyl, a substituted or unsubstituted indolyl, a substituted or unsubstituted indazolyl, a substituted or unsubstituted benzothiadiazolyl, a substituted or unsubstituted quinolyl, a substituted or unsubstituted isoquinolyl, a substituted or unsubstituted cinnolinyl, a substituted or unsubstituted quinazolinyl, a substituted or unsubstituted naphthyridinyl, a substituted or unsubstituted quinoxalinyl, a substituted or unsubstituted carbazolyl, and a substituted or unsubstituted phenanthridinyl; preferably, a monocyclic ring-type heteroaryl selected from the group consisting of a substituted or unsubstituted triazinyl, a substituted or unsubstituted pyridyl, and a substituted or unsubstituted pyrimidinyl, or a fused ring-type heteroaryl selected from the group consisting of a substituted or unsubstituted quinolyl, a substituted or unsubstituted quinazolinyl, a substituted or unsubstituted quinoxalinyl, and a substituted or unsubstituted carbazolyl; and more preferably, a monocyclic ring-type heteroaryl selected from the group consisting of a substituted triazinyl, a substituted pyridyl, and a substituted pyrimidinyl, or a fused ring-type heteroaryl selected from the group consisting of a substituted quinolyl, a substituted quinazolinyl, a substituted quinoxalinyl, and a substituted carbazolyl, in which the substituents of the substituted triazinyl, the substituted pyridyl, the substituted pyrimidinyl, the substituted quinolyl, the substituted quinazolinyl, the substituted quinoxalinyl, and a substituted carbazolyl may be at least one selected from a phenyl unsubstituted or substituted with a cyano, a naphthylphenyl, a biphenyl, a naphthyl, a fluorenyl substituted with at least one methyl, a fluorenyl substituted with at least one phenyl, a benzofluorenyl substituted with at least one methyl, a carbazolyl, a benzocarbazolyl substituted with at least one methyl, a pyridyl substituted with a phenyl, and a dibenzothiophenyl.

In formula 2, L₂ represents a single bond, a substituted or unsubstituted (C6-C30)arylene, or a substituted or unsubstituted nitrogen-containing (3- to 30-membered)heteroarylene; preferably, a single bond, a substituted or unsubstituted (C6-C25)arylene, or a substituted d or unsubstituted nitrogen-containing (5- to 25-membered)heteroarylene; and more preferably, a single bond, a substituted or unsubstituted (C6-C18)arylene, or a substituted or unsubstituted nitrogen-containing (5- to 18-membered)heteroarylene. Specifically, L₂ may be a single bond, an unsubstituted phenylene, an unsubstituted naphthylene, an unsubstituted biphenylene, a fluorenylene substituted with at least one methyl, an unsubstituted quinazolinylene, an unsubstituted pyridylene, or an unsubstituted quinolylene.

In formula 2 and formula 2-b, R₁ to R₃, each independently, represent hydrogen, deuterium, a substituted or unsubstituted (C1-C30)alkyl, a substituted or unsubstituted (C6-C30)aryl(C1-C30)alkyl, a substituted or unsubstituted (C6-C30)aryl, a substituted or unsubstituted (C1-C30)alkyl(C6-C30)aryl, a substituted or unsubstituted (3- to 30-membered)heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to adjacent R₁ to R₃ to form a substituted or unsubstituted, mono- or polycyclic, (C3-C30) alicyclic or aromatic ring, or the combination thereof, whose carbon atom(s) may be replaced with at least one heteroatom selected from nitrogen, oxygen, and sulfur. Preferably, R₁ to R₃, each independently, represent hydrogen, or a substituted or unsubstituted (C6-C25)aryl; or are linked to adjacent R₁ to R₃ to form a substituted or unsubstituted, mono- or polycyclic, (C3-C25) alicyclic or aromatic ring, or the combination thereof, whose carbon atom(s) may be replaced with at least one heteroatom selected from nitrogen, oxygen, and sulfur. More preferably, R₁ to R₃, each independently, represent hydrogen, or a substituted or unsubstituted (C6-C18)aryl; or are linked to adjacent R₁ to R₃ to form an unsubstituted, mono- or polycyclic, (C3-C18) aromatic ring. For example, R₁ to R₃, each independently, represent hydrogen or an unsubstituted phenyl; or are linked to adjacent R₁ to R₃ to form an unsubstituted benzene ring.

In formula 2-b, R represents hydrogen, deuterium, a substituted or unsubstituted (C1-C30)alkyl, a substituted or unsubstituted (C6-C30)aryl(C1-C30)alkyl, a substituted or unsubstituted (C6-C30)aryl, a substituted or unsubstituted (C1-C30)alkyl(C6-C30)aryl, a substituted or unsubstituted (3- to 30-membered)heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; preferably, a substituted or unsubstituted (C6-C25)aryl, or a substituted or unsubstituted (5- to 30-membered)heteroaryl; and more preferably, a substituted or unsubstituted (C6-C18)aryl, or a substituted or unsubstituted (5- to 25-membered)heteroaryl. Specifically, R may be an unsubstituted phenyl, an unsubstituted naphthyl, a fluorenyl substituted with at least one methyl, or an unsubstituted pyridyl.

In formulas 1 and 2, the heteroaryl(ene) contains at least one heteroatom selected from B, N, O, S, Si, and P, preferably, at least one heteroatom selected from N and S.

According to one embodiment of the present disclosure, in formula 1, Ar₁ and Ar₂, each independently, represent a substituted or unsubstituted (C6-C25)aryl, or a substituted or unsubstituted (5- to 30-membered)heteroaryl; L₁ represents a substituted or unsubstituted (C6-C25)arylene; and R₁₁ and R₁₂, each independently, represent hydrogen, or a substituted or unsubstituted (C6-C25)aryl, or are linked to adjacent R₁₁ and R₁₂ to form a substituted or unsubstituted, mono- or polycyclic, (C3-C25) aromatic ring.

According to another embodiment of the present disclosure, in formula 1, Ar₁ and Ar₂, each independently, represent a substituted or unsubstituted (C6-C20)aryl, or a substituted or unsubstituted (5- to 25-membered)heteroaryl; L₁ represents a substituted or unsubstituted (C6-C18)arylene; and R₁₁ and R₁₂, each independently, represent hydrogen, or a substituted or unsubstituted (C6-C18)aryl, or are linked to adjacent R₁₁ and R₁₂ to form at least one unsubstituted benzene ring.

According to one embodiment of the present disclosure, in formula 2, Ma represents a substituted or unsubstituted nitrogen-containing (5- to 25-membered)heteroaryl; L₂ represents a single bond, a substituted or unsubstituted (C6-C25)arylene, or a substituted or unsubstituted nitrogen-containing (5- to 25-membered)heteroarylene; R₁ to R₃, each independently, represent hydrogen, or a substituted or unsubstituted (C6-C25)aryl, or are linked to adjacent R₁ to R₃ to form a substituted or unsubstituted, mono- or polycyclic, (C3-C25) alicyclic or aromatic ring, or the combination thereof, whose carbon atom(s) may be replaced with at least one heteroatom selected from nitrogen, oxygen, and sulfur; and R represents a substituted or unsubstituted (C6-C25)aryl, or a substituted or unsubstituted (5- to 30-membered)heteroaryl.

According to another embodiment of the present disclosure, in formula 2, Ma represents a substituted nitrogen-containing (5- to 18-membered)heteroaryl; L₂ represents a single bond, a substituted or unsubstituted (C6-C18)arylene, or a substituted or unsubstituted nitrogen-containing (5- to 18-membered)heteroarylene; R₁ to R₃, each independently, represent hydrogen, or a substituted or unsubstituted (C6-C18)aryl, or are linked to adjacent R₁ to R₃ to form an unsubstituted, mono- or polycyclic, (C3-C18) aromatic ring; and R represents a substituted or unsubstituted (C6-C18)aryl, or a substituted or unsubstituted (5- to 25-membered)heteroaryl.

According to one aspect of the present disclosure, the plurality of host materials of the present disclosure comprise at least one first host compound and at least one second host compound, in which the first host compound is represented by formula 1, and the second host compound is represented by formula 2 and formula 2-a, which are fused at the positions of a and b, b and c, c and d, e and f, f and g, or g and h of formula 2 and at the positions of * of formula 2-a to form at least one ring:

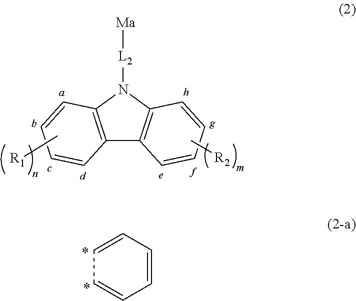

wherein Ma, L₂, R₁, R₂, n, and m are as defined above.

According to another aspect of the present disclosure, the plurality of host materials of the present disclosure comprise at least one first host compound and at least one second host compound, in which the first host compound is represented by formula 1, and the second host compound is represented by formula 2 and formula 2-b, which are fused at the positions of e and f, f and g, or g and h of formula 2 and at the positions of * of formula 2-b to form a ring:

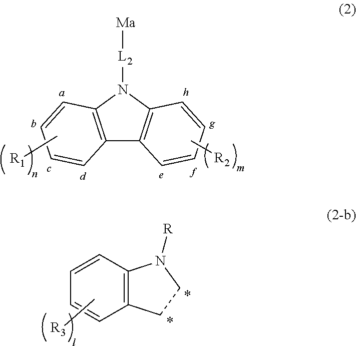

wherein Ma, L₂, R₁ to R₃, R, n, m, and l are as defined above.

According to further aspect of the present disclosure, formula 1 may be represented by any one of the following formulas 1-1 to 1-3:

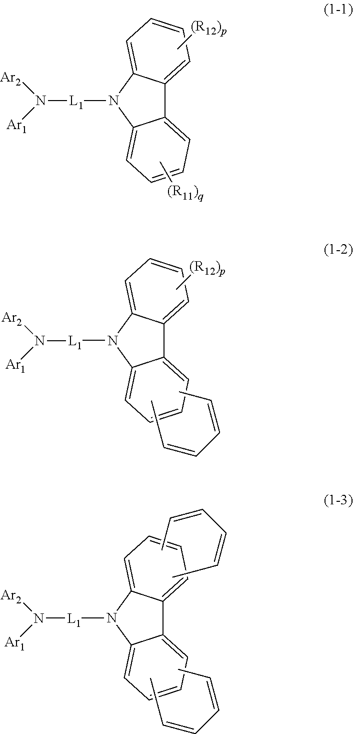

wherein Ar₁, Ar₂, L₁, R₁₁, R₁₂, p, and q are as defined in formula 1.

According to a further aspect of the present disclosure, formula 2 may be represented by any one of the following formulas 2-1 to 2-5:

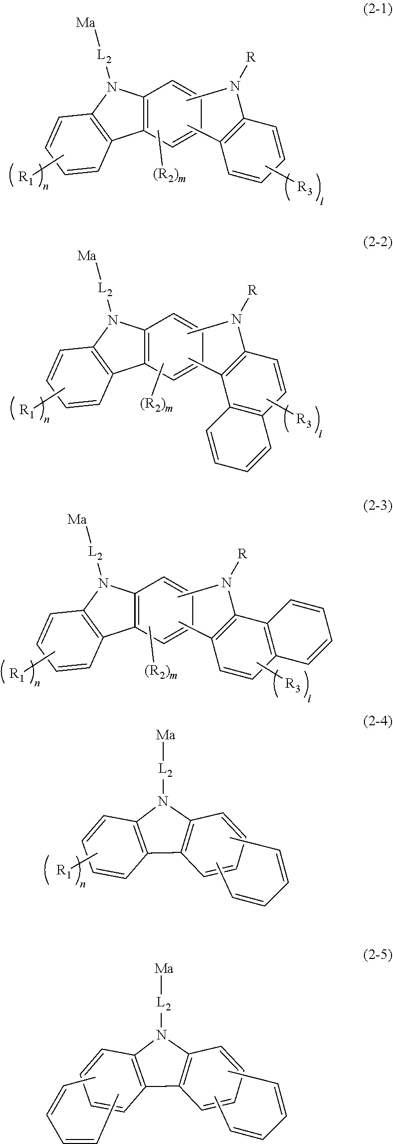

wherein Ma, L₂, R₁ to R₃, R, n, m, and l are as defined in formula 2.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, etc. The term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered) heterocycloalkyl” is a cycloalkyl having 3 to 7, preferably 5 to 7, ring backbone atoms, including at least one heteroatom selected from the group consisting of B, N, O, S, Si, and P, and preferably O, S, and N. The above heterocycloalkyl may include tetrahydrofuran, pyrrolidine, thiolan, tetrahydropyran, etc. The term “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, may be partially saturated, and may comprise a spiro structure. The above aryl may include phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, etc. The term “(3- to 30-membered)heteroaryl(ene)” is an aryl having 3 to 30 ring backbone atoms, including at least one, preferably 1 to 4 heteroatoms selected from the group consisting of B, N, O, S, Si, and P. The above heteroaryl may be a monocyclic ring, or a fused ring condensed with at least one benzene ring; may be partially saturated; may be one formed by linking at least one heteroaryl or aryl group to a heteroaryl group via a single bond. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, quinoxalinyl, naphthyridyl, carbazolyl, phenoxazinyl, phenanthridinyl, and benzodioxolyl. Furthermore, “halogen” includes F, Cl, Br, and I.

Herein, “substituted” in the expression “substituted or unsubstituted” means that a hydrogen atom in a certain functional group is replaced with another atom or another functional group, i.e. a substituent. The substituents of the substituted (C1-C30)alkyl, the substituted (C6-C30)aryl(ene), the substituted (3- to 30-membered)heteroaryl(ene), the substituted (C6-C30)aryl(C1-C30)alkyl, the substituted (C1-C30)alkyl(C6-C30)aryl, the substituted (C3-C30)cycloalkyl, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di-(C1-C30)alkylamino, the substituted mono- or di-(C6-C30)arylamino, the substituted (C1-C30)alkyl(C6-C30)arylamino, the substituted nitrogen-containing (3- to 30-membered)heteroaryl, and the substituted mono- or polycyclic, (C3-C30) alicyclic or aromatic ring, or the combination thereof, in Ar₁, Ar₂, L₁, R₁₁, R₁₂, Ma, L₂, R₁ to R₃, and R of formulas 1 and 2, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered) heterocycloalkyl; a (C6-C30)aryloxy; a (C6-C30)arylthio; a (C6-C30)aryl unsubstituted or substituted with a cyano; a (5- to 30-membered)heteroaryl unsubstituted or substituted with a (C1-C30)alkyl or a (C6-C30)aryl; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino; a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl; preferably, are at least one selected from the group consisting of deuterium, a (C1-C20)alkyl, a (C6-C25)aryl unsubstituted or substituted with a cyano, a (5- to 25-membered)heteroaryl unsubstituted or substituted with a (C1-C20)alkyl or a (C6-C25)aryl, a mono- or di-(C6-C25)arylamino, and a (C1-C20)alkyl(C6-C25)aryl; more preferably, are at least one selected from the group consisting of deuterium, a (C1-C10)alkyl, a (C6-C25)aryl unsubstituted or substituted with a cyano, a (5- to 18-membered)heteroaryl unsubstituted or substituted with a (C1-C10)alkyl or a (C6-C18)aryl, a di(C6-C18)arylamino, and a (C1-C10)alkyl(C6-C18)aryl; and for example, may be deuterium, an unsubstituted methyl, a phenyl unsubstituted or substituted with a cyano, an unsubstituted naphthylphenyl, an unsubstituted naphthyl, an unsubstituted biphenyl, a fluorenyl substituted with a dimethyl, a fluorenyl substituted with a diphenyl, a benzofluorenyl substituted with a dimethyl, an unsubstituted carbazolyl, a benzocarbazolyl substituted with a dimethyl, a pyridyl substituted with a phenyl, an unsubstituted dibenzothiophenyl, or an unsubstituted diphenylamino.

The first host compound represented by formula 1 includes the following compounds, but is not limited thereto:

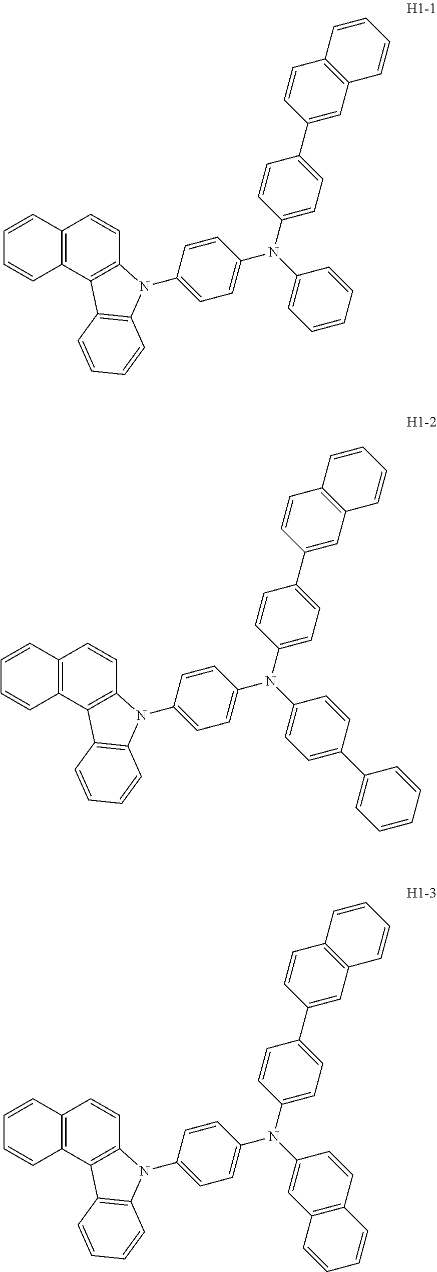 [SMILES: c1ccc(N(c2ccc(-c3ccc4ccccc4c3)cc2)c2ccc(-n3c4ccccc4c4c5ccccc5ccc43)cc2)cc1 ; c1ccc(-c2ccc(N(c3ccc(-c4ccc5ccccc5c4)cc3)c3ccc(-n4c5ccccc5c5c6ccccc6ccc54)cc3)cc2)cc1 ; c1ccc2cc(-c3ccc(N(c4ccc(-n5c6ccccc6c6c7ccccc7ccc65)cc4)c4ccc5ccccc5c4)cc3)ccc2c1]

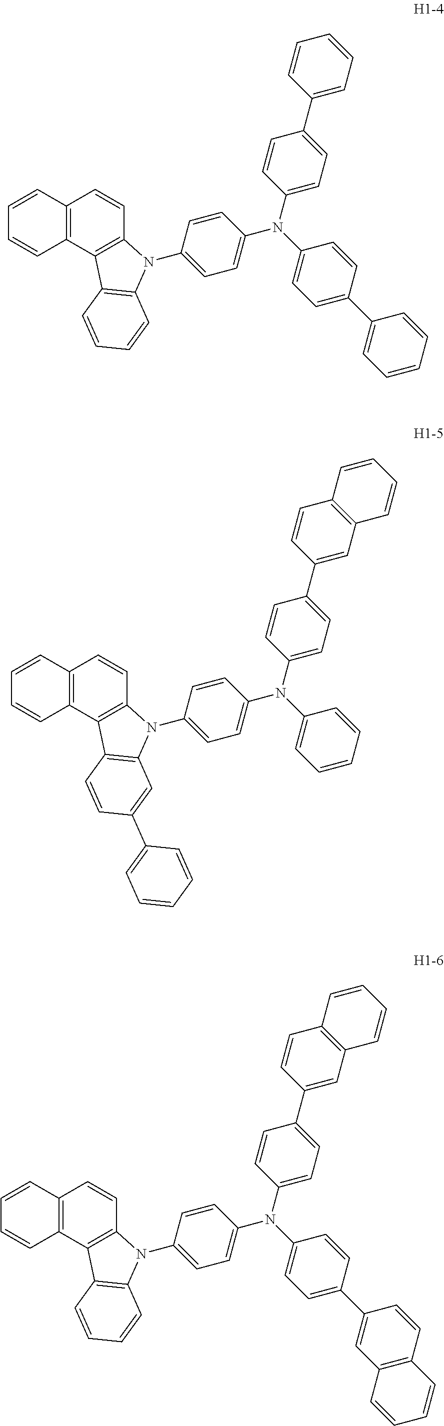 [SMILES: c1ccc(-c2ccc(N(c3ccc(-c4ccccc4)cc3)c3ccc(-n4c5ccccc5c5c6ccccc6ccc54)cc3)cc2)cc1 ; c1ccc(-c2ccc3c4c5ccccc5ccc4n(-c4ccc(N(c5ccccc5)c5ccc(-c6ccc7ccccc7c6)cc5)cc4)c3c2)cc1 ; c1ccc2cc(-c3ccc(N(c4ccc(-c5ccc6ccccc6c5)cc4)c4ccc(-n5c6ccccc6c6c7ccccc7ccc65)cc4)cc3)ccc2c1]

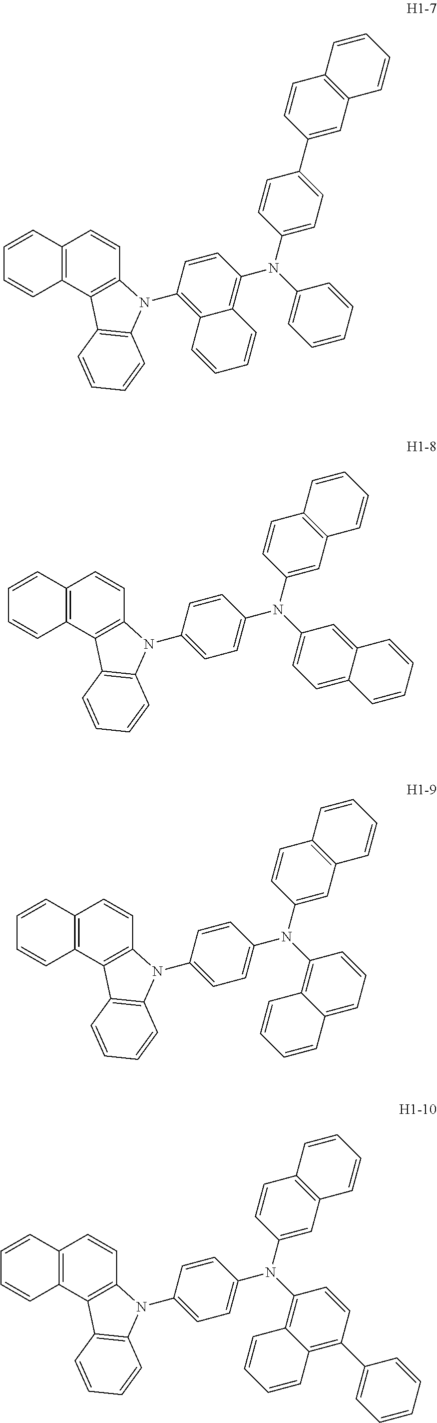 [SMILES: c1ccc(N(c2ccc(-c3ccc4ccccc4c3)cc2)c2ccc(-n3c4ccccc4c4c5ccccc5ccc43)c3ccccc23)cc1 ; c1ccc2cc(N(c3ccc(-n4c5ccccc5c5c6ccccc6ccc54)cc3)c3ccc4ccccc4c3)ccc2c1 ; c1ccc2cc(N(c3ccc(-n4c5ccccc5c5c6ccccc6ccc54)cc3)c3cccc4ccccc34)ccc2c1 ; c1ccc(-c2ccc(N(c3ccc(-n4c5ccccc5c5c6ccccc6ccc54)cc3)c3ccc4ccccc4c3)c3ccccc23)cc1]

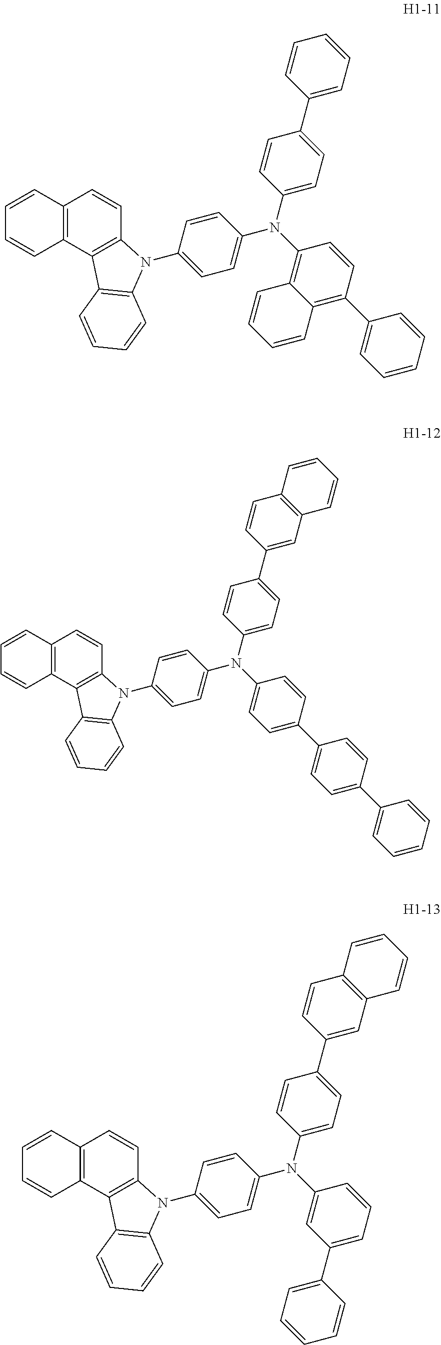 [SMILES: c1ccc(-c2ccc(N(c3ccc(-n4c5ccccc5c5c6ccccc6ccc54)cc3)c3ccc(-c4ccccc4)c4ccccc34)cc2)cc1 ; c1ccc(-c2ccc(-c3ccc(N(c4ccc(-c5ccc6ccccc6c5)cc4)c4ccc(-n5c6ccccc6c6c7ccccc7ccc65)cc4)cc3)cc2)cc1 ; c1ccc(-c2cccc(N(c3ccc(-c4ccc5ccccc5c4)cc3)c3ccc(-n4c5ccccc5c5c6ccccc6ccc54)cc3)c2)cc1]

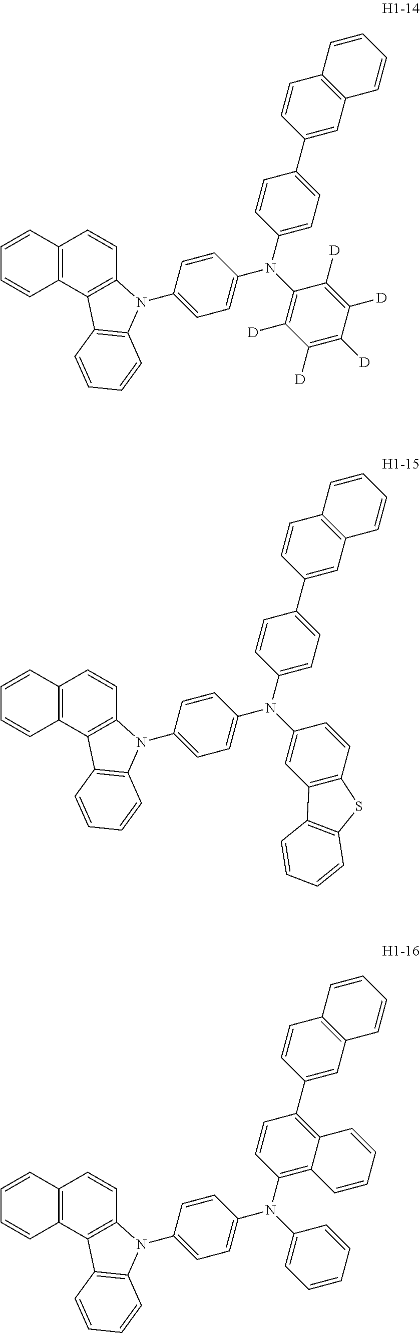 [SMILES: [2H]c1c([2H])c([2H])c(N(c2ccc(-c3ccc4ccccc4c3)cc2)c2ccc(-n3c4ccccc4c4c5ccccc5ccc43)cc2)c([2H])c1[2H] ; c1ccc2cc(-c3ccc(N(c4ccc(-n5c6ccccc6c6c7ccccc7ccc65)cc4)c4ccc5sc6ccccc6c5c4)cc3)ccc2c1 ; c1ccc(N(c2ccc(-n3c4ccccc4c4c5ccccc5ccc43)cc2)c2ccc(-c3ccc4ccccc4c3)c3ccccc23)cc1]

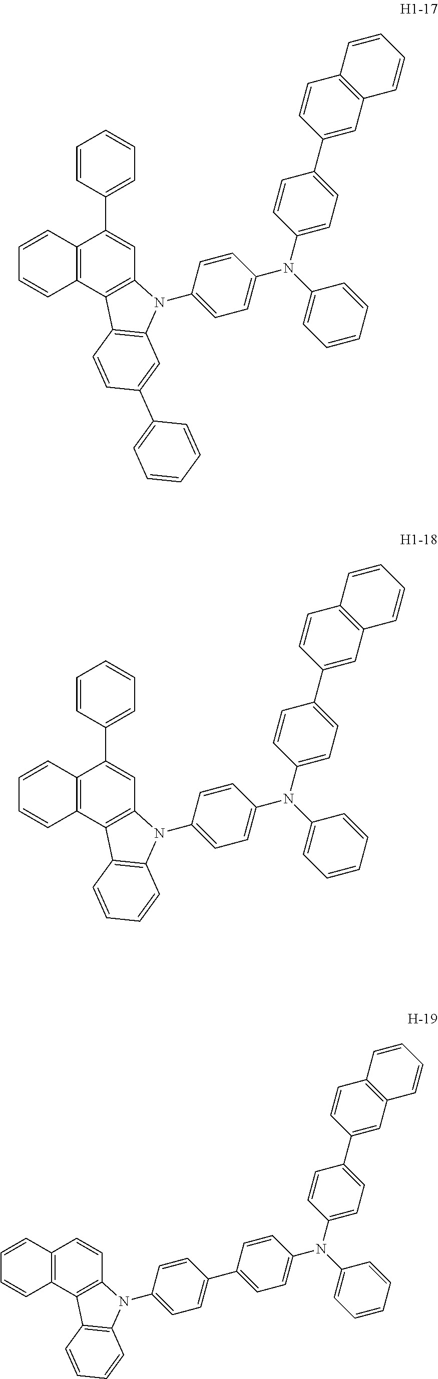 [SMILES: c1ccc(-c2ccc3c4c5ccccc5c(-c5ccccc5)cc4n(-c4ccc(N(c5ccccc5)c5ccc(-c6ccc7ccccc7c6)cc5)cc4)c3c2)cc1 ; c1ccc(-c2cc3c(c4ccccc24)c2ccccc2n3-c2ccc(N(c3ccccc3)c3ccc(-c4ccc5ccccc5c4)cc3)cc2)cc1 ; c1ccc(N(c2ccc(-c3ccc(-n4c5ccccc5c5c6ccccc6ccc54)cc3)cc2)c2ccc(-c3ccc4ccccc4c3)cc2)cc1]

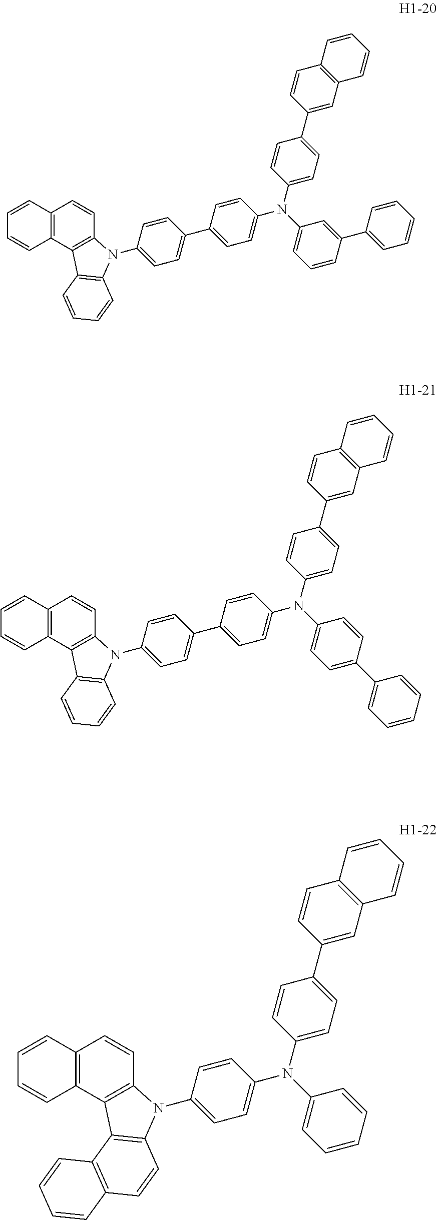 [SMILES: c1ccc(-c2cccc(N(c3ccc(-c4ccc(-n5c6ccccc6c6c7ccccc7ccc65)cc4)cc3)c3ccc(-c4ccc5ccccc5c4)cc3)c2)cc1 ; c1ccc(-c2ccc(N(c3ccc(-c4ccc(-n5c6ccccc6c6c7ccccc7ccc65)cc4)cc3)c3ccc(-c4ccc5ccccc5c4)cc3)cc2)cc1 ; c1ccc(N(c2ccc(-c3ccc4ccccc4c3)cc2)c2ccc(-n3c4ccc5ccccc5c4c4c5ccccc5ccc43)cc2)cc1]

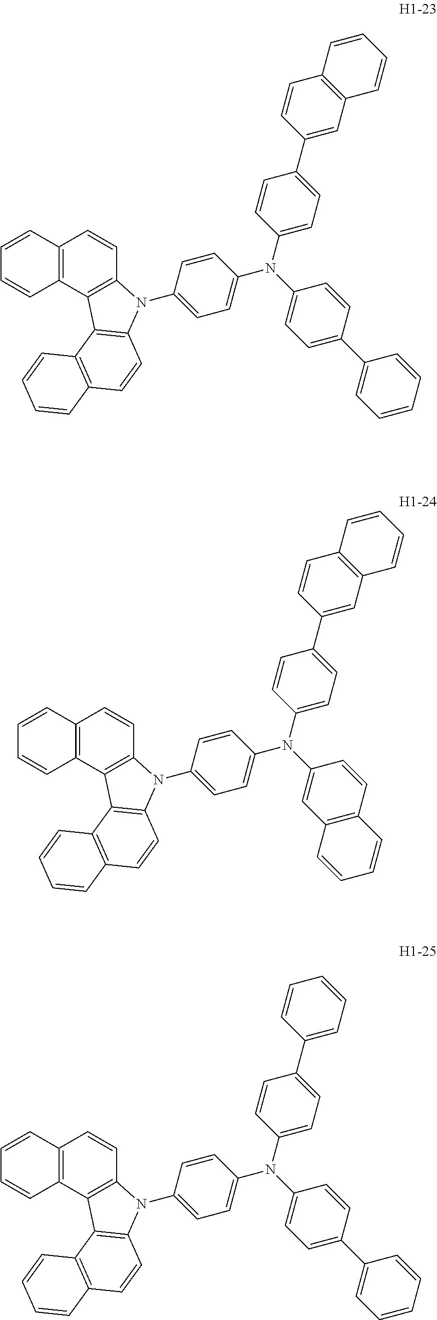 [SMILES: c1ccc(-c2ccc(N(c3ccc(-c4ccc5ccccc5c4)cc3)c3ccc(-n4c5ccc6ccccc6c5c5c6ccccc6ccc54)cc3)cc2)cc1 ; c1ccc2cc(-c3ccc(N(c4ccc(-n5c6ccc7ccccc7c6c6c7ccccc7ccc65)cc4)c4ccc5ccccc5c4)cc3)ccc2c1 ; c1ccc(-c2ccc(N(c3ccc(-c4ccccc4)cc3)c3ccc(-n4c5ccc6ccccc6c5c5c6ccccc6ccc54)cc3)cc2)cc1]

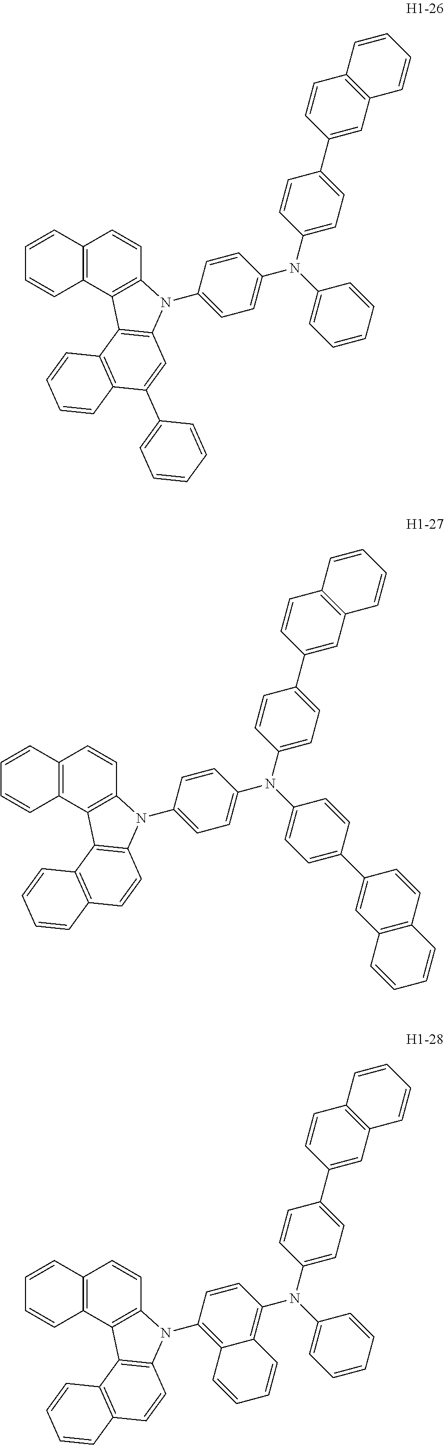 [SMILES: c1ccc(-c2cc3c(c4ccccc24)c2c4ccccc4ccc2n3-c2ccc(N(c3ccccc3)c3ccc(-c4ccc5ccccc5c4)cc3)cc2)cc1 ; c1ccc2cc(-c3ccc(N(c4ccc(-c5ccc6ccccc6c5)cc4)c4ccc(-n5c6ccc7ccccc7c6c6c7ccccc7ccc65)cc4)cc3)ccc2c1 ; c1ccc(N(c2ccc(-c3ccc4ccccc4c3)cc2)c2ccc(-n3c4ccc5ccccc5c4c4c5ccccc5ccc43)c3ccccc23)cc1]

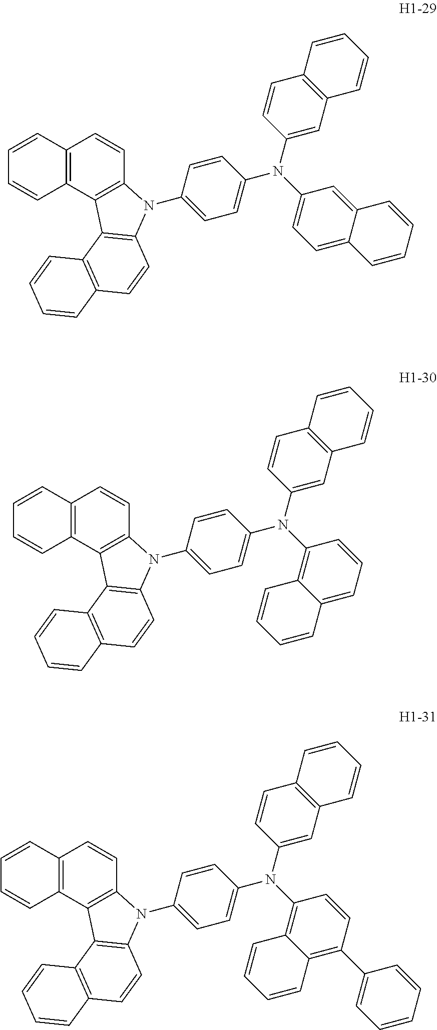 [SMILES: c1ccc2cc(N(c3ccc(-n4c5ccc6ccccc6c5c5c6ccccc6ccc54)cc3)c3ccc4ccccc4c3)ccc2c1 ; c1ccc2cc(N(c3ccc(-n4c5ccc6ccccc6c5c5c6ccccc6ccc54)cc3)c3cccc4ccccc34)ccc2c1 ; c1ccc(-c2ccc(N(c3ccc(-n4c5ccc6ccccc6c5c5c6ccccc6ccc54)cc3)c3ccc4ccccc4c3)c3ccccc23)cc1]

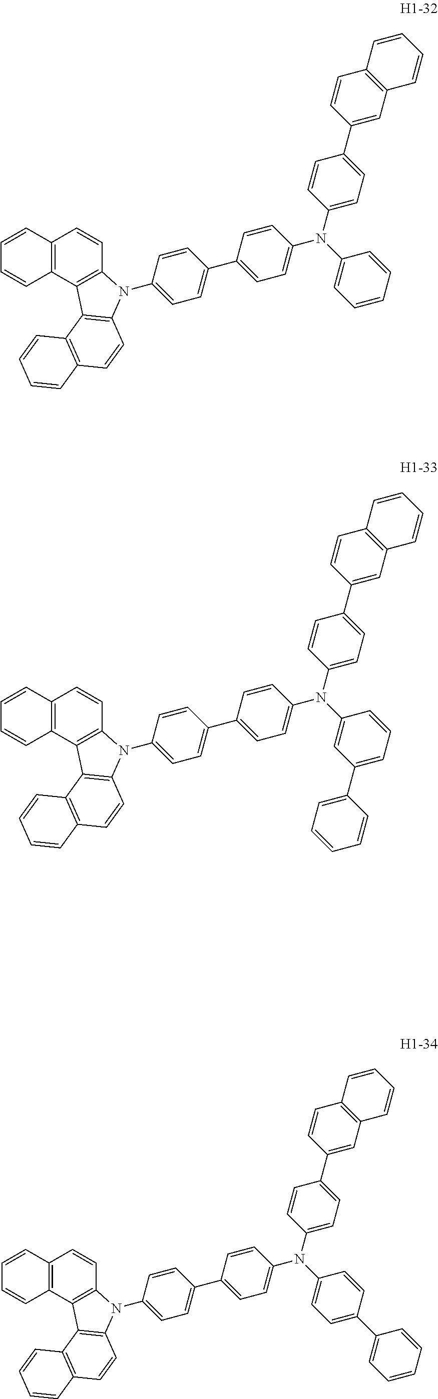 [SMILES: c1ccc(N(c2ccc(-c3ccc(-n4c5ccc6ccccc6c5c5c6ccccc6ccc54)cc3)cc2)c2ccc(-c3ccc4ccccc4c3)cc2)cc1 ; c1ccc(-c2cccc(N(c3ccc(-c4ccc(-n5c6ccc7ccccc7c6c6c7ccccc7ccc65)cc4)cc3)c3ccc(-c4ccc5ccccc5c4)cc3)c2)cc1 ; c1ccc(-c2ccc(N(c3ccc(-c4ccc(-n5c6ccc7ccccc7c6c6c7ccccc7ccc65)cc4)cc3)c3ccc(-c4ccc5ccccc5c4)cc3)cc2)cc1]

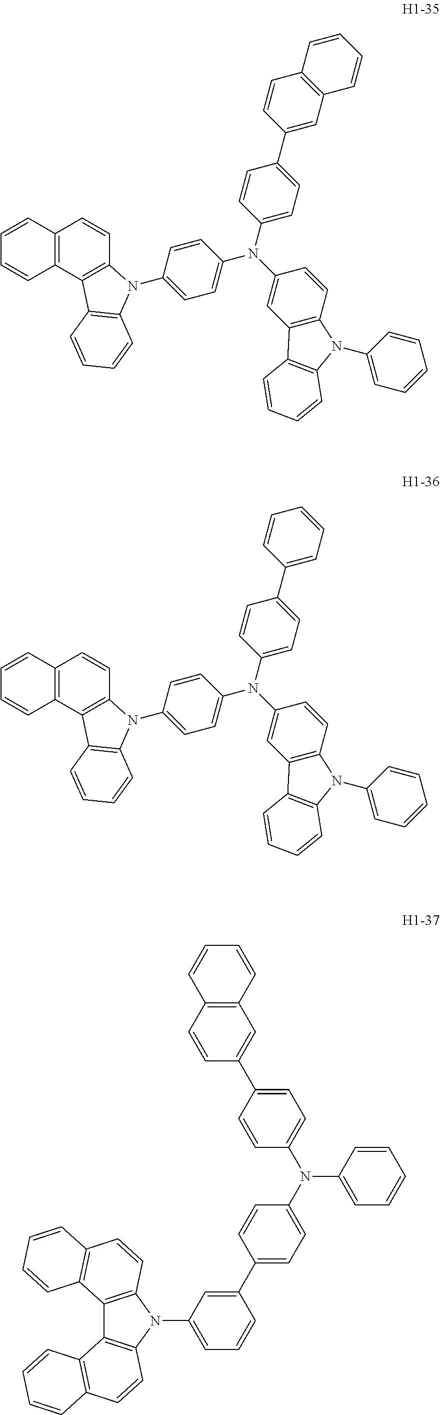 [SMILES: c1ccc(-n2c3ccccc3c3cc(N(c4ccc(-c5ccc6ccccc6c5)cc4)c4ccc(-n5c6ccccc6c6c7ccccc7ccc65)cc4)ccc32)cc1 ; c1ccc(-c2ccc(N(c3ccc(-n4c5ccccc5c5c6ccccc6ccc54)cc3)c3ccc4c(c3)c3ccccc3n4-c3ccccc3)cc2)cc1 ; c1ccc(N(c2ccc(-c3cccc(-n4c5ccc6ccccc6c5c5c6ccccc6ccc54)c3)cc2)c2ccc(-c3ccc4ccccc4c3)cc2)cc1]

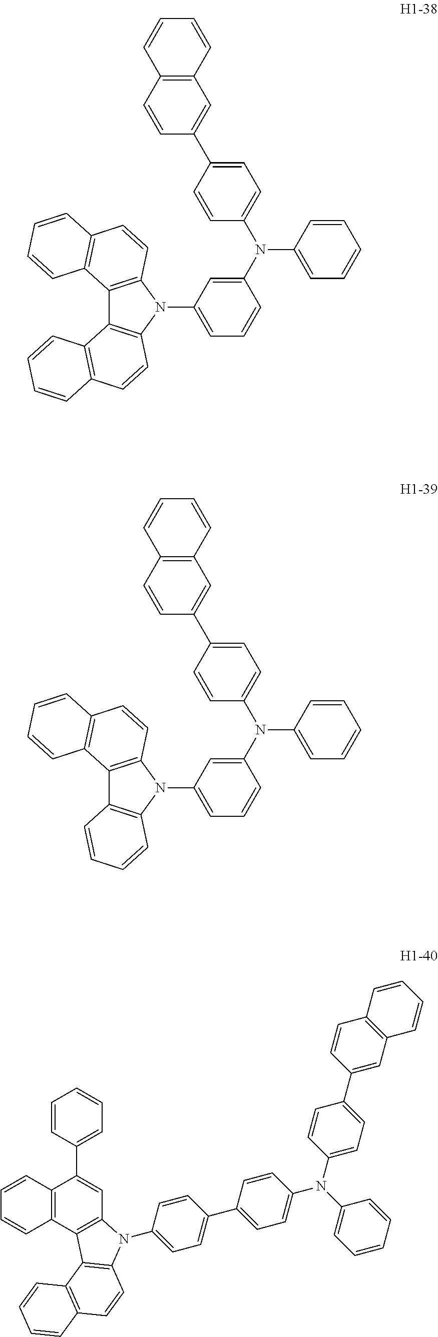 [SMILES: c1ccc(N(c2ccc(-c3ccc4ccccc4c3)cc2)c2cccc(-n3c4ccc5ccccc5c4c4c5ccccc5ccc43)c2)cc1 ; c1ccc(N(c2ccc(-c3ccc4ccccc4c3)cc2)c2cccc(-n3c4ccccc4c4c5ccccc5ccc43)c2)cc1 ; c1ccc(-c2cc3c(c4ccccc24)c2c4ccccc4ccc2n3-c2ccc(-c3ccc(N(c4ccccc4)c4ccc(-c5ccc6ccccc6c5)cc4)cc3)cc2)cc1]

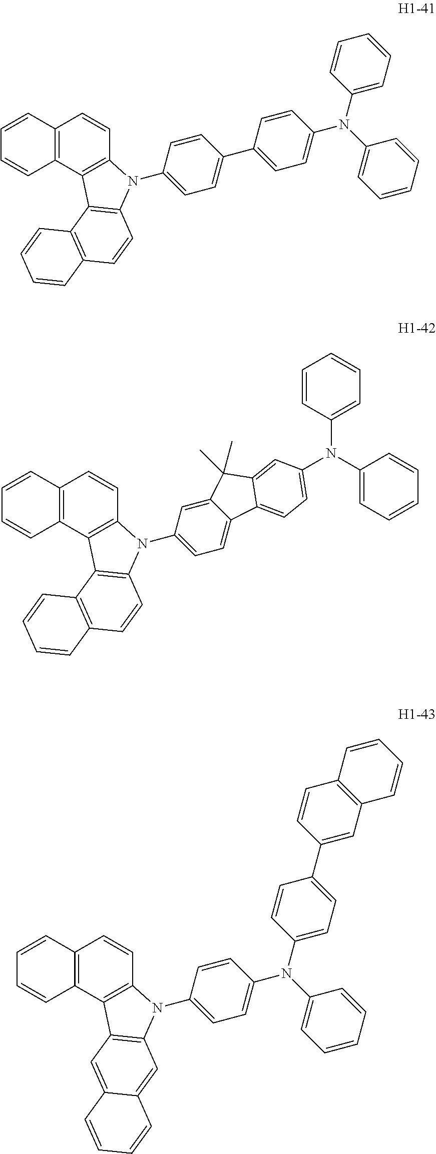 [SMILES: c1ccc(N(c2ccccc2)c2ccc(-c3ccc(-n4c5ccc6ccccc6c5c5c6ccccc6ccc54)cc3)cc2)cc1 ; CC1(C)c2cc(N(c3ccccc3)c3ccccc3)ccc2-c2ccc(-n3c4ccc5ccccc5c4c4c5ccccc5ccc43)cc21 ; c1ccc(N(c2ccc(-c3ccc4ccccc4c3)cc2)c2ccc(-n3c4cc5ccccc5cc4c4c5ccccc5ccc43)cc2)cc1]

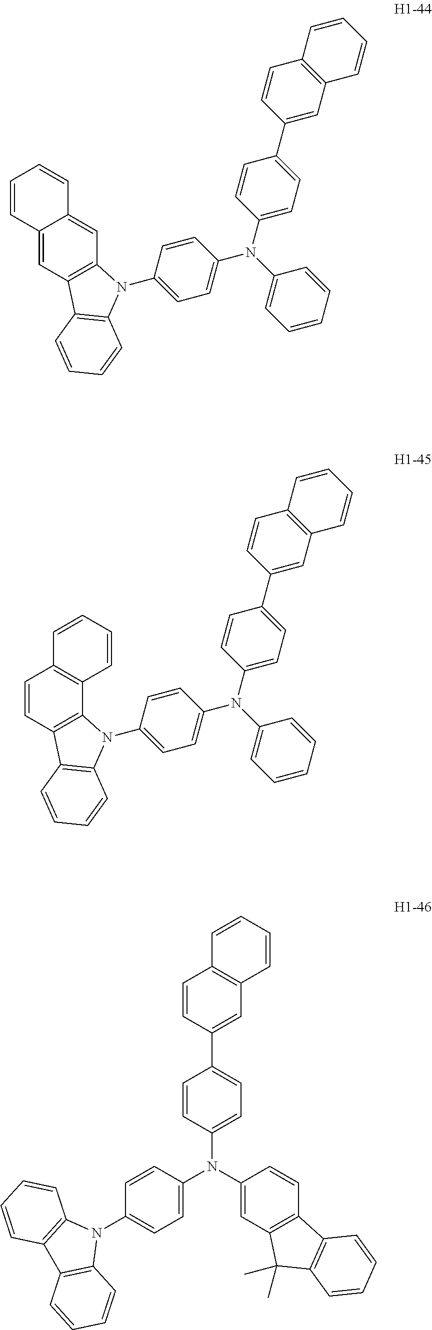 [SMILES: c1ccc(N(c2ccc(-c3ccc4ccccc4c3)cc2)c2ccc(-n3c4ccccc4c4cc5ccccc5cc43)cc2)cc1 ; c1ccc(N(c2ccc(-c3ccc4ccccc4c3)cc2)c2ccc(-n3c4ccccc4c4ccc5ccccc5c43)cc2)cc1 ; CC1(C)c2ccccc2-c2ccc(N(c3ccc(-c4ccc5ccccc5c4)cc3)c3ccc(-n4c5ccccc5c5ccccc54)cc3)cc21]

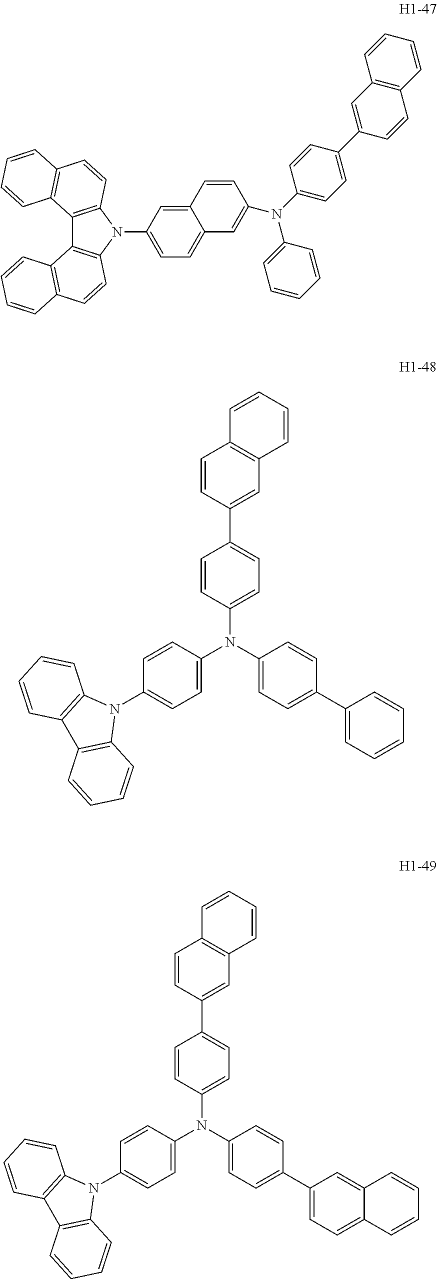 [SMILES: c1ccc(N(c2ccc(-c3ccc4ccccc4c3)cc2)c2ccc3cc(-n4c5ccc6ccccc6c5c5c6ccccc6ccc54)ccc3c2)cc1 ; c1ccc(-c2ccc(N(c3ccc(-c4ccc5ccccc5c4)cc3)c3ccc(-n4c5ccccc5c5ccccc54)cc3)cc2)cc1 ; c1ccc2cc(-c3ccc(N(c4ccc(-c5ccc6ccccc6c5)cc4)c4ccc(-n5c6ccccc6c6ccccc65)cc4)cc3)ccc2c1]

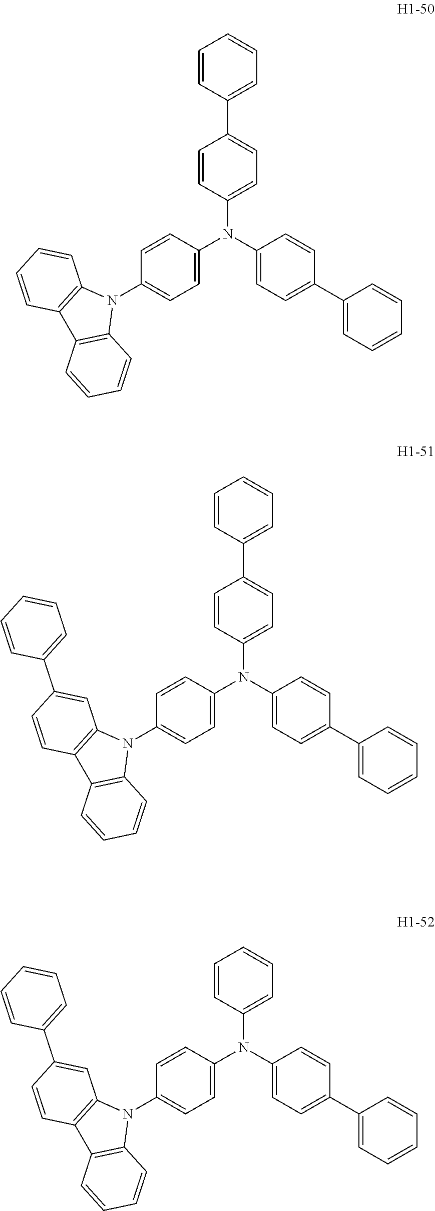 [SMILES: c1ccc(-c2ccc(N(c3ccc(-c4ccccc4)cc3)c3ccc(-n4c5ccccc5c5ccccc54)cc3)cc2)cc1 ; c1ccc(-c2ccc(N(c3ccc(-c4ccccc4)cc3)c3ccc(-n4c5ccccc5c5ccc(-c6ccccc6)cc54)cc3)cc2)cc1 ; c1ccc(-c2ccc(N(c3ccccc3)c3ccc(-n4c5ccccc5c5ccc(-c6ccccc6)cc54)cc3)cc2)cc1]

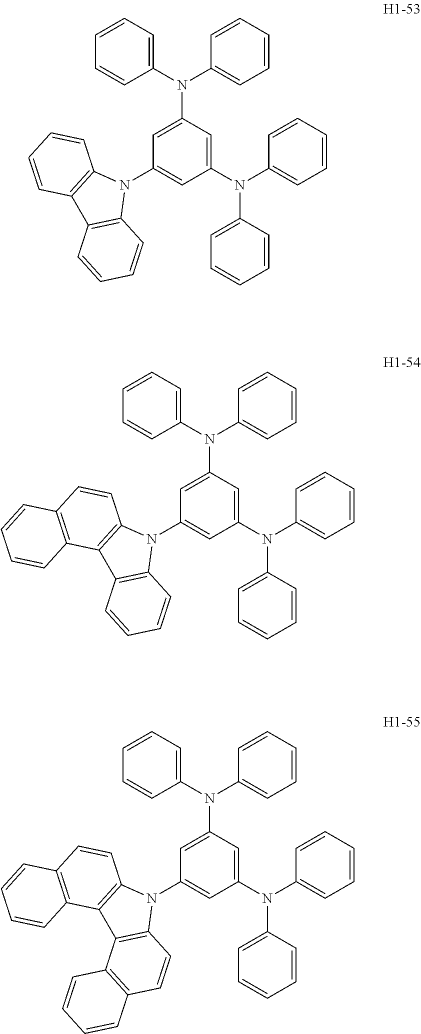 [SMILES: c1ccc(N(c2ccccc2)c2cc(N(c3ccccc3)c3ccccc3)cc(-n3c4ccccc4c4ccccc43)c2)cc1 ; c1ccc(N(c2ccccc2)c2cc(N(c3ccccc3)c3ccccc3)cc(-n3c4ccccc4c4c5ccccc5ccc43)c2)cc1 ; c1ccc(N(c2ccccc2)c2cc(N(c3ccccc3)c3ccccc3)cc(-n3c4ccc5ccccc5c4c4c5ccccc5ccc43)c2)cc1]

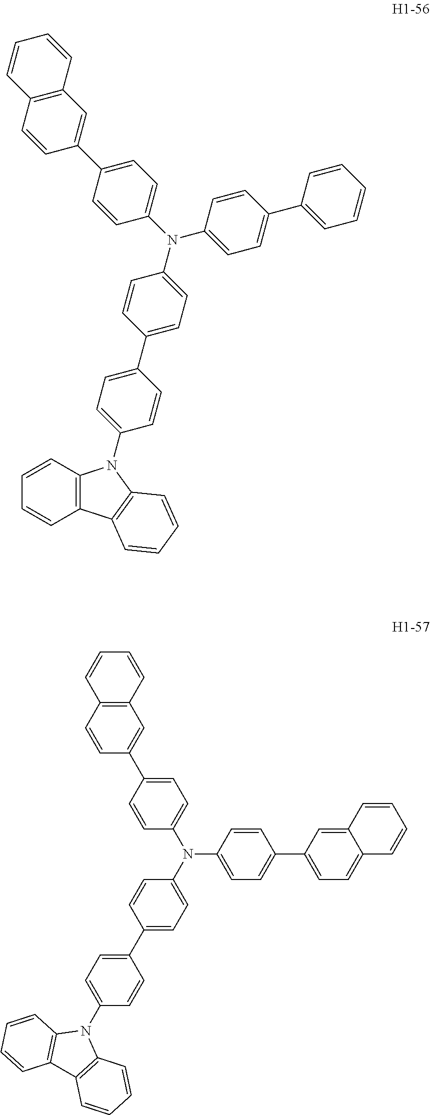 [SMILES: c1ccc(-c2ccc(N(c3ccc(-c4ccc(-n5c6ccccc6c6ccccc65)cc4)cc3)c3ccc(-c4ccc5ccccc5c4)cc3)cc2)cc1 ; c1ccc2cc(-c3ccc(N(c4ccc(-c5ccc(-n6c7ccccc7c7ccccc76)cc5)cc4)c4ccc(-c5ccc6ccccc6c5)cc4)cc3)ccc2c1]

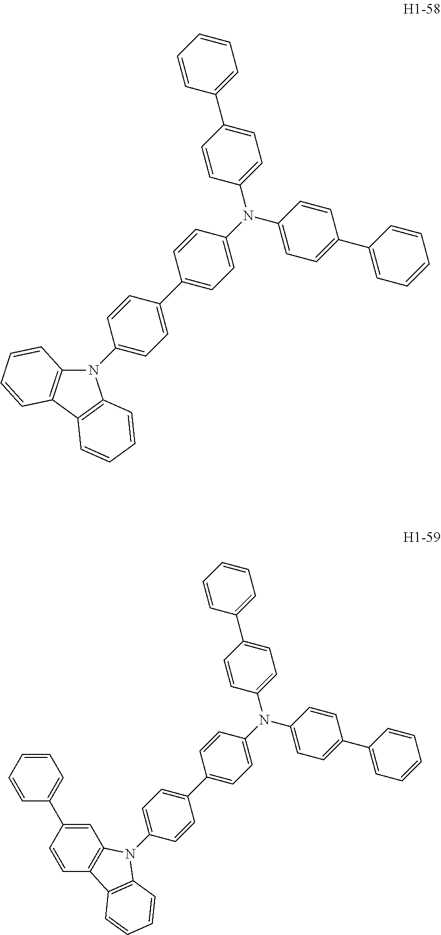 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1]

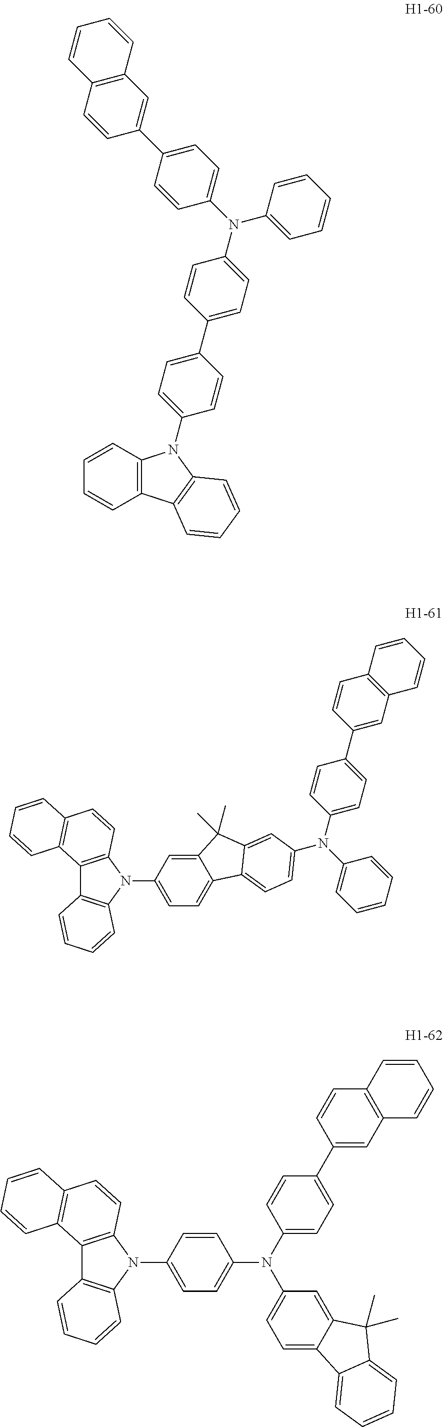 [SMILES: c1ccc(N(c2ccc(-c3ccc(-n4c5ccccc5c5ccccc54)cc3)cc2)c2ccc(-c3ccc4ccccc4c3)cc2)cc1 ; CC1(C)c2cc(N(c3ccccc3)c3ccc(-c4ccc5ccccc5c4)cc3)ccc2-c2ccc(-n3c4ccccc4c4c5ccccc5ccc43)cc21 ; CC1(C)c2ccccc2-c2ccc(N(c3ccc(-c4ccc5ccccc5c4)cc3)c3ccc(-n4c5ccccc5c5c6ccccc6ccc54)cc3)cc21]

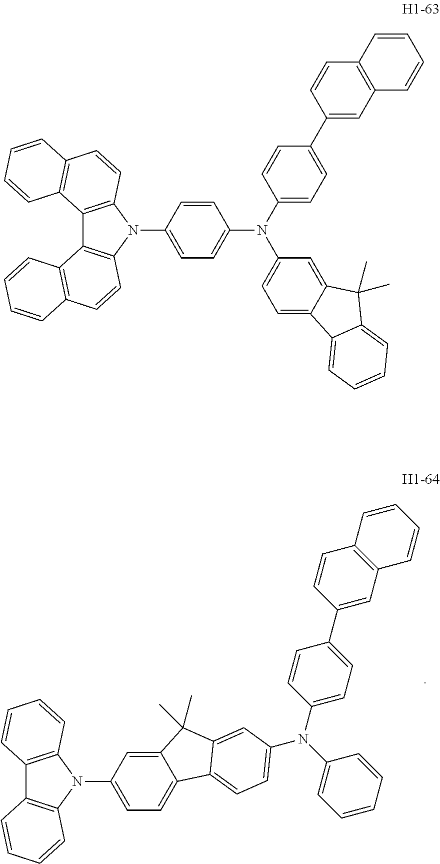 [SMILES: CC1(C)c2ccccc2-c2ccc(N(c3ccc(-c4ccc5ccccc5c4)cc3)c3ccc(-n4c5ccc6ccccc6c5c5c6ccccc6ccc54)cc3)cc21 ; CC1(C)c2cc(N(c3ccccc3)c3ccc(-c4ccc5ccccc5c4)cc3)ccc2-c2ccc(-n3c4ccccc4c4ccccc43)cc21]

The compound represented by formula 1 according to the present disclosure may be produced by a synthetic method known to a person skilled in the art, for example, referring to the methods disclosed in Korean Patent Application Laid-Open No. 2013-0084960 (Jul. 26, 2013) and Korean Patent Application Laid-Open No. 2013-0106255 (Sep. 27, 2013), but is not limited thereto.

The second host compound represented by formula 2 includes the following compounds, but is not limited thereto:

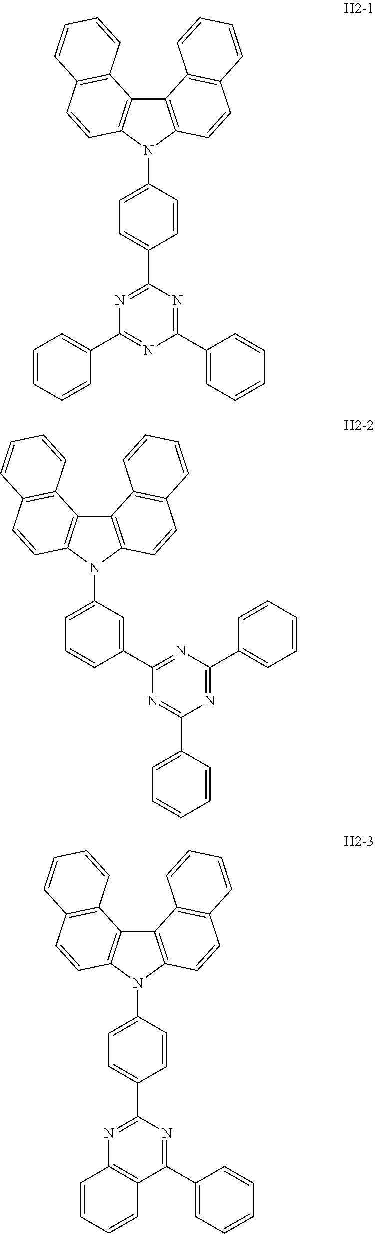

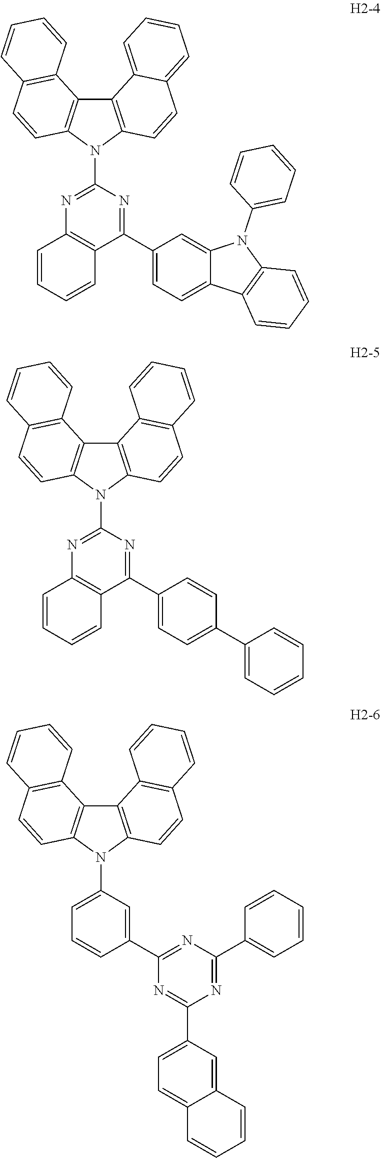 [SMILES: c1ccc(-n2c3ccccc3c3ccc(-c4nc(-n5c6ccc7ccccc7c6c6c7ccccc7ccc65)nc5ccccc45)cc32)cc1 ; c1ccc(-c2ccc(-c3nc(-n4c5ccc6ccccc6c5c5c6ccccc6ccc54)nc4ccccc34)cc2)cc1 ; c1ccc(-c2nc(-c3cccc(-n4c5ccc6ccccc6c5c5c6ccccc6ccc54)c3)nc(-c3ccc4ccccc4c3)n2)cc1]

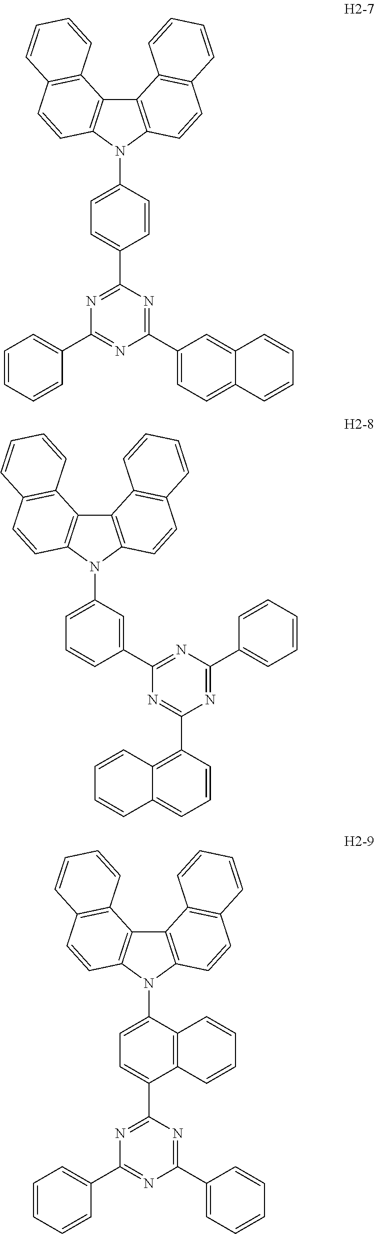

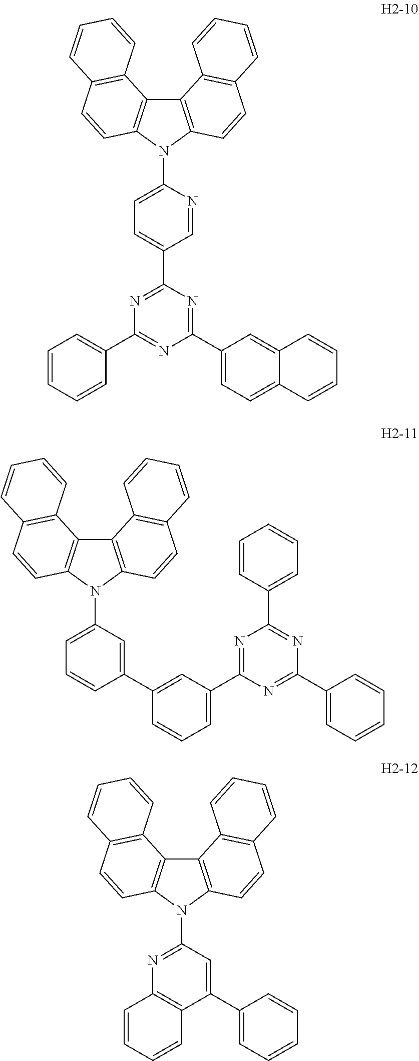 [SMILES: c1ccc(-c2nc(-c3ccc(-n4c5ccc6ccccc6c5c5c6ccccc6ccc54)nc3)nc(-c3ccc4ccccc4c3)n2)cc1 ; c1ccc(-c2nc(-c3ccccc3)nc(-c3cccc(-c4cccc(-n5c6ccc7ccccc7c6c6c7ccccc7ccc65)c4)c3)n2)cc1 ; c1ccc(-c2cc(-n3c4ccc5ccccc5c4c4c5ccccc5ccc43)nc3ccccc23)cc1]

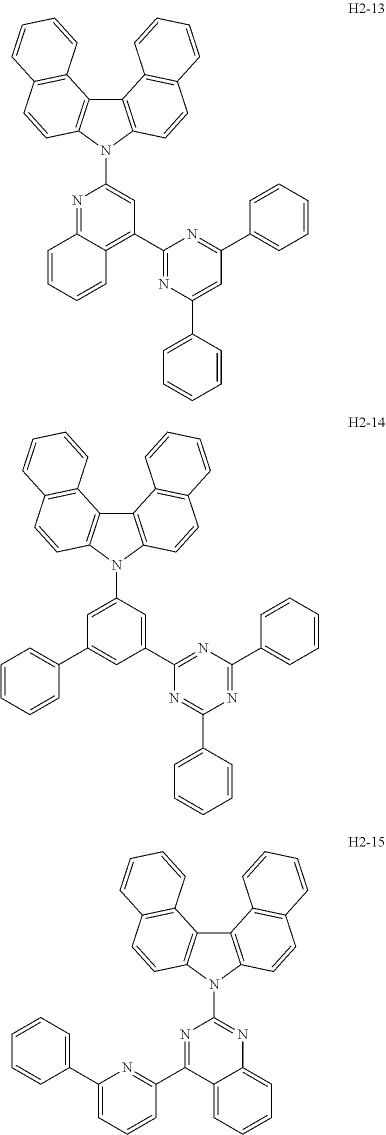

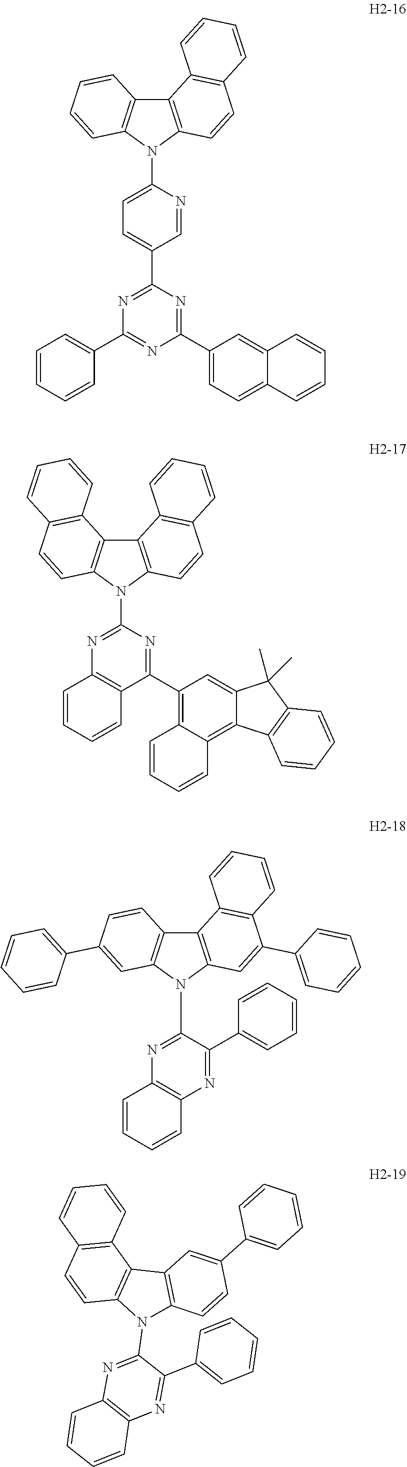 [SMILES: c1ccc(-c2nc(-c3ccc(-n4c5ccccc5c5c6ccccc6ccc54)nc3)nc(-c3ccc4ccccc4c3)n2)cc1 ; CC1(C)c2ccccc2-c2c1cc(-c1nc(-n3c4ccc5ccccc5c4c4c5ccccc5ccc43)nc3ccccc13)c1ccccc21 ; c1ccc(-c2ccc3c4c5ccccc5c(-c5ccccc5)cc4n(-c4nc5ccccc5nc4-c4ccccc4)c3c2)cc1 ; c1ccc(-c2ccc3c(c2)c2c4ccccc4ccc2n3-c2nc3ccccc3nc2-c2ccccc2)cc1]

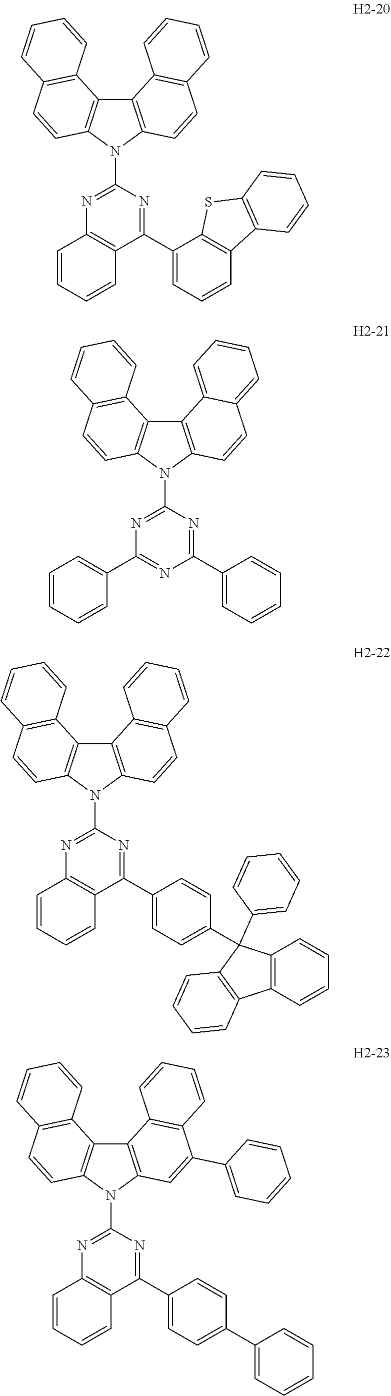

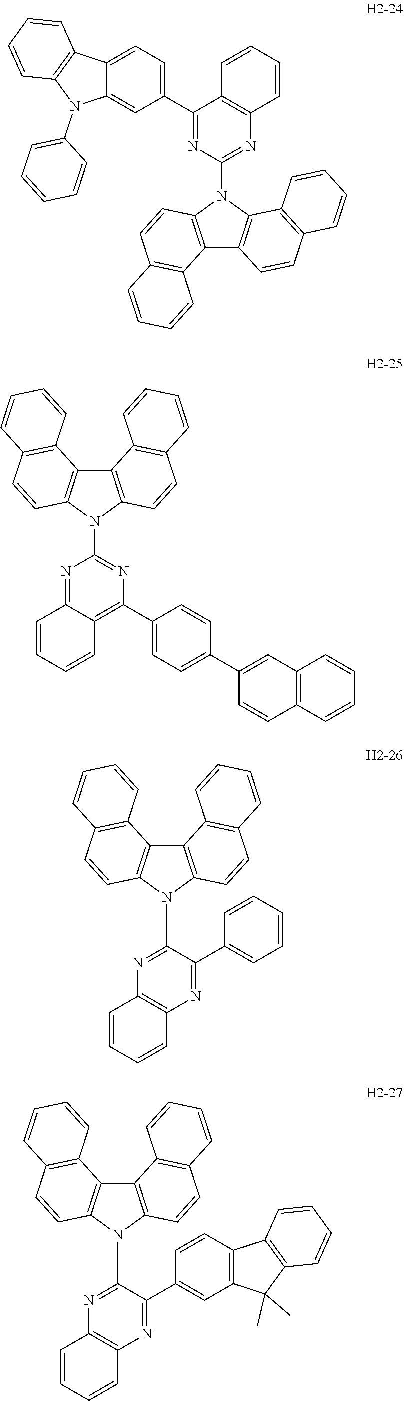 [SMILES: c1ccc(-n2c3ccccc3c3ccc(-c4nc(-n5c6ccc7ccccc7c6c6ccc7ccccc7c65)nc5ccccc45)cc32)cc1 ; c1ccc2cc(-c3ccc(-c4nc(-n5c6ccc7ccccc7c6c6c7ccccc7ccc65)nc5ccccc45)cc3)ccc2c1 ; c1ccc(-c2nc3ccccc3nc2-n2c3ccc4ccccc4c3c3c4ccccc4ccc32)cc1 ; CC1(C)c2ccccc2-c2ccc(-c3nc4ccccc4nc3-n3c4ccc5ccccc5c4c4c5ccccc5ccc43)cc21]

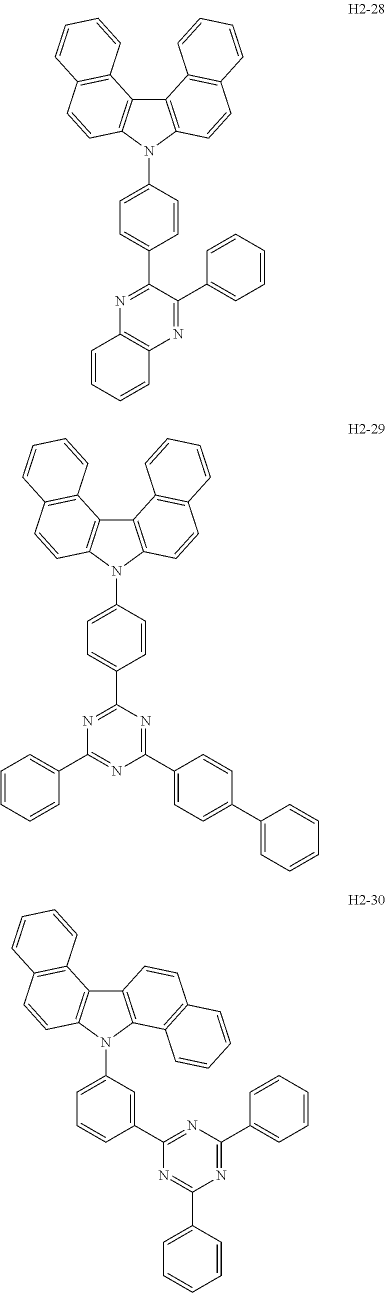 [SMILES: c1ccc(-c2nc3ccccc3nc2-c2ccc(-n3c4ccc5ccccc5c4c4c5ccccc5ccc43)cc2)cc1 ; c1ccc(-c2ccc(-c3nc(-c4ccccc4)nc(-c4ccc(-n5c6ccc7ccccc7c6c6c7ccccc7ccc65)cc4)n3)cc2)cc1 ; c1ccc(-c2nc(-c3ccccc3)nc(-c3cccc(-n4c5ccc6ccccc6c5c5ccc6ccccc6c54)c3)n2)cc1]

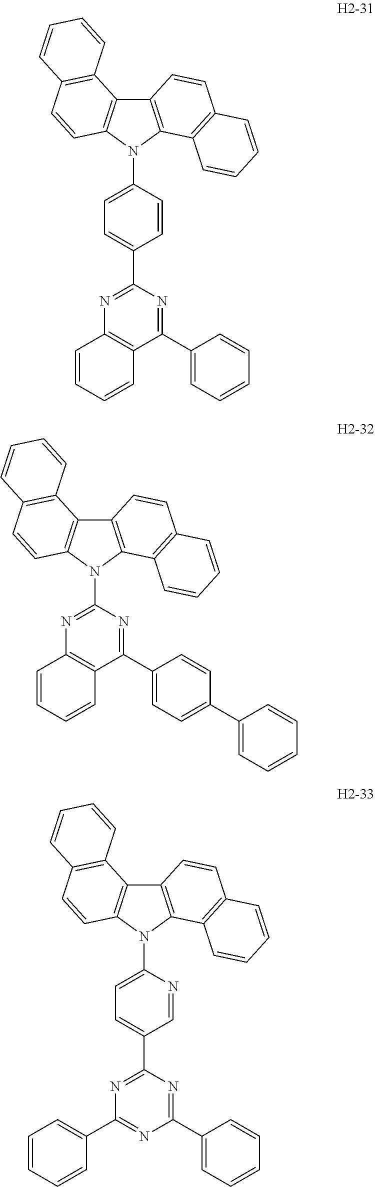 [SMILES: c1ccc(-c2nc(-c3ccc(-n4c5ccc6ccccc6c5c5ccc6ccccc6c54)cc3)nc3ccccc23)cc1 ; c1ccc(-c2ccc(-c3nc(-n4c5ccc6ccccc6c5c5ccc6ccccc6c54)nc4ccccc34)cc2)cc1 ; c1ccc(-c2nc(-c3ccccc3)nc(-c3ccc(-n4c5ccc6ccccc6c5c5ccc6ccccc6c54)nc3)n2)cc1]

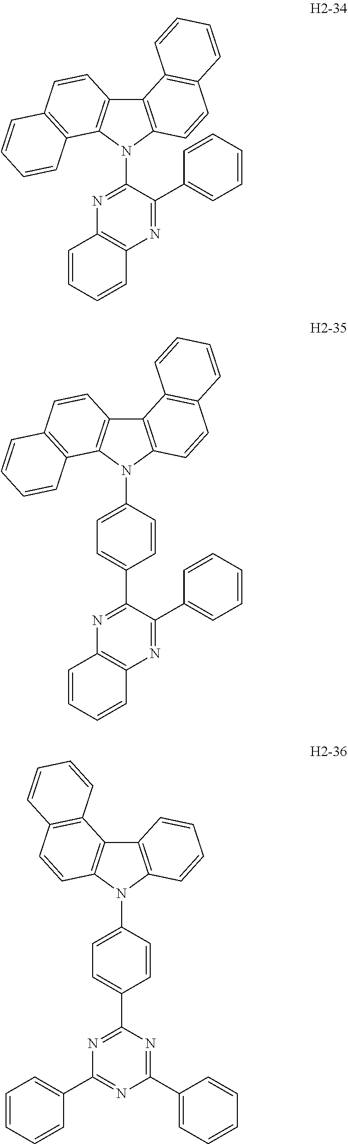 [SMILES: c1ccc(-c2nc3ccccc3nc2-n2c3ccc4ccccc4c3c3ccc4ccccc4c32)cc1 ; c1ccc(-c2nc3ccccc3nc2-c2ccc(-n3c4ccc5ccccc5c4c4ccc5ccccc5c43)cc2)cc1 ; c1ccc(-c2nc(-c3ccccc3)nc(-c3ccc(-n4c5ccccc5c5c6ccccc6ccc54)cc3)n2)cc1]

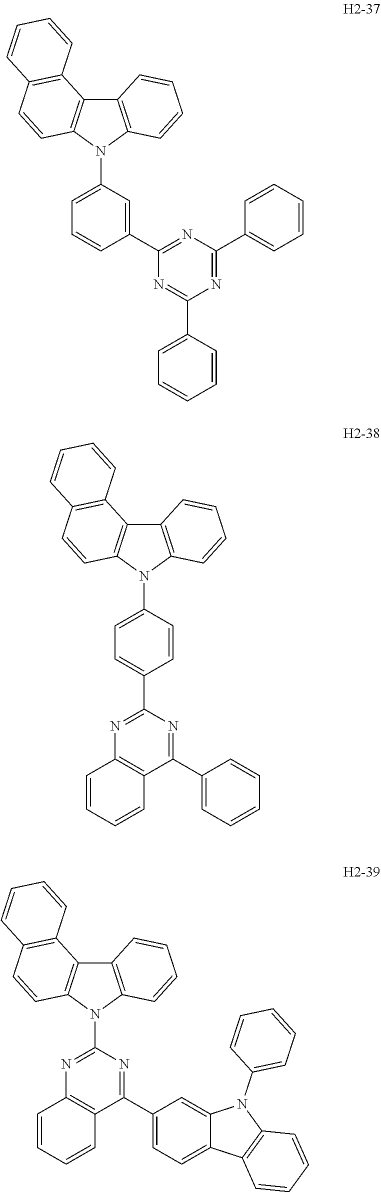 [SMILES: c1ccc(-c2nc(-c3ccccc3)nc(-c3cccc(-n4c5ccccc5c5c6ccccc6ccc54)c3)n2)cc1 ; c1ccc(-c2nc(-c3ccc(-n4c5ccccc5c5c6ccccc6ccc54)cc3)nc3ccccc23)cc1 ; c1ccc(-n2c3ccccc3c3ccc(-c4nc(-n5c6ccccc6c6c7ccccc7ccc65)nc5ccccc45)cc32)cc1]

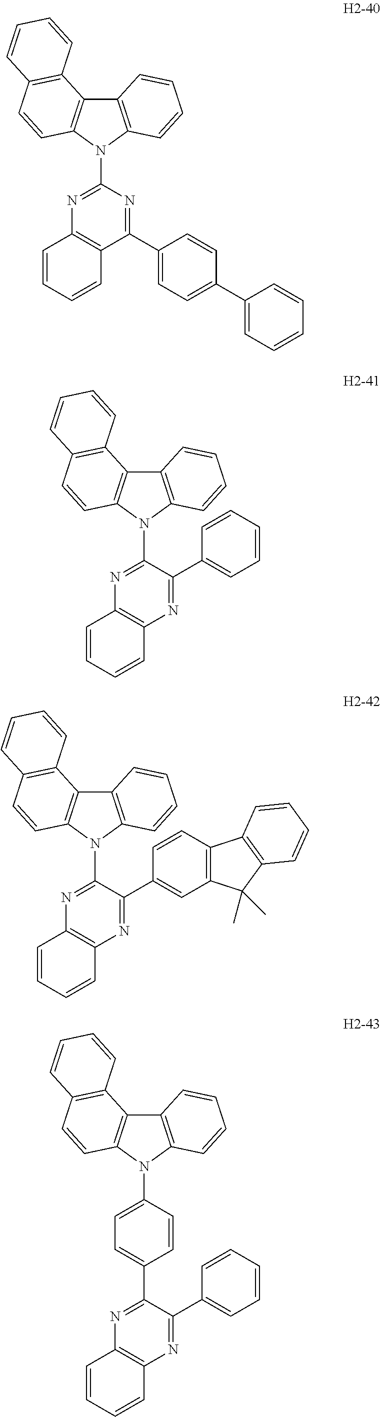

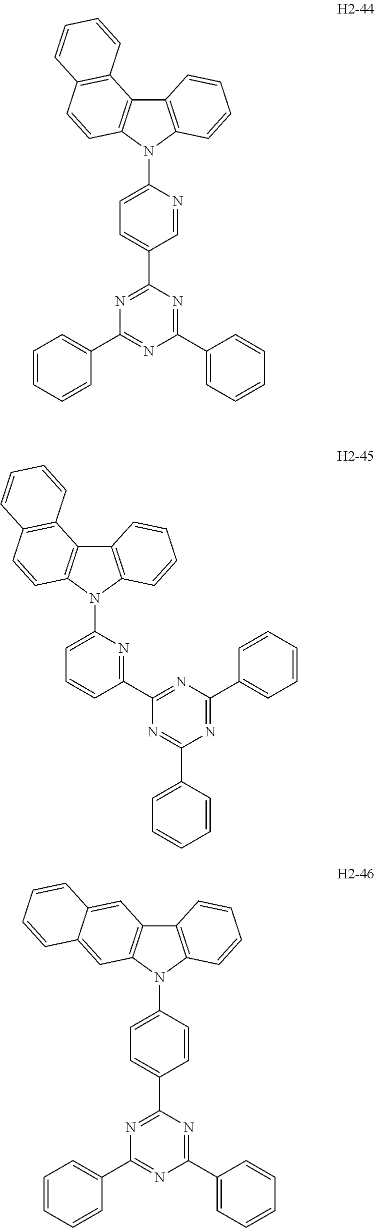 [SMILES: c1ccc(-c2nc(-c3ccccc3)nc(-c3ccc(-n4c5ccccc5c5c6ccccc6ccc54)nc3)n2)cc1 ; c1ccc(-c2nc(-c3ccccc3)nc(-c3cccc(-n4c5ccccc5c5c6ccccc6ccc54)n3)n2)cc1 ; c1ccc(-c2nc(-c3ccccc3)nc(-c3ccc(-n4c5ccccc5c5cc6ccccc6cc54)cc3)n2)cc1]

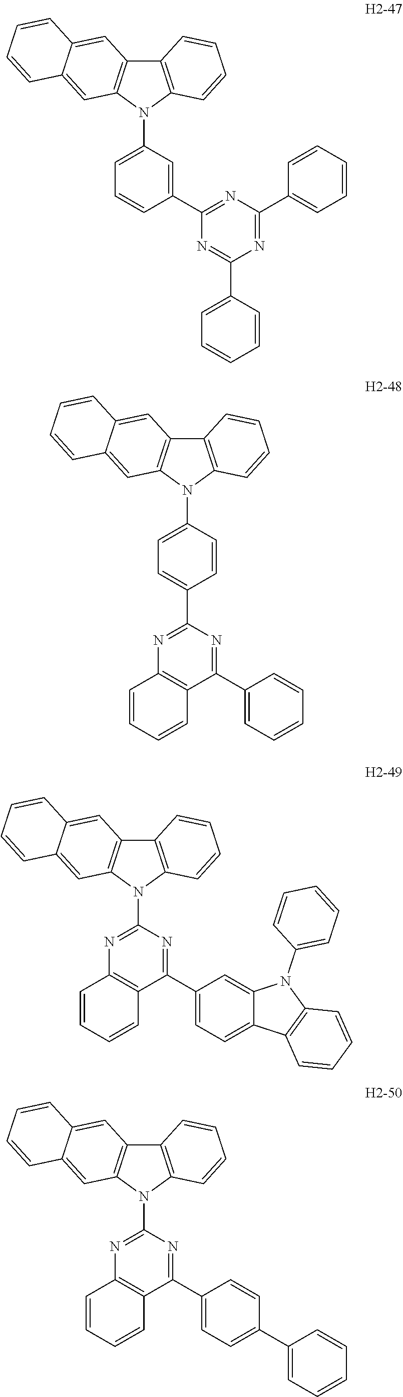 [SMILES: c1ccc(-c2nc(-c3ccccc3)nc(-c3cccc(-n4c5ccccc5c5cc6ccccc6cc54)c3)n2)cc1 ; c1ccc(-c2nc(-c3ccc(-n4c5ccccc5c5cc6ccccc6cc54)cc3)nc3ccccc23)cc1 ; c1ccc(-n2c3ccccc3c3ccc(-c4nc(-n5c6ccccc6c6cc7ccccc7cc65)nc5ccccc45)cc32)cc1 ; c1ccc(-c2ccc(-c3nc(-n4c5ccccc5c5cc6ccccc6cc54)nc4ccccc34)cc2)cc1]

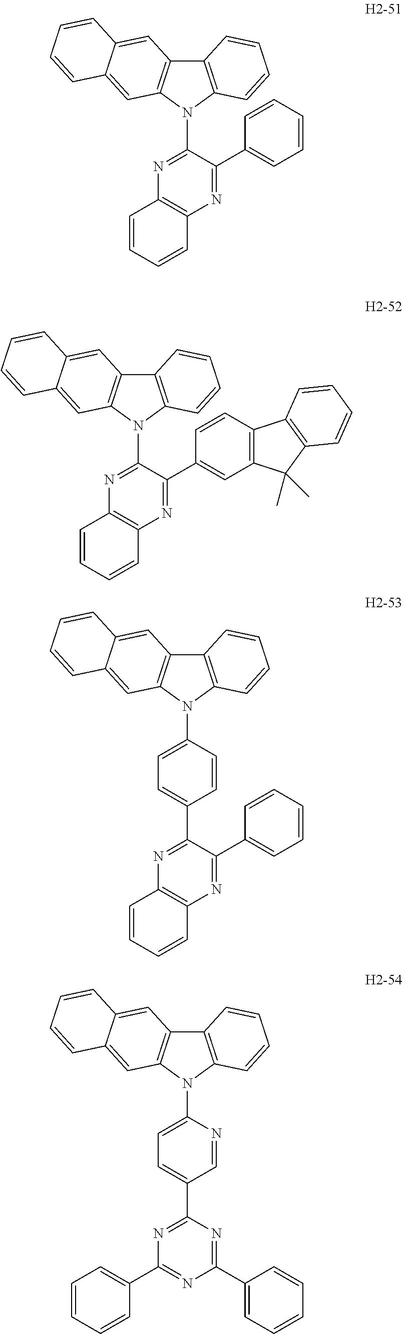

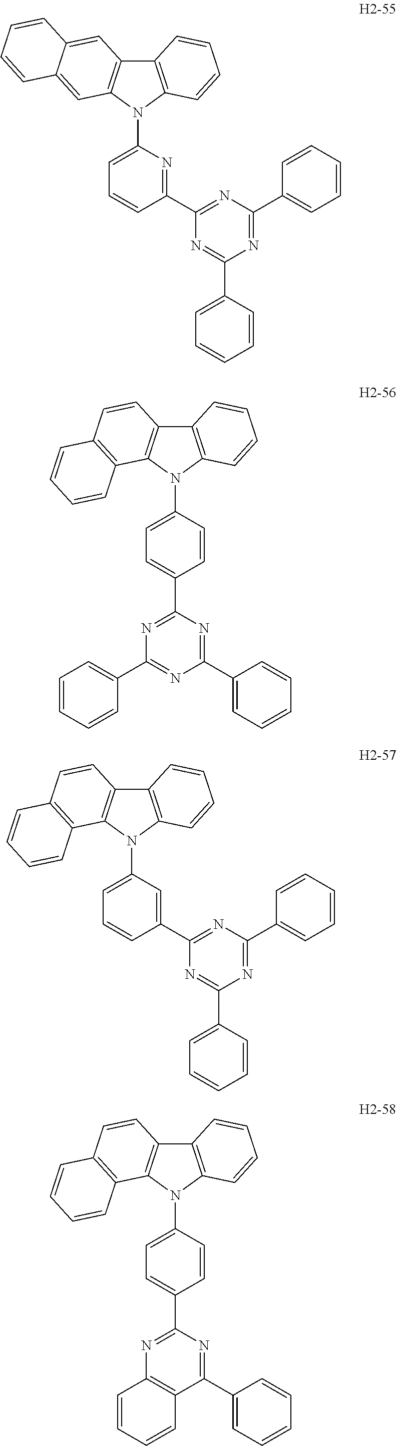

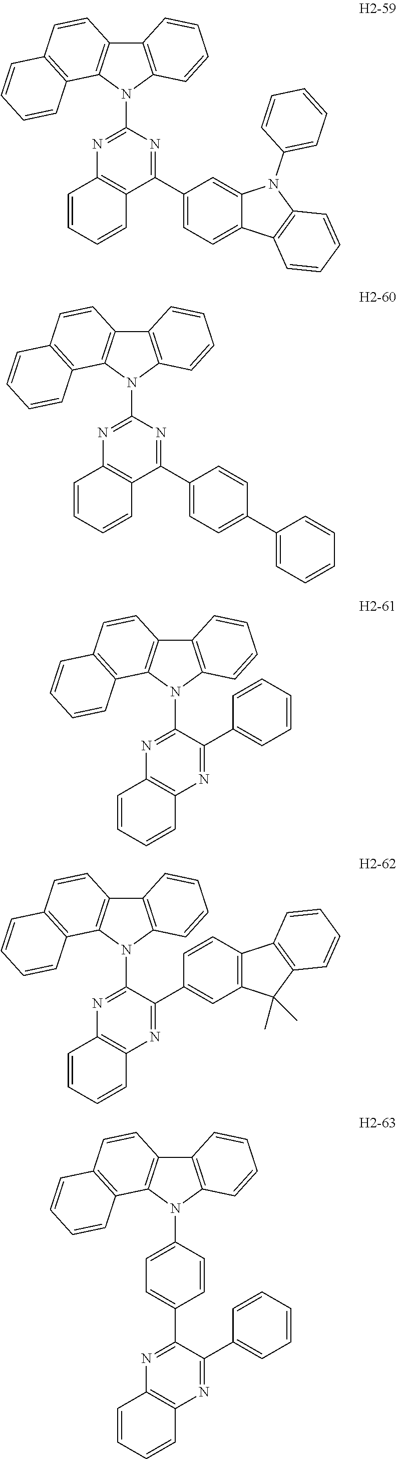

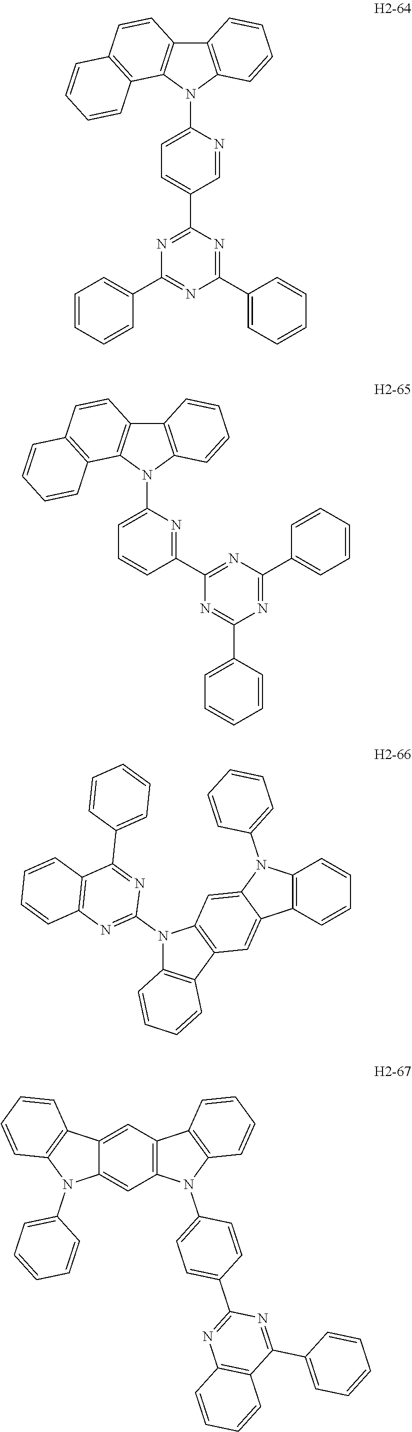

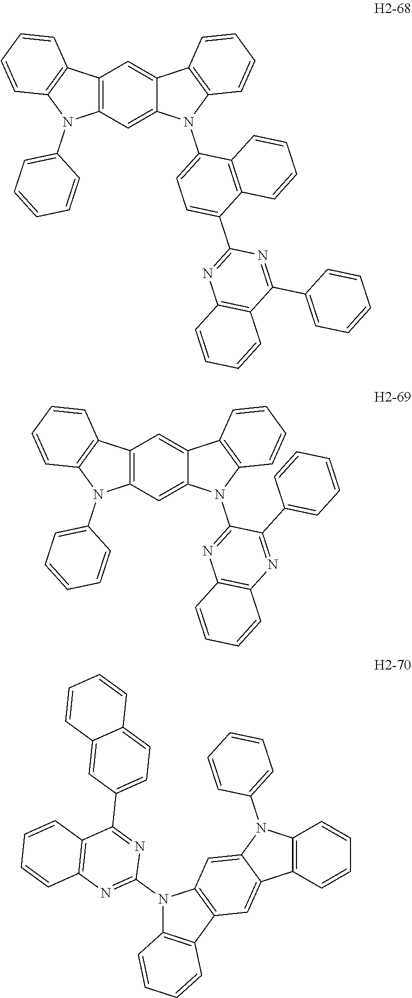

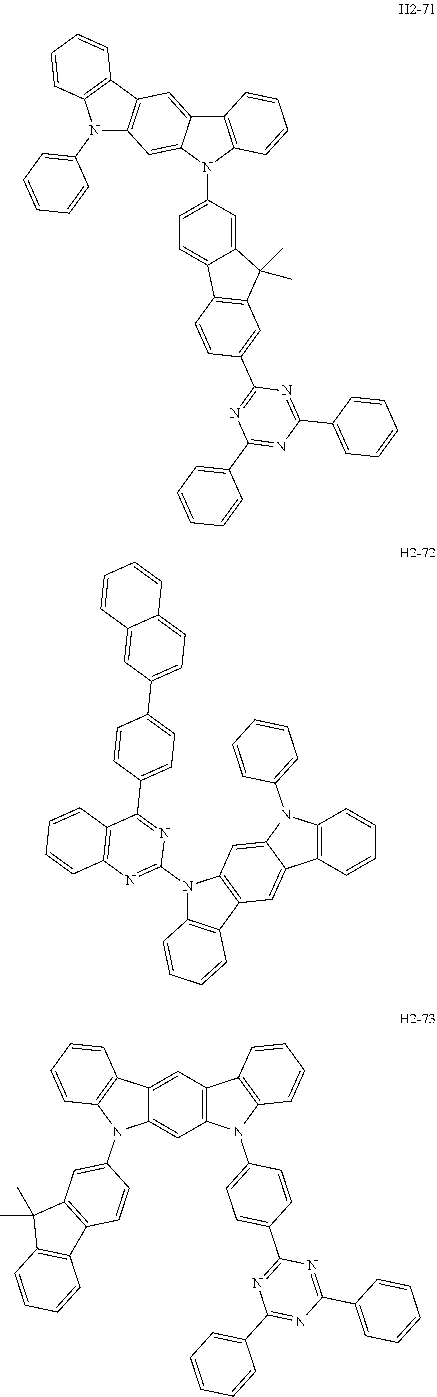 [SMILES: CC1(C)c2cc(-c3nc(-c4ccccc4)nc(-c4ccccc4)n3)ccc2-c2ccc(-n3c4ccccc4c4cc5c6ccccc6n(-c6ccccc6)c5cc43)cc21 ; c1ccc(-n2c3ccccc3c3cc4c5ccccc5n(-c5nc(-c6ccc(-c7ccc8ccccc8c7)cc6)c6ccccc6n5)c4cc32)cc1 ; CC1(C)c2ccccc2-c2ccc(-n3c4ccccc4c4cc5c6ccccc6n(-c6ccc(-c7nc(-c8ccccc8)nc(-c8ccccc8)n7)cc6)c5cc43)cc21]

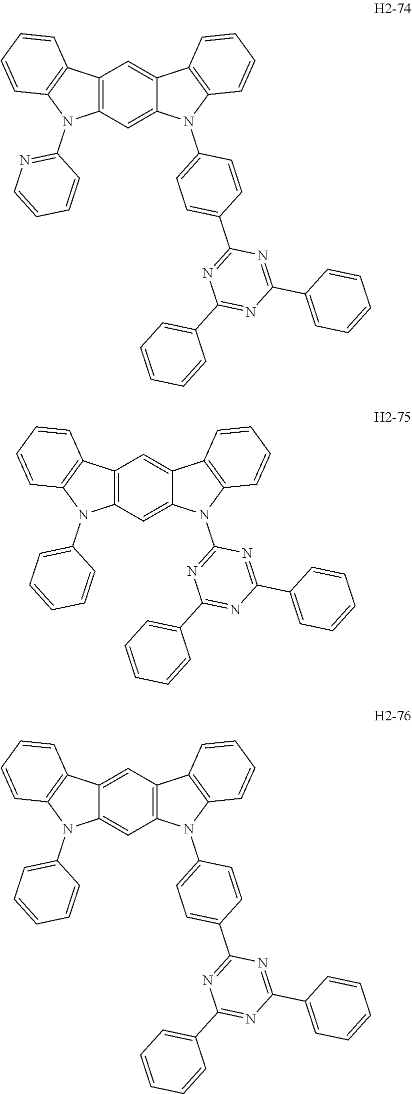 [SMILES: c1ccc(-c2nc(-c3ccccc3)nc(-c3ccc(-n4c5ccccc5c5cc6c7ccccc7n(-c7ccccn7)c6cc54)cc3)n2)cc1 ; c1ccc(-c2nc(-c3ccccc3)nc(-n3c4ccccc4c4cc5c6ccccc6n(-c6ccccc6)c5cc43)n2)cc1 ; c1ccc(-c2nc(-c3ccccc3)nc(-c3ccc(-n4c5ccccc5c5cc6c7ccccc7n(-c7ccccc7)c6cc54)cc3)n2)cc1]

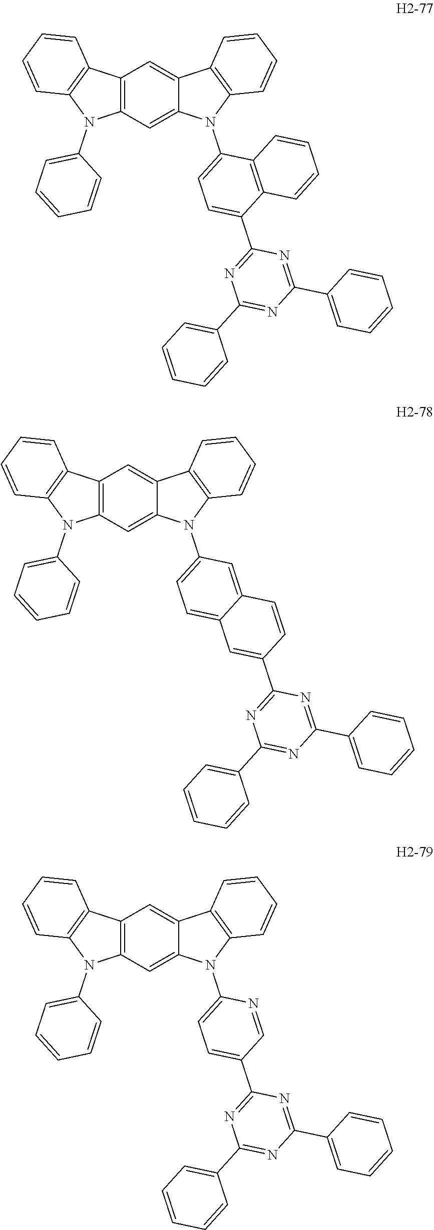 [SMILES: c1ccc(-c2nc(-c3ccccc3)nc(-c3ccc(-n4c5ccccc5c5cc6c7ccccc7n(-c7ccccc7)c6cc54)c4ccccc34)n2)cc1 ; c1ccc(-c2nc(-c3ccccc3)nc(-c3ccc4cc(-n5c6ccccc6c6cc7c8ccccc8n(-c8ccccc8)c7cc65)ccc4c3)n2)cc1 ; c1ccc(-c2nc(-c3ccccc3)nc(-c3ccc(-n4c5ccccc5c5cc6c7ccccc7n(-c7ccccc7)c6cc54)nc3)n2)cc1]

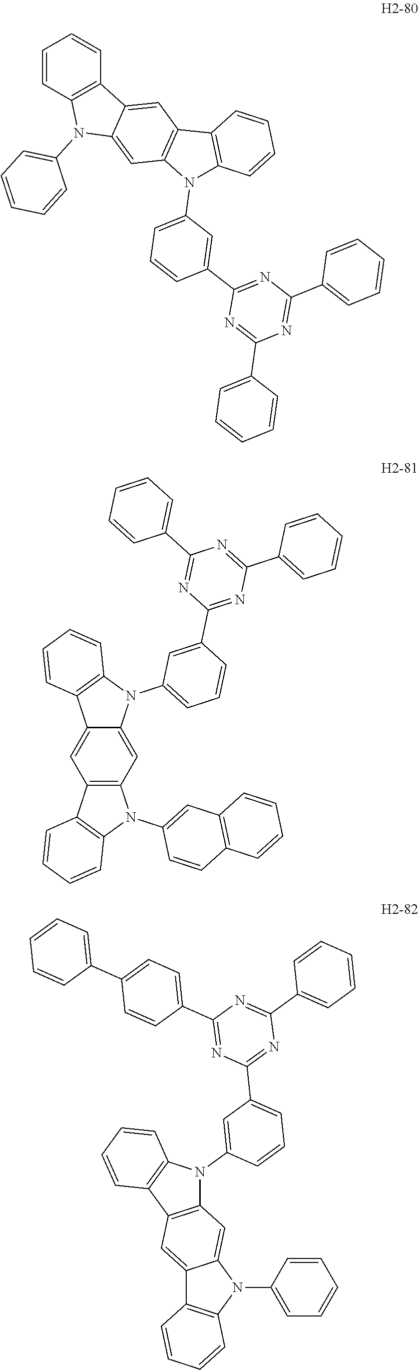

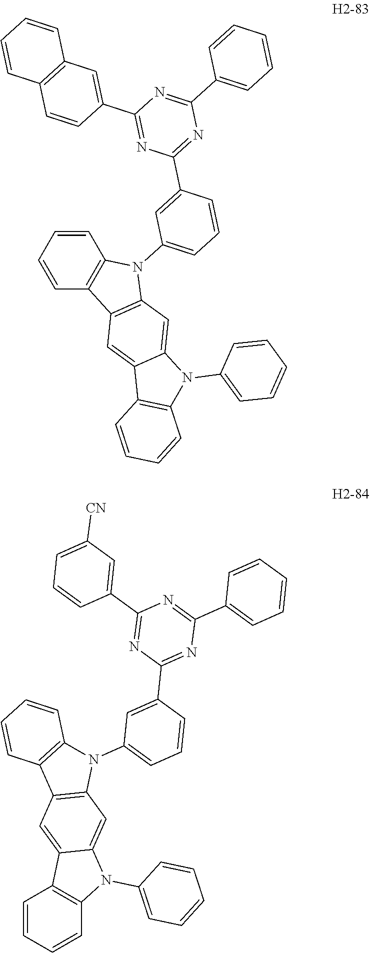 [SMILES: c1ccc(-c2nc(-c3cccc(-n4c5ccccc5c5cc6c7ccccc7n(-c7ccccc7)c6cc54)c3)nc(-c3ccc4ccccc4c3)n2)cc1 ; N#Cc1cccc(-c2nc(-c3ccccc3)nc(-c3cccc(-n4c5ccccc5c5cc6c7ccccc7n(-c7ccccc7)c6cc54)c3)n2)c1]

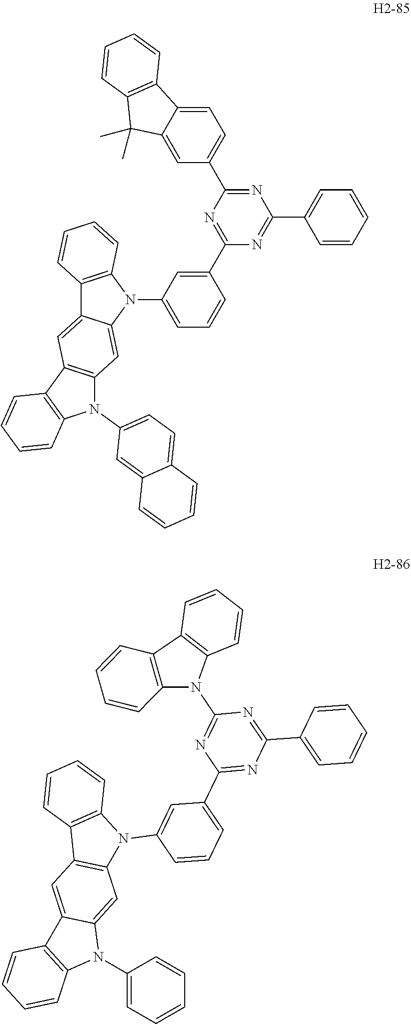 [SMILES: CC1(C)c2ccccc2-c2ccc(-c3nc(-c4ccccc4)nc(-c4cccc(-n5c6ccccc6c6cc7c8ccccc8n(-c8ccc9ccccc9c8)c7cc65)c4)n3)cc21 ; c1ccc(-c2nc(-c3cccc(-n4c5ccccc5c5cc6c7ccccc7n(-c7ccccc7)c6cc54)c3)nc(-n3c4ccccc4c4ccccc43)n2)cc1]

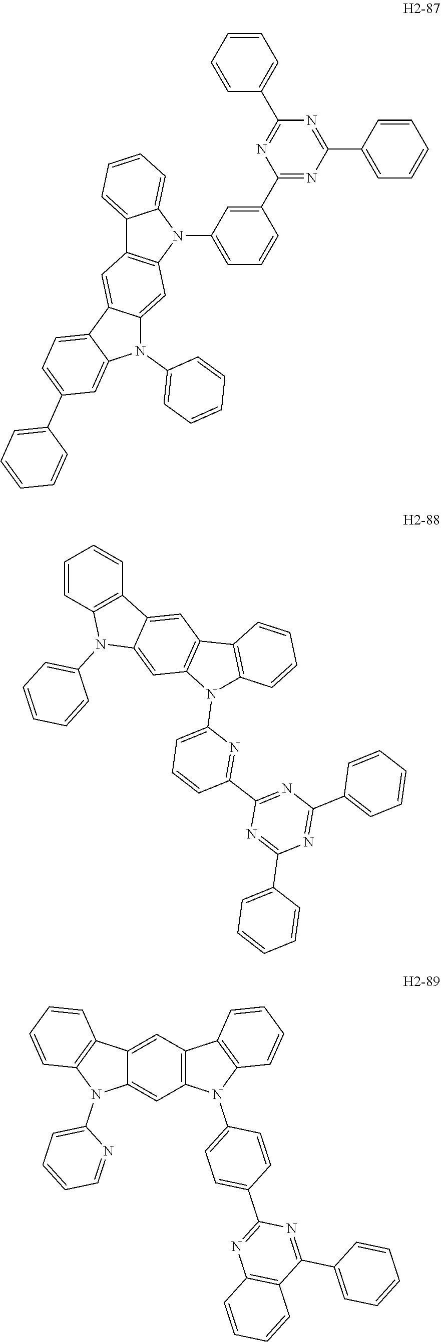 [SMILES: c1ccc(-c2ccc3c4cc5c6ccccc6n(-c6cccc(-c7nc(-c8ccccc8)nc(-c8ccccc8)n7)c6)c5cc4n(-c4ccccc4)c3c2)cc1 ; c1ccc(-c2nc(-c3ccccc3)nc(-c3cccc(-n4c5ccccc5c5cc6c7ccccc7n(-c7ccccc7)c6cc54)n3)n2)cc1 ; c1ccc(-c2nc(-c3ccc(-n4c5ccccc5c5cc6c7ccccc7n(-c7ccccn7)c6cc54)cc3)nc3ccccc23)cc1]

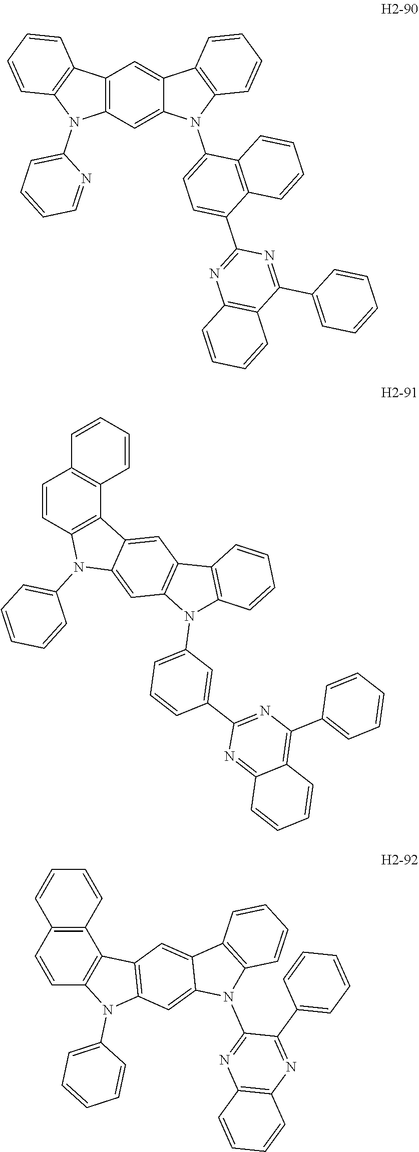 [SMILES: c1ccc(-c2nc(-c3ccc(-n4c5ccccc5c5cc6c7ccccc7n(-c7ccccn7)c6cc54)c4ccccc34)nc3ccccc23)cc1 ; c1ccc(-c2nc(-c3cccc(-n4c5ccccc5c5cc6c7c8ccccc8ccc7n(-c7ccccc7)c6cc54)c3)nc3ccccc23)cc1 ; c1ccc(-c2nc3ccccc3nc2-n2c3ccccc3c3cc4c5c6ccccc6ccc5n(-c5ccccc5)c4cc32)cc1]

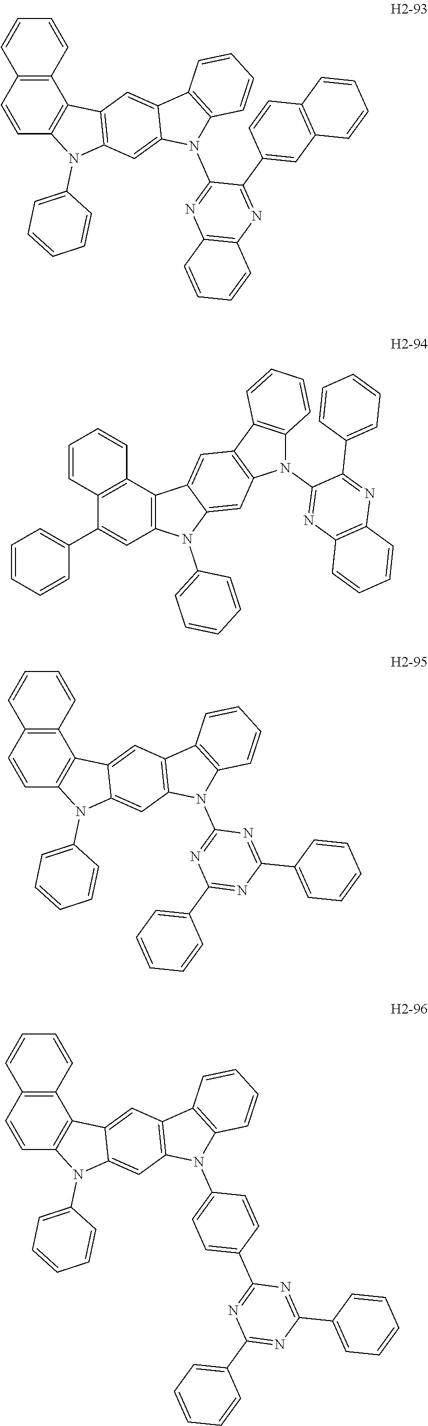 [SMILES: c1ccc(-n2c3cc4c(cc3c3c5ccccc5ccc32)c2ccccc2n4-c2nc3ccccc3nc2-c2ccc3ccccc3c2)cc1 ; c1ccc(-c2nc3ccccc3nc2-n2c3ccccc3c3cc4c5c6ccccc6c(-c6ccccc6)cc5n(-c5ccccc5)c4cc32)cc1 ; c1ccc(-c2nc(-c3ccccc3)nc(-n3c4ccccc4c4cc5c6c7ccccc7ccc6n(-c6ccccc6)c5cc43)n2)cc1 ; c1ccc(-c2nc(-c3ccccc3)nc(-c3ccc(-n4c5ccccc5c5cc6c7c8ccccc8ccc7n(-c7ccccc7)c6cc54)cc3)n2)cc1]

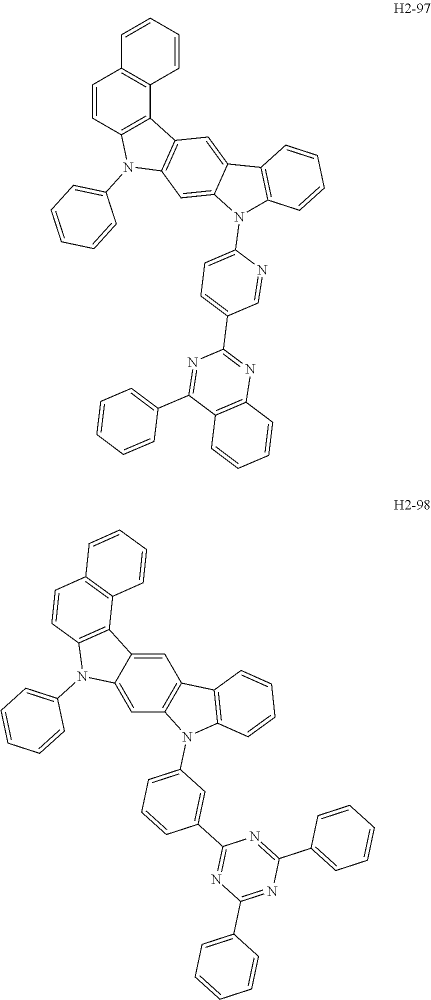 [SMILES: c1ccc(-c2nc(-c3ccc(-n4c5ccccc5c5cc6c7c8ccccc8ccc7n(-c7ccccc7)c6cc54)nc3)nc3ccccc23)cc1 ; c1ccc(-c2nc(-c3ccccc3)nc(-c3cccc(-n4c5ccccc5c5cc6c7c8ccccc8ccc7n(-c7ccccc7)c6cc54)c3)n2)cc1]

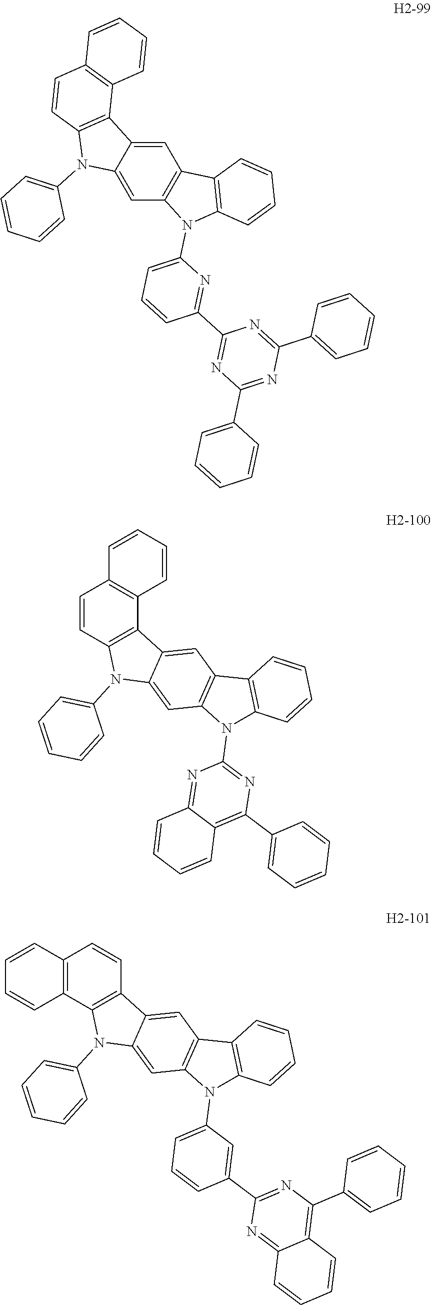 [SMILES: c1ccc(-c2nc(-c3ccccc3)nc(-c3cccc(-n4c5ccccc5c5cc6c7c8ccccc8ccc7n(-c7ccccc7)c6cc54)n3)n2)cc1 ; c1ccc(-c2nc(-n3c4ccccc4c4cc5c6c7ccccc7ccc6n(-c6ccccc6)c5cc43)nc3ccccc23)cc1 ; c1ccc(-c2nc(-c3cccc(-n4c5ccccc5c5cc6c7ccc8ccccc8c7n(-c7ccccc7)c6cc54)c3)nc3ccccc23)cc1]

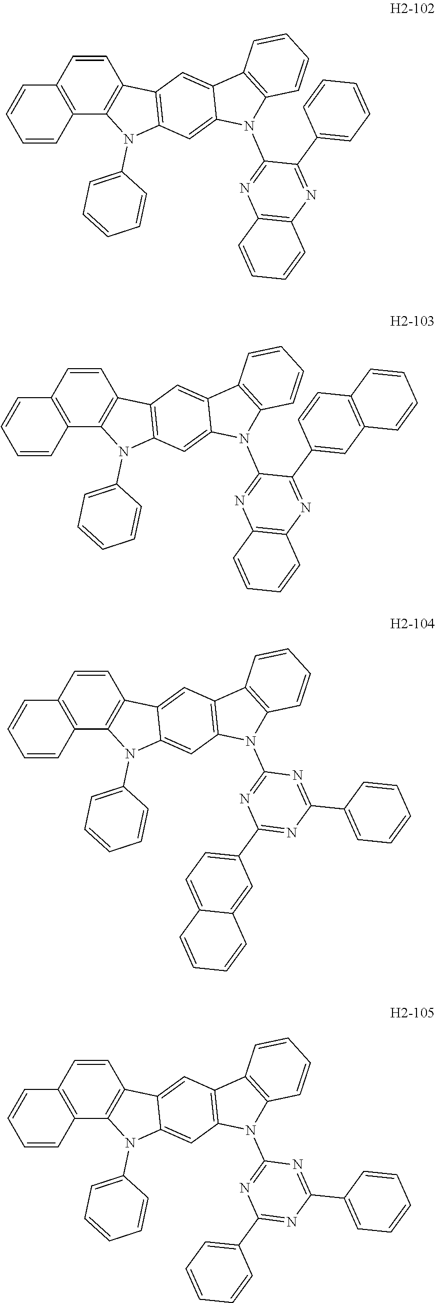 [SMILES: c1ccc(-c2nc3ccccc3nc2-n2c3ccccc3c3cc4c5ccc6ccccc6c5n(-c5ccccc5)c4cc32)cc1 ; c1ccc(-n2c3cc4c(cc3c3ccc5ccccc5c32)c2ccccc2n4-c2nc3ccccc3nc2-c2ccc3ccccc3c2)cc1 ; c1ccc(-c2nc(-c3ccc4ccccc4c3)nc(-n3c4ccccc4c4cc5c6ccc7ccccc7c6n(-c6ccccc6)c5cc43)n2)cc1 ; c1ccc(-c2nc(-c3ccccc3)nc(-n3c4ccccc4c4cc5c6ccc7ccccc7c6n(-c6ccccc6)c5cc43)n2)cc1]

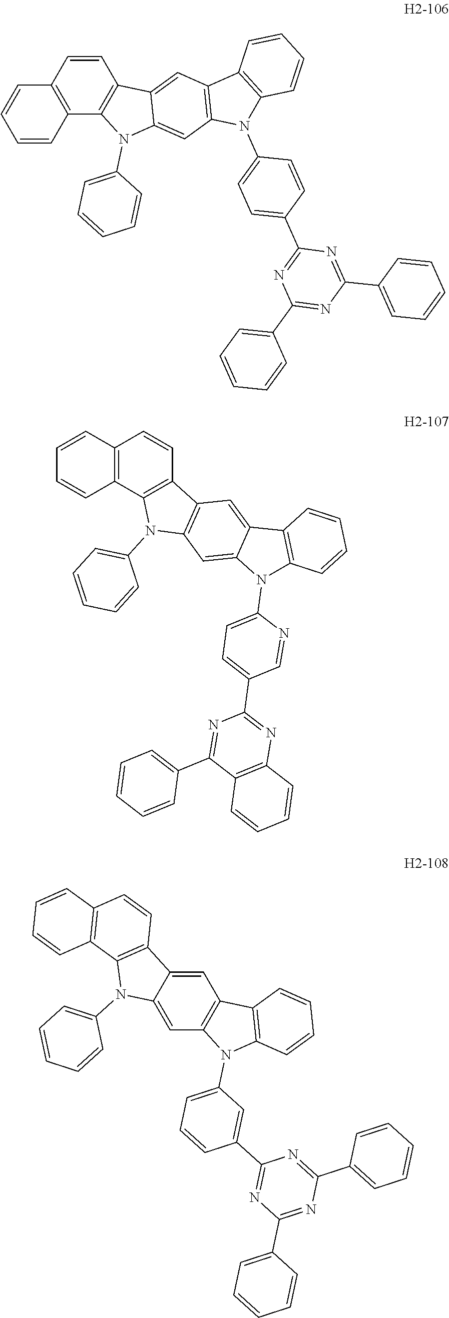 [SMILES: c1ccc(-c2nc(-c3ccccc3)nc(-c3ccc(-n4c5ccccc5c5cc6c7ccc8ccccc8c7n(-c7ccccc7)c6cc54)cc3)n2)cc1 ; c1ccc(-c2nc(-c3ccc(-n4c5ccccc5c5cc6c7ccc8ccccc8c7n(-c7ccccc7)c6cc54)nc3)nc3ccccc23)cc1 ; c1ccc(-c2nc(-c3ccccc3)nc(-c3cccc(-n4c5ccccc5c5cc6c7ccc8ccccc8c7n(-c7ccccc7)c6cc54)c3)n2)cc1]

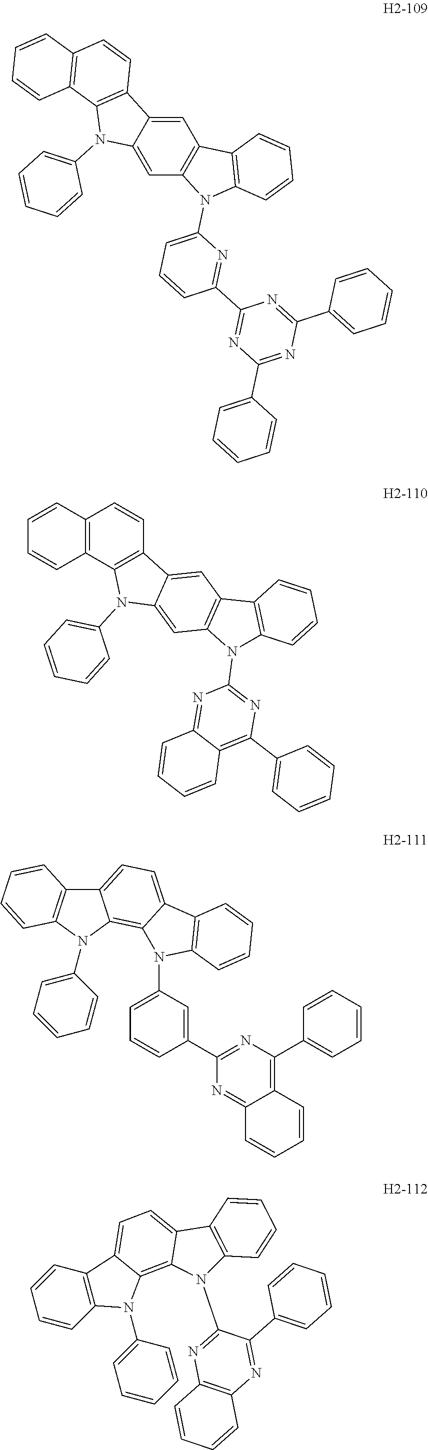

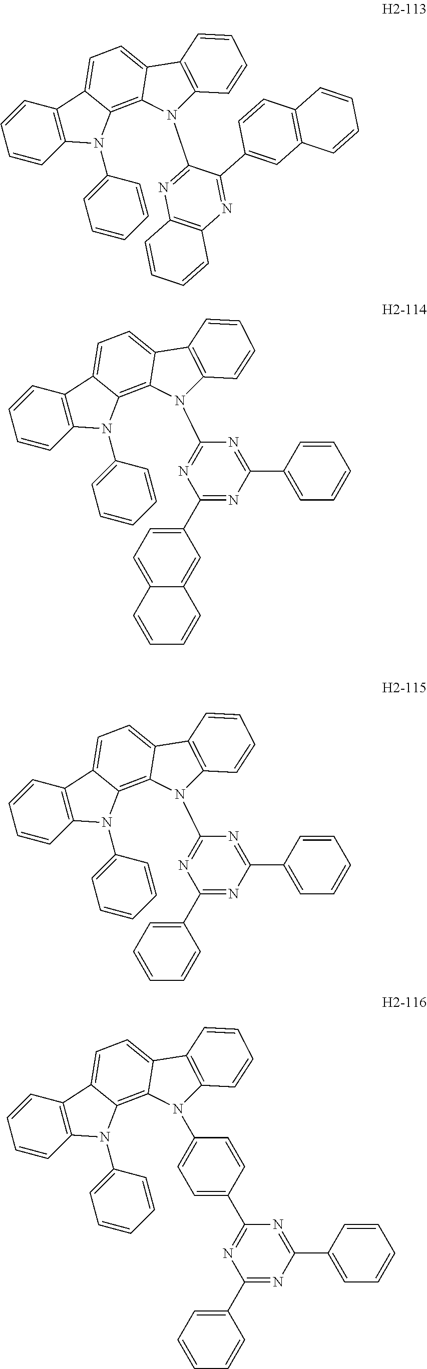

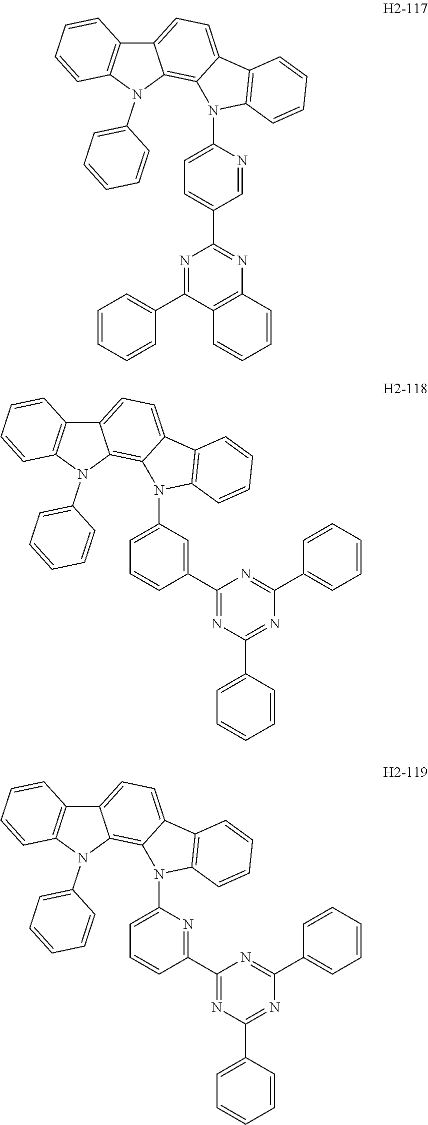

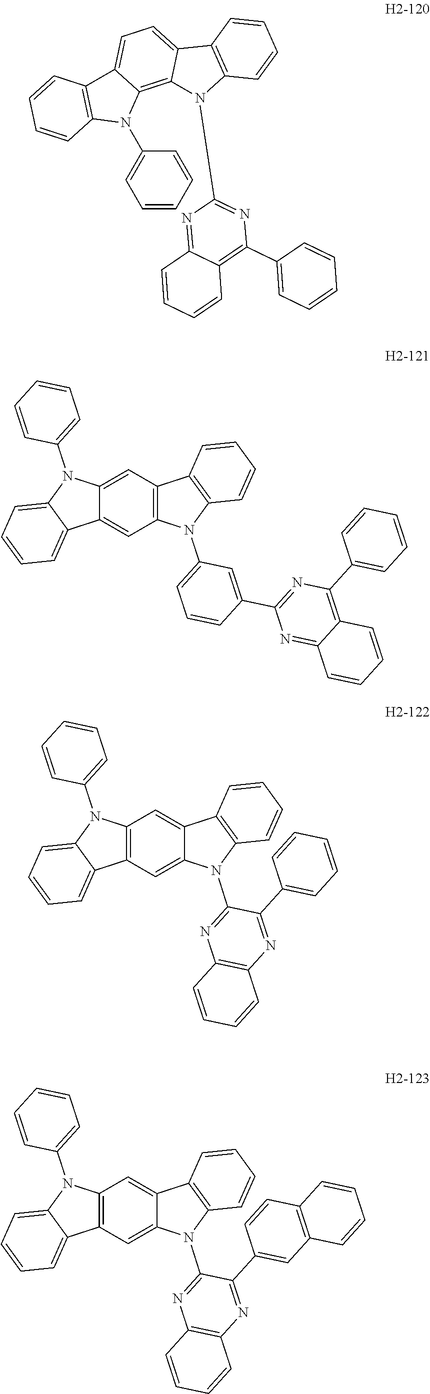 [SMILES: c1ccc(-c2nc(-n3c4ccccc4c4ccc5c6ccccc6n(-c6ccccc6)c5c43)nc3ccccc23)cc1 ; c1ccc(-c2nc(-c3cccc(-n4c5ccccc5c5cc6c(cc54)c4ccccc4n6-c4ccccc4)c3)nc3ccccc23)cc1 ; c1ccc(-c2nc3ccccc3nc2-n2c3ccccc3c3cc4c(cc32)c2ccccc2n4-c2ccccc2)cc1 ; c1ccc(-n2c3ccccc3c3cc4c(cc32)c2ccccc2n4-c2nc3ccccc3nc2-c2ccc3ccccc3c2)cc1]

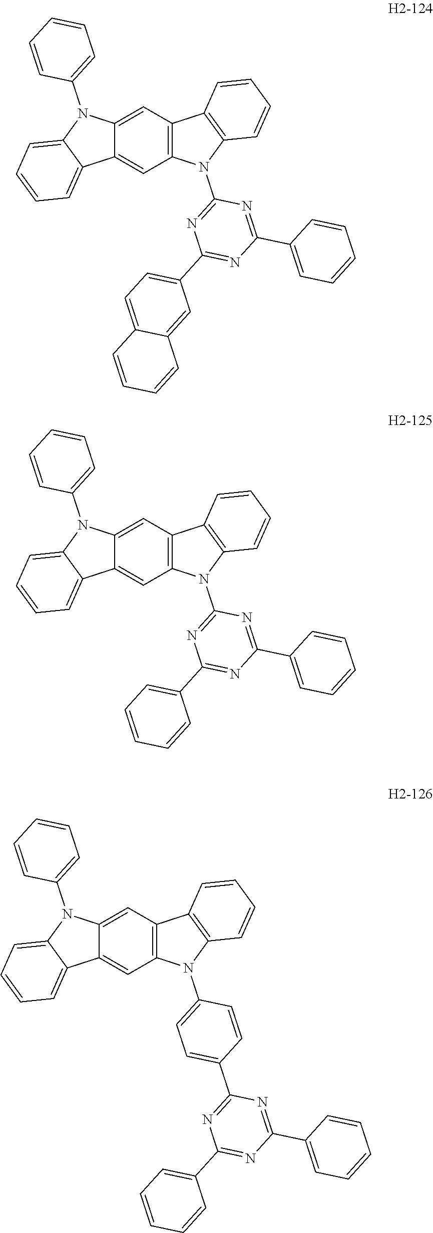 [SMILES: c1ccc(-c2nc(-c3ccc4ccccc4c3)nc(-n3c4ccccc4c4cc5c(cc43)c3ccccc3n5-c3ccccc3)n2)cc1 ; c1ccc(-c2nc(-c3ccccc3)nc(-n3c4ccccc4c4cc5c(cc43)c3ccccc3n5-c3ccccc3)n2)cc1 ; c1ccc(-c2nc(-c3ccccc3)nc(-c3ccc(-n4c5ccccc5c5cc6c(cc54)c4ccccc4n6-c4ccccc4)cc3)n2)cc1]

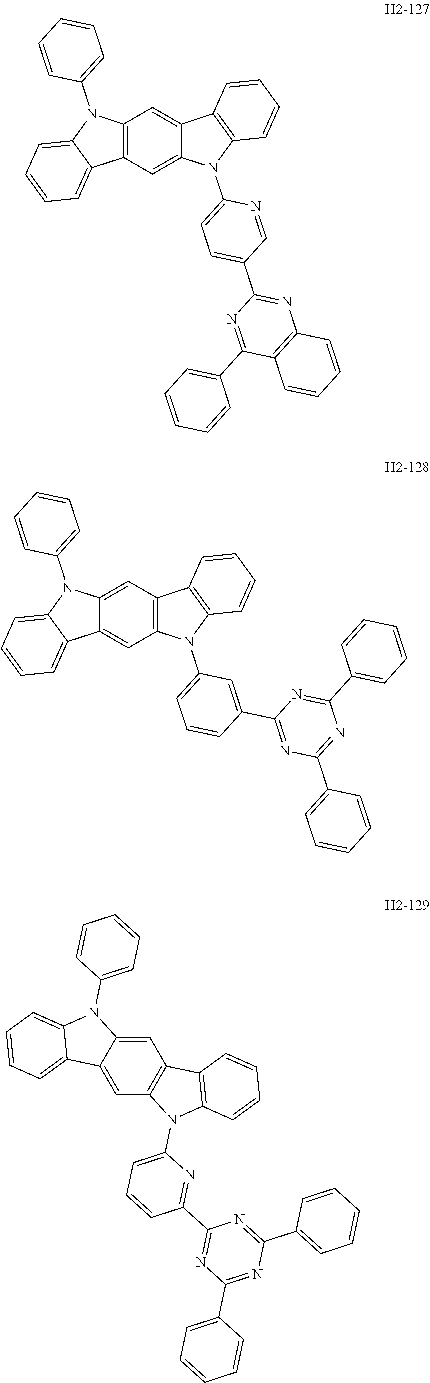 [SMILES: c1ccc(-c2nc(-c3ccc(-n4c5ccccc5c5cc6c(cc54)c4ccccc4n6-c4ccccc4)nc3)nc3ccccc23)cc1 ; c1ccc(-c2nc(-c3ccccc3)nc(-c3cccc(-n4c5ccccc5c5cc6c(cc54)c4ccccc4n6-c4ccccc4)c3)n2)cc1 ; c1ccc(-c2nc(-c3ccccc3)nc(-c3cccc(-n4c5ccccc5c5cc6c(cc54)c4ccccc4n6-c4ccccc4)n3)n2)cc1]

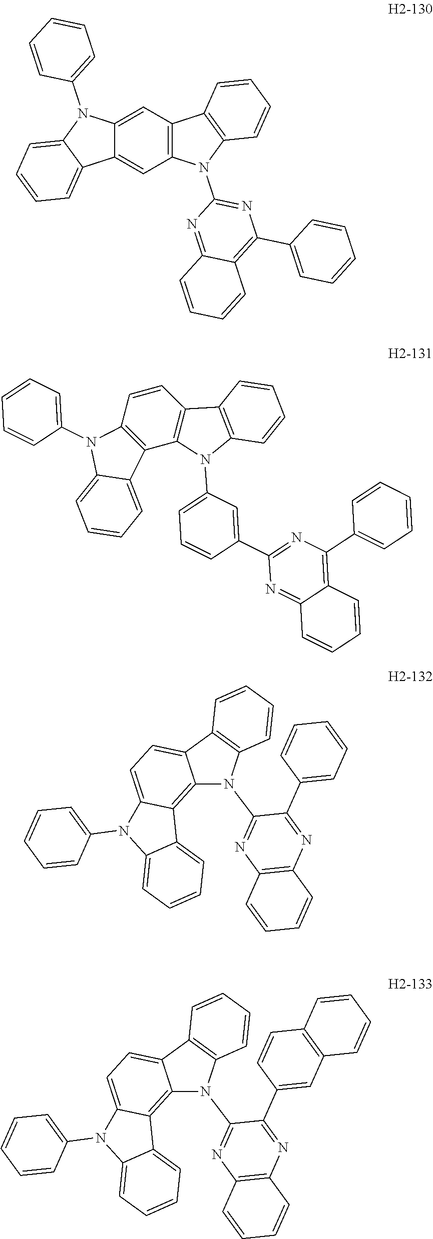 [SMILES: c1ccc(-c2nc(-n3c4ccccc4c4cc5c(cc43)c3ccccc3n5-c3ccccc3)nc3ccccc23)cc1 ; c1ccc(-c2nc(-c3cccc(-n4c5ccccc5c5ccc6c(c7ccccc7n6-c6ccccc6)c54)c3)nc3ccccc23)cc1 ; c1ccc(-c2nc3ccccc3nc2-n2c3ccccc3c3ccc4c(c5ccccc5n4-c4ccccc4)c32)cc1 ; c1ccc(-n2c3ccccc3c3c2ccc2c4ccccc4n(-c4nc5ccccc5nc4-c4ccc5ccccc5c4)c23)cc1]

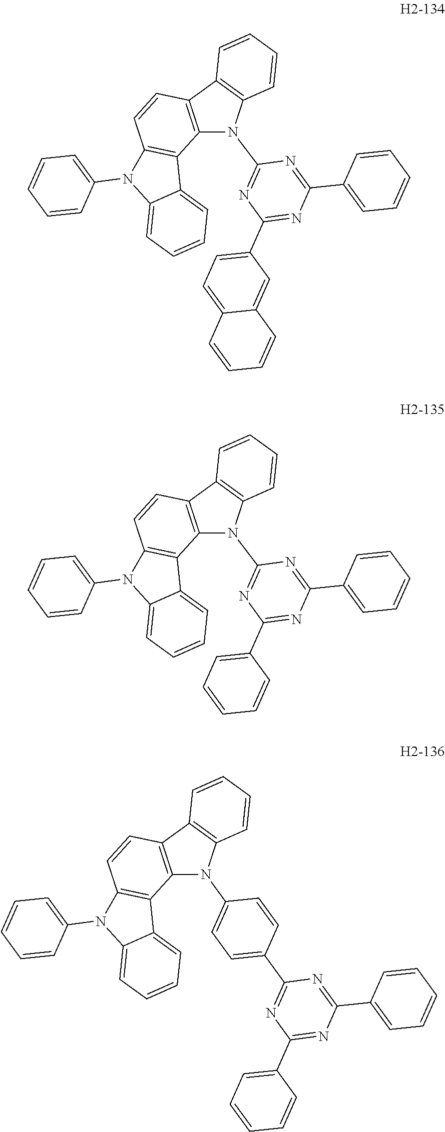 [SMILES: c1ccc(-c2nc(-c3ccc4ccccc4c3)nc(-n3c4ccccc4c4ccc5c(c6ccccc6n5-c5ccccc5)c43)n2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(-n4c5ccccc5c5ccc6c(c7ccccc7n6-c6ccccc6)c54)cc3)n2)cc1]

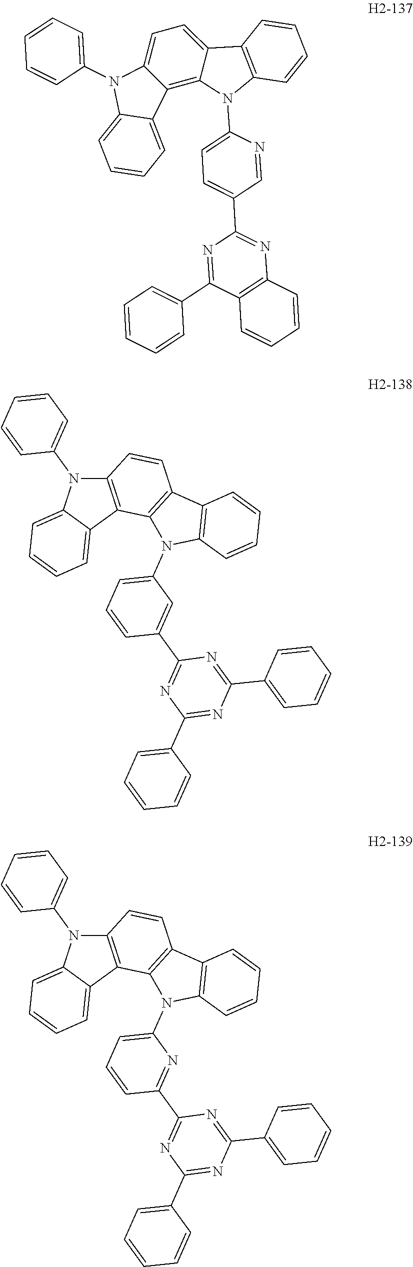 [SMILES: c1ccc(-c2nc(-c3ccc(-n4c5ccccc5c5ccc6c(c7ccccc7n6-c6ccccc6)c54)nc3)nc3ccccc23)cc1 ; c1ccc(-c2nc(-c3ccccc3)nc(-c3cccc(-n4c5ccccc5c5ccc6c(c7ccccc7n6-c6ccccc6)c54)c3)n2)cc1 ; c1ccc(-c2nc(-c3ccccc3)nc(-c3cccc(-n4c5ccccc5c5ccc6c(c7ccccc7n6-c6ccccc6)c54)n3)n2)cc1]

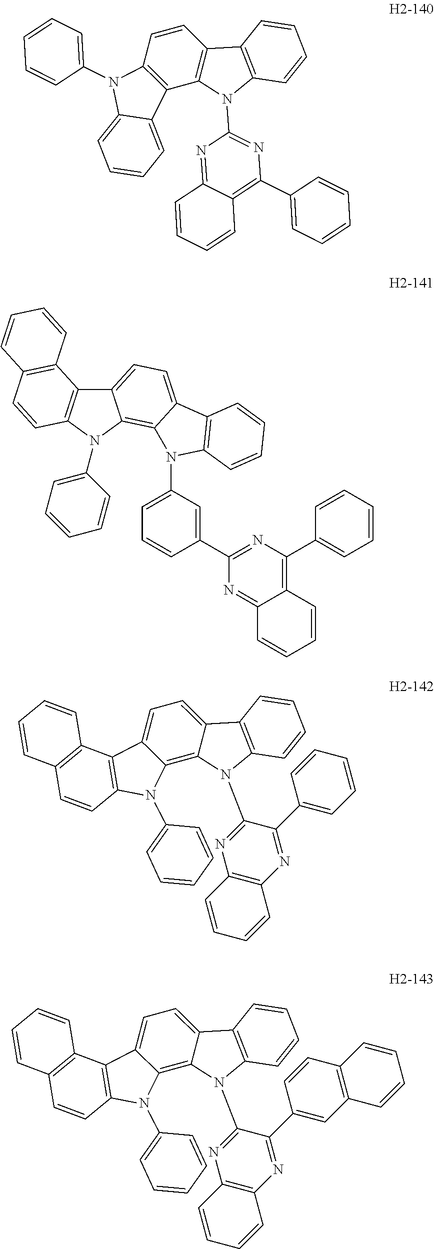

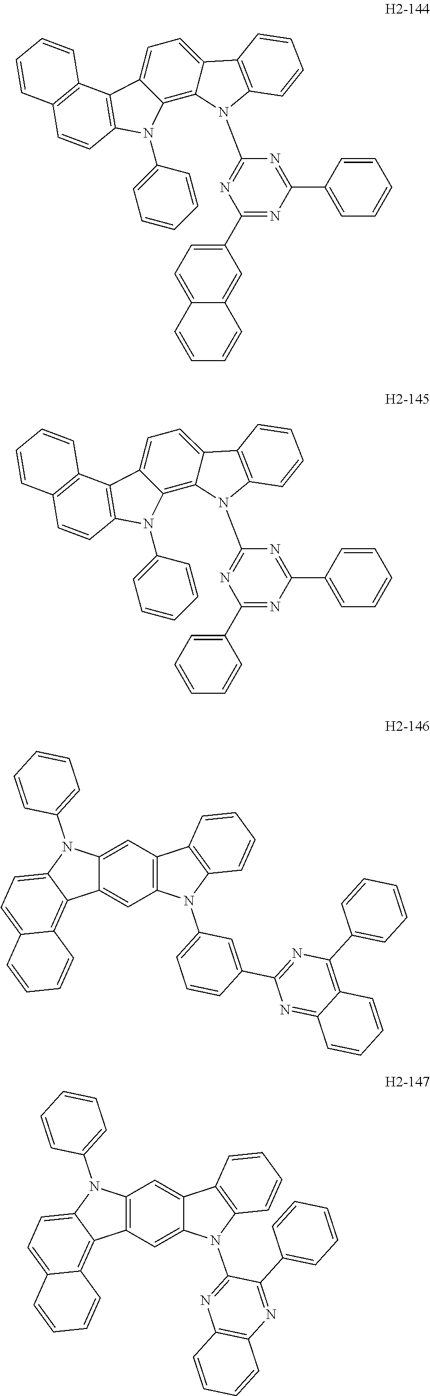 [SMILES: c1ccc(-c2nc(-c3ccc4ccccc4c3)nc(-n3c4ccccc4c4ccc5c6c7ccccc7ccc6n(-c6ccccc6)c5c43)n2)cc1 ; c1ccc(-c2nc(-c3ccccc3)nc(-n3c4ccccc4c4ccc5c6c7ccccc7ccc6n(-c6ccccc6)c5c43)n2)cc1 ; c1ccc(-c2nc(-c3cccc(-n4c5ccccc5c5cc6c(cc54)c4c5ccccc5ccc4n6-c4ccccc4)c3)nc3ccccc23)cc1 ; c1ccc(-c2nc3ccccc3nc2-n2c3ccccc3c3cc4c(cc32)c2c3ccccc3ccc2n4-c2ccccc2)cc1]

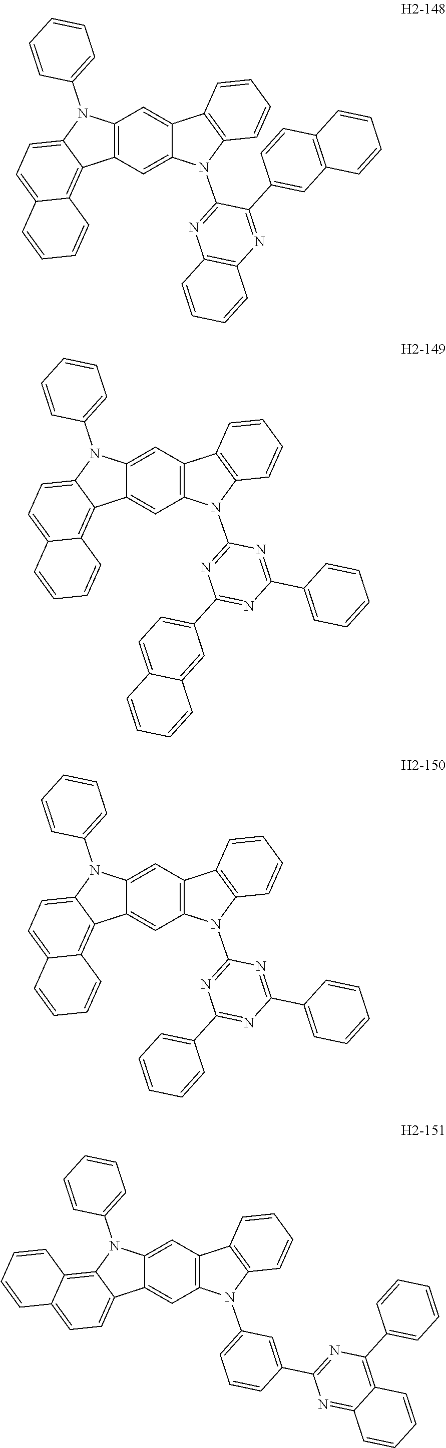 [SMILES: c1ccc(-n2c3cc4c5ccccc5n(-c5nc6ccccc6nc5-c5ccc6ccccc6c5)c4cc3c3c4ccccc4ccc32)cc1 ; c1ccc(-c2nc(-c3ccc4ccccc4c3)nc(-n3c4ccccc4c4cc5c(cc43)c3c4ccccc4ccc3n5-c3ccccc3)n2)cc1 ; c1ccc(-c2nc(-c3ccccc3)nc(-n3c4ccccc4c4cc5c(cc43)c3c4ccccc4ccc3n5-c3ccccc3)n2)cc1 ; c1ccc(-c2nc(-c3cccc(-n4c5ccccc5c5cc6c(cc54)c4ccc5ccccc5c4n6-c4ccccc4)c3)nc3ccccc23)cc1]

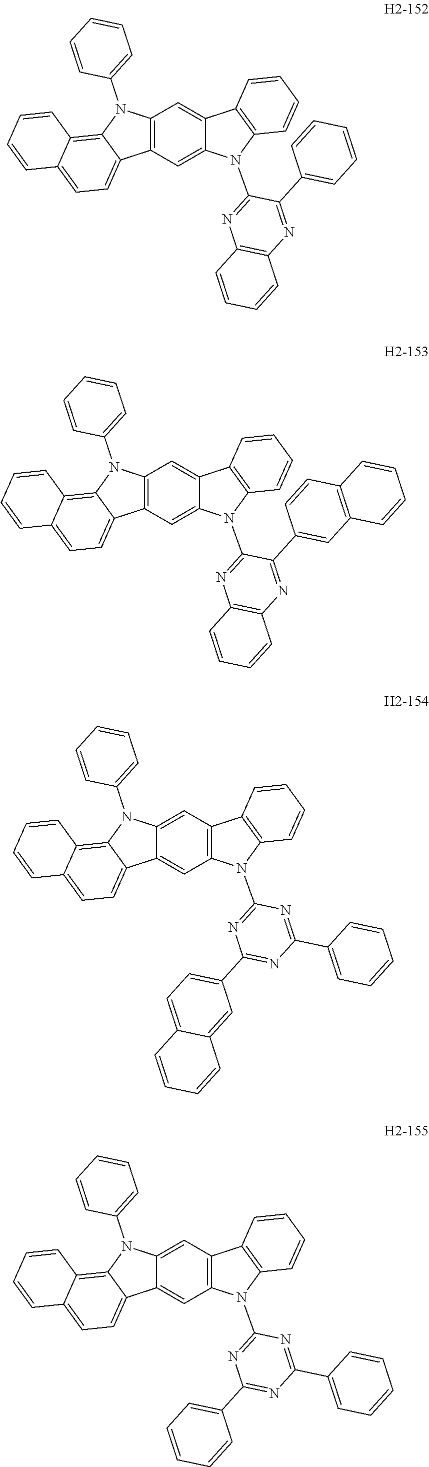 [SMILES: c1ccc(-c2nc3ccccc3nc2-n2c3ccccc3c3cc4c(cc32)c2ccc3ccccc3c2n4-c2ccccc2)cc1 ; c1ccc(-n2c3cc4c5ccccc5n(-c5nc6ccccc6nc5-c5ccc6ccccc6c5)c4cc3c3ccc4ccccc4c32)cc1 ; c1ccc(-c2nc(-c3ccc4ccccc4c3)nc(-n3c4ccccc4c4cc5c(cc43)c3ccc4ccccc4c3n5-c3ccccc3)n2)cc1 ; c1ccc(-c2nc(-c3ccccc3)nc(-n3c4ccccc4c4cc5c(cc43)c3ccc4ccccc4c3n5-c3ccccc3)n2)cc1]

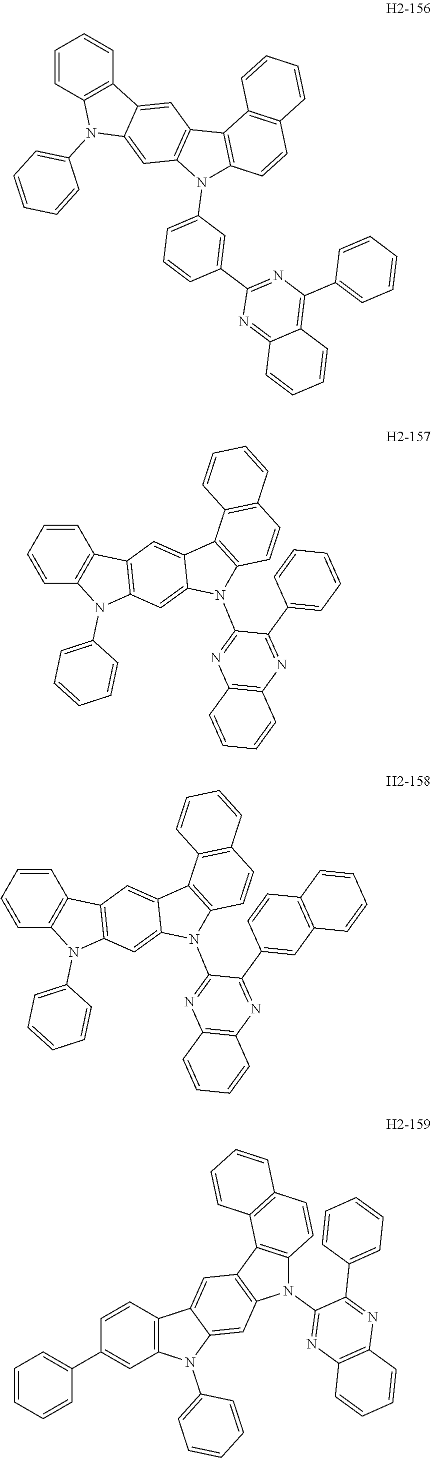

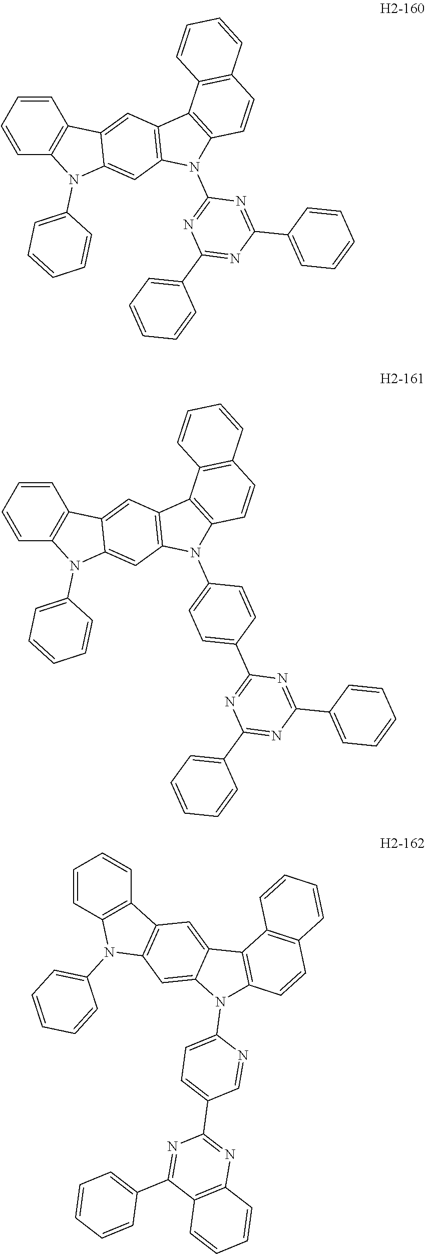 [SMILES: c1ccc(-c2nc(-c3ccccc3)nc(-n3c4cc5c(cc4c4c6ccccc6ccc43)c3ccccc3n5-c3ccccc3)n2)cc1 ; c1ccc(-c2nc(-c3ccccc3)nc(-c3ccc(-n4c5cc6c(cc5c5c7ccccc7ccc54)c4ccccc4n6-c4ccccc4)cc3)n2)cc1 ; c1ccc(-c2nc(-c3ccc(-n4c5cc6c(cc5c5c7ccccc7ccc54)c4ccccc4n6-c4ccccc4)nc3)nc3ccccc23)cc1]

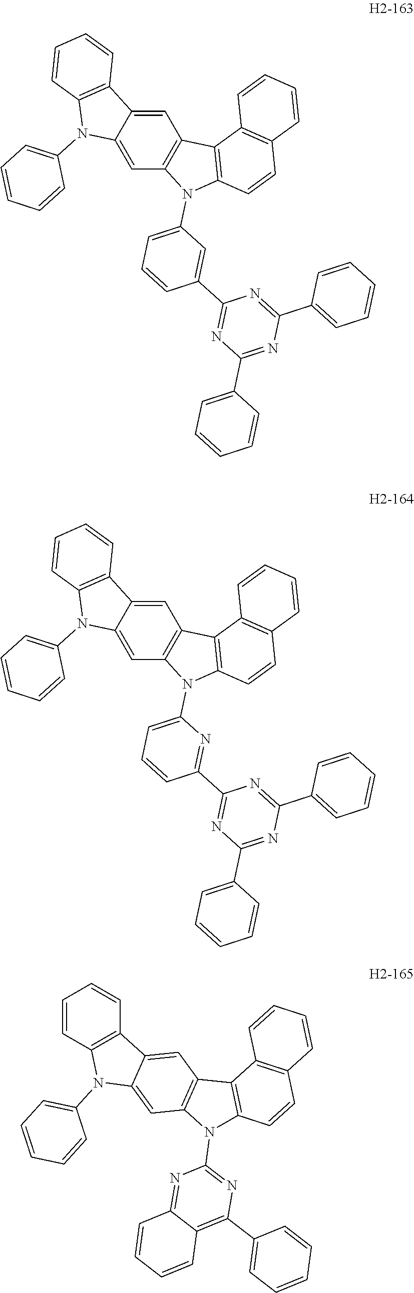 [SMILES: c1ccc(-c2nc(-c3ccccc3)nc(-c3cccc(-n4c5cc6c(cc5c5c7ccccc7ccc54)c4ccccc4n6-c4ccccc4)c3)n2)cc1 ; c1ccc(-c2nc(-c3ccccc3)nc(-c3cccc(-n4c5cc6c(cc5c5c7ccccc7ccc54)c4ccccc4n6-c4ccccc4)n3)n2)cc1 ; c1ccc(-c2nc(-n3c4cc5c(cc4c4c6ccccc6ccc43)c3ccccc3n5-c3ccccc3)nc3ccccc23)cc1]

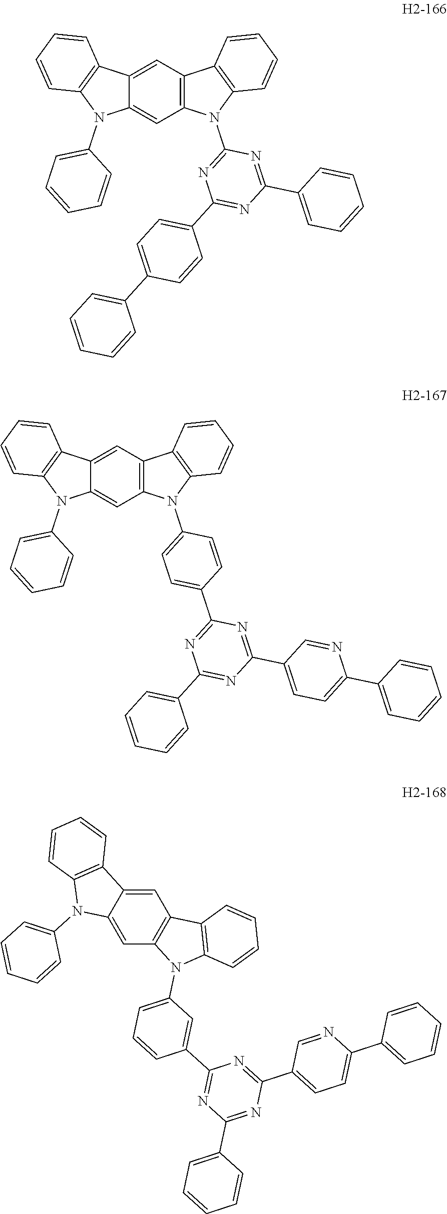 [SMILES: c1ccc(-c2ccc(-c3nc(-c4ccccc4)nc(-n4c5ccccc5c5cc6c7ccccc7n(-c7ccccc7)c6cc54)n3)cc2)cc1 ; c1ccc(-c2ccc(-c3nc(-c4ccccc4)nc(-c4ccc(-n5c6ccccc6c6cc7c8ccccc8n(-c8ccccc8)c7cc65)cc4)n3)cn2)cc1 ; c1ccc(-c2ccc(-c3nc(-c4ccccc4)nc(-c4cccc(-n5c6ccccc6c6cc7c8ccccc8n(-c8ccccc8)c7cc65)c4)n3)cn2)cc1]

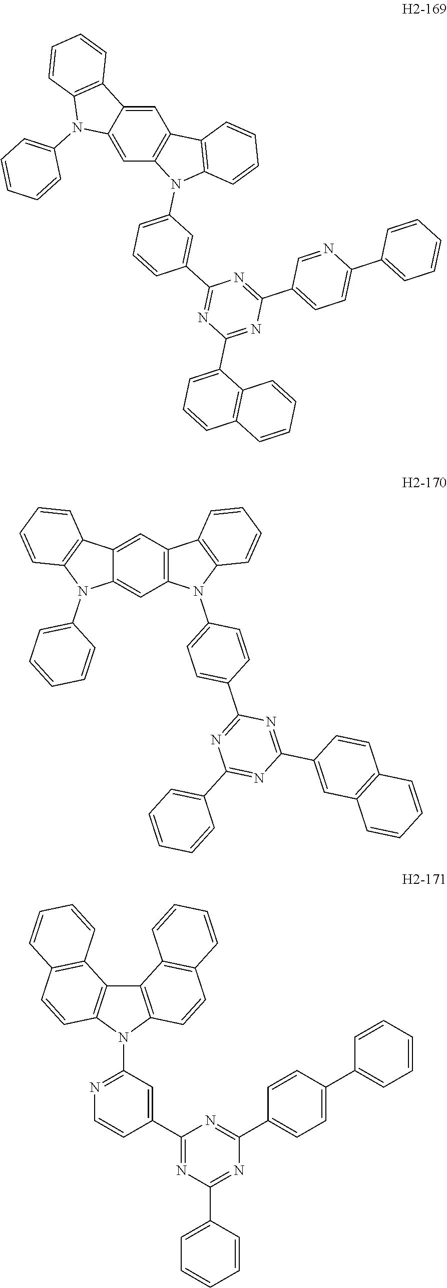 [SMILES: c1ccc(-c2ccc(-c3nc(-c4cccc(-n5c6ccccc6c6cc7c8ccccc8n(-c8ccccc8)c7cc65)c4)nc(-c4cccc5ccccc45)n3)cn2)cc1 ; c1ccc(-c2nc(-c3ccc(-n4c5ccccc5c5cc6c7ccccc7n(-c7ccccc7)c6cc54)cc3)nc(-c3ccc4ccccc4c3)n2)cc1 ; c1ccc(-c2ccc(-c3nc(-c4ccccc4)nc(-c4ccnc(-n5c6ccc7ccccc7c6c6c7ccccc7ccc65)c4)n3)cc2)cc1]

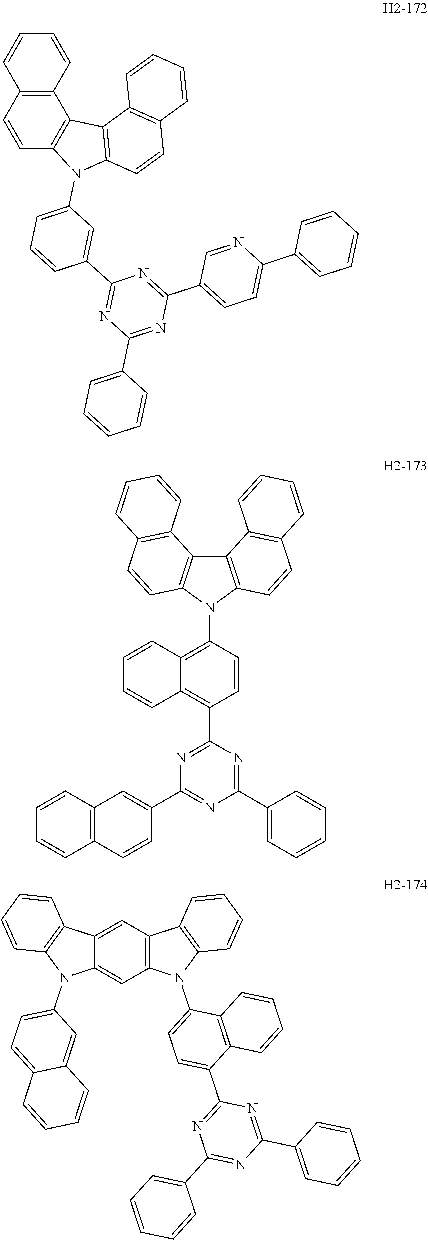

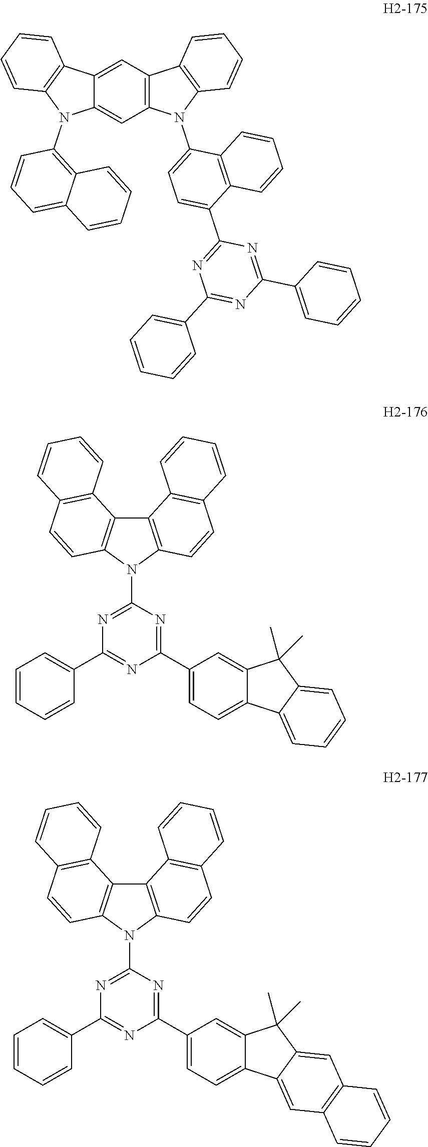 [SMILES: c1ccc(-c2nc(-c3ccccc3)nc(-c3ccc(-n4c5ccccc5c5cc6c7ccccc7n(-c7cccc8ccccc78)c6cc54)c4ccccc34)n2)cc1 ; CC1(C)c2ccccc2-c2ccc(-c3nc(-c4ccccc4)nc(-n4c5ccc6ccccc6c5c5c6ccccc6ccc54)n3)cc21 ; CC1(C)c2cc(-c3nc(-c4ccccc4)nc(-n4c5ccc6ccccc6c5c5c6ccccc6ccc54)n3)ccc2-c2cc3ccccc3cc21]

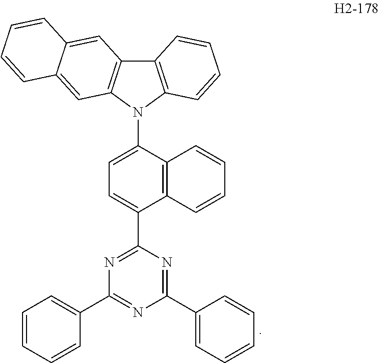 [SMILES: c1ccc(-c2nc(-c3ccccc3)nc(-c3ccc(-n4c5ccccc5c5cc6ccccc6cc54)c4ccccc34)n2)cc1]

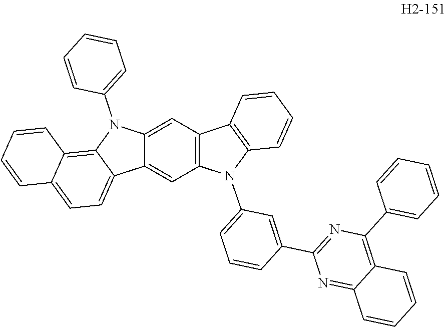 [SMILES: c1ccc(-c2nc(-c3cccc(-n4c5ccccc5c5cc6c(cc54)c4ccc5ccccc5c4n6-c4ccccc4)c3)nc3ccccc23)cc1]

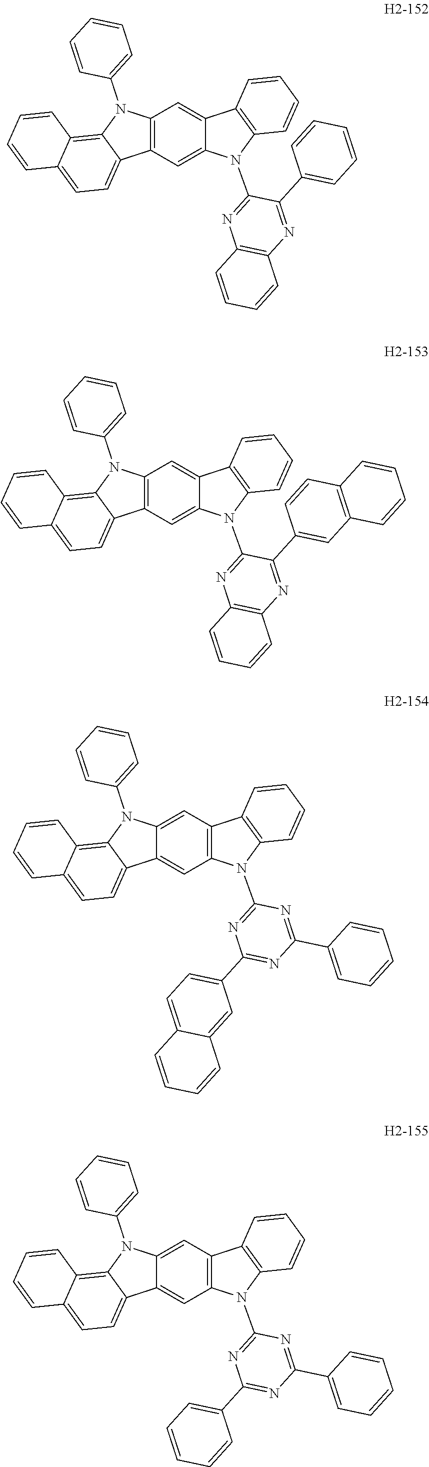 [SMILES: c1ccc(-c2nc3ccccc3nc2-n2c3ccccc3c3cc4c(cc32)c2ccc3ccccc3c2n4-c2ccccc2)cc1 ; c1ccc(-n2c3cc4c5ccccc5n(-c5nc6ccccc6nc5-c5ccc6ccccc6c5)c4cc3c3ccc4ccccc4c32)cc1 ; c1ccc(-c2nc(-c3ccc4ccccc4c3)nc(-n3c4ccccc4c4cc5c(cc43)c3ccc4ccccc4c3n5-c3ccccc3)n2)cc1 ; c1ccc(-c2nc(-c3ccccc3)nc(-n3c4ccccc4c4cc5c(cc43)c3ccc4ccccc4c3n5-c3ccccc3)n2)cc1]

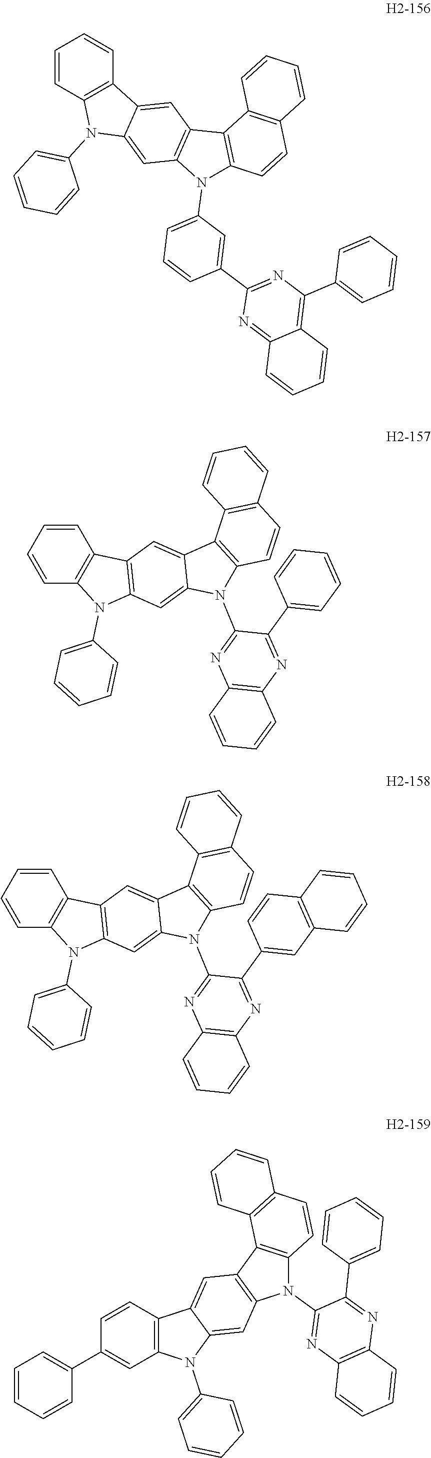

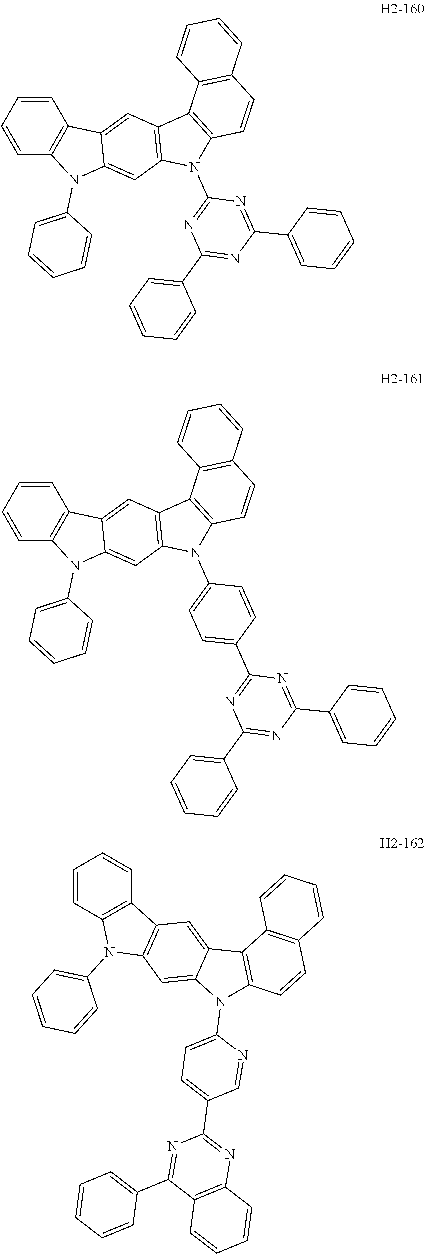 [SMILES: c1ccc(-c2nc(-c3ccccc3)nc(-n3c4cc5c(cc4c4c6ccccc6ccc43)c3ccccc3n5-c3ccccc3)n2)cc1 ; c1ccc(-c2nc(-c3ccccc3)nc(-c3ccc(-n4c5cc6c(cc5c5c7ccccc7ccc54)c4ccccc4n6-c4ccccc4)cc3)n2)cc1 ; c1ccc(-c2nc(-c3ccc(-n4c5cc6c(cc5c5c7ccccc7ccc54)c4ccccc4n6-c4ccccc4)nc3)nc3ccccc23)cc1]

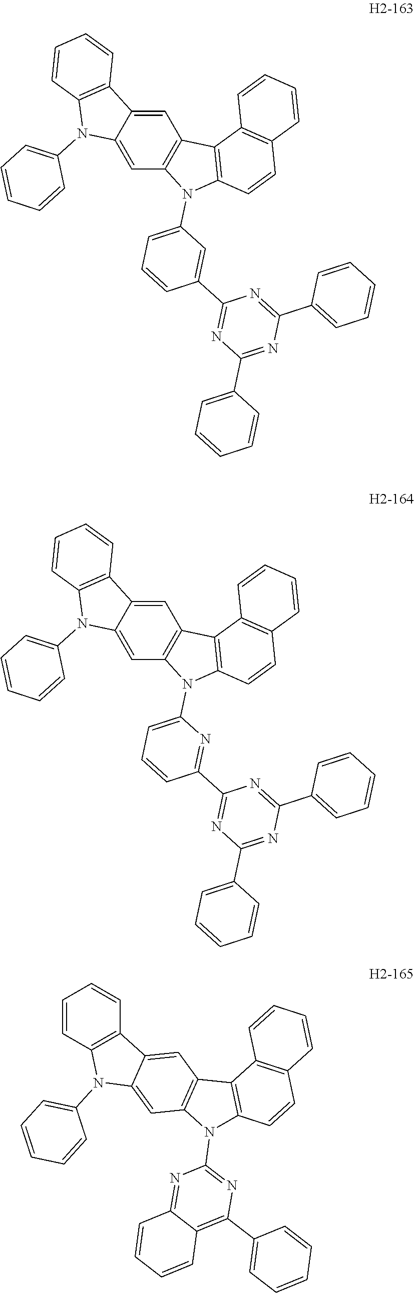 [SMILES: c1ccc(-c2nc(-c3ccccc3)nc(-c3cccc(-n4c5cc6c(cc5c5c7ccccc7ccc54)c4ccccc4n6-c4ccccc4)c3)n2)cc1 ; c1ccc(-c2nc(-c3ccccc3)nc(-c3cccc(-n4c5cc6c(cc5c5c7ccccc7ccc54)c4ccccc4n6-c4ccccc4)n3)n2)cc1 ; c1ccc(-c2nc(-n3c4cc5c(cc4c4c6ccccc6ccc43)c3ccccc3n5-c3ccccc3)nc3ccccc23)cc1]

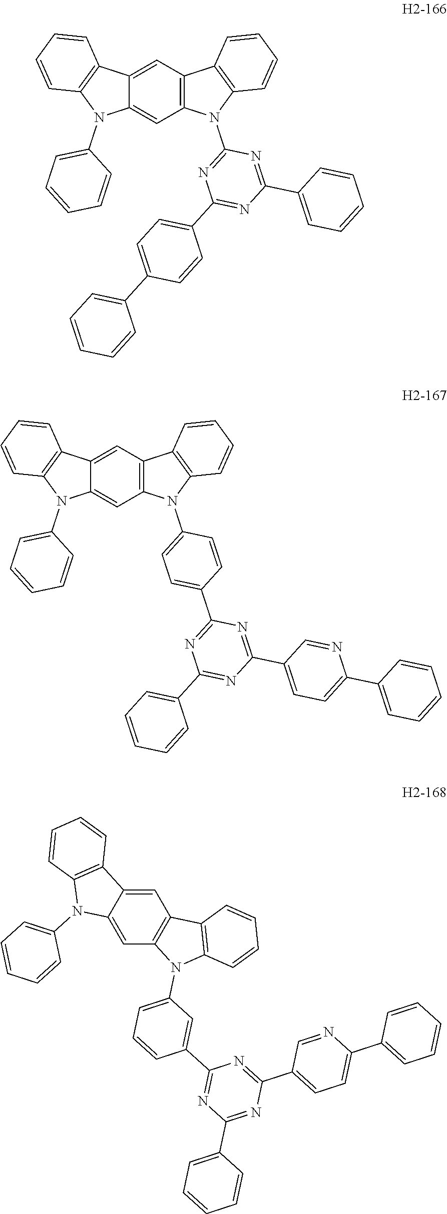 [SMILES: c1ccc(-c2ccc(-c3nc(-c4ccccc4)nc(-n4c5ccccc5c5cc6c7ccccc7n(-c7ccccc7)c6cc54)n3)cc2)cc1 ; c1ccc(-c2ccc(-c3nc(-c4ccccc4)nc(-c4ccc(-n5c6ccccc6c6cc7c8ccccc8n(-c8ccccc8)c7cc65)cc4)n3)cn2)cc1 ; c1ccc(-c2ccc(-c3nc(-c4ccccc4)nc(-c4cccc(-n5c6ccccc6c6cc7c8ccccc8n(-c8ccccc8)c7cc65)c4)n3)cn2)cc1]

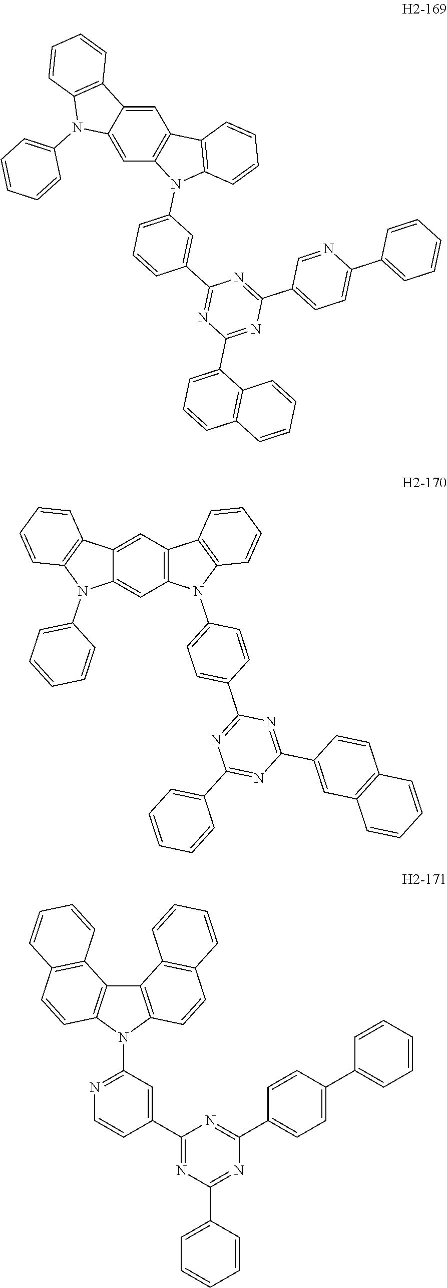 [SMILES: c1ccc(-c2ccc(-c3nc(-c4cccc(-n5c6ccccc6c6cc7c8ccccc8n(-c8ccccc8)c7cc65)c4)nc(-c4cccc5ccccc45)n3)cn2)cc1 ; c1ccc(-c2nc(-c3ccc(-n4c5ccccc5c5cc6c7ccccc7n(-c7ccccc7)c6cc54)cc3)nc(-c3ccc4ccccc4c3)n2)cc1 ; c1ccc(-c2ccc(-c3nc(-c4ccccc4)nc(-c4ccnc(-n5c6ccc7ccccc7c6c6c7ccccc7ccc65)c4)n3)cc2)cc1]

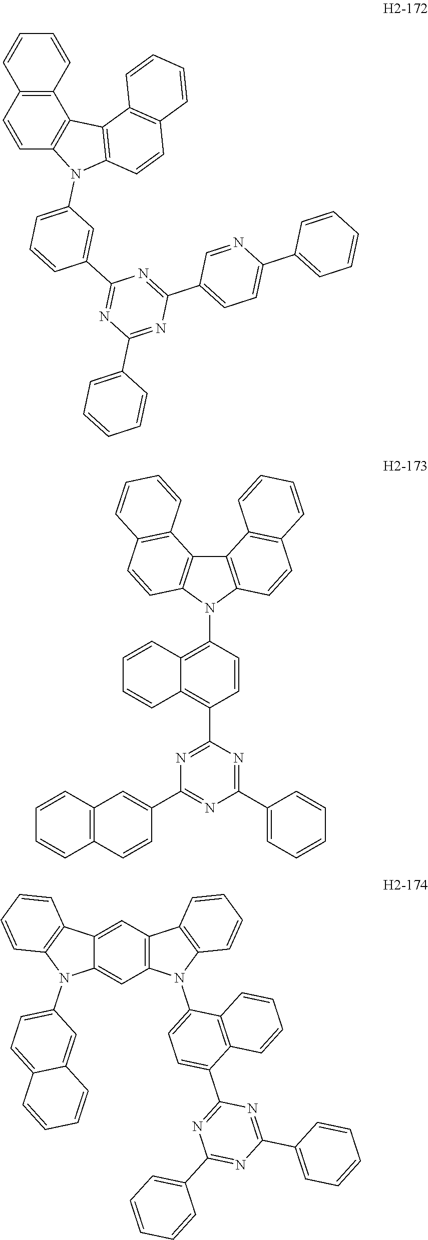

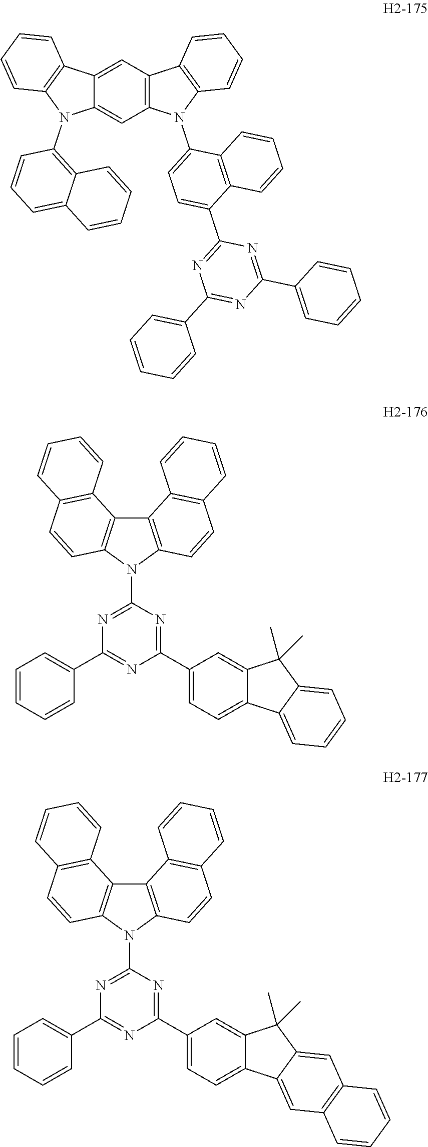 [SMILES: c1ccc(-c2nc(-c3ccccc3)nc(-c3ccc(-n4c5ccccc5c5cc6c7ccccc7n(-c7cccc8ccccc78)c6cc54)c4ccccc34)n2)cc1 ; CC1(C)c2ccccc2-c2ccc(-c3nc(-c4ccccc4)nc(-n4c5ccc6ccccc6c5c5c6ccccc6ccc54)n3)cc21 ; CC1(C)c2cc(-c3nc(-c4ccccc4)nc(-n4c5ccc6ccccc6c5c5c6ccccc6ccc54)n3)ccc2-c2cc3ccccc3cc21]

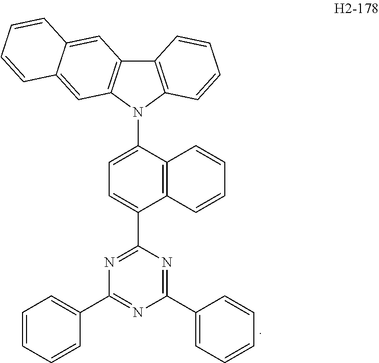 [SMILES: c1ccc(-c2nc(-c3ccccc3)nc(-c3ccc(-n4c5ccccc5c5cc6ccccc6cc54)c4ccccc34)n2)cc1]

The compound represented by formula 2 according to the present disclosure may be produced by a synthetic method known to a person skilled in the art, in particular, using the methods disclosed in many patent literatures, for example, Korean Patent Application Laid-Open No. 2016-0099471 (Aug. 22, 2016), Korean Patent Application Laid-Open No. 2015-0135109 (Dec. 2, 2015), Korean Patent No. 1603070 (Mar. 8, 2016), Korean Patent No. 1477613 (Dec. 23, 2014), Korean Patent Application Laid-Open No. 2015-0077513 (Jul. 8, 2015), Korean Patent No. 1511072 (Apr. 6, 2015), and Korean Patent No. 1531904 (Jun. 22, 2015), but is not limited thereto.

The organic electroluminescent device according to the present disclosure comprises an anode, a cathode, and at least one light-emitting layer between the anode and the cathode. The light-emitting layer comprises a host and a phosphorescent dopant. The host comprises a plurality of host compounds, at least a first host compound of the plurality of host compounds may be represented by formula 1, and a second host compound may be represented by formula 2.

The light-emitting layer is a layer from which light is emitted, and can be a single layer or a multi-layer of which two or more layers are stacked. In the light-emitting layer, it is preferable that the doping concentration of the dopant compound based on the host compound is less than 20 wt %.

The dopant comprised in the organic electroluminescent device according to the present disclosure is preferably at least one phosphorescent dopant. The phosphorescent dopant material comprised in the organic electroluminescent device according to the present disclosure are not particularly limited, but may be preferably selected from metallated complex compounds of iridium (Ir), osmium (Os), copper (Cu), and platinum (Pt), more preferably selected from ortho-metallated complex compounds of iridium (Ir), osmium (Os), copper (Cu) and platinum (Pt), and even more preferably ortho-metallated iridium complex compounds.

The dopant comprised in the organic electroluminescent device according to the present disclosure is preferably selected from the compounds represented by the following formulas 101 to 104.

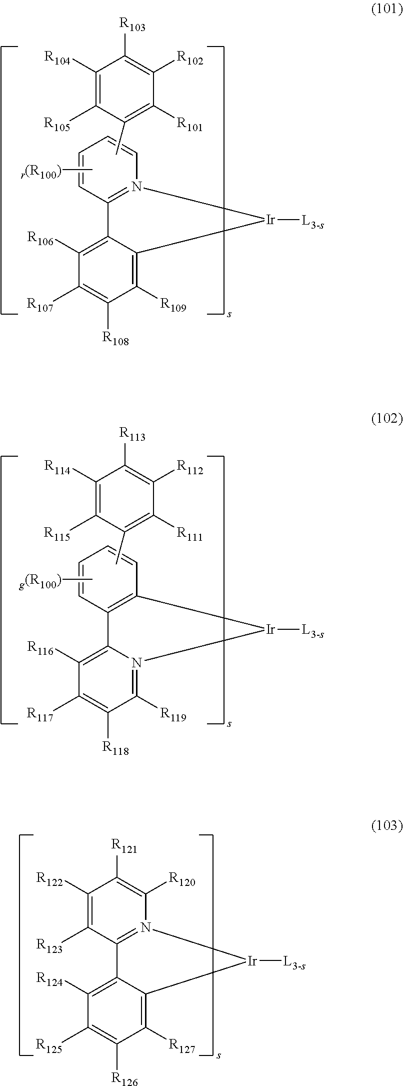

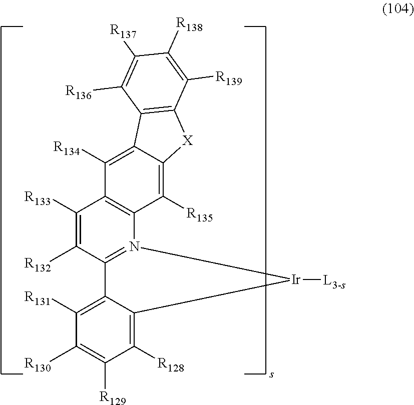

wherein L is selected from the following structures:

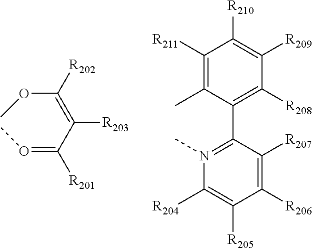

R₁₀₀, R₁₃₄, and R₁₃₅, each independently, represent hydrogen, deuterium, a substituted or unsubstituted (C1-C30)alkyl, or a substituted or unsubstituted (C3-C30)cycloalkyl;
R₁₀₁ to R₁₀₉ and R₁₁₁ to R₁₂₃, each independently, represent hydrogen, deuterium, a halogen, a (C1-C30)alkyl unsubstituted or substituted with deuterium or a halogen, a substituted or unsubstituted (C3-C30)cycloalkyl, a substituted or unsubstituted (C6-C30)aryl, a cyano, or a substituted or unsubstituted (C1-C30)alkoxy, where R₁₀₆ to R₁₀₉ may be linked to adjacent R₁₀₆ to R₁₀₉ to form a substituted or unsubstituted fused ring, e.g., a fluorene unsubstituted or substituted with an alkyl, a dibenzothiophene unsubstituted or substituted with an alkyl, or a dibenzofuran unsubstituted or substituted with an alkyl; and R₁₂₀ to R₁₂₃ may be linked to adjacent R₁₂₀ to R₁₂₃ to form a substituted or unsubstituted fused ring, e.g., a quinoline unsubstituted or substituted with at least one selected from an alkyl, an aryl, a arylalkyl, and alkylaryl;
R₁₂₄ to R₁₃₃ and R₁₃₆ to R₁₃₉, each independently, represent hydrogen, deuterium, a halogen, a substituted or unsubstituted (C1-C30)alkyl, or a substituted or unsubstituted (C6-C30)aryl, where R₁₂₄ to R₁₂₇ may be linked to adjacent R₁₂₄ to R₁₂₇ to form a substituted or unsubstituted fused ring, e.g., a fluorene unsubstituted or substituted with an alkyl, a dibenzothiophene unsubstituted or substituted with an alkyl, or a dibenzofuran unsubstituted or substituted with an alkyl;
X represents CR₂₁R₂₂, O, or S;
R₂₁ and R₂₂, each independently, represent a substituted or unsubstituted (C1-C10)alkyl, or a substituted or unsubstituted (C6-C30)aryl;
R₂₀₁ to R₂₁₁, each independently, represent hydrogen, deuterium, a halogen, a (C1-C30)alkyl unsubstituted or substituted with deuterium or a halogen, a substituted or unsubstituted (C3-C30)cycloalkyl, or a (C6-C30)aryl unsubstituted or substituted with an alkyl or deuterium, where R₂₀₈ to R₂₁₁ may be linked to adjacent R₂₀₈ to R₂₁₁ to form a substituted or unsubstituted fused ring, e.g., a fluorene unsubstituted or substituted with an alkyl, a dibenzothiophene unsubstituted or substituted with an alkyl, or a dibenzofuran unsubstituted or substituted with an alkyl;
f and g, each independently, represent an integer of 1 to 3; where if f or g is an integer of 2 or more, each R₁₀₀ may be the same or different; and
s represents an integer of 1 to 3.

Specifically, the dopant material includes the following:

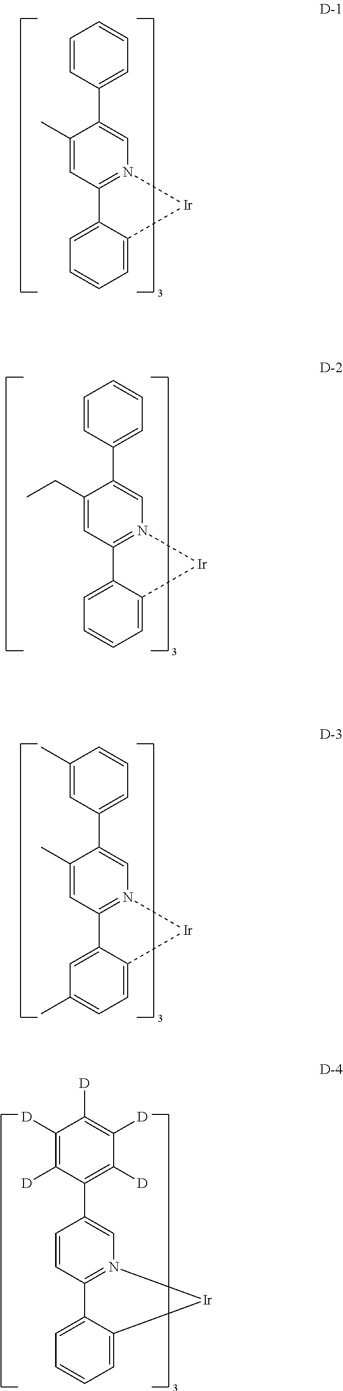

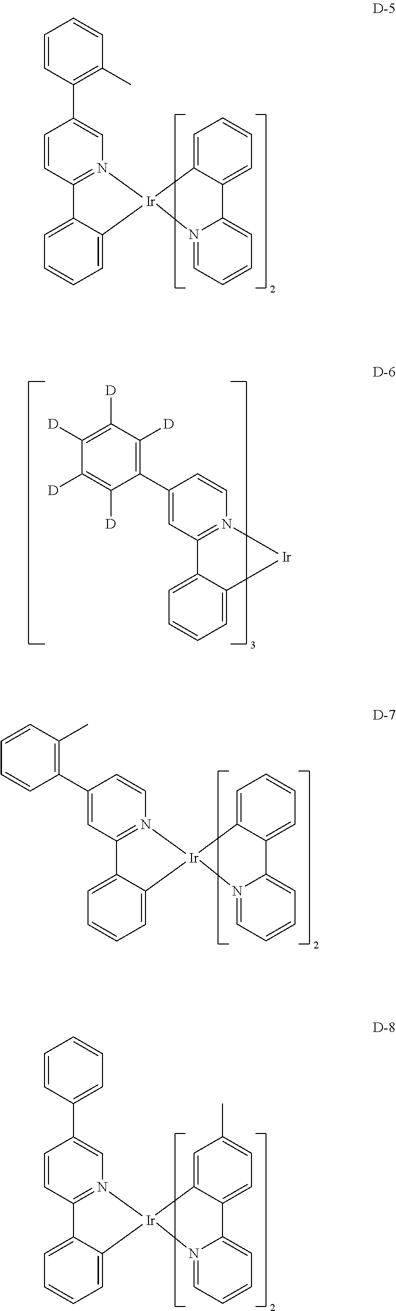

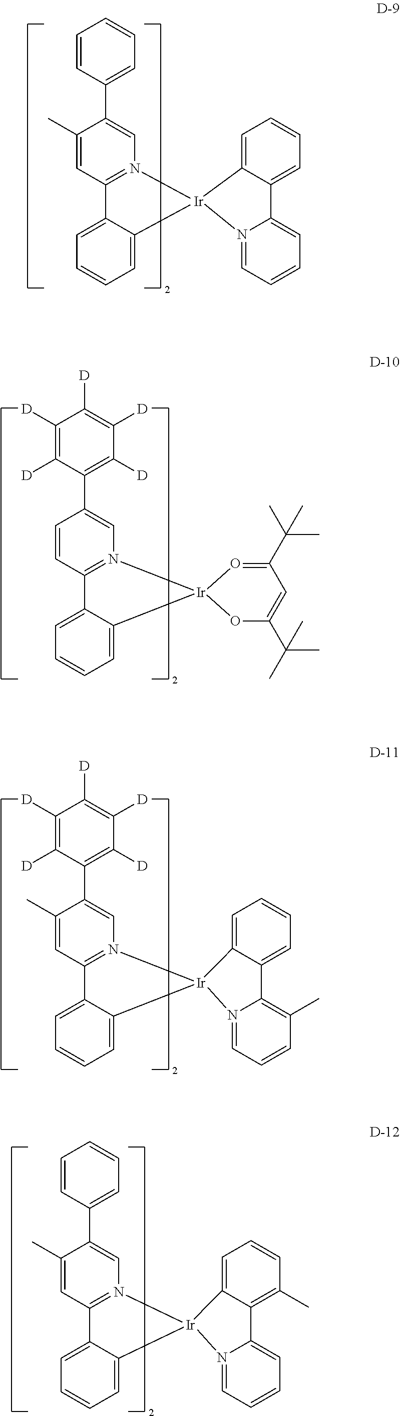

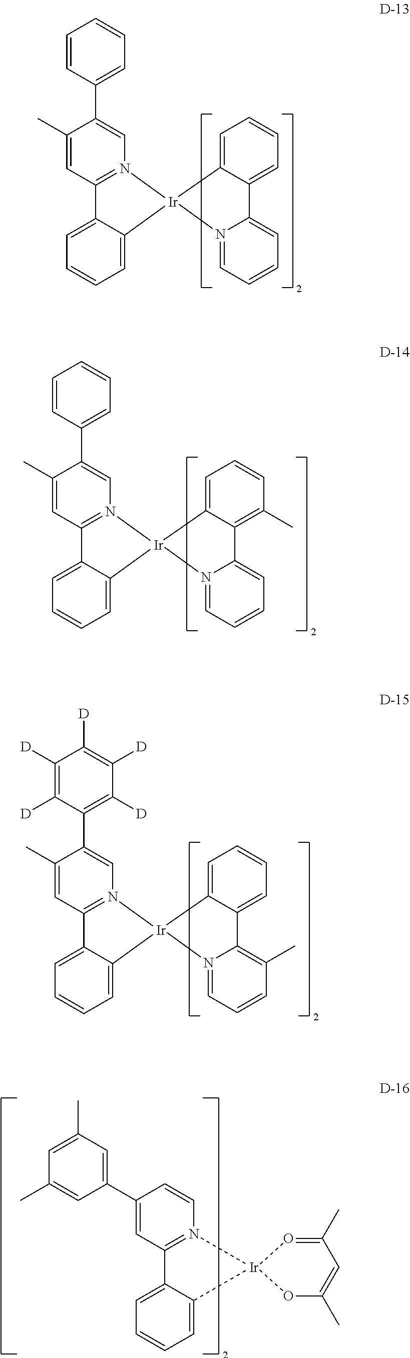

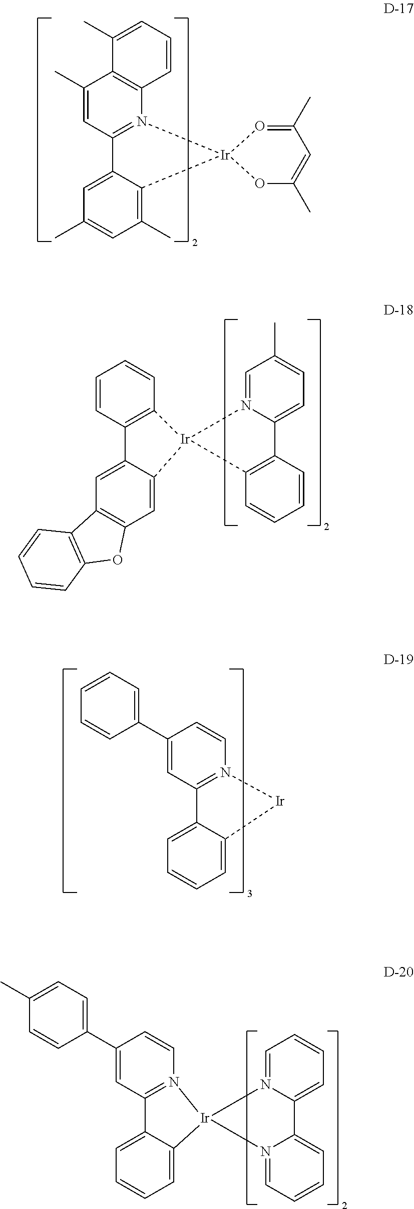

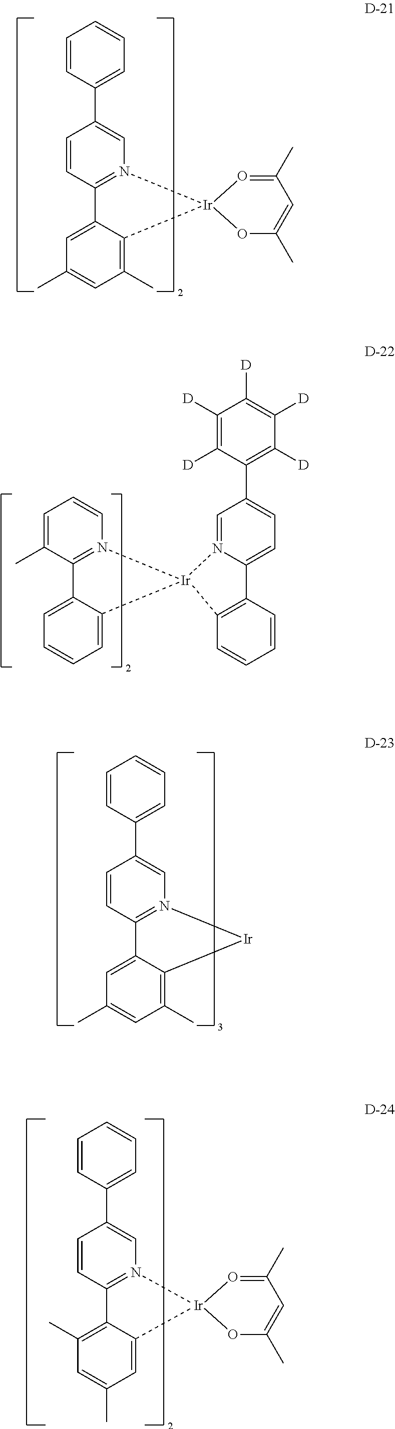

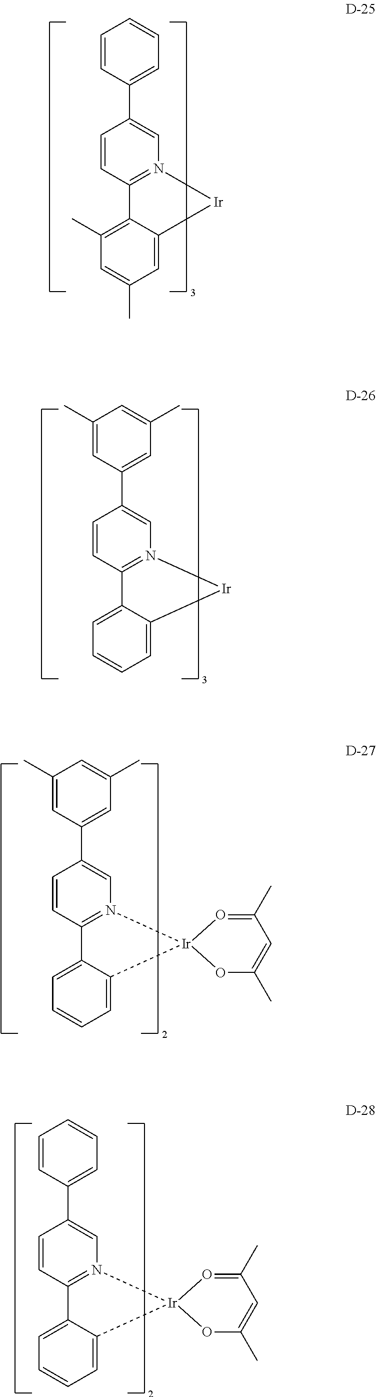

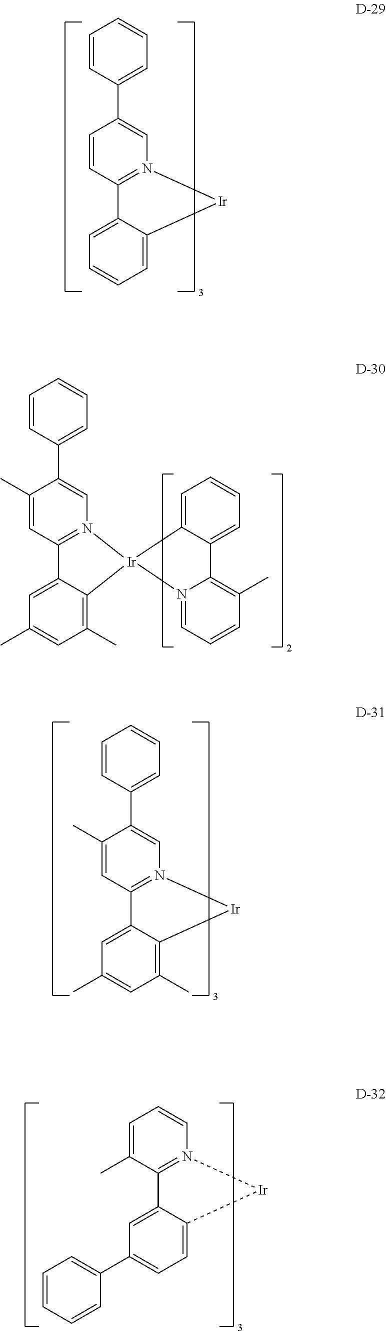

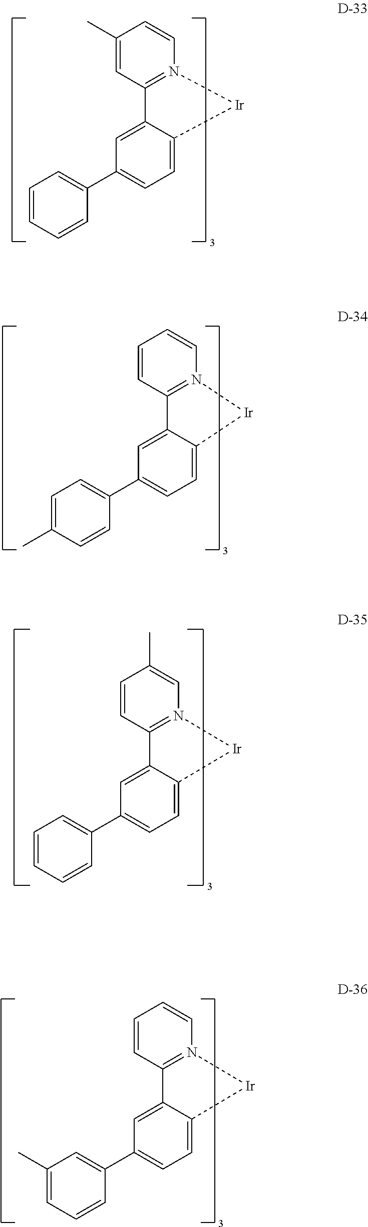

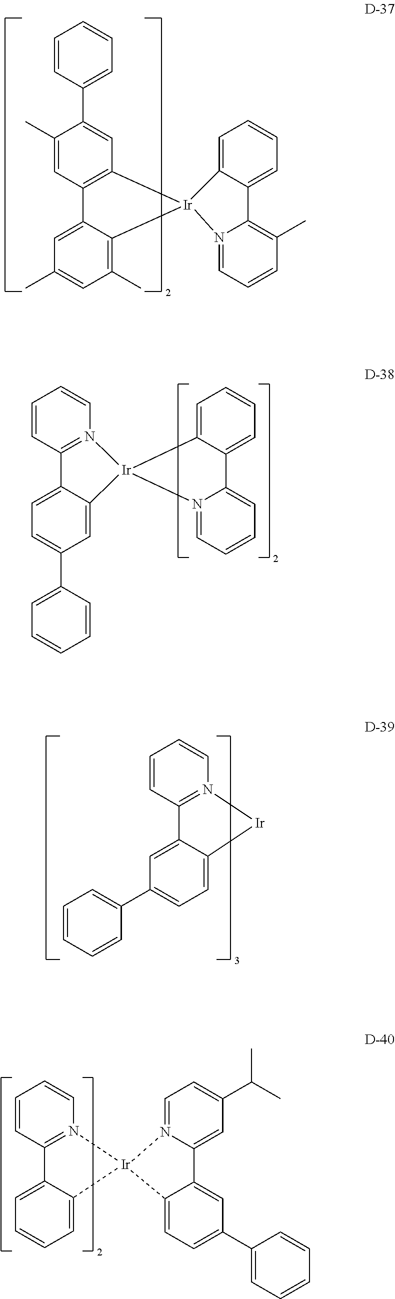

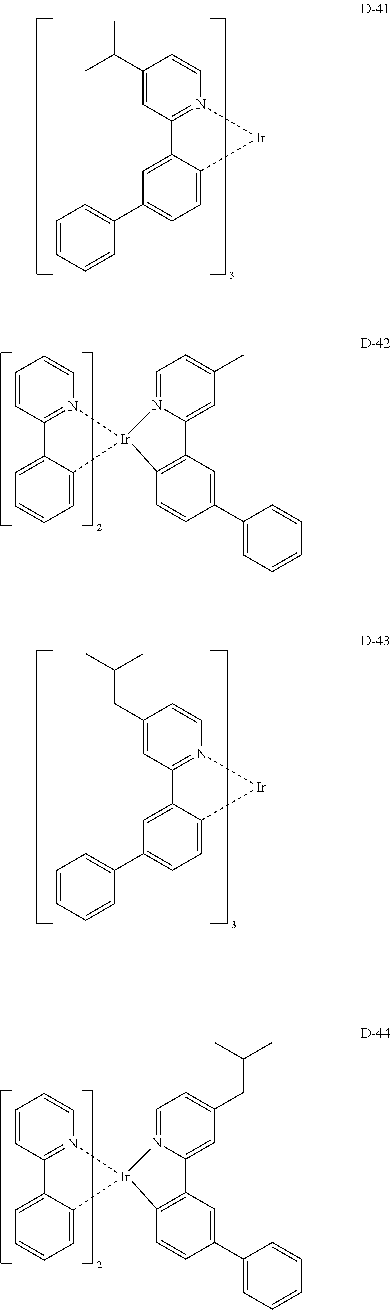

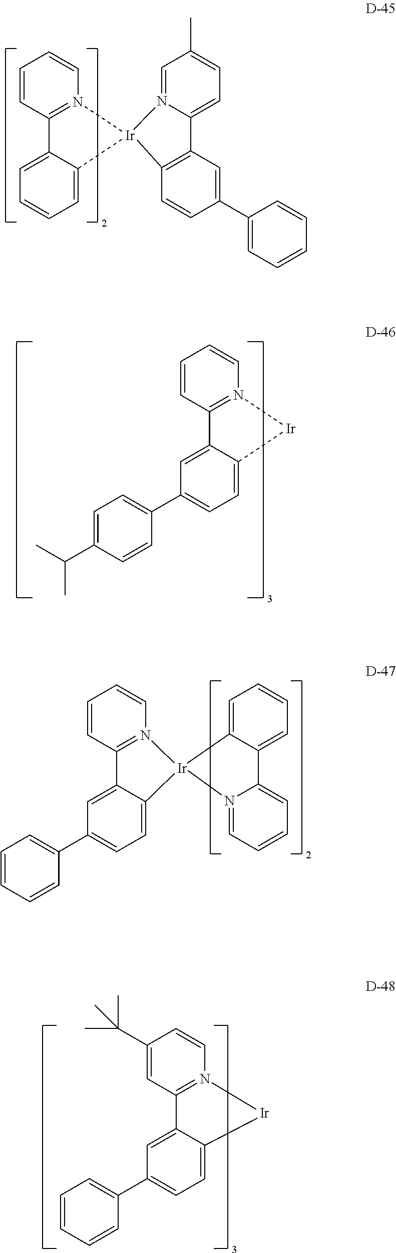

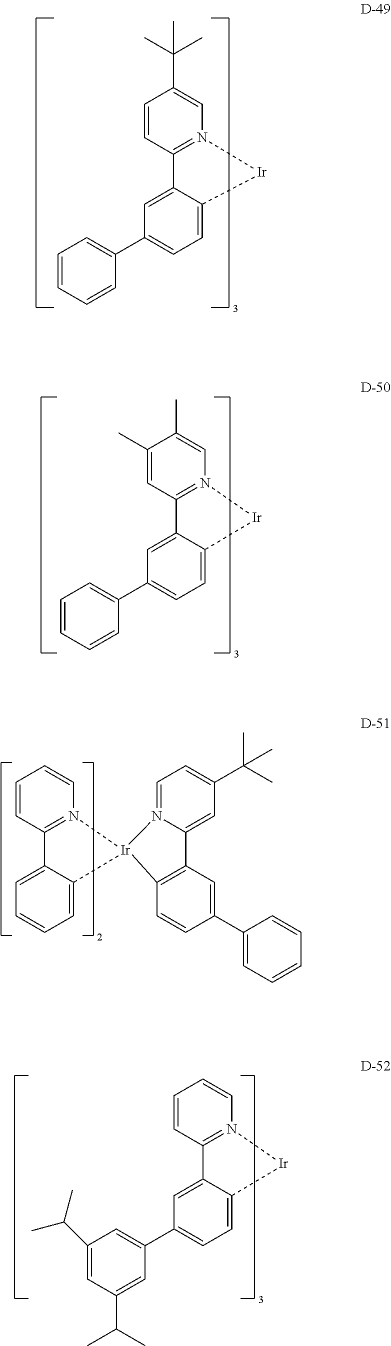

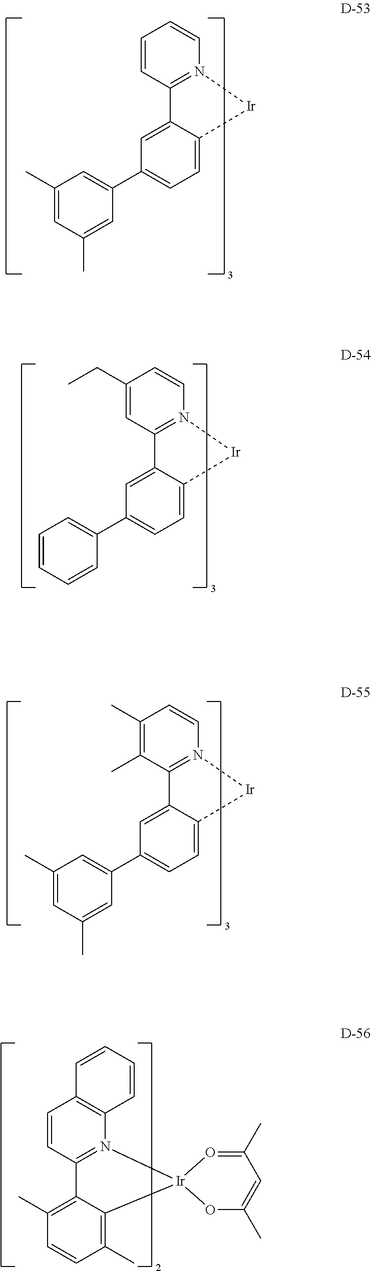

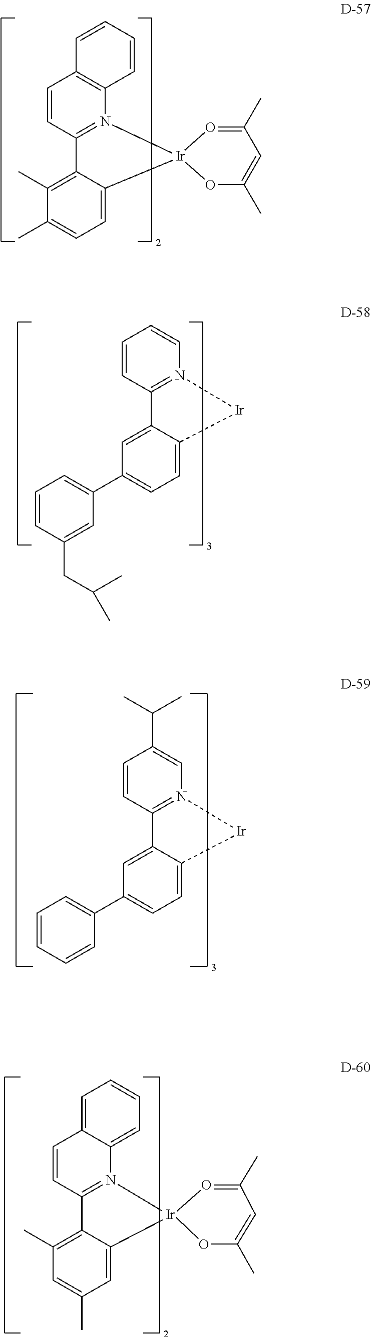

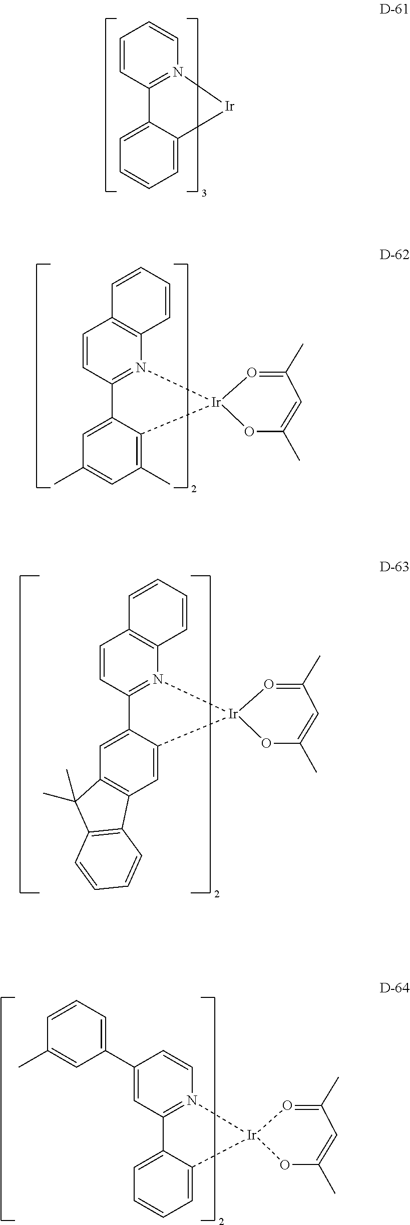

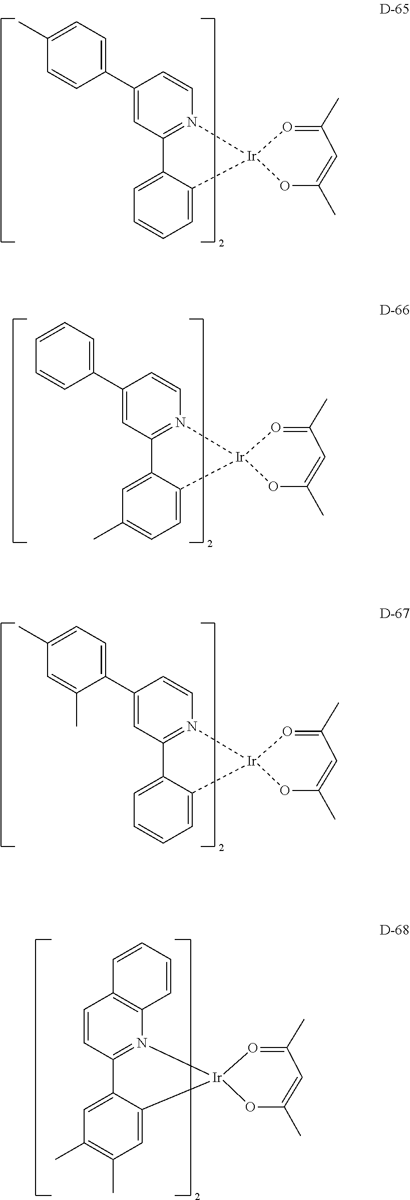

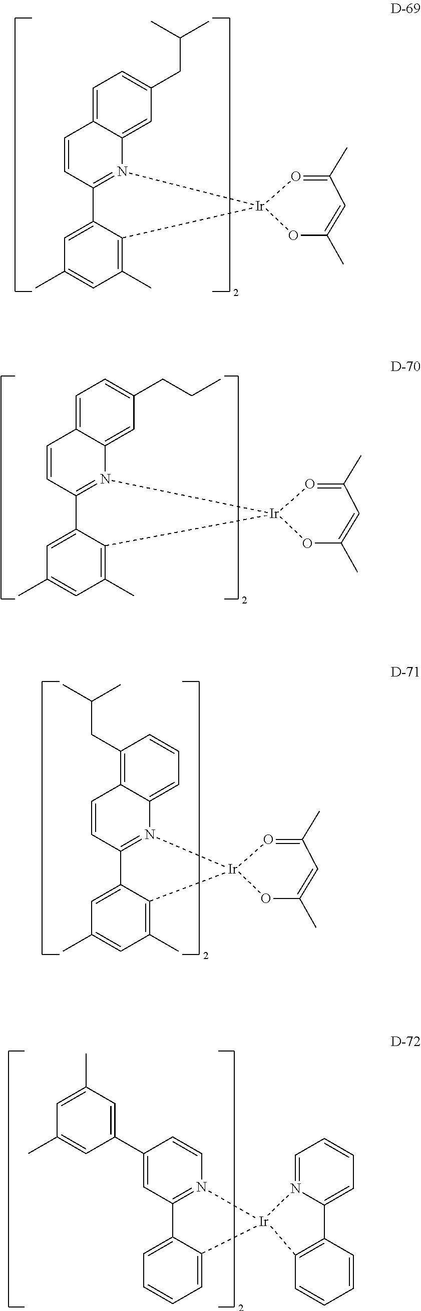

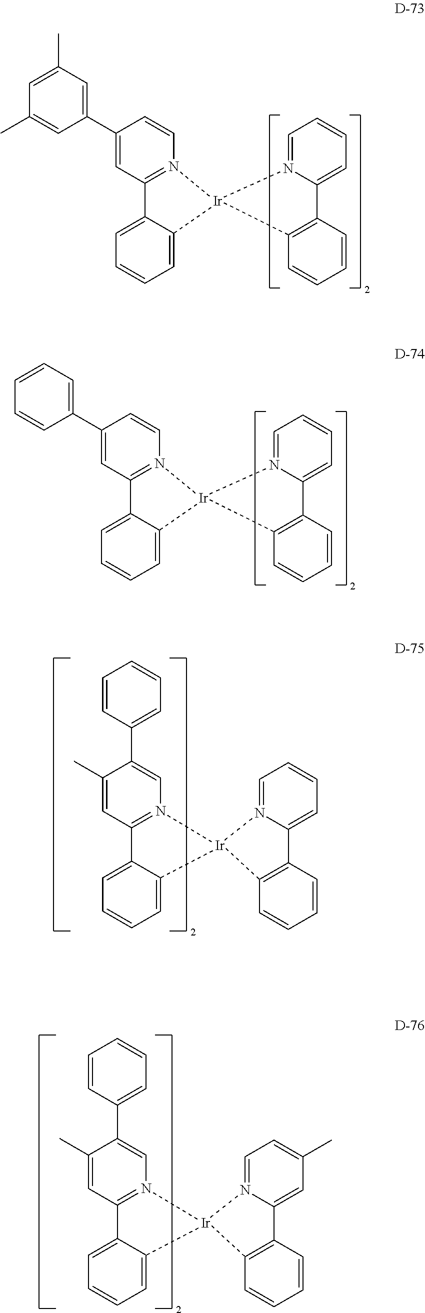

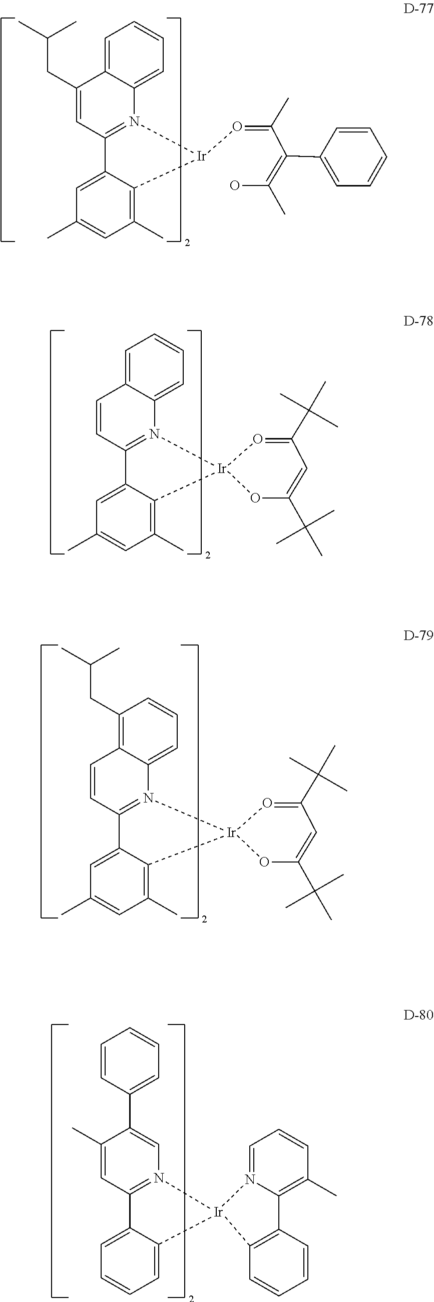

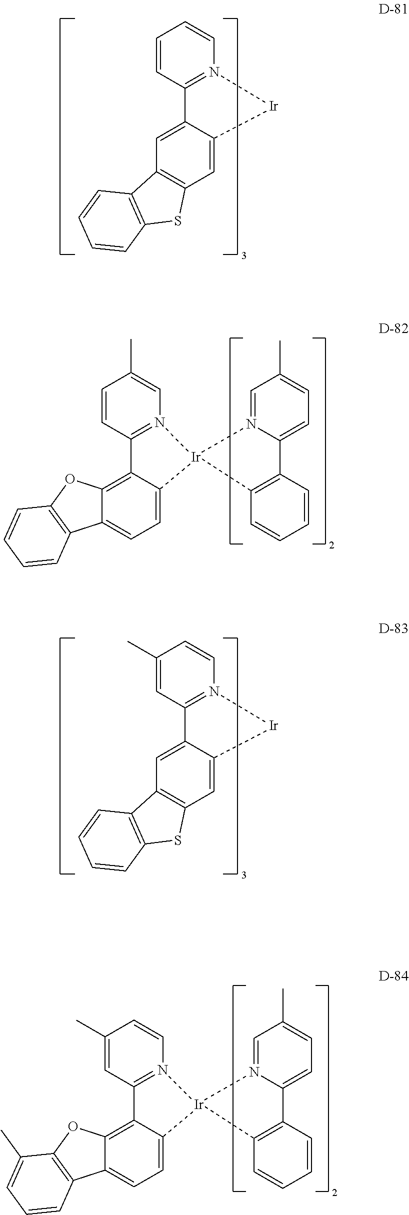

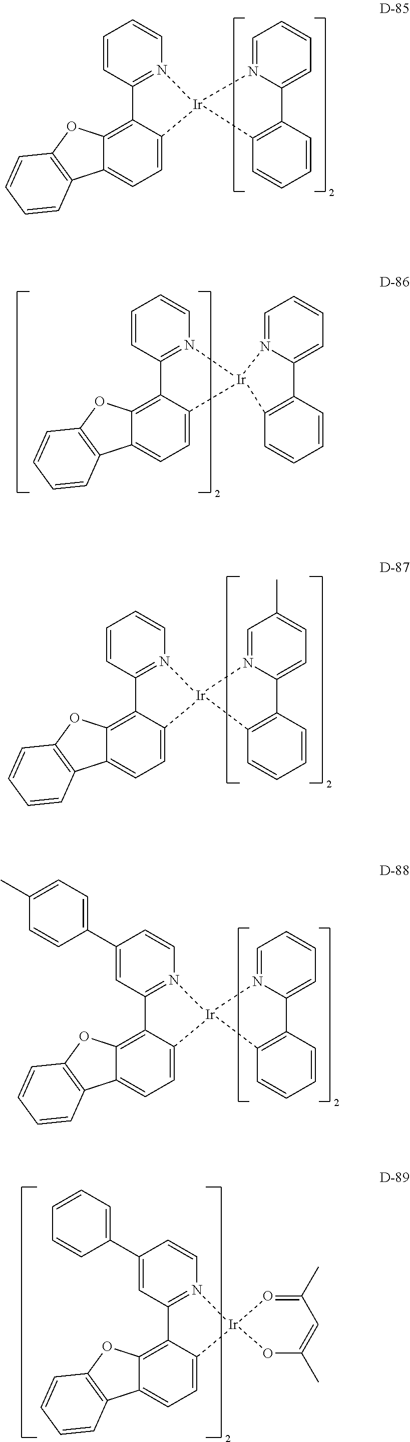

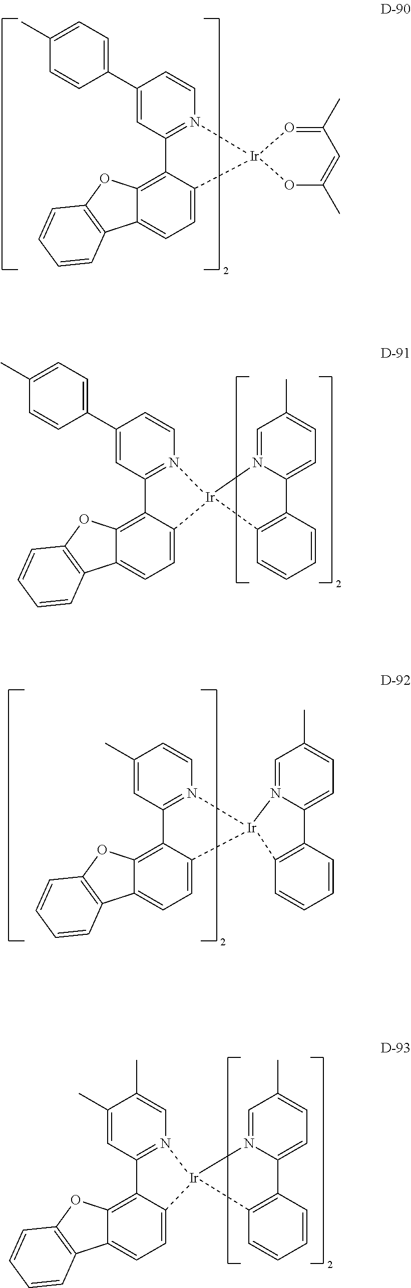

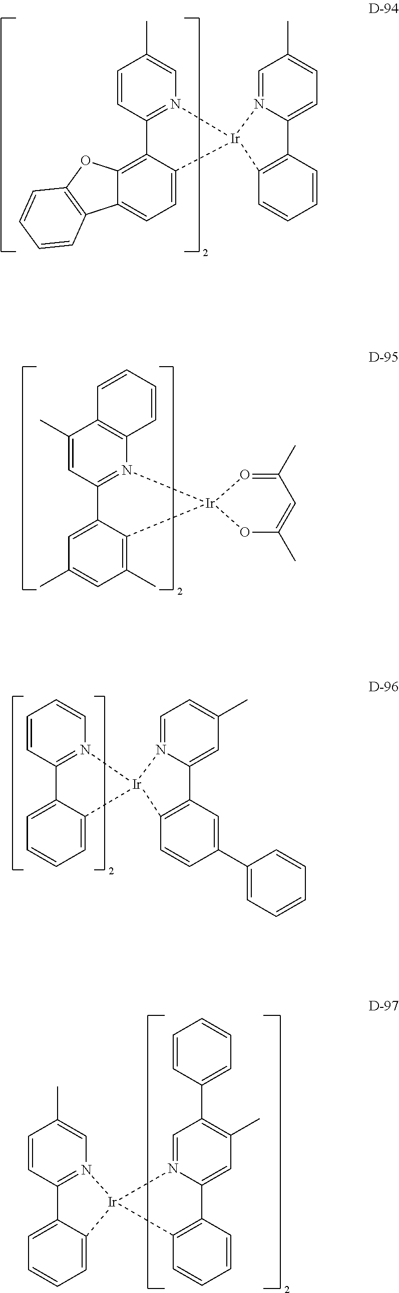

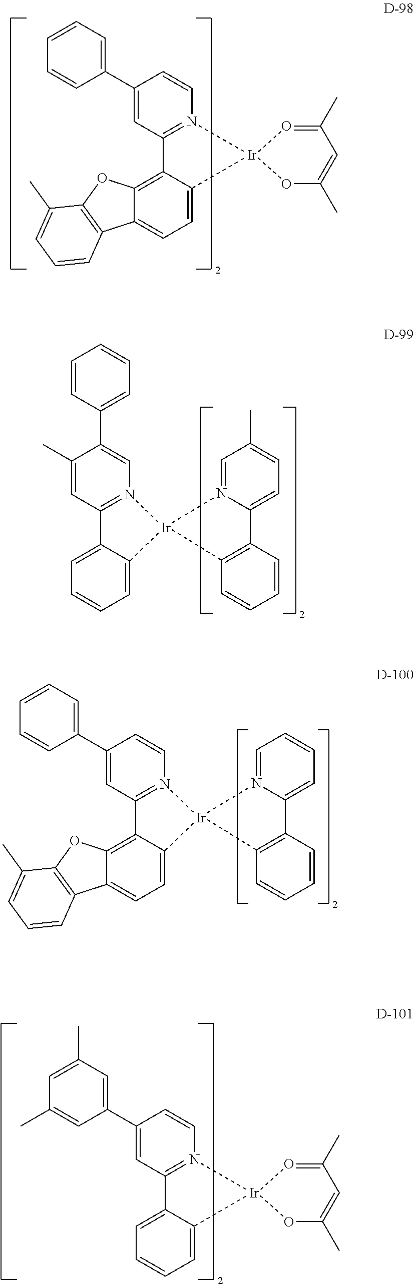

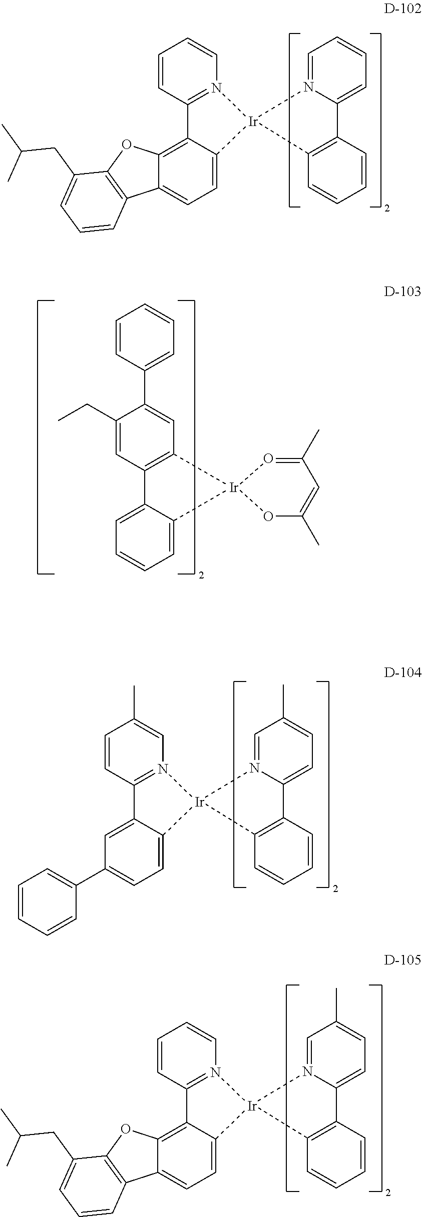

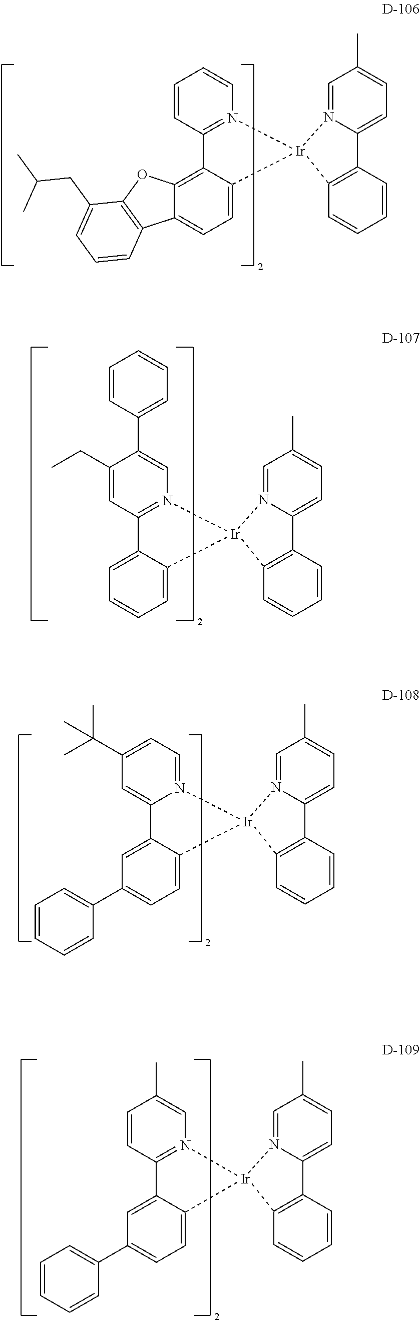

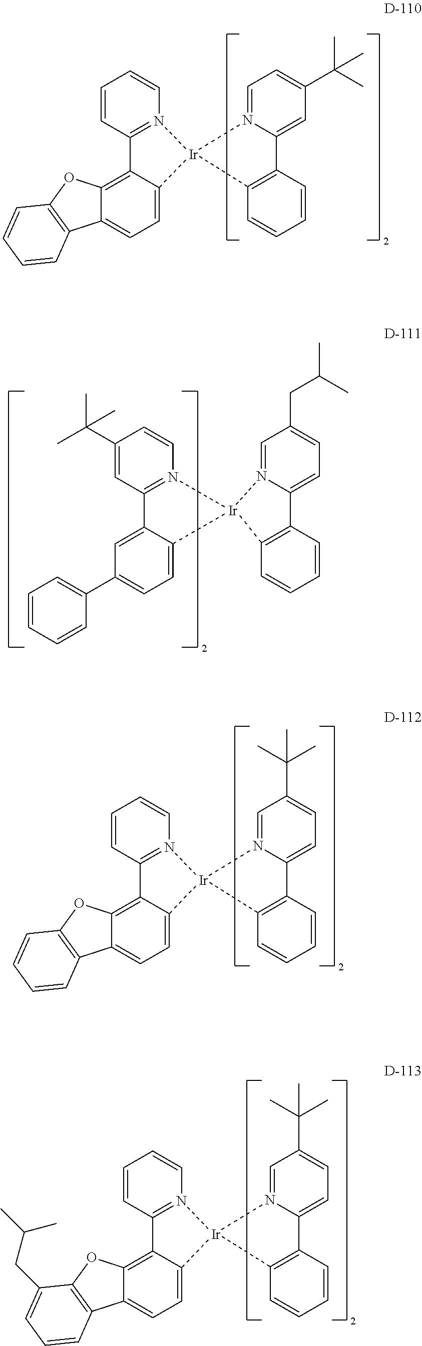

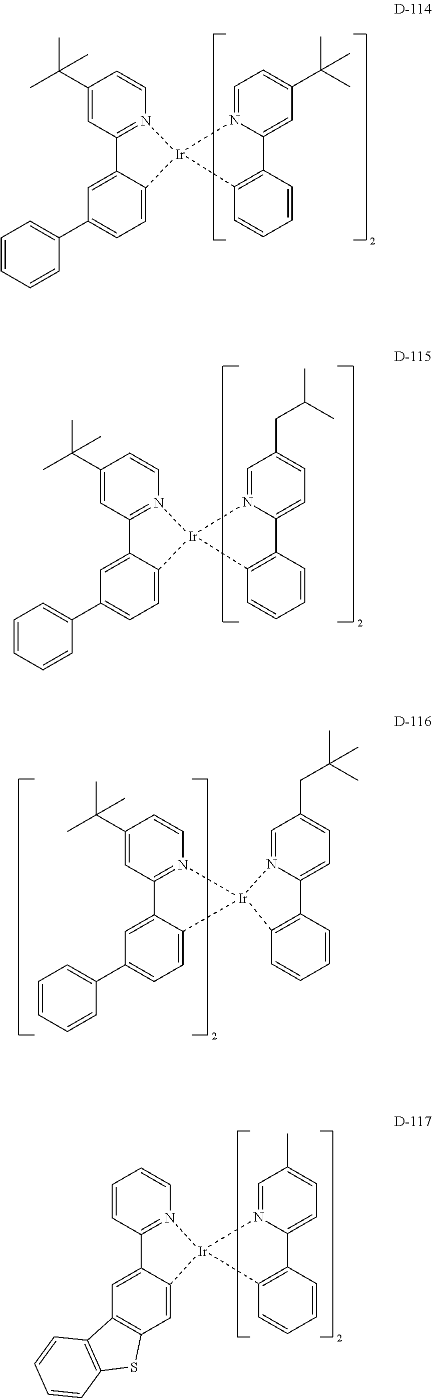

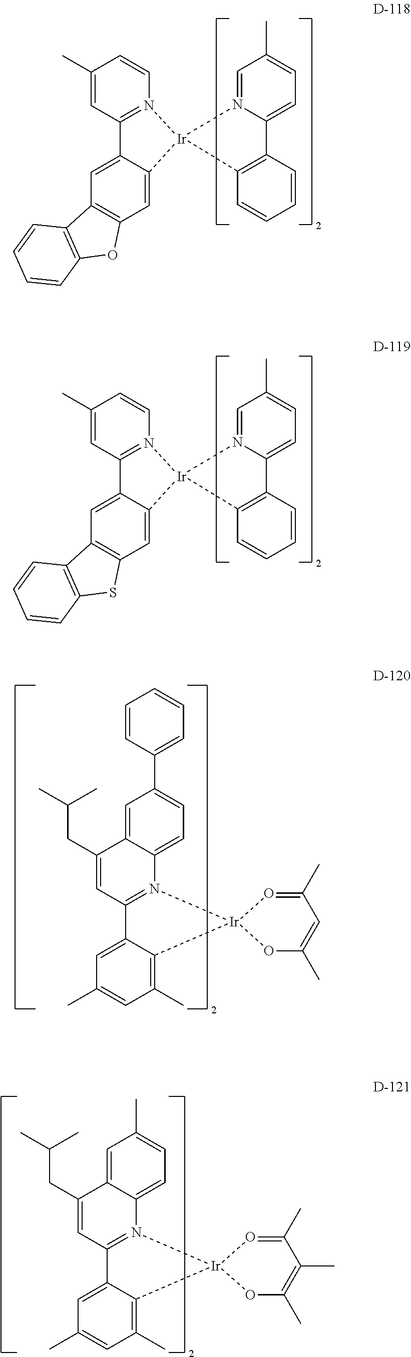

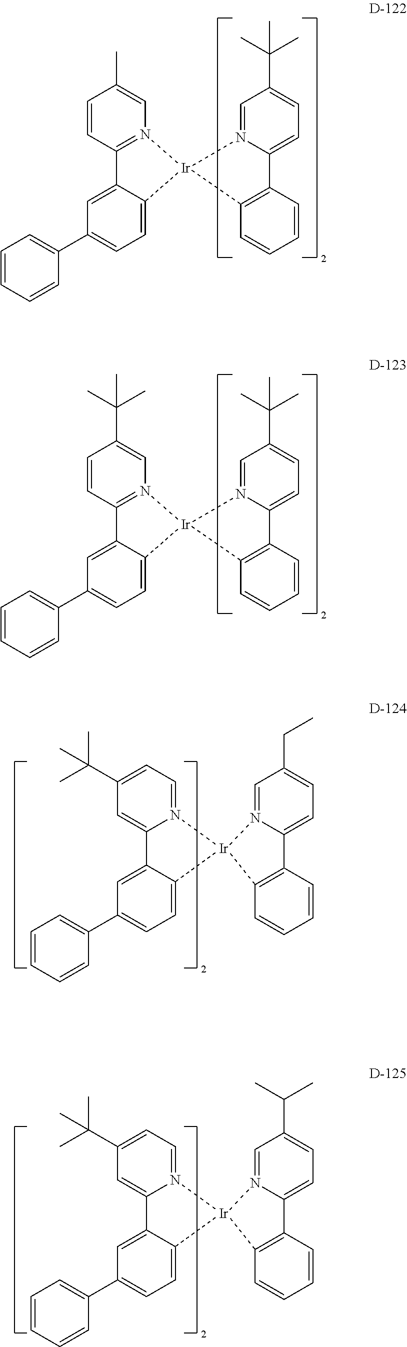

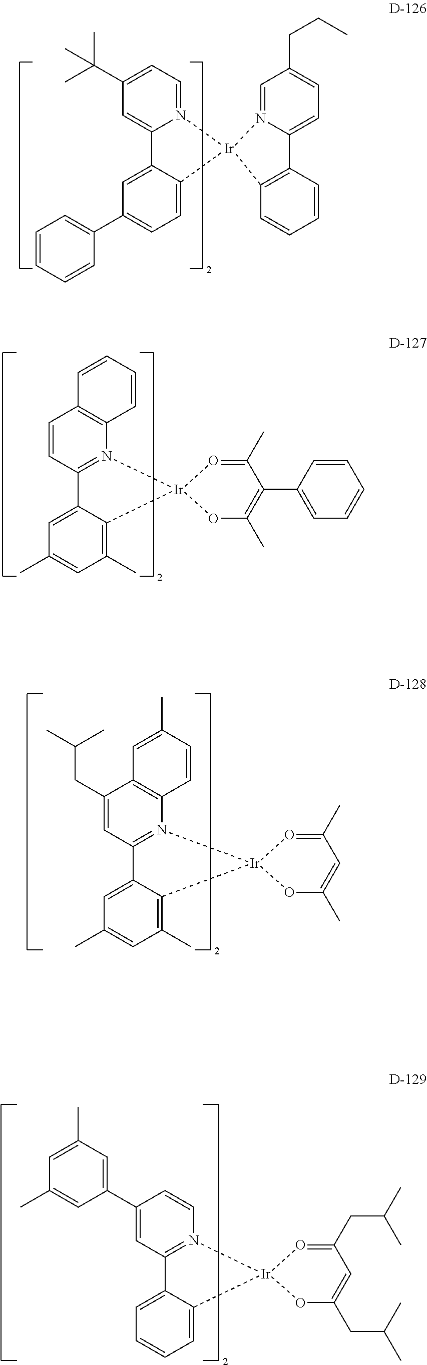

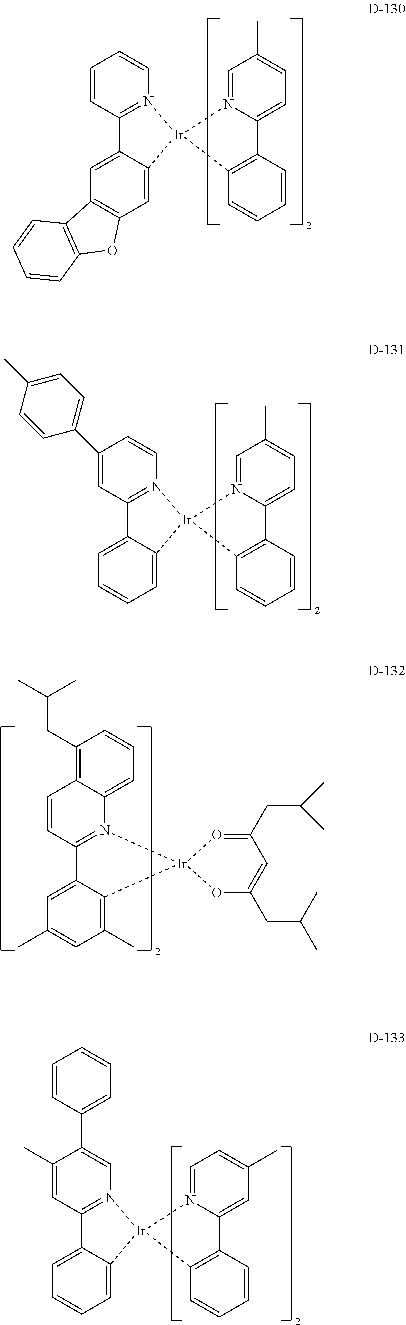

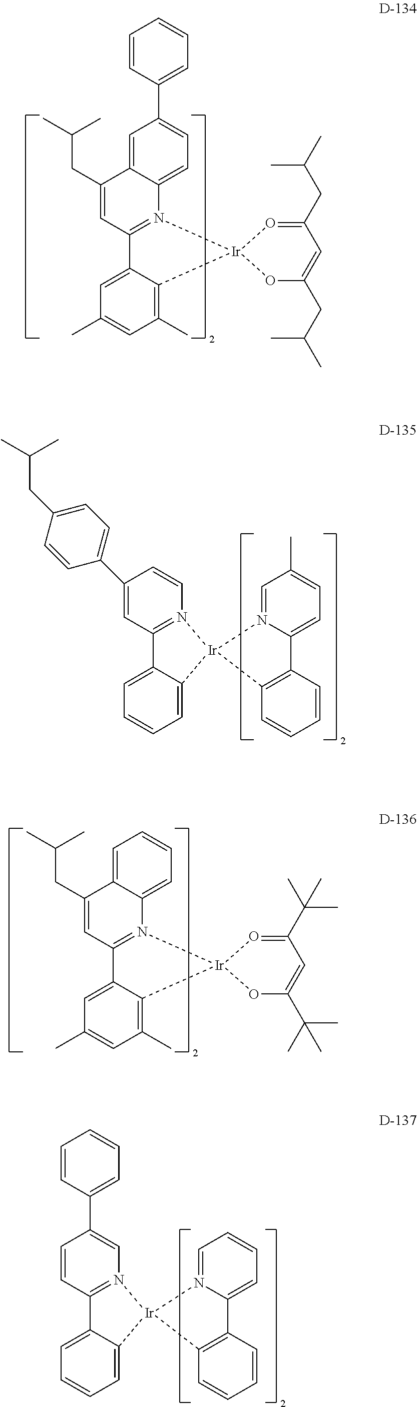

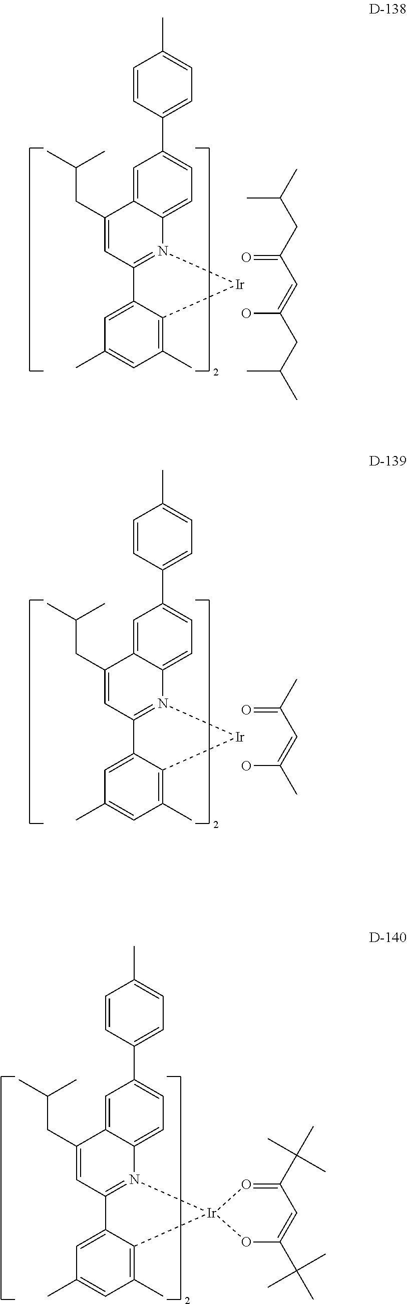

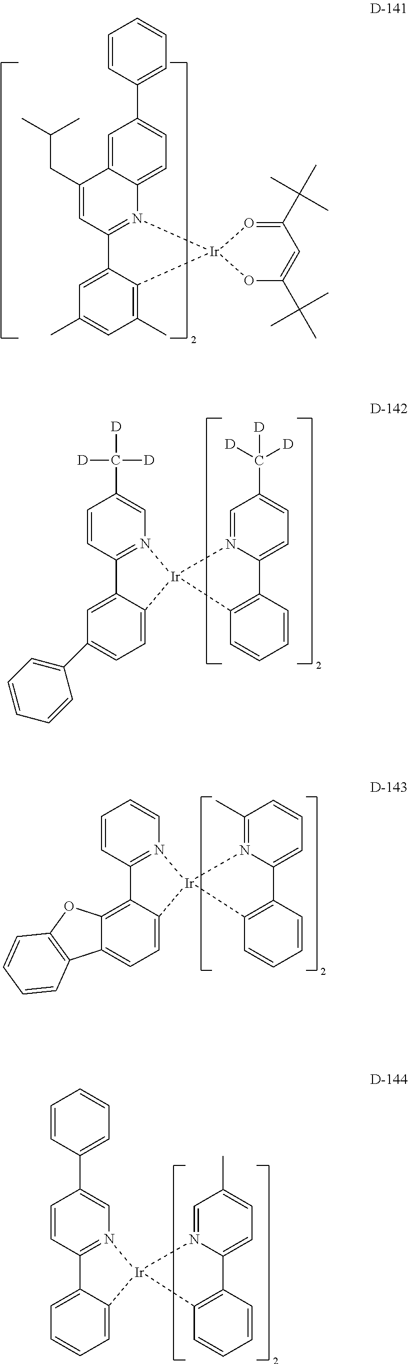

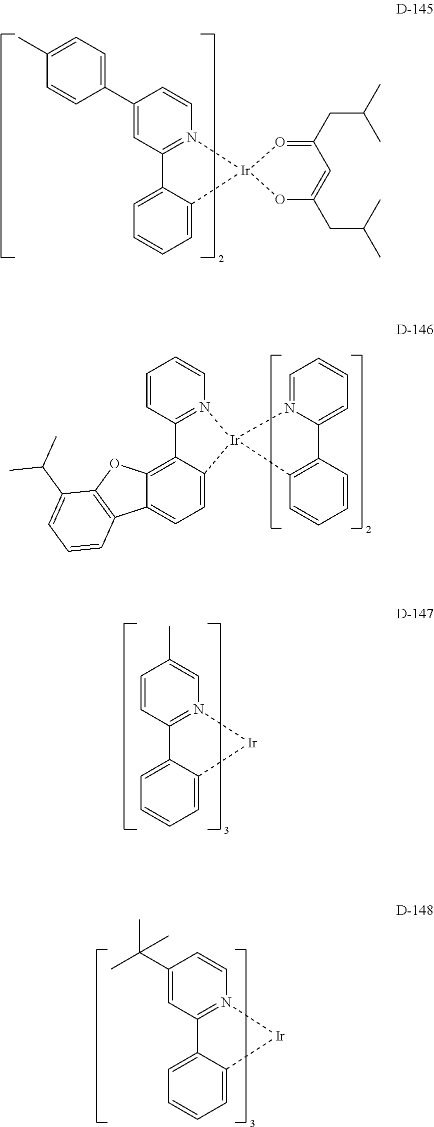

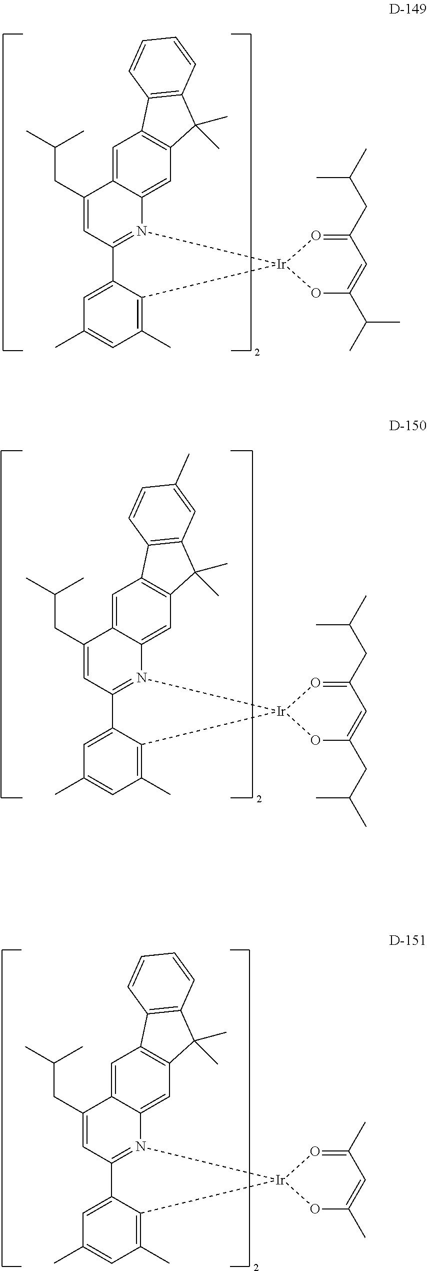

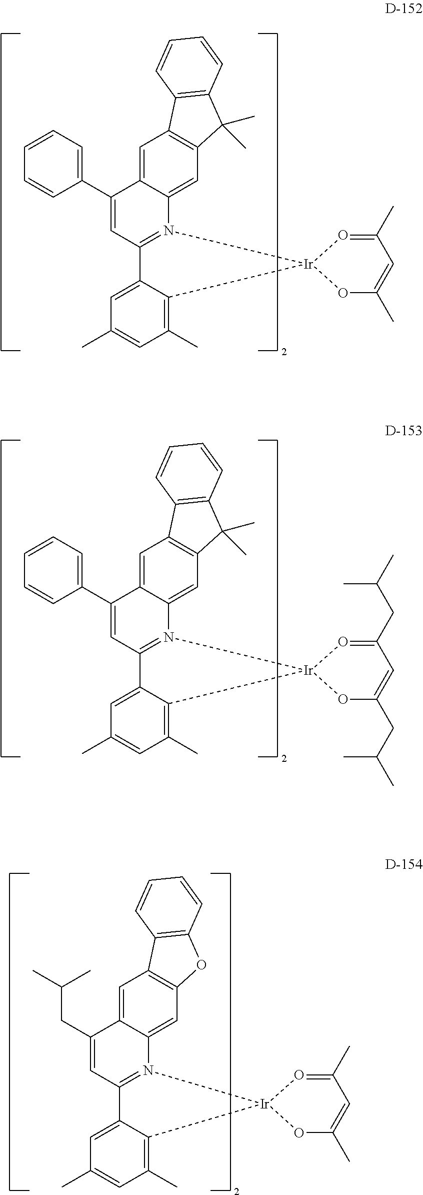

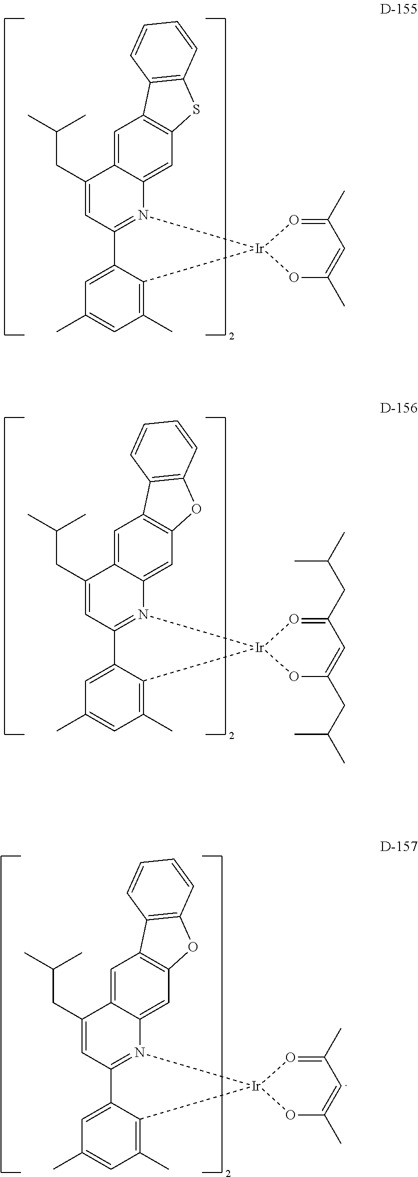

The organic electroluminescent device of the present disclosure may further comprise at least one compound selected from the group consisting of arylamine-based compounds and styrylarylamine-based compounds in the organic layer.

In addition, in the organic electroluminescent device of the present disclosure, the organic layer may further comprise at least one metal selected from the group consisting of metals of Group 1, metals of Group 2, transition metals of the 4th period, transition metals of the 5th period, lanthanides and organic metals of d-transition elements of the Periodic Table, or at least one complex compound comprising said metal.

In the organic electroluminescent device of the present disclosure, at least one layer (hereinafter, “a surface layer”) selected from a chalcogenide layer, a metal halide layer and a metal oxide layer may be preferably placed on an inner surface(s) of one or both electrodes. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer may provide operation stability for the organic electroluminescent device. Preferably, the chalcogenide includes SiO_{X}(1≤X≤2), AlO_{X}(1≤X≤1.5), SiON, SiAlON, etc.; said metal halide includes LiF, MgF₂, CaF₂, a rare earth metal fluoride, etc.; and said metal oxide includes Cs₂O, Li₂O, MgO, SrO, BaO, CaO, etc.

Between the anode and the light-emitting layer, a hole injection layer, a hole transport layer, a hole auxiliary layer, a light-emitting auxiliary layer, or an electron blocking layer, or a combination thereof may be used. Multi-layers can be used for the hole injection layer in order to lower the hole injection barrier (or hole injection voltage) from the anode to the hole transport layer or the electron blocking layer. Two compounds can be simultaneously used in each layer. The hole transport layer or the electron blocking layer may also be formed of multi-layers. The hole auxiliary layer or the light-emitting auxiliary layer may be placed between the hole transport layer and the light-emitting layer, and may control the hole transport rate. The hole auxiliary layer or the light-emitting auxiliary layer may have an effect of improving the efficiency and/or the lifespan of the organic electroluminescent device.

Between the light-emitting layer and the cathode, a layer selected from an electron buffer layer, a hole blocking layer, an electron transport layer, or an electron injection layer, or a combination thereof can be used. Multi-layers can be used for the electron buffer layer in order to control the injection of the electrons and enhance the interfacial characteristics between the light-emitting layer and the electron injection layer. Two compounds may be simultaneously used in each layer. The hole blocking layer or the electron transport layer may also be formed of multi-layers, and each layer can comprise two or more compounds.

In the organic electroluminescent device of the present disclosure, a mixed region of an electron transport compound and a reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge-generating layer to prepare an organic electroluminescent device having two or more light-emitting layers and emitting white light.

In order to form each layer of the organic electroluminescent device of the present disclosure, dry film-forming methods such as vacuum evaporation, sputtering, plasma and ion plating methods, or wet film-forming methods such as spin coating, dip coating, and flow coating methods may be used. The first and second host compounds of the present disclosure may be co-evaporated or mixture-evaporated.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

By using the organic electroluminescent device of the present disclosure, a display system or a lighting system can be produced.

Hereinafter, the preparation method of the host compounds of the present disclosure, and the properties of the device comprising the compounds will be explained in detail with reference to the representative compounds of the present disclosure. However, the present disclosure is not limited by the following examples.

### Example 1: Preparation of Compound H1-1

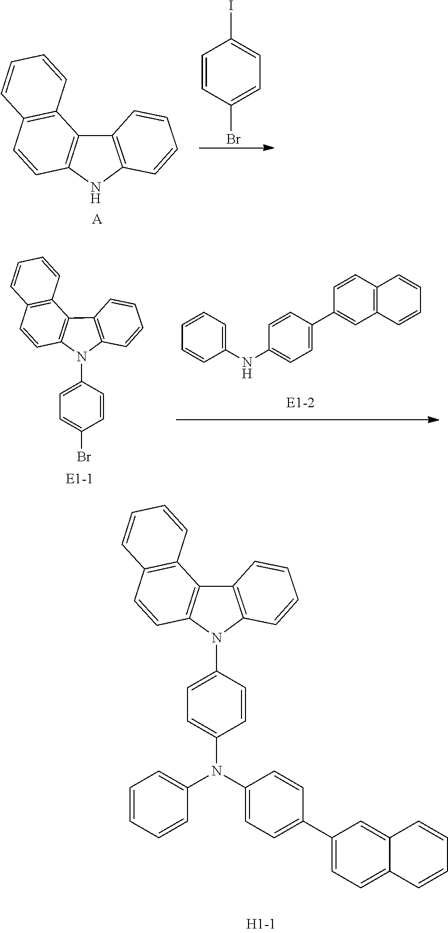

### 1) Preparation of Compound E1-1

30 g of compound A (138 mmol), 58.6 g of 1-bromo-4-iodobenzene (207 mmol), 13.1 g of CuI (69 mmol), 18.6 mL of ethylenediamine (276 mmol), 73 g of K₃PO₄ (345 mmol), and 700 mL of toluene were poured into a reaction vessel, and the mixture was stirred for 12 hours at 120° C. After completion of the reaction, the reaction product was washed with distilled water and extracted with ethyl acetate. The extracted organic layer was dried with magnesium sulfate. The solvent was removed with a rotary evaporator, and the resulting product was purified by column chromatography to obtain 42 g of compound E1-1 (yield: 82%).

### 2) Preparation of Compound H1-1

15 g of compound E1-1 (40 mmol), 10.8 g of compound E1-2 (36 mmol), 0.4 g of palladium (II) acetate (1.8 mmol), 1.8 mL of tri-tert-butyl phosphine (3.6 mmol), 10.5 g of sodium tert-butoxide (110 mmol), and 185 mL of o-xylene were poured into a reaction vessel, and the mixture was stirred under reflux for 2 hours at 150° C. After completion of the reaction, the reaction product was washed with distilled water and extracted with ethyl acetate. The extracted organic layer was dried with magnesium sulfate. The solvent was removed with a rotary evaporator, and the resulting product was purified by column chromatography to obtain 8.9 g of compound H1-1 (yield: 42%).

| | MW | UV | PL | M.P. |
|---|---|---|---|---|
| H1-1 | 586.74 | 384 nm | 403 nm | 219° C. |

### Example 2: Preparation of Compound H1-56

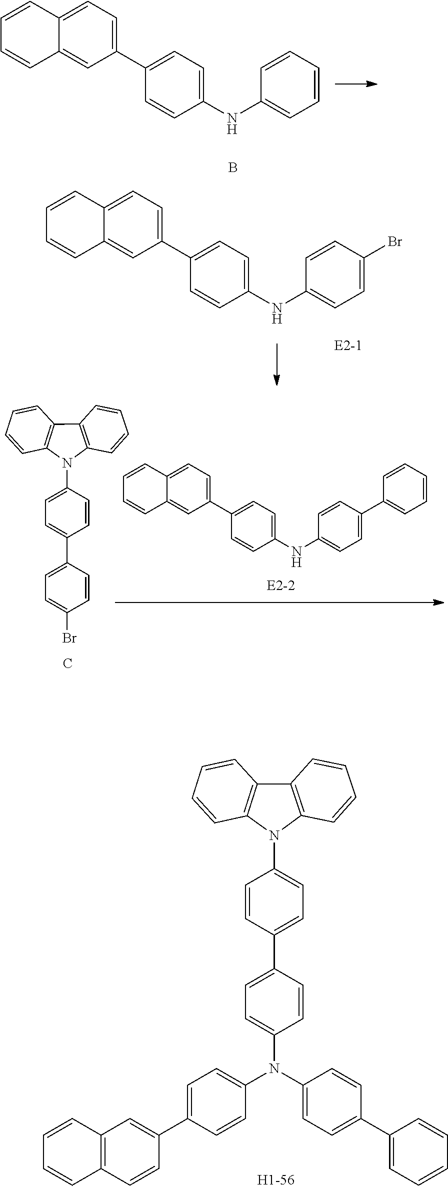

### 1) Preparation of Compound E2-1

30 g of compound B (101 mmol), and 500 ml of dimethylformamide (DMF) were introduced into a reaction vessel, and the mixture was stirred under a nitrogen atmosphere for 30 minutes at −5° C. A solution of 15.7 g of N-bromosuccinamide (NBS) (91 mmol) dissolved in 500 mL of DMF was slowly added dropwise to the reaction vessel. After completion of the reaction, the reaction product was washed with sodium thiosulfate and extracted with ethyl acetate. The extracted organic layer was dried with magnesium sulfate. The solvent was removed with a rotary evaporator, and the resulting product was purified by column chromatography to obtain 30 g of compound E2-1 (yield: 79%).

### 2) Preparation of Compound E2-2

6 g of compound E2-1 (16 mmol), 3.9 g of phenylboronic acid (32 mmol), 0.93 g of tetrakis(triphenylphosphine)palladium (0.8 mmol), 8.52 g of potassium hydrogen phosphate (40 mmol), 53 mL of toluene, 13 mL of ethanol, and 13 mL of distilled water were poured into a reaction vessel, and the mixture was stirred under reflux for 2 hours at 150° C. After completion of the reaction, the reaction product was washed with distilled water and extracted with ethyl acetate. The extracted organic layer was dried with magnesium sulfate. The solvent was removed with a rotary evaporator, and the resulting product was purified by column chromatography to obtain 5.5 g of compound E2-2 (yield: 92%).

### 3) Preparation of Compound H1-56

8.6 g of compound C (21.5 mmol), 8.0 g of compound E2-2 (21.5 mmol), 0.39 g of tris(dibenzylideneacetone)dipalladium(O) (Pd₂(dba)₃) (0.43 mmol), 0.52 g of tri(o-tolyl)phosphine (1.72 mmol), 3.1 g of sodium tert-butoxide (32.3 mmol), and 108 mL of toluene were poured into a reaction vessel, and the mixture was stirred under reflux for 3 hours at 120° C. After completion of the reaction, the reaction product was washed with distilled water and extracted with ethyl acetate. The extracted organic layer was dried with magnesium sulfate. The solvent was removed with a rotary evaporator, and the resulting product was purified by column chromatography to obtain 8.3 g of compound H1-56 (yield: 56%).

| | MW | UV | PL | M.P. |
|---|---|---|---|---|
| H1-56 | 688.87 | 393.9 nm | 410.9 nm | 150° C. |

### Example 3: Preparation of Compound H1-48

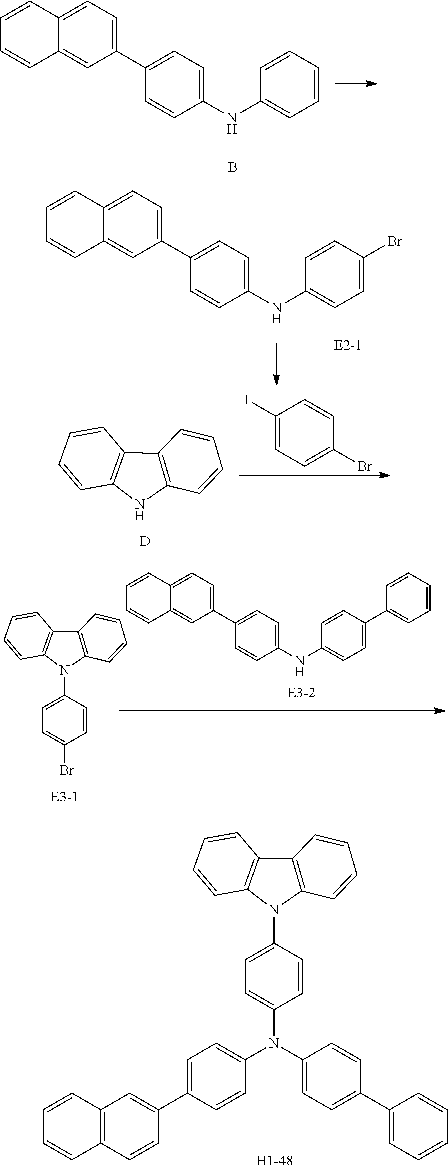

### 1) Preparation of Compound E3-1

50 g of compound D (299 mmol), 127 g of 1-bromo-4-iodobenzene (449 mmol), 28.4 g of CuI (150 mmol), 40 mL of ethylenediamine (598 mmol), 159 g of potassium hydrogen phosphate (747 mmol), and 1500 mL of toluene were poured into a reaction vessel, and the mixture was stirred for 12 hours at 120° C. After completion of the reaction, the reaction product was washed with distilled water, and extracted with ethyl acetate. The extracted organic layer was dried with magnesium sulfate. The solvent was removed with a rotary evaporator, and the resulting product was purified by column chromatography to obtain 74 g of compound E3-1 (yield: 77%).

### 2) Preparation of Compound E2-1

30 g of compound B (101 mmol), and 500 ml of dimethylformamide (DMF) were introduced into a reaction vessel, and the mixture was stirred under a nitrogen atmosphere for 30 minutes at −5° C. A solution of 15.7 g of N-bromosuccinamide (NBS) (91 mmol) dissolved in 500 mL of DMF was slowly added dropwise to the reaction vessel. After completion of the reaction, the reaction product was washed with sodium thiosulfate and extracted with ethyl acetate. The extracted organic layer was dried with magnesium sulfate. The solvent was removed with a rotary evaporator, and the resulting product was purified by column chromatography to obtain 30 g of compound E2-1 (yield: 79%).

### 3) Preparation of Compound E3-2

6 g of compound E2-1 (16 mmol), 3.9 g of phenylboronic acid (32 mmol), 0.93 g of tetrakis(triphenylphosphine)palladium (0.8 mmol), 8.52 g of potassium hydrogen phosphate (40 mmol), 53 mL of toluene, 13 mL of ethanol, and 13 mL of distilled water were poured into a reaction vessel, and the mixture was stirred under reflux for 2 hours at 125° C. After completion of the reaction, the reaction product was washed with distilled water and extracted with ethyl acetate. The extracted organic layer was dried with magnesium sulfate. The solvent was removed with a rotary evaporator, and the resulting product was purified by column chromatography to obtain 5.5 g of compound E3-2 (yield: 92%).

### 4) Preparation of Compound H1-48

4.8 g of compound E3-1 (15 mmol), 5.5 g of compound E3-2 (15 mmol), 0.27 g of tris(dibenzylideneacetone)dipalladium(O) (Pd₂(dba)₃) (0.30 mmol), 0.36 g of tri(o-tolyl)phosphine (1.0 mmol), 2.86 g of sodium tert-butoxide (30 mmol), and 75 mL of toluene were poured into a reaction vessel, and the mixture was stirred under reflux for 3 hours at 125° C. After completion of the reaction, the reaction product was washed with distilled water and extracted with ethyl acetate. The extracted organic layer was dried with magnesium sulfate. The solvent was removed with a rotary evaporator, and the resulting product was purified by column chromatography to obtain 5.5 g of compound H1-48 (yield: 60.2%).

| | MW | UV | PL | M.P. |
|---|---|---|---|---|
| H1-48 | 612.78 | 386 nm | 411 nm | 240° C. |

### Example 4: Preparation of Compound H1-22

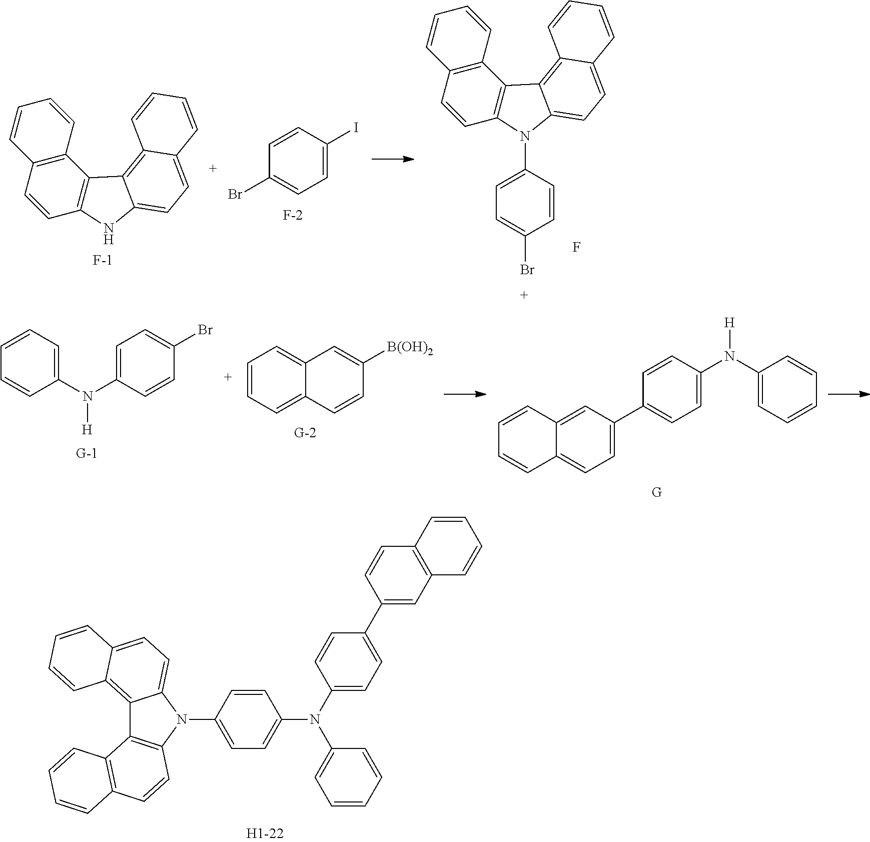

### 1) Preparation of Compound F

10 g of 7H-dibenzo[c,g]carbazole (37.4 mmol), 21.1 g of 1-bromo-4-iodobenzen (74.8 mmol), 3.56 g of CuI (18.7 mmol), 2.5 mL of ethylenediamine (37.4 mmol), 23.8 g of K₃PO₄ (112.2 mmol), and 187 mL of toluene were poured into a flask, and the mixture was dissolved and stirred under reflux for 3 hours at 120° C. After completion of the reaction, the reaction was terminated by adding water and the organic layer was extracted with ethyl acetate. The remaining moisture was removed by using magnesium sulfate. The resulting product was dried and purified by column chromatography to obtain 12 g of compound F (yield: 76.4%).

### 2) Preparation of Compound G

20 g of 4-bromo-N-phenylaniline (80.6 mmol), 17.2 g of naphthalene-2-ylboronic acid (96.7 mmol), 4.65 g of Pd(PPh₃)₄ (4.03 mmol), 33.4 g of K₂CO₃ (241.8 mmol), 240 mL of toluene, 124 mL of ethanol, and 124 mL of distilled water were poured into a flask, and the mixture was stirred under reflux for 5 hours at 120° C. The resulting solid was filtrated and purified by column chromatography to obtain 18 g of compound G (yield: 75.6%).

### 3) Preparation of Compound H1-22

6 g of compound F (14.2 mmol), 3.8 g of compound G (15.6 mmol), 159 mg of Pd(OAc)₂ (0.71 mmol), 0.7 mL of P(t-Bu)₃ (1.42 mmol), 4.2 g of NatOBu (42.6 mmol), and 70 mL of xylene were poured into a flask, and the mixture was stirred under reflux for 5 hours at 150° C. After completion of the reaction, the reaction was terminated by adding water and the organic layer was extracted with ethyl acetate. The remaining moisture was removed by using magnesium sulfate. The resulting product was dried and purified by column chromatography to obtain 5 g of compound HI-22 (yield: 55%).

| | MW | UV | PL | M.P. |
|---|---|---|---|---|
| H1-22 | 636.78 | 330 nm | 399 nm | 181° C. |

### Example 5: Preparation of Compound H1-49

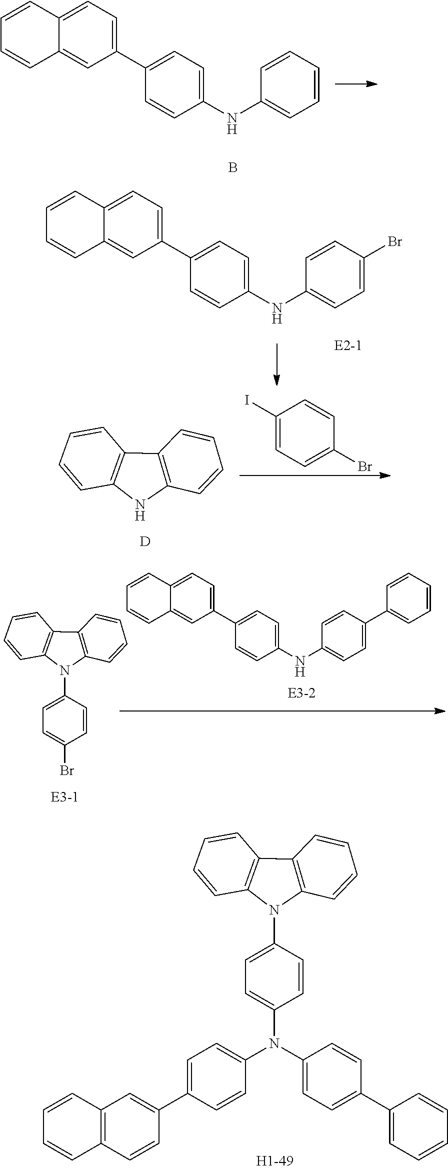

### 1) Preparation of Compound E3-1

50 g of compound D (299 mmol), 127 g of 1-bromo-4-iodobenzene (449 mmol), 28.4 g of CuI (150 mmol), 40 mL of ethylenediamine (598 mmol), 159 g of potassium hydrogen phosphate (747 mmol), and 1500 mL of toluene were poured into a reaction vessel, and the mixture was stirred for 12 hours at 120° C. After completion of the reaction, the reaction product was washed with distilled water and extracted with ethyl acetate. The extracted organic layer was dried with magnesium sulfate. The solvent was removed with a rotary evaporator, and the resulting product was purified by column chromatography to obtain 74 g of compound E3-1 (yield: 77%).

### 2) Preparation of Compound E2-1

30 g of compound B (101 mmol) and 500 ml of dimethylformamide (DMF) were introduced into a reaction vessel, and the mixture was stirred under a nitrogen atmosphere for 30 minutes at −5° C. A solution of 15.7 g of N-bromosuccinamide (NBS) (91 mmol) dissolved in 500 mL of DMF was slowly added dropwise to the reaction vessel. After completion of the reaction, the reaction product was washed with sodium thiosulfate and extracted with ethyl acetate. The extracted organic layer was dried with magnesium sulfate. The solvent was removed with a rotary evaporator, and the resulting product was purified by column chromatography to obtain 30 g of compound E2-1 (yield: 79%).

### 3) Preparation of Compound E2-3

23 g of compound E2-1 (61 mmol), 15.9 g of naphthalene-2-boronic acid (92 mmol), 3.6 g of tetrakis(triphenylphosphine) palladium (3.1 mmol), 32.6 g of potassium hydrogen phosphate (154 mmol), 300 mL of toluene, 75 mL of ethanol, and 75 mL of distilled water were poured into a reaction vessel, and the mixture was stirred under reflux for 2 hours at 125° C. After completion of the reaction, the reaction product was washed with distilled water and extracted with ethyl acetate. The extracted organic layer was dried with magnesium sulfate. The solvent was removed with a rotary evaporator, and the resulting product was purified by column chromatography to obtain 25 g of compound E2-3 (yield: 96%).

### 4) Preparation of Compound H1-49

6.5 g of compound E3-1 (20 mmol), 7.8 g of compound E2-3 (18.5 mmol), 0.27 g of palladium (II) acetate (0.9 mmol), 0.9 mL of tri-tert-butyl phosphine (1.8 mmol), 5.3 g of sodium tert-butoxide (55 mmol), and 90 mL of o-xylene were poured into a reaction vessel, and the mixture was stirred under reflux for 3 hours at 150° C. After completion of the reaction, the reaction product was washed with distilled water and extracted with ethyl acetate. The extracted organic layer was dried with magnesium sulfate. The solvent was removed with a rotary evaporator, and the resulting product was purified by column chromatography to obtain 4 g of compound H1-49 (yield: 33%).

| | MW | UV | PL | M.P. |
|---|---|---|---|---|
| H1-49 | 662.82 | 388 nm | 413 nm | 209° C. |

### Device Examples 1-1 to 1-22 and 1-24 to 1-30: Producing an OLED Device by Co-Evaporating the First and Second Host Compounds of the Present Disclosure as a Host

OLED devices were produced by using the host compounds according to the present disclosure. A transparent electrode indium tin oxide (ITO) thin film (10 Ω/sq) on a glass substrate for an OLED device (GEOMATEC CO., LTD., Japan) was subjected to an ultrasonic washing with acetone, ethanol, and distilled water, sequentially, and then was stored in isopropanol. The ITO substrate was then mounted on a substrate holder of a vacuum vapor deposition apparatus. Compound HI-1 was introduced into a cell of the vacuum vapor deposition apparatus, and then the pressure in the chamber of the apparatus was controlled to 10⁻⁶ torr. Thereafter, an electric current was applied to the cell to evaporate the above-introduced material, thereby forming a first hole injection layer having a thickness of 80 nm on the ITO substrate. Next, compound HI-2 was introduced into another cell of the vacuum vapor deposition apparatus, and was evaporated by applying an electric current to the cell, thereby forming a second hole injection layer having a thickness of 5 nm on the first hole injection layer. Compound HT-1 was then introduced into another cell of the vacuum vapor deposition apparatus, and was evaporated by applying an electric current to the cell, thereby forming a first hole transport layer having a thickness of 10 nm on the second hole injection layer. Compound HT-3 was then introduced into another cell of the vacuum vapor deposition apparatus, and was evaporated by applying an electric current to the cell, thereby forming a second hole transport layer having a thickness of 60 nm on the first hole transport layer. After forming the hole injection layer and the hole transport layer, a light-emitting layer was formed thereon as follows: The first host material shown in Table 1 and Table 2 was introduced into one cell of the vacuum vapor depositing apparatus as a host, and the second host material shown in Table 1 and Table 2 was introduced into another cell as a host, and the dopant material shown in Table 1 and Table 2 was introduced into the other cell as a dopant. The two materials were evaporated at a different rate, the two hosts were evaporated at the same rate of 1:1, and the dopant was deposited in a doping amount of 3 wt % based on the total amount of the host and dopant to form a light-emitting layer having a thickness of 40 nm on the second hole transport layer. Compound ET-1 and compound E1-1 were then introduced into the other two cells, and respectively evaporated at a rate of 1:1 to form an electron transport layer having a thickness of 30 nm on the light-emitting layer. After depositing compound E1-1 as an electron injection layer having a thickness of 2 nm on the electron transport layer, an Al cathode having a thickness of 80 nm was deposited on the electron injection layer by another vacuum vapor deposition apparatus. Thus, an OLED device was produced.

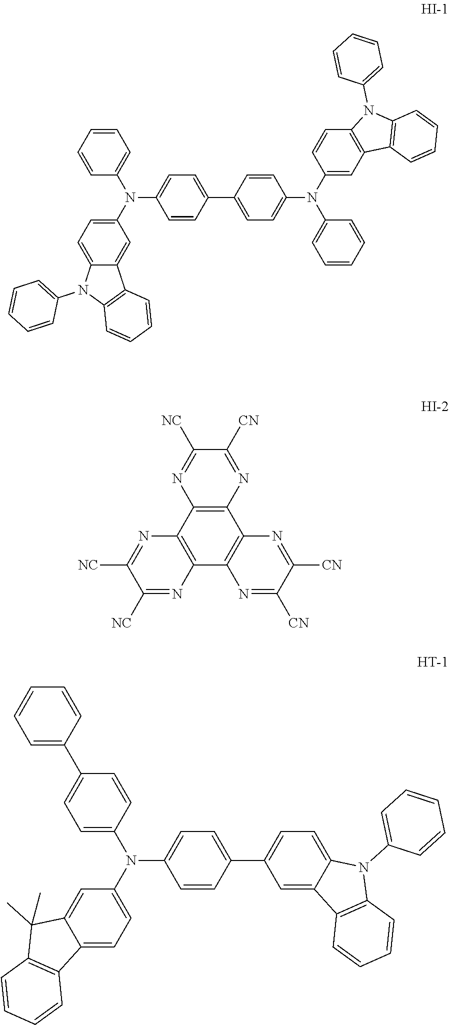 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21]

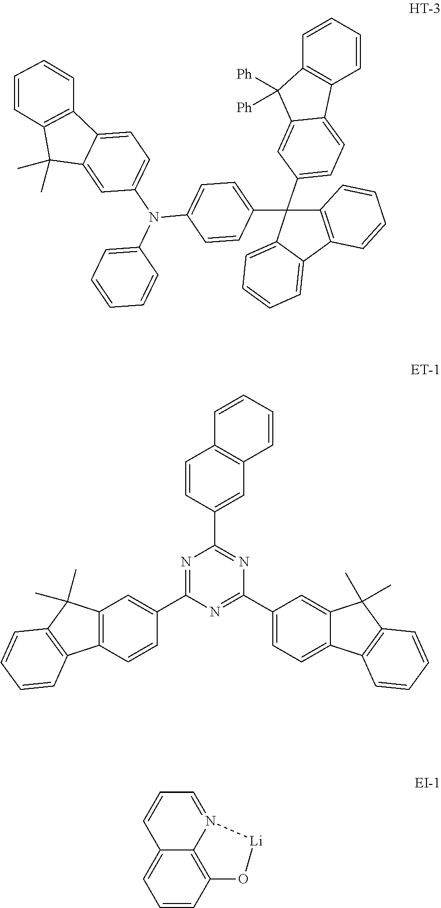

### Device Example 1-23: Producing an OLED Device by Co-Evaporating the First and Second Host Compounds of the Present Disclosure as a Host

An OLED device was produced in the same manner as in Device Example 1-1, except that a first hole injection layer having a thickness of 90 nm was formed, the first and second hosts shown in Table 1 below were used to form a light-emitting layer, and an electron transport layer having a thickness of 35 nm was formed.

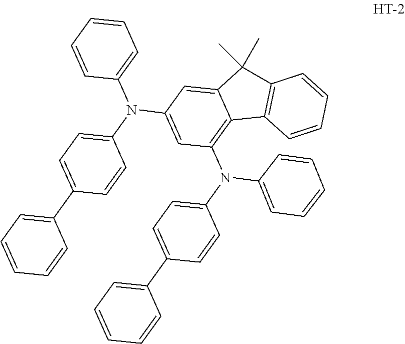 [SMILES: CC1(C)c2ccccc2-c2c(N(c3ccccc3)c3ccc(-c4ccccc4)cc3)cc(N(c3ccccc3)c3ccc(-c4ccccc4)cc3)cc21]

### Comparative Examples 1-1 to 1-14, 1-16, and 1-17: Producing an OLED Device Comprising Only the Second Host Compound of the Present Disclosure as a Host

OLED devices were produced in the same manner as in Device Example 1-1, except for using only the second host shown in Table 1 or Table 2 below as a host of the light-emitting layer.

### Comparative Example 1-15: Producing an OLED Device Comprising Only the Second Host Compound of the Present Disclosure as a Host

An OLED device was produced in the same manner as in Device Example 1-23, except for using only the second host shown in Table 1 below as a host to form a light-emitting layer.

### Comparative Examples 2-1 and 2-2: Producing an OLED Device Comprising Only the First Host Compound of the Present Disclosure as a Host

OLED devices were produced in the same manner as in Device Example 1-1, except for using only the first host shown in Table 1 below as a host of the light-emitting layer.

The lifespan (T97) in Table 1 below was measured as the time taken to be reduced from 100% to 97% of the luminance at 5,000 nits and a constant current.

TABLE 1

| | | | | | Light- |
|---|---|---|---|---|---|
| | First | Second | | Lifespan | Emitting |
| | Host | Host | Dopant | T97 (hr) | Color |
| Device Example | H1-22 | H2-6 | D-71 | 280 | Red |
| 1-1 | | | | | |
| Device Example | H1-1 | H2-6 | D-134 | 230 | Red |
| 1-2 | | | | | |
| Device Example | H1-56 | H2-6 | D-134 | 120 | Red |
| 1-3 | | | | | |
| Comparative | — | H2-6 | D-71 | 40 | Red |
| Example 1-1 | | | | | |
| Device Example | H1-22 | H2-76 | D-134 | 81 | Red |
| 1-4 | | | | | |
| Device Example | H1-1 | H2-76 | D-134 | 145 | Red |
| 1-5 | | | | | |
| Device Example | H1-60 | H2-76 | D-134 | 100 | Red |
| 1-6 | | | | | |
| Device Example | H1-56 | H2-76 | D-134 | 77 | Red |
| 1-7 | | | | | |
| Comparative | — | H2-76 | D-134 | 52 | Red |
| Example 1-2 | | | | | |
| Device Example | H1-22 | H2-75 | D-71 | 83 | Red |
| 1-8 | | | | | |
| Comparative | — | H2-75 | D-71 | 5 | Red |
| Example 1-3 | | | | | |
| Device Example | H1-22 | H2-82 | D-71 | 250 | Red |
| 1-9 | | | | | |
| Comparative | — | H2-82 | D-71 | 63 | Red |
| Example 1-4 | | | | | |
| Device Example | H1-1 | H2-3 | D-134 | 200 | Red |
| 1-10 | | | | | |
| Device Example | H1-60 | H2-3 | D-134 | 246 | Red |
| 1-11 | | | | | |
| Device Example | H1-22 | H2-10 | D-71 | 95 | Red |
| 1-12 | | | | | |
| Comparative | — | H2-10 | D-71 | 40 | Red |
| Example 1-5 | | | | | |
| Device Example | H1-22 | H2-7 | D-71 | 105 | Red |
| 1-13 | | | | | |
| Comparative | — | H2-7 | D-71 | 25 | Red |
| Example 1-6 | | | | | |
| Device Example | H1-22 | H2-8 | D-71 | 65 | Red |
| 1-14 | | | | | |
| Comparative | — | H2-8 | D-71 | 30 | Red |
| Example 1-7 | | | | | |
| Device Example | H1-22 | H2-29 | D-71 | 95 | Red |
| 1-15 | | | | | |
| Device Example | — | H2-29 | D-71 | 20 | Red |
| 1-8 | | | | | |
| Device Example | H1-22 | H2-16 | D-71 | 103 | Red |
| 1-16 | | | | | |
| Comparative | — | H2-16 | D-71 | 16 | Red |
| Example 1-9 | | | | | |
| Device Example | H1-56 | H2-170 | D-134 | 98 | Red |
| 1-17 | | | | | |
| Comparative | — | H2-170 | D-134 | 69 | Red |
| Example 1-10 | | | | | |
| Device Example | H1-56 | H2-168 | D-134 | 63 | Red |
| 1-18 | | | | | |
| Device Example | H1-22 | H2-166 | D-134 | 130 | Red |
| 1-19 | | | | | |
| Comparative | — | H2-166 | D-134 | 80 | Red |
| Example 1-11 | | | | | |
| Device Example | H1-22 | H2-81 | D-134 | 110 | Red |
| 1-20 | | | | | |
| Comparative | — | H2-81 | D-134 | 83 | Red |
| Example 1-12 | | | | | |
| Device Example | H1-56 | H2-174 | D-134 | 200 | Red |
| 1-21 | | | | | |
| Comparative | — | H2-174 | D-134 | 187 | Red |
| Example 1-13 | | | | | |
| Device Example | H1-60 | H2-173 | D-71 | 221 | Red |
| 1-22 | | | | | |
| Comparative | — | H2-173 | D-71 | 65 | Red |
| Example 1-14 | | | | | |
| Comparative | H1-22 | — | D-71 | 1 | Red |
| Example 2-1 | | | | | |
| Comparative | H1-60 | — | D-71 | X | Red |
| Example 2-2 | | | | | |
| Device Example | H1-60 | H2-96 | D-71 | 137 | Red |
| 1-23 | | | | | |
| Comparative | — | H2-96 | D-71 | 103 | Red |
| Example 1-15 | | | | | |

* Due to too low efficiency of Comparative Example 2-2 at 5,000 nits, its lifespan characteristic could not be measured.

The lifespan (T99) in Table 2 below was measured as the time taken to be reduced from 100% to 99% of the luminance at 5,000 nits and a constant current.

TABLE 2

| | | | | | Light- |
|---|---|---|---|---|---|
| | First | Second | | Lifespan | Emitting |
| | Host | Host | Dopant | T99 (hr) | Color |
| Device Example | H1-22 | H2-9 | D-134 | 72 | Red |
| 1-24 | | | | | |
| Device Example | H1-1 | H2-9 | D-134 | 90 | Red |
| 1-25 | | | | | |
| Device Example | H1-49 | H2-9 | D-134 | 134 | Red |
| 1-26 | | | | | |
| Device Example | H1-56 | H2-9 | D-134 | 62 | Red |
| 1-27 | | | | | |
| Device Example | H1-48 | H2-9 | D-134 | 97 | Red |
| 1-28 | | | | | |
| Comparative | — | H2-9 | D-134 | 8 | Red |
| Example 1-16 | | | | | |
| Device Example | H1-56 | H2-171 | D-134 | 91 | Red |
| 1-29 | | | | | |
| Device Example | H1-60 | H2-171 | D-71 | 73 | Red |
| 1-30 | | | | | |
| Comparative | — | H2-171 | D-71 | 7 | Red |
| Example 1-17 | | | | | |

### Device Examples 2-1 to 2-3: Producing an OLED Device by Co-Evaporating the First and Second Host Compounds of the Present Disclosure as a Host

OLED devices were produced in the same manner as in Device Example 1-1, except that compound HT-2 was used instead of compound HT-3 as a hole transport layer, the first and second hosts shown in Table 3 below were used to form a light-emitting layer, and an electron transport layer having a thickness of 35 nm was formed. Further, the lifespan (T99) in Table 3 below was measured in the same manner as in Table 2 above.

### Comparative Examples 3-1 to 3-3: Producing an OLED Device Comprising Only the Second Host Compound of the Present Disclosure as a Host

OLED devices were produced in the same manner as in Device Example 2-1, except for using only the second host shown in Table 3 below as a host of the light-emitting layer.

TABLE 3

| | | | | | Light- |
|---|---|---|---|---|---|
| | First | Second | | Lifespan | Emitting |
| | Host | Host | Dopant | T99 (hr) | Color |
| Device Example 2-1 | H1-60 | H2-176 | D-71 | 71 | Red |
| Comparative Example | — | H2-176 | D-71 | 33 | Red |
| 3-1 | | | | | |
| Device Example 2-2 | H1-60 | H2-177 | D-71 | 54 | Red |
| Comparative Example | — | H2-177 | D-71 | 10 | Red |
| 3-2 | | | | | |
| Device Example 2-3 | H1-60 | H2-178 | D-71 | 68 | Red |
| Comparative Example | — | H2-178 | D-71 | 2 | Red |
| 3-3 | | | | | |

From the Device Examples and the Comparative Examples above, it can be seen that the OLED device comprising the plurality of host materials of the present disclosure has improved lifespan properties, compared to the OLED device comprising only the first host material disclosed in the present disclosure or comprising only the second host material disclosed in the present disclosure.

1. A plurality of host materials comprising at least one first host compound and at least one second host compound, wherein the first host compound is represented by the following formula 1: 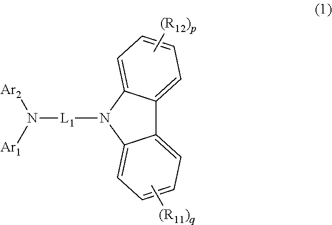
wherein
Ar₁ and Ar₂, each independently, represent a substituted or unsubstituted (C6-C30)aryl, or a substituted or unsubstituted (3- to 30-membered)heteroaryl;
L₁ represents a substituted or unsubstituted (C6-C30)arylene;
R₁₁ and R₁₂, each independently, represent hydrogen, deuterium, a substituted or unsubstituted (C1-C30)alkyl, a substituted or unsubstituted (C6-C30)aryl(C1-C30)alkyl, a substituted or unsubstituted (C6-C30)aryl, a substituted or unsubstituted (C1-C30)alkyl(C6-C30)aryl, a substituted or unsubstituted (3- to 30-membered)heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to adjacent R₁₁ and R₁₂ to form an unsubstituted benzene ring; and
p and q, each independently, represent an integer of 1 to 4, where if p and q, each independently, are an integer of 2 or more, each of R₁₁ and R₁₂ may be the same or different;
and the second host compound is represented by the following formulas 2-1 to 2-3: 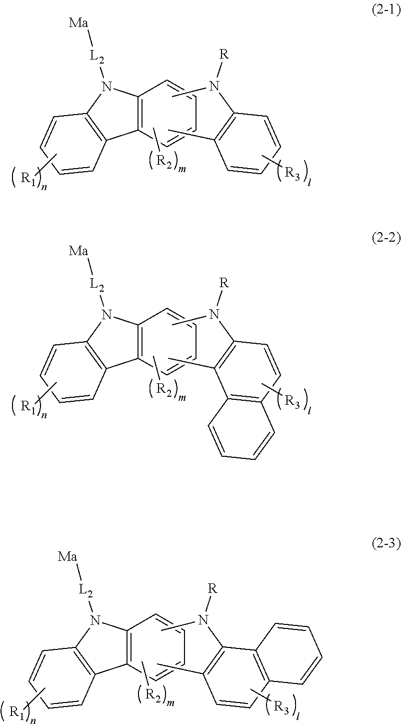
wherein
Ma represents a substituted or unsubstituted nitrogen-containing (3- to 30-membered)heteroaryl;
L₂ represents a single bond, a substituted or unsubstituted (C6-C30)arylene, or a substituted or unsubstituted nitrogen-containing (3- to 30-membered)heteroarylene;
R₁ to R₃, each independently, represent hydrogen, deuterium, a substituted or unsubstituted (C1-C30)alkyl, a substituted or unsubstituted (C6-C30)aryl(C1-C30)alkyl, a substituted or unsubstituted (C6-C30)aryl, a substituted or unsubstituted (C1-C30)alkyl(C6-C30)aryl, a substituted or unsubstituted (3- to 30-membered)heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to adjacent R₁ to R₃ to form a substituted or unsubstituted, mono- or polycyclic, (C3-C30) alicyclic or aromatic ring, or the combination thereof, whose carbon atom(s) may be replaced with at least one heteroatom selected from nitrogen, oxygen, and sulfur;
R represents hydrogen, deuterium, a substituted or unsubstituted (C1-C30)alkyl, a substituted or unsubstituted (C6-C30)aryl(C1-C30)alkyl, a substituted or unsubstituted (C6-C30)aryl, a substituted or unsubstituted (C1-C30)alkyl(C6-C30)aryl, a substituted or unsubstituted (3- to 30-membered)heteroaryl, a substituted or unsubstituted (C3-C30)cycloalkyl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
n, m, and l, each independently, represent an integer of 1 to 4, where if n, m, and l, each independently, are an integer of 2 or more, each of R₁ to R₃ may be the same or different; and
the heteroaryl(ene) contains at least one heteroatom selected from B, N, O, S, Si, and P.

2. The host materials according to claim 1, wherein formula 1 is represented by any one of the following formulas 1-1 to 1-3: 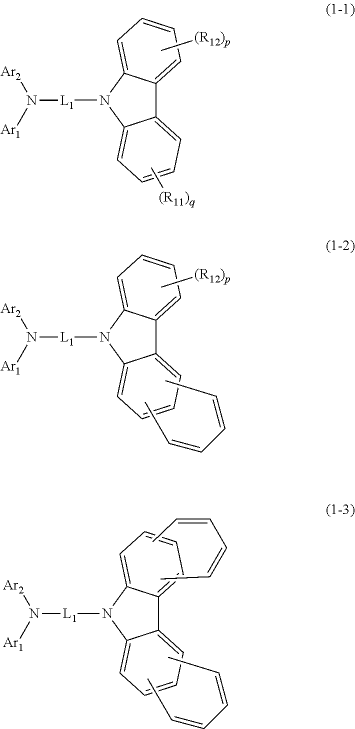
wherein Ar₁, Ar₂, L₁, R₁₁, R₁₂, p, and q are as defined in claim 1.

3. The host materials according to claim 1, wherein Ma in formula 2 is a monocyclic ring-type heteroaryl selected from the group consisting of a substituted or unsubstituted pyrrolyl, a substituted or unsubstituted imidazolyl, a substituted or unsubstituted pyrazolyl, a substituted or unsubstituted triazinyl, a substituted or unsubstituted tetrazinyl, a substituted or unsubstituted triazolyl, a substituted or unsubstituted tetrazolyl, a substituted or unsubstituted pyridyl, a substituted or unsubstituted pyrazinyl, a substituted or unsubstituted pyrimidinyl, and a substituted or unsubstituted pyridazinyl, or a fused ring-type heteroaryl selected from the group consisting of a substituted or unsubstituted benzimidazolyl, a substituted or unsubstituted isoindolyl, a substituted or unsubstituted indolyl, a substituted or unsubstituted indazolyl, a substituted or unsubstituted benzothiadiazolyl, a substituted or unsubstituted quinolyl, a substituted or unsubstituted isoquinolyl, a substituted or unsubstituted cinnolinyl, a substituted or unsubstituted quinazolinyl, a substituted or unsubstituted naphthyridinyl, a substituted or unsubstituted quinoxalinyl, a substituted or unsubstituted carbazolyl, and a substituted or unsubstituted phenanthridinyl.

4. The host materials according to claim 1, wherein the substituents of the substituted (C1-C30)alkyl, the substituted (C6-C30)aryl(ene), the substituted (3- to 30-membered)heteroaryl(ene), the substituted (C6-C30)aryl(C1-C30)alkyl, the substituted (C1-C30)alkyl(C6-C30)aryl, the substituted (C3-C30)cycloalkyl, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di-(C1-C30)alkylamino, the substituted mono- or di-(C6-C30)arylamino, the substituted (C1-C30)alkyl(C6-C30)arylamino, the substituted nitrogen-containing (3- to 30-membered)heteroaryl, and the substituted mono- or polycyclic, (C3-C30) alicyclic or aromatic ring, or the combination thereof, in Ar₁, Ar₂, L₁, R₁₁, R₁₂, Ma, L₂, R₁ to R₃, and R, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered) heterocycloalkyl; a (C6-C30)aryloxy; a (C6-C30)arylthio; a (C6-C30)aryl unsubstituted or substituted with a cyano; a (5- to 30-membered)heteroaryl unsubstituted or substituted with a (C1-C30)alkyl or a (C6-C30)aryl; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino; a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

5. The host materials according to claim 1, wherein the first host compound represented by formula 1 is selected from the group consisting of:

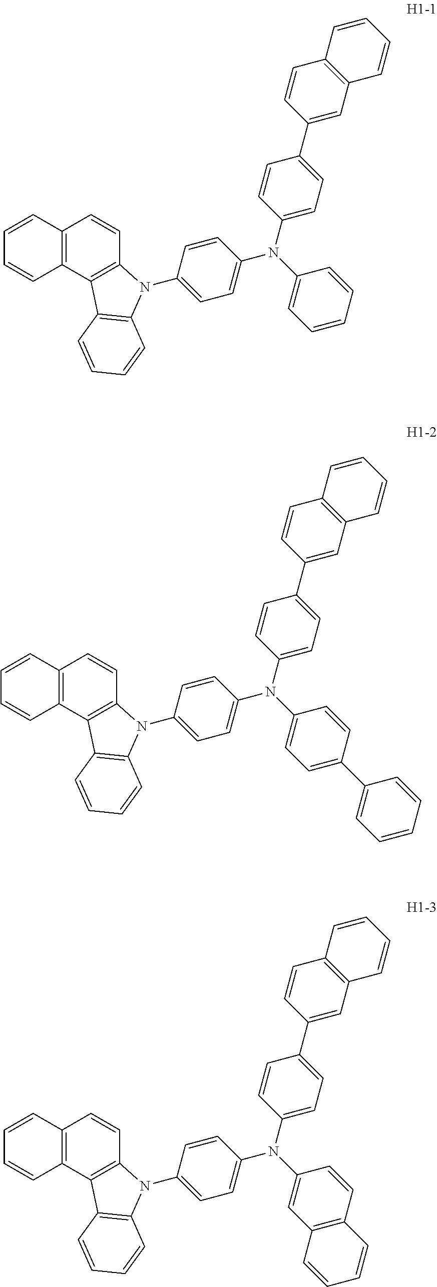 [SMILES: c1ccc(N(c2ccc(-c3ccc4ccccc4c3)cc2)c2ccc(-n3c4ccccc4c4c5ccccc5ccc43)cc2)cc1 ; c1ccc(-c2ccc(N(c3ccc(-c4ccc5ccccc5c4)cc3)c3ccc(-n4c5ccccc5c5c6ccccc6ccc54)cc3)cc2)cc1 ; c1ccc2cc(-c3ccc(N(c4ccc(-n5c6ccccc6c6c7ccccc7ccc65)cc4)c4ccc5ccccc5c4)cc3)ccc2c1]

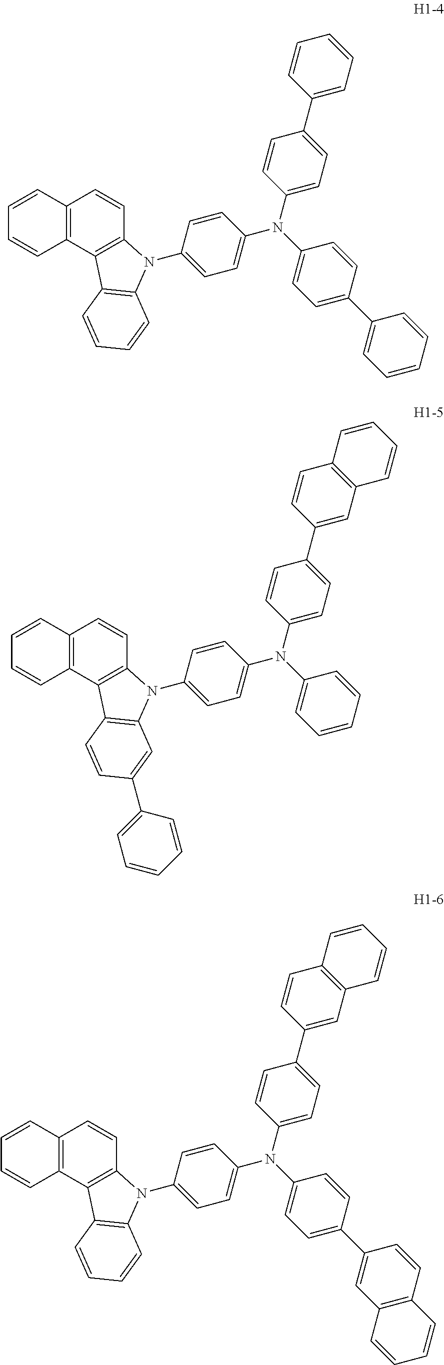 [SMILES: c1ccc(-c2ccc(N(c3ccc(-c4ccccc4)cc3)c3ccc(-n4c5ccccc5c5c6ccccc6ccc54)cc3)cc2)cc1 ; c1ccc(-c2ccc3c4c5ccccc5ccc4n(-c4ccc(N(c5ccccc5)c5ccc(-c6ccc7ccccc7c6)cc5)cc4)c3c2)cc1 ; c1ccc2cc(-c3ccc(N(c4ccc(-c5ccc6ccccc6c5)cc4)c4ccc(-n5c6ccccc6c6c7ccccc7ccc65)cc4)cc3)ccc2c1]

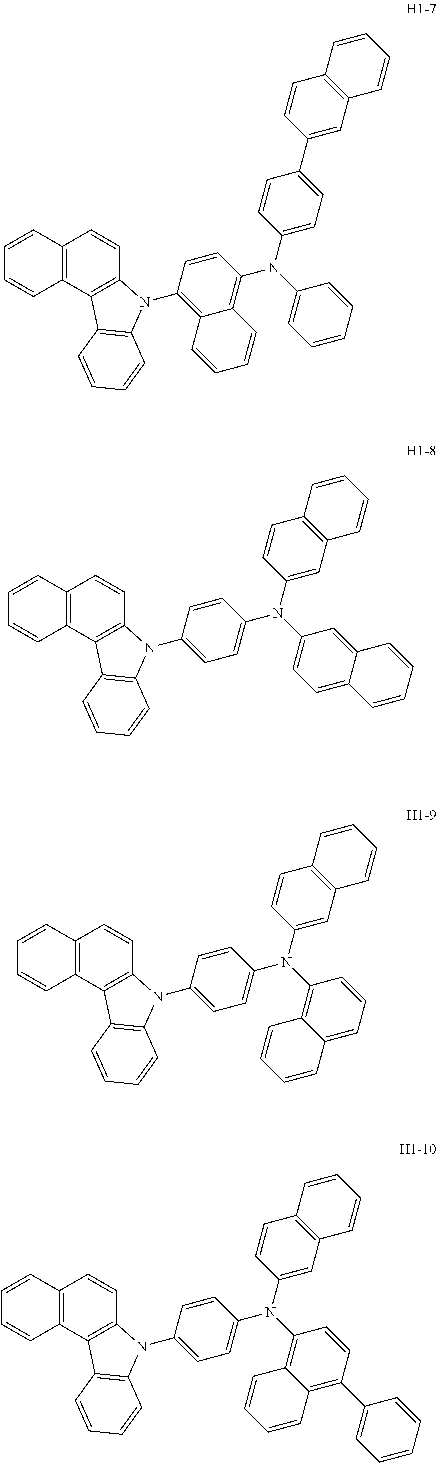 [SMILES: c1ccc(N(c2ccc(-c3ccc4ccccc4c3)cc2)c2ccc(-n3c4ccccc4c4c5ccccc5ccc43)c3ccccc23)cc1 ; c1ccc2cc(N(c3ccc(-n4c5ccccc5c5c6ccccc6ccc54)cc3)c3ccc4ccccc4c3)ccc2c1 ; c1ccc2cc(N(c3ccc(-n4c5ccccc5c5c6ccccc6ccc54)cc3)c3cccc4ccccc34)ccc2c1 ; c1ccc(-c2ccc(N(c3ccc(-n4c5ccccc5c5c6ccccc6ccc54)cc3)c3ccc4ccccc4c3)c3ccccc23)cc1]

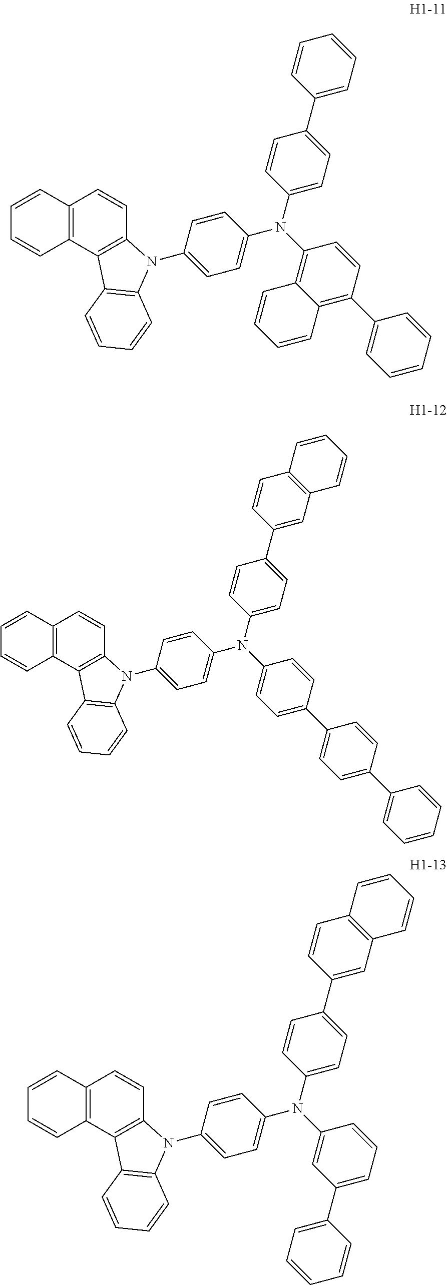

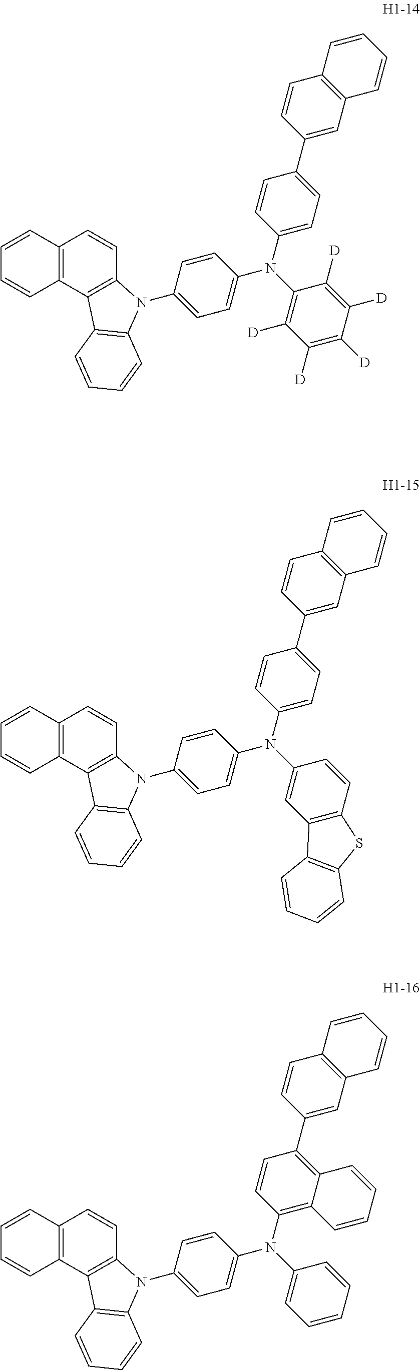 [SMILES: [2H]c1c([2H])c([2H])c(N(c2ccc(-c3ccc4ccccc4c3)cc2)c2ccc(-n3c4ccccc4c4c5ccccc5ccc43)cc2)c([2H])c1[2H] ; c1ccc2cc(-c3ccc(N(c4ccc(-n5c6ccccc6c6c7ccccc7ccc65)cc4)c4ccc5sc6ccccc6c5c4)cc3)ccc2c1 ; c1ccc(N(c2ccc(-n3c4ccccc4c4c5ccccc5ccc43)cc2)c2ccc(-c3ccc4ccccc4c3)c3ccccc23)cc1]

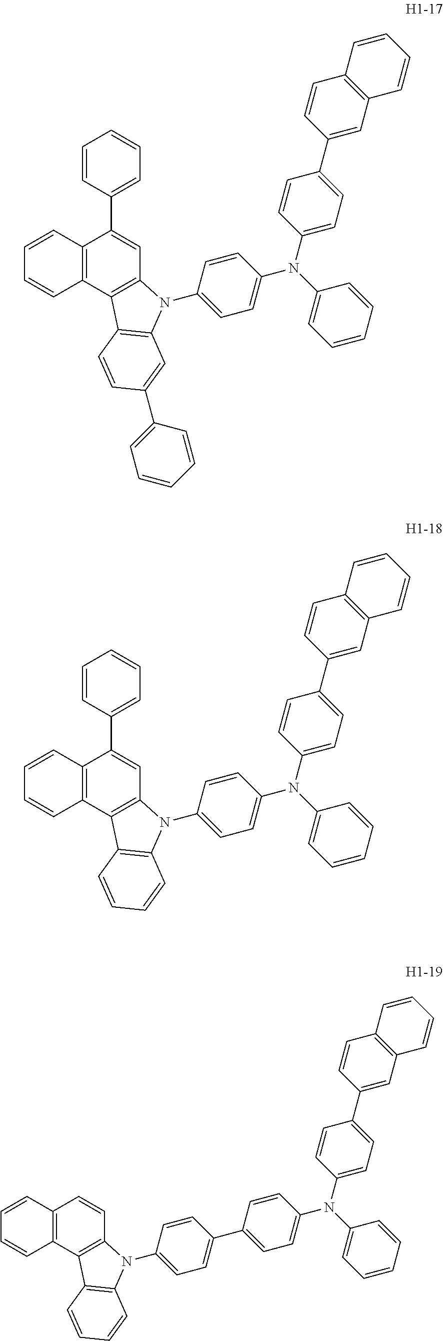 [SMILES: c1ccc(-c2ccc3c4c5ccccc5c(-c5ccccc5)cc4n(-c4ccc(N(c5ccccc5)c5ccc(-c6ccc7ccccc7c6)cc5)cc4)c3c2)cc1 ; c1ccc(-c2cc3c(c4ccccc24)c2ccccc2n3-c2ccc(N(c3ccccc3)c3ccc(-c4ccc5ccccc5c4)cc3)cc2)cc1 ; c1ccc(N(c2ccc(-c3ccc(-n4c5ccccc5c5c6ccccc6ccc54)cc3)cc2)c2ccc(-c3ccc4ccccc4c3)cc2)cc1]

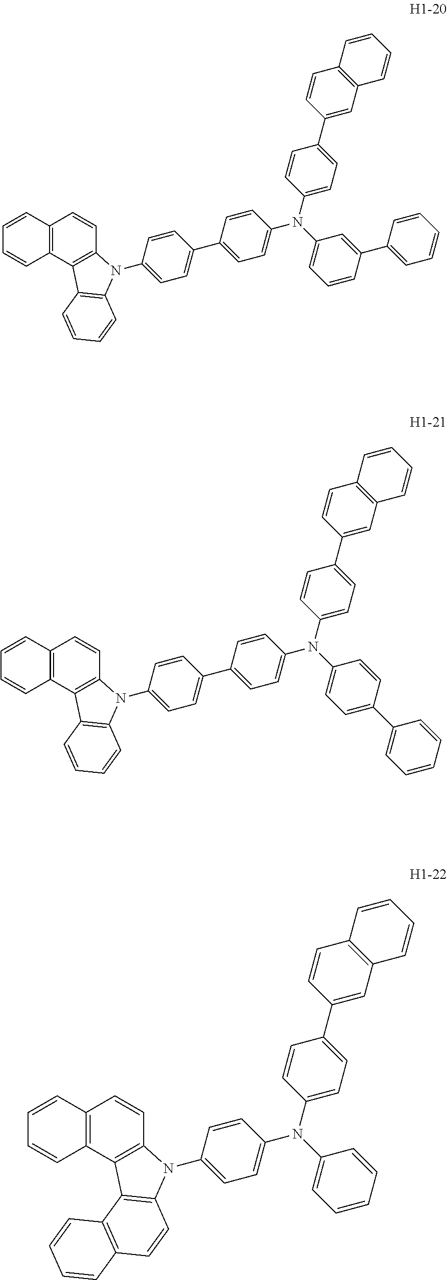 [SMILES: c1ccc(-c2cccc(N(c3ccc(-c4ccc(-n5c6ccccc6c6c7ccccc7ccc65)cc4)cc3)c3ccc(-c4ccc5ccccc5c4)cc3)c2)cc1 ; c1ccc(-c2ccc(N(c3ccc(-c4ccc(-n5c6ccccc6c6c7ccccc7ccc65)cc4)cc3)c3ccc(-c4ccc5ccccc5c4)cc3)cc2)cc1 ; c1ccc(N(c2ccc(-c3ccc4ccccc4c3)cc2)c2ccc(-n3c4ccc5ccccc5c4c4c5ccccc5ccc43)cc2)cc1]

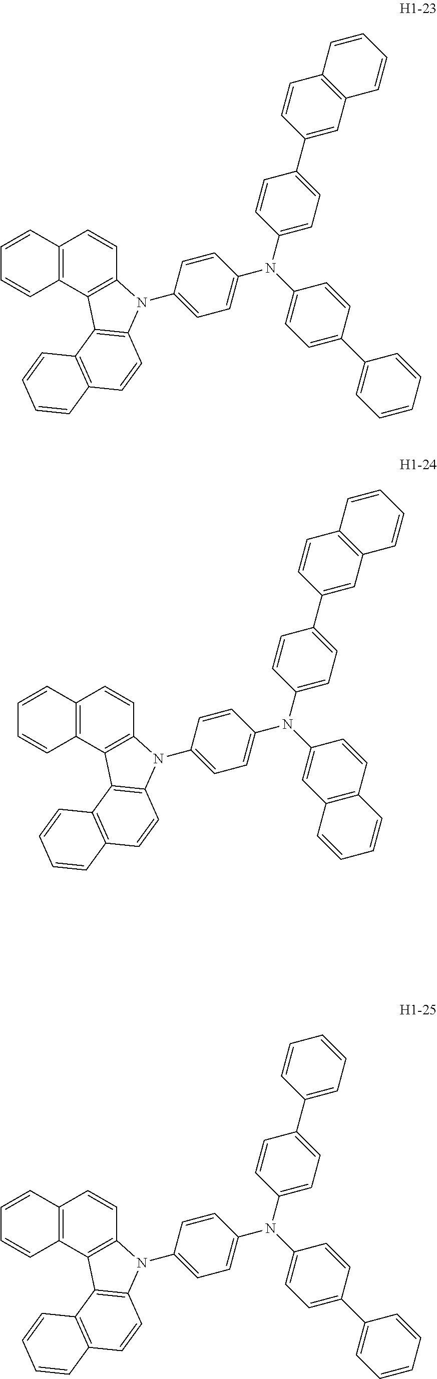 [SMILES: c1ccc(-c2ccc(N(c3ccc(-c4ccc5ccccc5c4)cc3)c3ccc(-n4c5ccc6ccccc6c5c5c6ccccc6ccc54)cc3)cc2)cc1 ; c1ccc2cc(-c3ccc(N(c4ccc(-n5c6ccc7ccccc7c6c6c7ccccc7ccc65)cc4)c4ccc5ccccc5c4)cc3)ccc2c1 ; c1ccc(-c2ccc(N(c3ccc(-c4ccccc4)cc3)c3ccc(-n4c5ccc6ccccc6c5c5c6ccccc6ccc54)cc3)cc2)cc1]

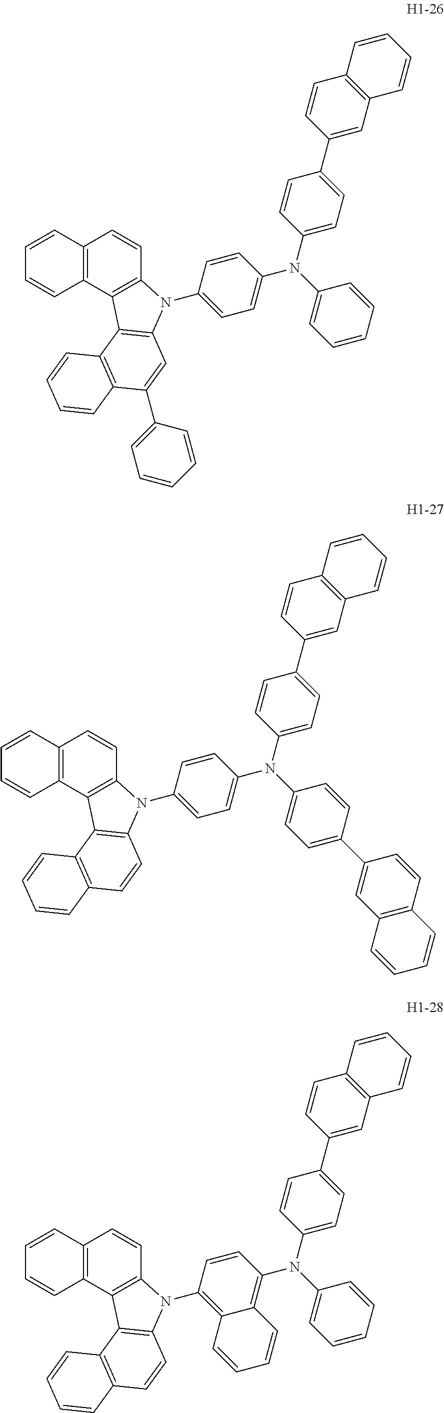 [SMILES: c1ccc(-c2cc3c(c4ccccc24)c2c4ccccc4ccc2n3-c2ccc(N(c3ccccc3)c3ccc(-c4ccc5ccccc5c4)cc3)cc2)cc1 ; c1ccc2cc(-c3ccc(N(c4ccc(-c5ccc6ccccc6c5)cc4)c4ccc(-n5c6ccc7ccccc7c6c6c7ccccc7ccc65)cc4)cc3)ccc2c1 ; c1ccc(N(c2ccc(-c3ccc4ccccc4c3)cc2)c2ccc(-n3c4ccc5ccccc5c4c4c5ccccc5ccc43)c3ccccc23)cc1]

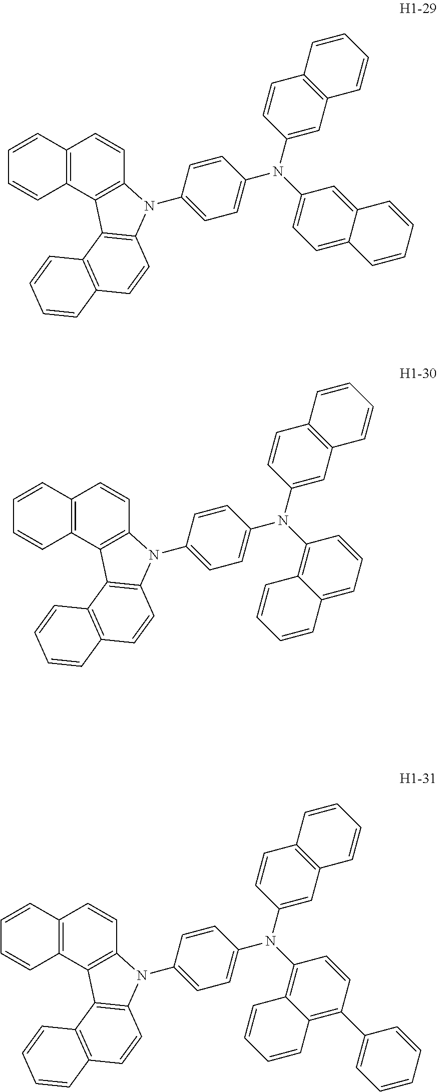 [SMILES: c1ccc2cc(N(c3ccc(-n4c5ccc6ccccc6c5c5c6ccccc6ccc54)cc3)c3ccc4ccccc4c3)ccc2c1 ; c1ccc2cc(N(c3ccc(-n4c5ccc6ccccc6c5c5c6ccccc6ccc54)cc3)c3cccc4ccccc34)ccc2c1 ; c1ccc(-c2ccc(N(c3ccc(-n4c5ccc6ccccc6c5c5c6ccccc6ccc54)cc3)c3ccc4ccccc4c3)c3ccccc23)cc1]

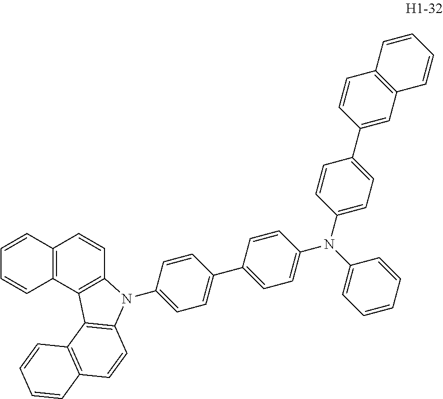 [SMILES: c1ccc(N(c2ccc(-c3ccc(-n4c5ccc6ccccc6c5c5c6ccccc6ccc54)cc3)cc2)c2ccc(-c3ccc4ccccc4c3)cc2)cc1]

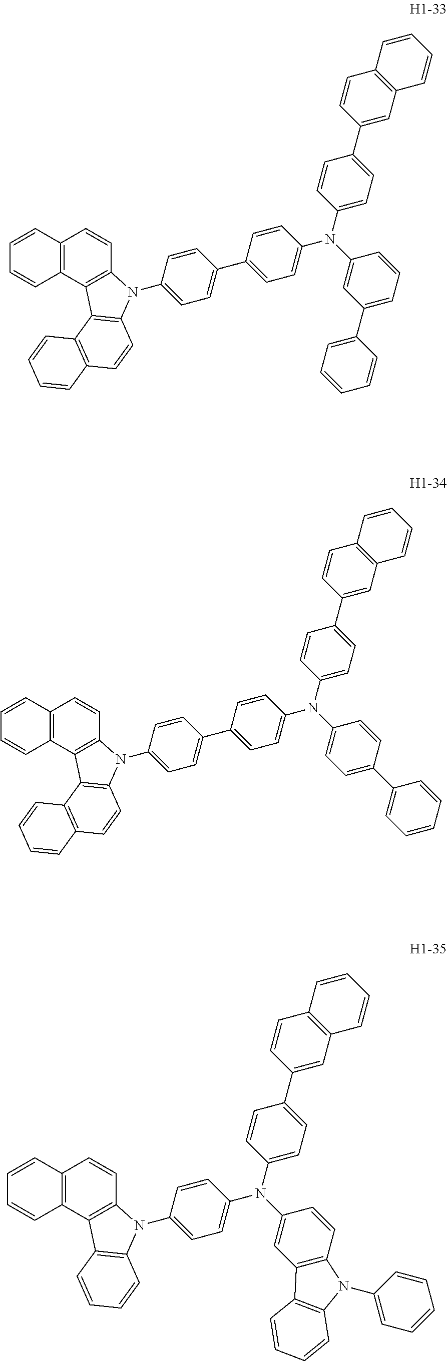 [SMILES: c1ccc(-c2cccc(N(c3ccc(-c4ccc(-n5c6ccc7ccccc7c6c6c7ccccc7ccc65)cc4)cc3)c3ccc(-c4ccc5ccccc5c4)cc3)c2)cc1 ; c1ccc(-c2ccc(N(c3ccc(-c4ccc(-n5c6ccc7ccccc7c6c6c7ccccc7ccc65)cc4)cc3)c3ccc(-c4ccc5ccccc5c4)cc3)cc2)cc1 ; c1ccc(-n2c3ccccc3c3cc(N(c4ccc(-c5ccc6ccccc6c5)cc4)c4ccc(-n5c6ccccc6c6c7ccccc7ccc65)cc4)ccc32)cc1]

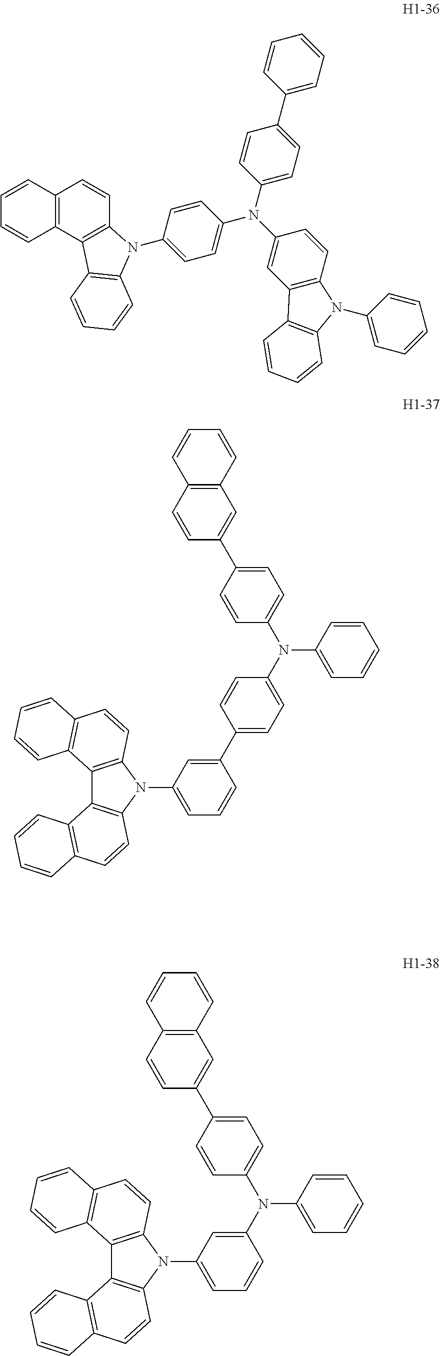 [SMILES: c1ccc(-c2ccc(N(c3ccc(-n4c5ccccc5c5c6ccccc6ccc54)cc3)c3ccc4c(c3)c3ccccc3n4-c3ccccc3)cc2)cc1 ; c1ccc(N(c2ccc(-c3cccc(-n4c5ccc6ccccc6c5c5c6ccccc6ccc54)c3)cc2)c2ccc(-c3ccc4ccccc4c3)cc2)cc1 ; c1ccc(N(c2ccc(-c3ccc4ccccc4c3)cc2)c2cccc(-n3c4ccc5ccccc5c4c4c5ccccc5ccc43)c2)cc1]

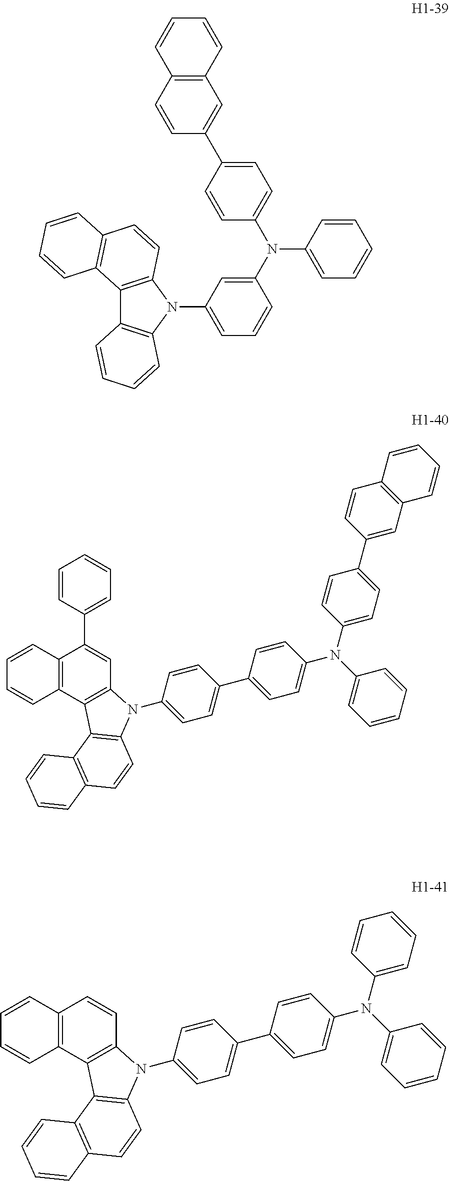 [SMILES: c1ccc(N(c2ccc(-c3ccc4ccccc4c3)cc2)c2cccc(-n3c4ccccc4c4c5ccccc5ccc43)c2)cc1 ; c1ccc(-c2cc3c(c4ccccc24)c2c4ccccc4ccc2n3-c2ccc(-c3ccc(N(c4ccccc4)c4ccc(-c5ccc6ccccc6c5)cc4)cc3)cc2)cc1 ; c1ccc(N(c2ccccc2)c2ccc(-c3ccc(-n4c5ccc6ccccc6c5c5c6ccccc6ccc54)cc3)cc2)cc1]

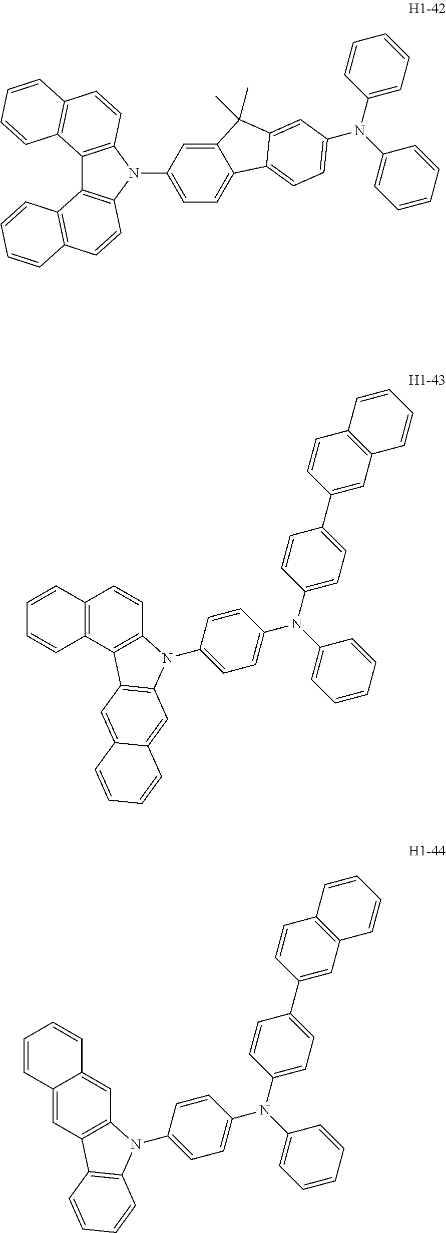 [SMILES: CC1(C)c2cc(N(c3ccccc3)c3ccccc3)ccc2-c2ccc(-n3c4ccc5ccccc5c4c4c5ccccc5ccc43)cc21 ; c1ccc(N(c2ccc(-c3ccc4ccccc4c3)cc2)c2ccc(-n3c4cc5ccccc5cc4c4c5ccccc5ccc43)cc2)cc1 ; c1ccc(N(c2ccc(-c3ccc4ccccc4c3)cc2)c2ccc(-n3c4ccccc4c4cc5ccccc5cc43)cc2)cc1]

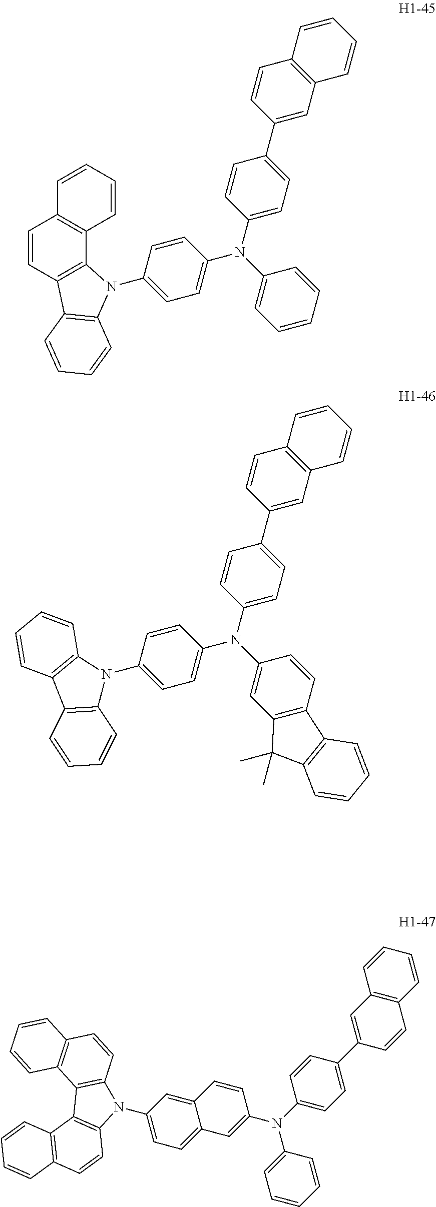 [SMILES: c1ccc(N(c2ccc(-c3ccc4ccccc4c3)cc2)c2ccc(-n3c4ccccc4c4ccc5ccccc5c43)cc2)cc1 ; CC1(C)c2ccccc2-c2ccc(N(c3ccc(-c4ccc5ccccc5c4)cc3)c3ccc(-n4c5ccccc5c5ccccc54)cc3)cc21 ; c1ccc(N(c2ccc(-c3ccc4ccccc4c3)cc2)c2ccc3cc(-n4c5ccc6ccccc6c5c5c6ccccc6ccc54)ccc3c2)cc1]

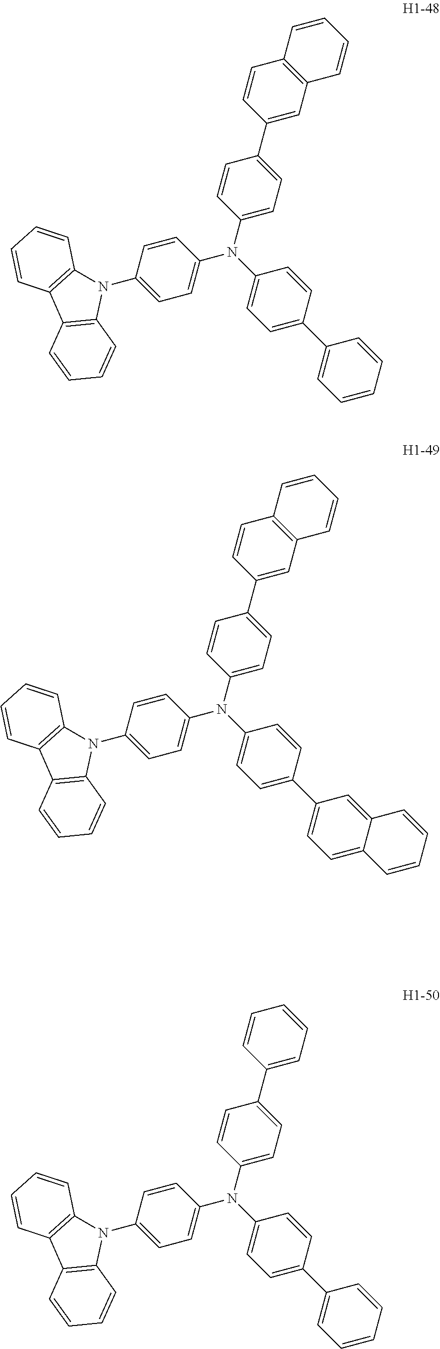 [SMILES: c1ccc(-c2ccc(N(c3ccc(-c4ccc5ccccc5c4)cc3)c3ccc(-n4c5ccccc5c5ccccc54)cc3)cc2)cc1 ; c1ccc2cc(-c3ccc(N(c4ccc(-c5ccc6ccccc6c5)cc4)c4ccc(-n5c6ccccc6c6ccccc65)cc4)cc3)ccc2c1 ; c1ccc(-c2ccc(N(c3ccc(-c4ccccc4)cc3)c3ccc(-n4c5ccccc5c5ccccc54)cc3)cc2)cc1]

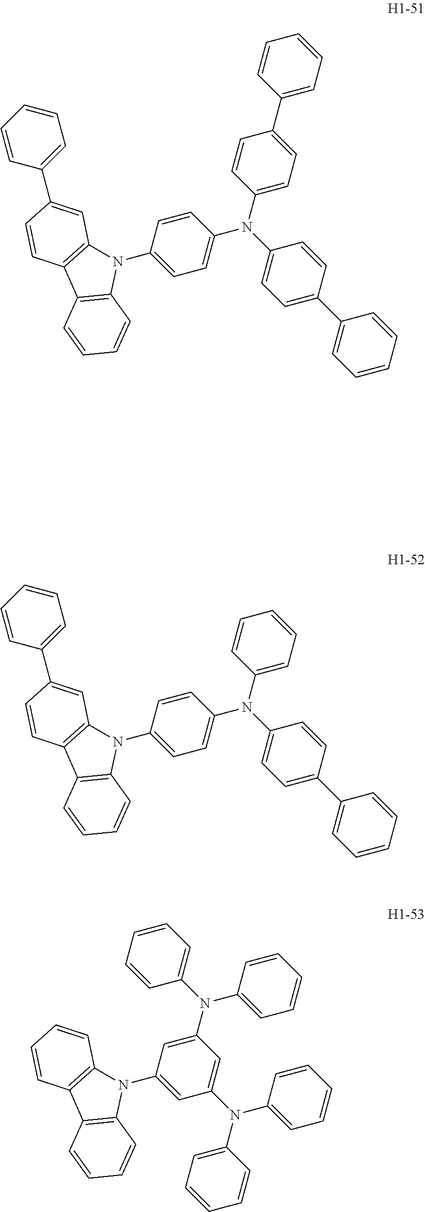 [SMILES: c1ccc(-c2ccc(N(c3ccc(-c4ccccc4)cc3)c3ccc(-n4c5ccccc5c5ccc(-c6ccccc6)cc54)cc3)cc2)cc1 ; c1ccc(-c2ccc(N(c3ccccc3)c3ccc(-n4c5ccccc5c5ccc(-c6ccccc6)cc54)cc3)cc2)cc1 ; c1ccc(N(c2ccccc2)c2cc(N(c3ccccc3)c3ccccc3)cc(-n3c4ccccc4c4ccccc43)c2)cc1]

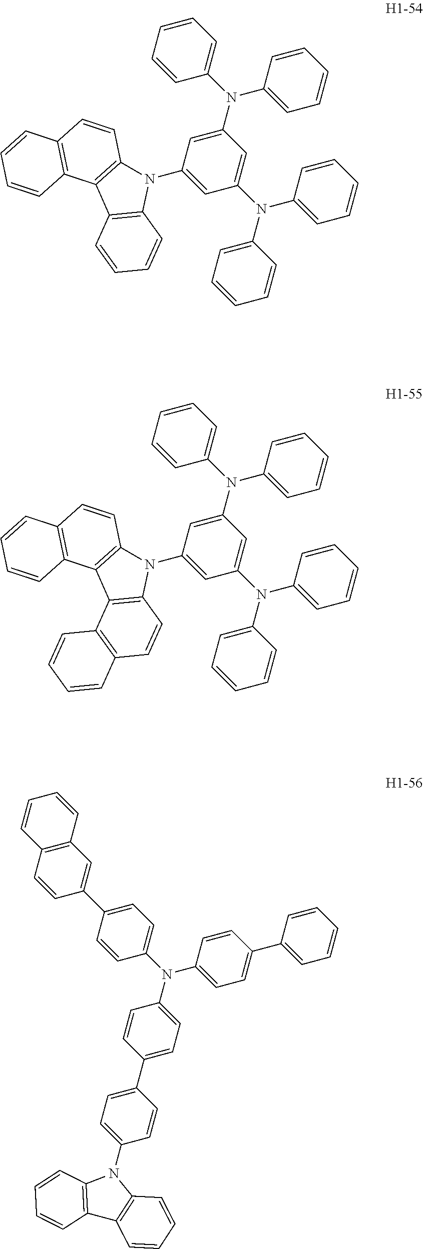 [SMILES: c1ccc(N(c2ccccc2)c2cc(N(c3ccccc3)c3ccccc3)cc(-n3c4ccccc4c4c5ccccc5ccc43)c2)cc1 ; c1ccc(N(c2ccccc2)c2cc(N(c3ccccc3)c3ccccc3)cc(-n3c4ccc5ccccc5c4c4c5ccccc5ccc43)c2)cc1 ; c1ccc(-c2ccc(N(c3ccc(-c4ccc(-n5c6ccccc6c6ccccc65)cc4)cc3)c3ccc(-c4ccc5ccccc5c4)cc3)cc2)cc1]

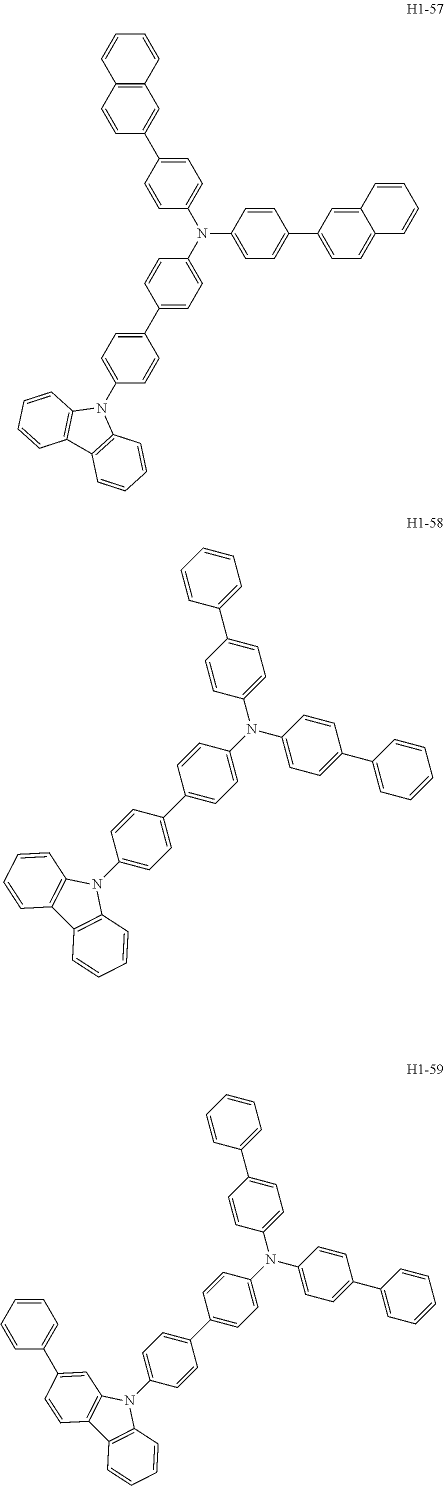 [SMILES: c1ccc2cc(-c3ccc(N(c4ccc(-c5ccc(-n6c7ccccc7c7ccccc76)cc5)cc4)c4ccc(-c5ccc6ccccc6c5)cc4)cc3)ccc2c1 ; c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1]

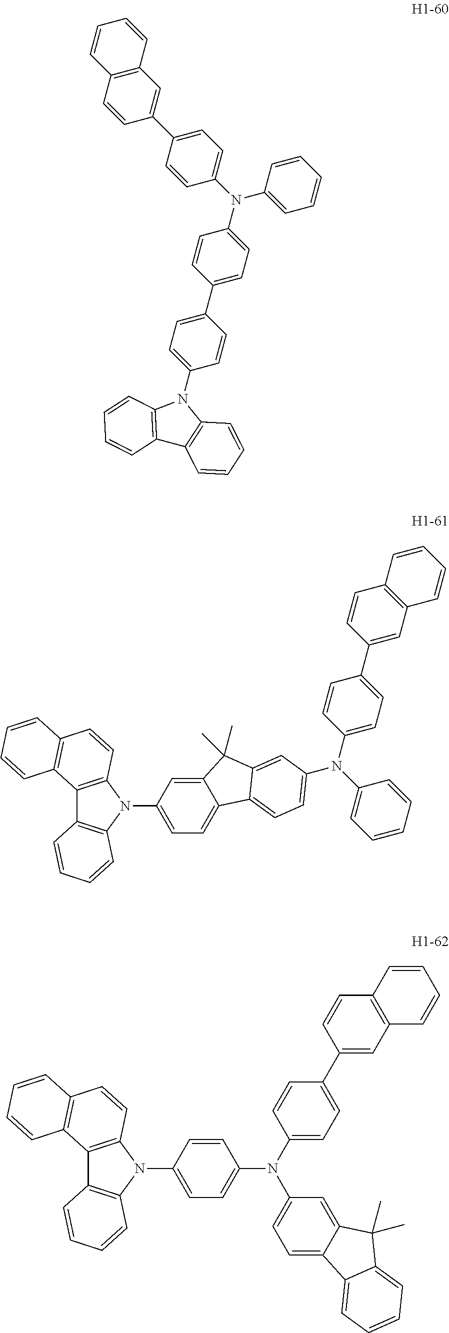 [SMILES: c1ccc(N(c2ccc(-c3ccc(-n4c5ccccc5c5ccccc54)cc3)cc2)c2ccc(-c3ccc4ccccc4c3)cc2)cc1 ; CC1(C)c2cc(N(c3ccccc3)c3ccc(-c4ccc5ccccc5c4)cc3)ccc2-c2ccc(-n3c4ccccc4c4c5ccccc5ccc43)cc21 ; CC1(C)c2ccccc2-c2ccc(N(c3ccc(-c4ccc5ccccc5c4)cc3)c3ccc(-n4c5ccccc5c5c6ccccc6ccc54)cc3)cc21]

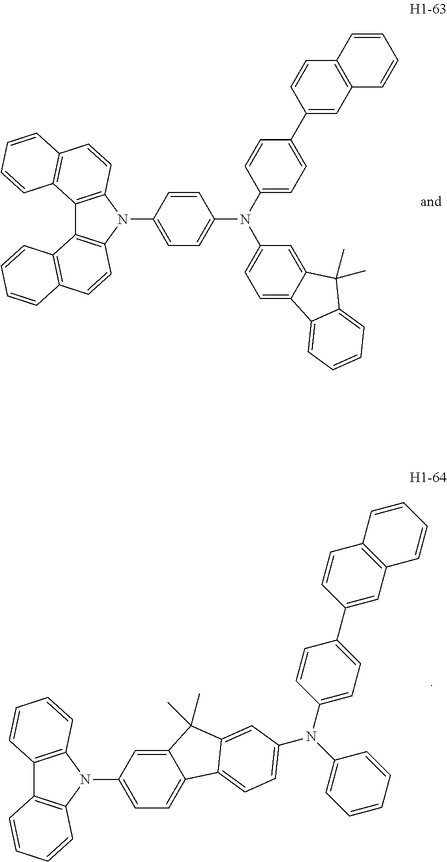 [SMILES: CC1(C)c2ccccc2-c2ccc(N(c3ccc(-c4ccc5ccccc5c4)cc3)c3ccc(-n4c5ccc6ccccc6c5c5c6ccccc6ccc54)cc3)cc21 ; CC1(C)c2cc(N(c3ccccc3)c3ccc(-c4ccc5ccccc5c4)cc3)ccc2-c2ccc(-n3c4ccccc4c4ccccc43)cc21]

6. The host materials according to claim 1, wherein the second host compound represented by formula 2 is selected from the group consisting of:

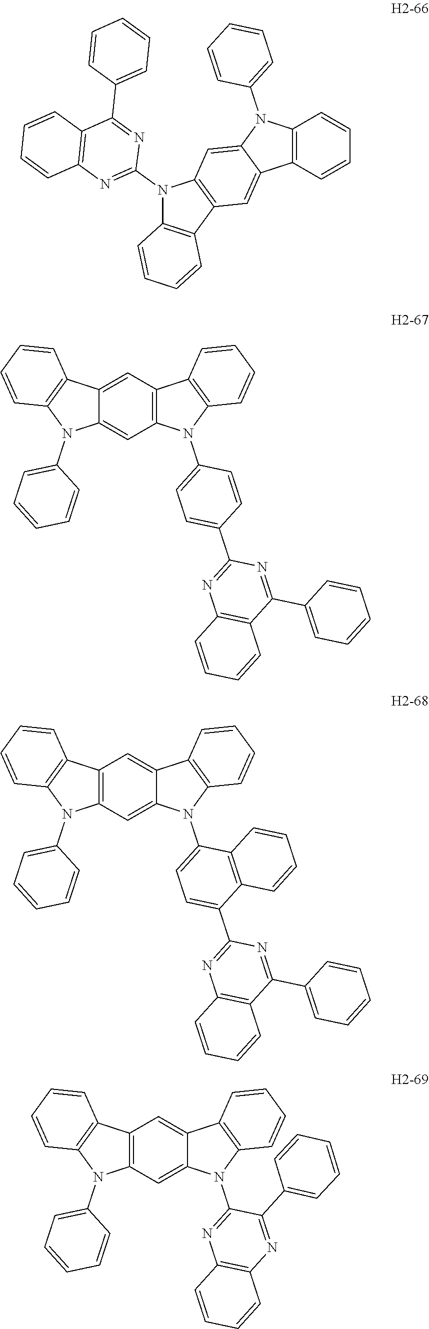

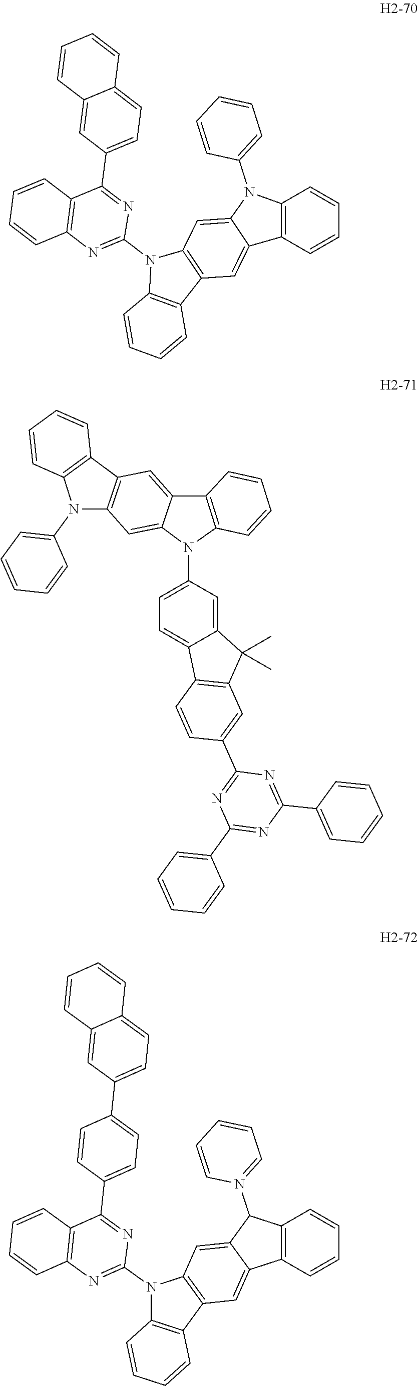

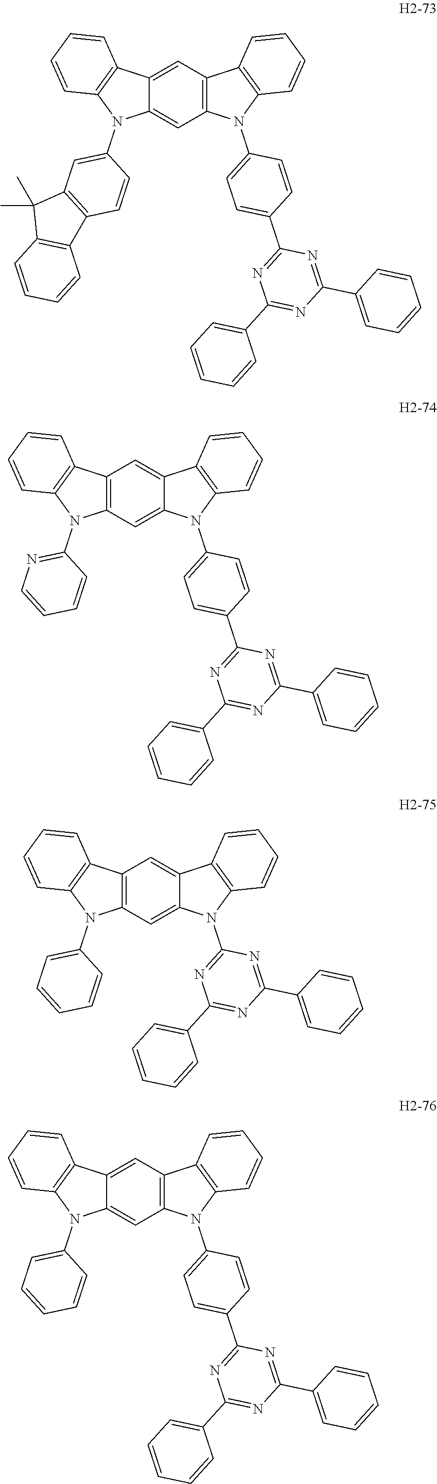 [SMILES: CC1(C)c2ccccc2-c2ccc(-n3c4ccccc4c4cc5c6ccccc6n(-c6ccc(-c7nc(-c8ccccc8)nc(-c8ccccc8)n7)cc6)c5cc43)cc21 ; c1ccc(-c2nc(-c3ccccc3)nc(-c3ccc(-n4c5ccccc5c5cc6c7ccccc7n(-c7ccccn7)c6cc54)cc3)n2)cc1 ; c1ccc(-c2nc(-c3ccccc3)nc(-n3c4ccccc4c4cc5c6ccccc6n(-c6ccccc6)c5cc43)n2)cc1 ; c1ccc(-c2nc(-c3ccccc3)nc(-c3ccc(-n4c5ccccc5c5cc6c7ccccc7n(-c7ccccc7)c6cc54)cc3)n2)cc1]

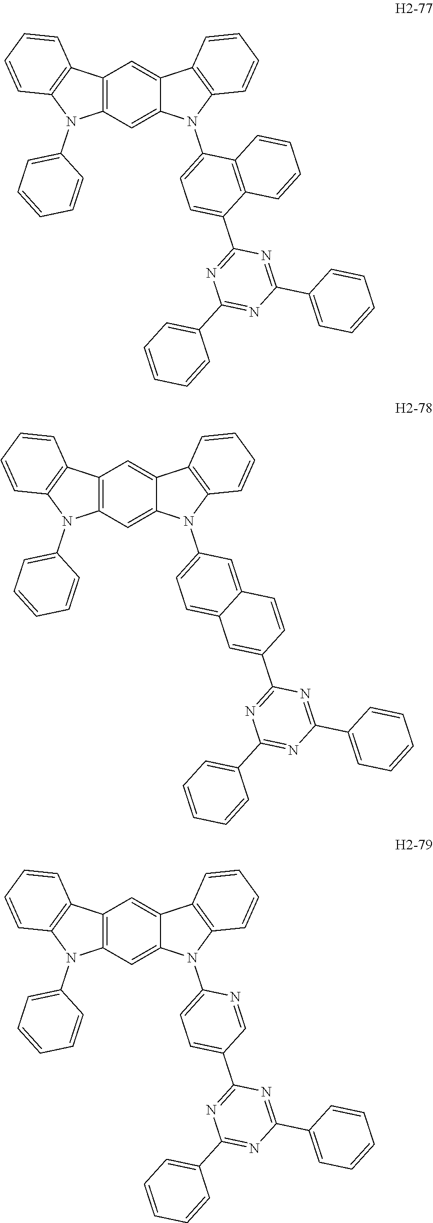 [SMILES: c1ccc(-c2nc(-c3ccccc3)nc(-c3ccc(-n4c5ccccc5c5cc6c7ccccc7n(-c7ccccc7)c6cc54)c4ccccc34)n2)cc1 ; c1ccc(-c2nc(-c3ccccc3)nc(-c3ccc4cc(-n5c6ccccc6c6cc7c8ccccc8n(-c8ccccc8)c7cc65)ccc4c3)n2)cc1 ; c1ccc(-c2nc(-c3ccccc3)nc(-c3ccc(-n4c5ccccc5c5cc6c7ccccc7n(-c7ccccc7)c6cc54)nc3)n2)cc1]

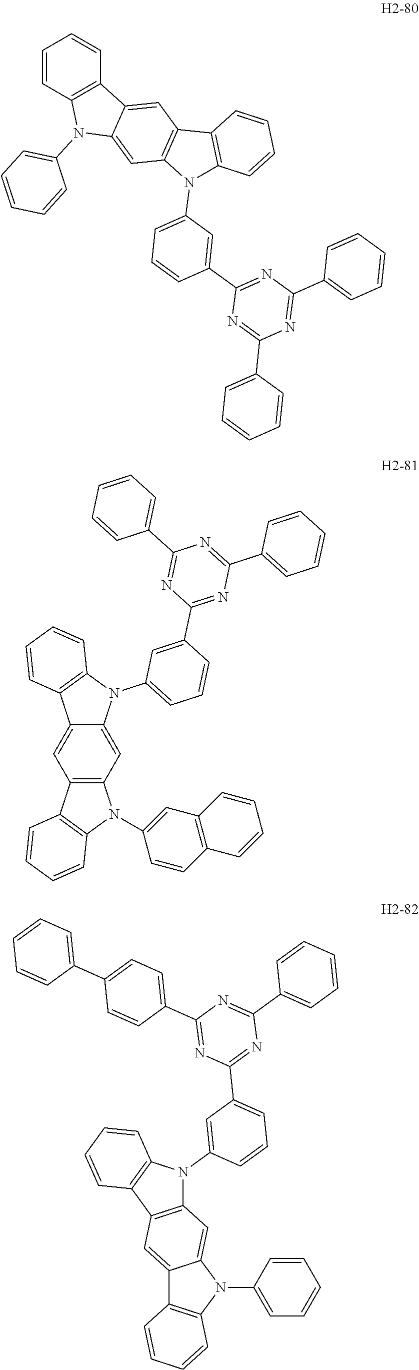

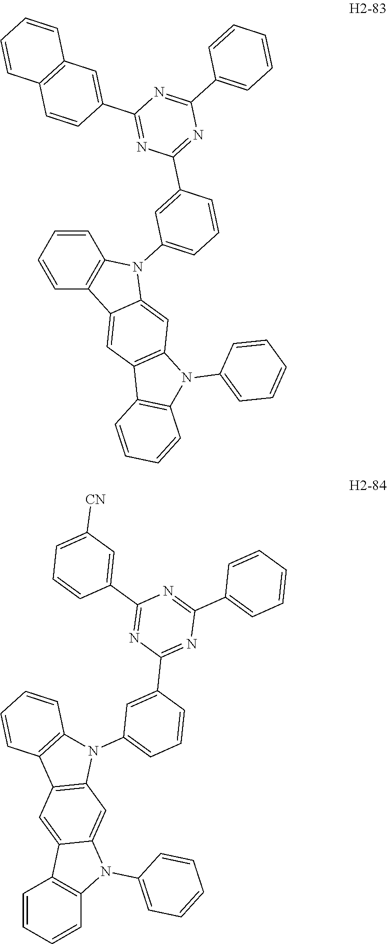 [SMILES: c1ccc(-c2nc(-c3cccc(-n4c5ccccc5c5cc6c7ccccc7n(-c7ccccc7)c6cc54)c3)nc(-c3ccc4ccccc4c3)n2)cc1 ; N#Cc1cccc(-c2nc(-c3ccccc3)nc(-c3cccc(-n4c5ccccc5c5cc6c7ccccc7n(-c7ccccc7)c6cc54)c3)n2)c1]

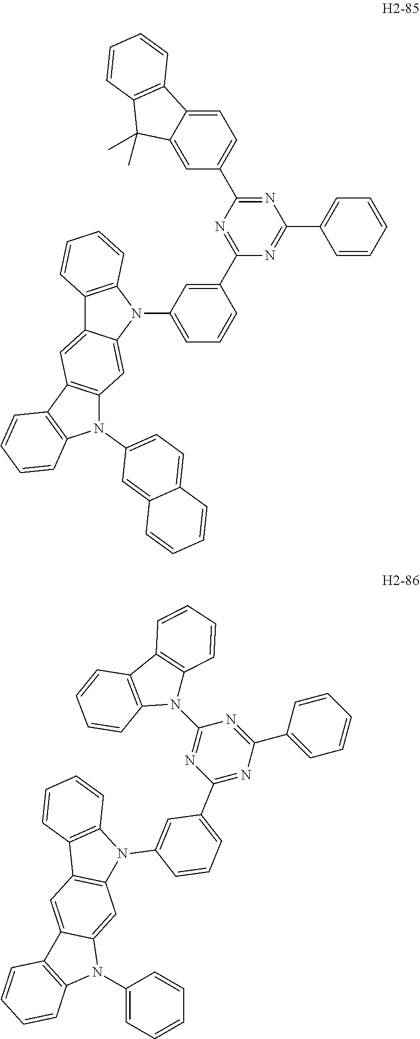 [SMILES: CC1(C)c2ccccc2-c2ccc(-c3nc(-c4ccccc4)nc(-c4cccc(-n5c6ccccc6c6cc7c8ccccc8n(-c8ccc9ccccc9c8)c7cc65)c4)n3)cc21 ; c1ccc(-c2nc(-c3cccc(-n4c5ccccc5c5cc6c7ccccc7n(-c7ccccc7)c6cc54)c3)nc(-n3c4ccccc4c4ccccc43)n2)cc1]

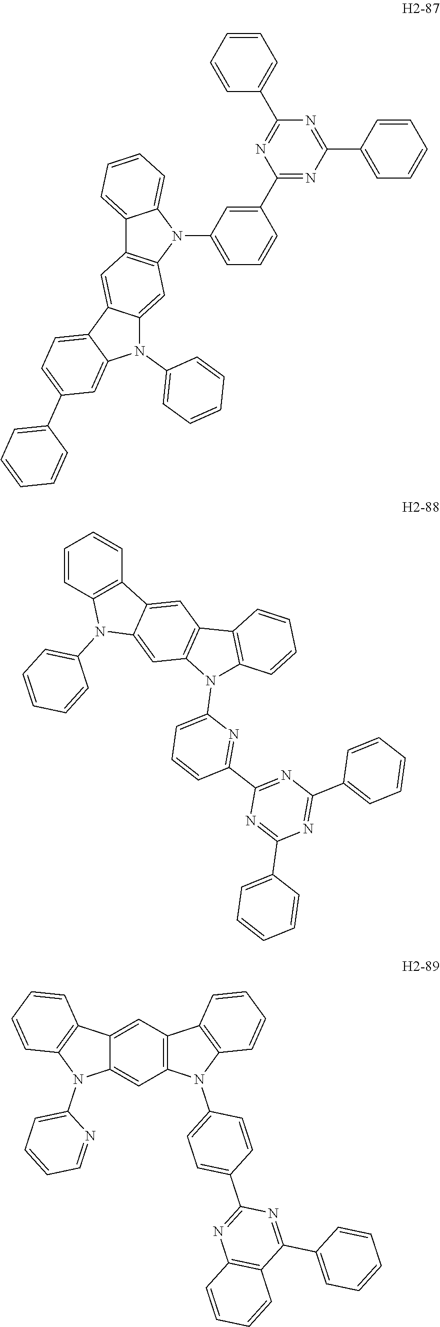 [SMILES: c1ccc(-c2ccc3c4cc5c6ccccc6n(-c6cccc(-c7nc(-c8ccccc8)nc(-c8ccccc8)n7)c6)c5cc4n(-c4ccccc4)c3c2)cc1 ; c1ccc(-c2nc(-c3ccccc3)nc(-c3cccc(-n4c5ccccc5c5cc6c7ccccc7n(-c7ccccc7)c6cc54)n3)n2)cc1 ; c1ccc(-c2nc(-c3ccc(-n4c5ccccc5c5cc6c7ccccc7n(-c7ccccn7)c6cc54)cc3)nc3ccccc23)cc1]

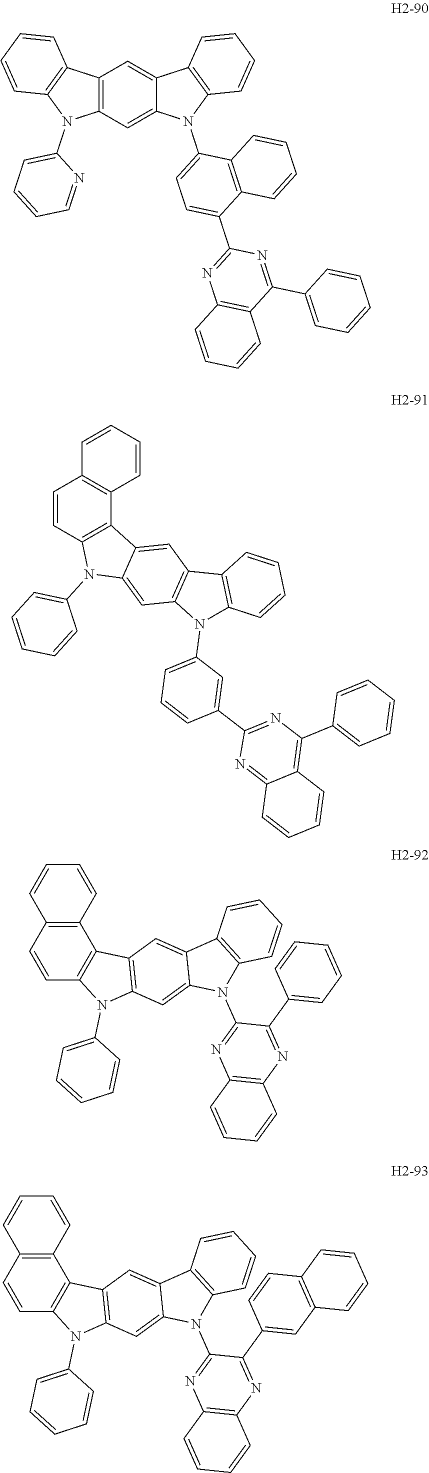 [SMILES: c1ccc(-c2nc(-c3ccc(-n4c5ccccc5c5cc6c7ccccc7n(-c7ccccn7)c6cc54)c4ccccc34)nc3ccccc23)cc1 ; c1ccc(-c2nc(-c3cccc(-n4c5ccccc5c5cc6c7c8ccccc8ccc7n(-c7ccccc7)c6cc54)c3)nc3ccccc23)cc1 ; c1ccc(-c2nc3ccccc3nc2-n2c3ccccc3c3cc4c5c6ccccc6ccc5n(-c5ccccc5)c4cc32)cc1 ; c1ccc(-n2c3cc4c(cc3c3c5ccccc5ccc32)c2ccccc2n4-c2nc3ccccc3nc2-c2ccc3ccccc3c2)cc1]

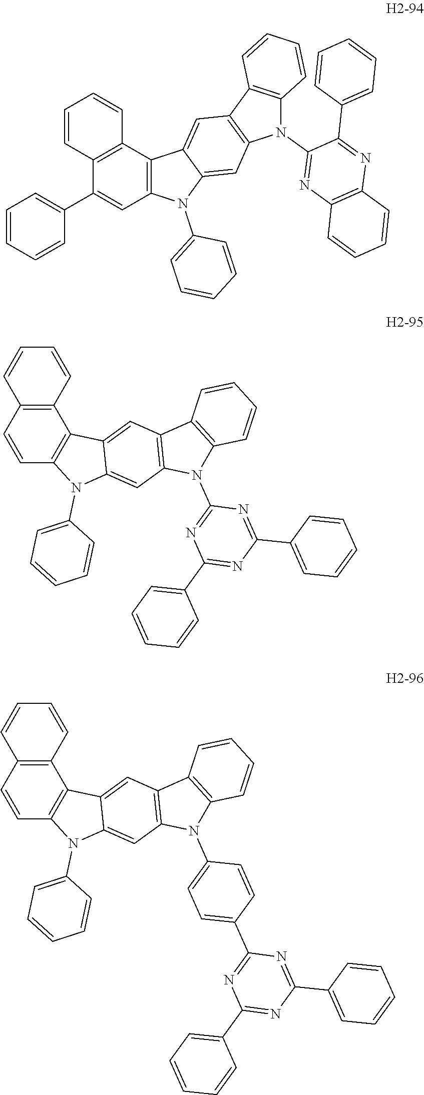 [SMILES: c1ccc(-c2nc3ccccc3nc2-n2c3ccccc3c3cc4c5c6ccccc6c(-c6ccccc6)cc5n(-c5ccccc5)c4cc32)cc1 ; c1ccc(-c2nc(-c3ccccc3)nc(-n3c4ccccc4c4cc5c6c7ccccc7ccc6n(-c6ccccc6)c5cc43)n2)cc1 ; c1ccc(-c2nc(-c3ccccc3)nc(-c3ccc(-n4c5ccccc5c5cc6c7c8ccccc8ccc7n(-c7ccccc7)c6cc54)cc3)n2)cc1]

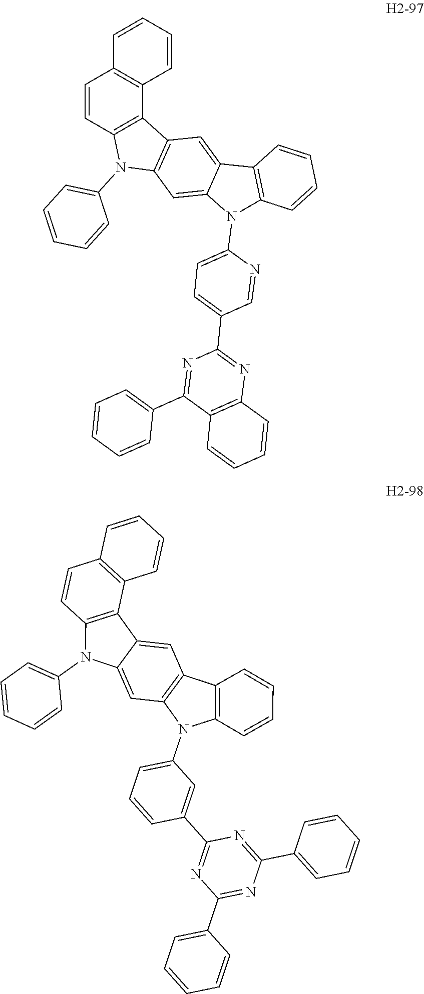 [SMILES: c1ccc(-c2nc(-c3ccc(-n4c5ccccc5c5cc6c7c8ccccc8ccc7n(-c7ccccc7)c6cc54)nc3)nc3ccccc23)cc1 ; c1ccc(-c2nc(-c3ccccc3)nc(-c3cccc(-n4c5ccccc5c5cc6c7c8ccccc8ccc7n(-c7ccccc7)c6cc54)c3)n2)cc1]

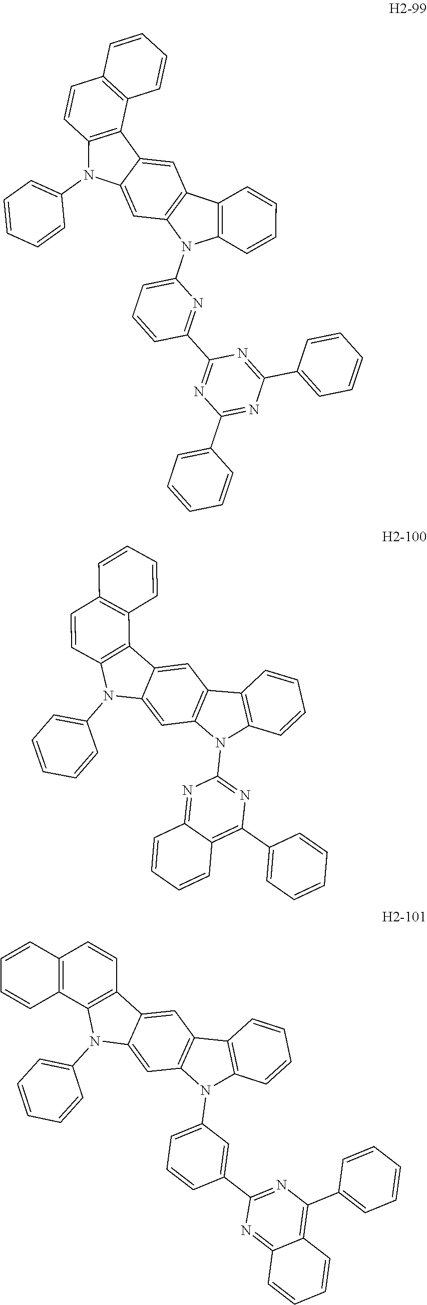 [SMILES: c1ccc(-c2nc(-c3ccccc3)nc(-c3cccc(-n4c5ccccc5c5cc6c7c8ccccc8ccc7n(-c7ccccc7)c6cc54)n3)n2)cc1 ; c1ccc(-c2nc(-n3c4ccccc4c4cc5c6c7ccccc7ccc6n(-c6ccccc6)c5cc43)nc3ccccc23)cc1 ; c1ccc(-c2nc(-c3cccc(-n4c5ccccc5c5cc6c7ccc8ccccc8c7n(-c7ccccc7)c6cc54)c3)nc3ccccc23)cc1]

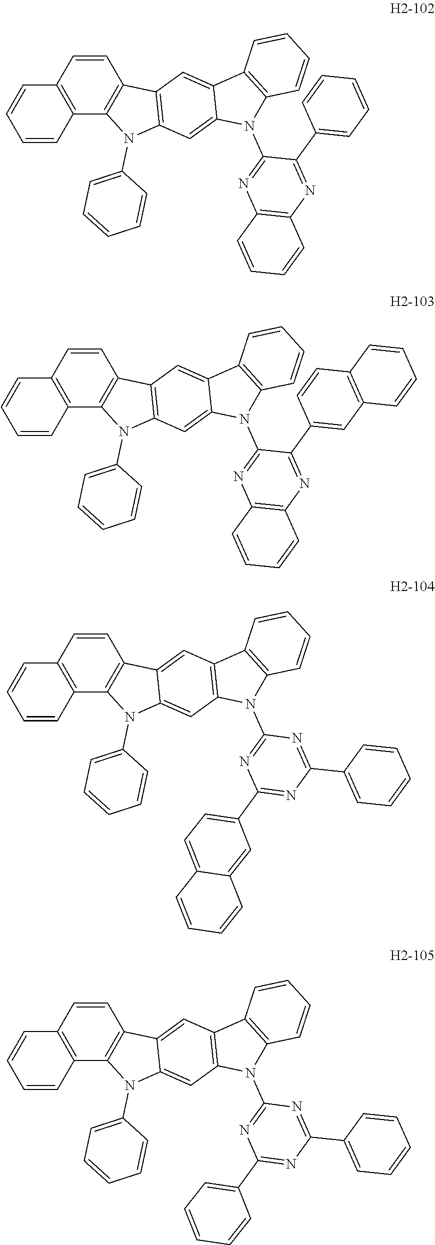

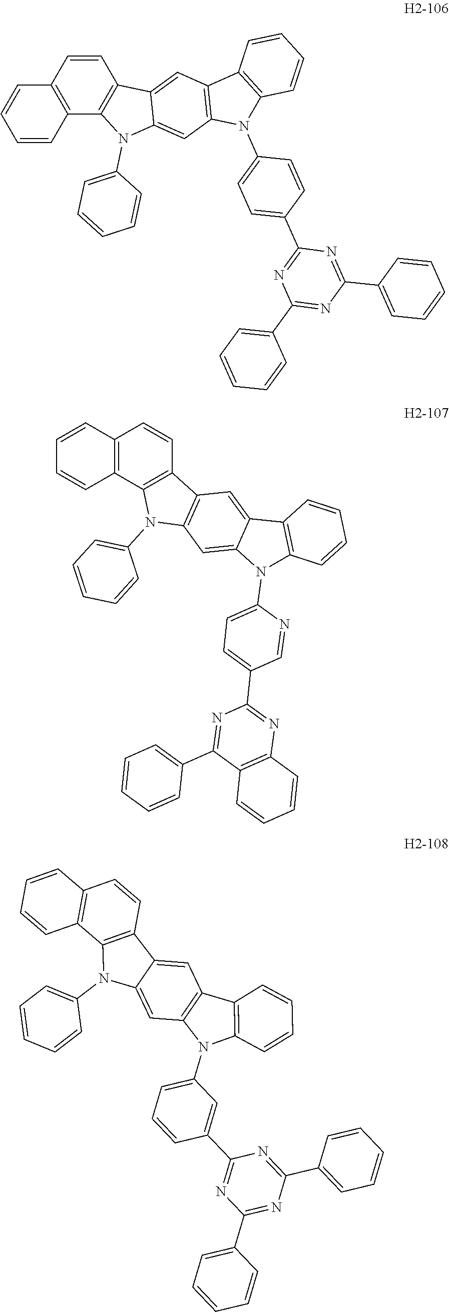 [SMILES: c1ccc(-c2nc(-c3ccccc3)nc(-c3ccc(-n4c5ccccc5c5cc6c7ccc8ccccc8c7n(-c7ccccc7)c6cc54)cc3)n2)cc1 ; c1ccc(-c2nc(-c3ccc(-n4c5ccccc5c5cc6c7ccc8ccccc8c7n(-c7ccccc7)c6cc54)nc3)nc3ccccc23)cc1 ; c1ccc(-c2nc(-c3ccccc3)nc(-c3cccc(-n4c5ccccc5c5cc6c7ccc8ccccc8c7n(-c7ccccc7)c6cc54)c3)n2)cc1]

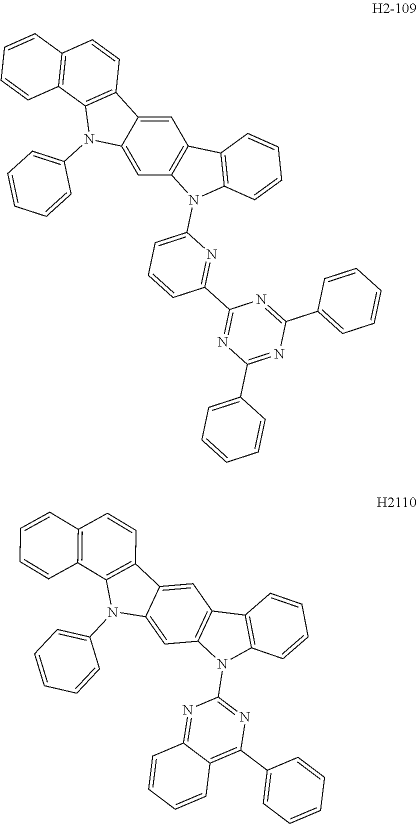 [SMILES: c1ccc(-c2nc(-c3ccccc3)nc(-c3cccc(-n4c5ccccc5c5cc6c7ccc8ccccc8c7n(-c7ccccc7)c6cc54)n3)n2)cc1 ; c1ccc(-c2nc(-n3c4ccccc4c4cc5c6ccc7ccccc7c6n(-c6ccccc6)c5cc43)nc3ccccc23)cc1]

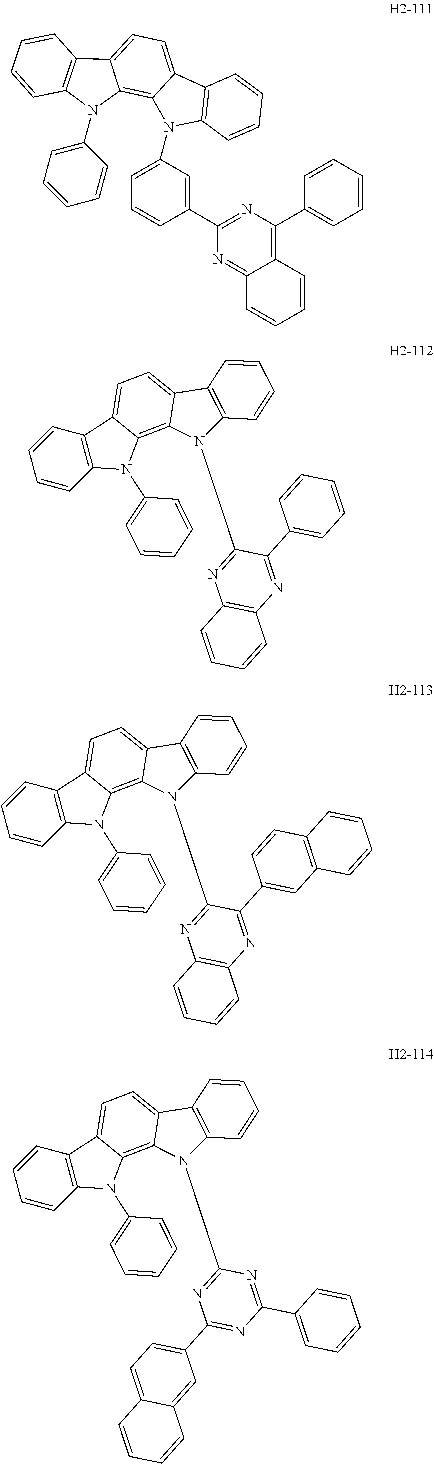

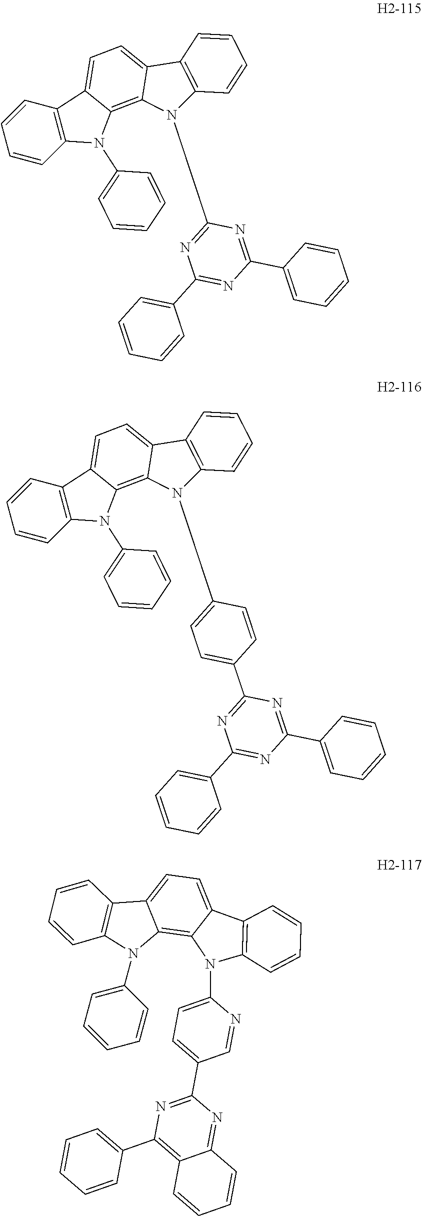

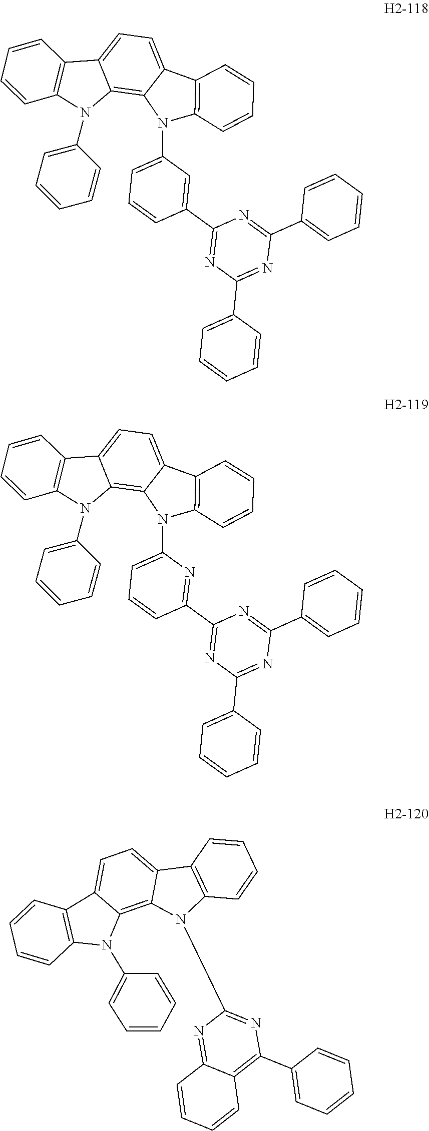 [SMILES: c1ccc(-c2nc(-c3ccccc3)nc(-c3cccc(-n4c5ccccc5c5ccc6c7ccccc7n(-c7ccccc7)c6c54)c3)n2)cc1 ; c1ccc(-c2nc(-c3ccccc3)nc(-c3cccc(-n4c5ccccc5c5ccc6c7ccccc7n(-c7ccccc7)c6c54)n3)n2)cc1 ; c1ccc(-c2nc(-n3c4ccccc4c4ccc5c6ccccc6n(-c6ccccc6)c5c43)nc3ccccc23)cc1]

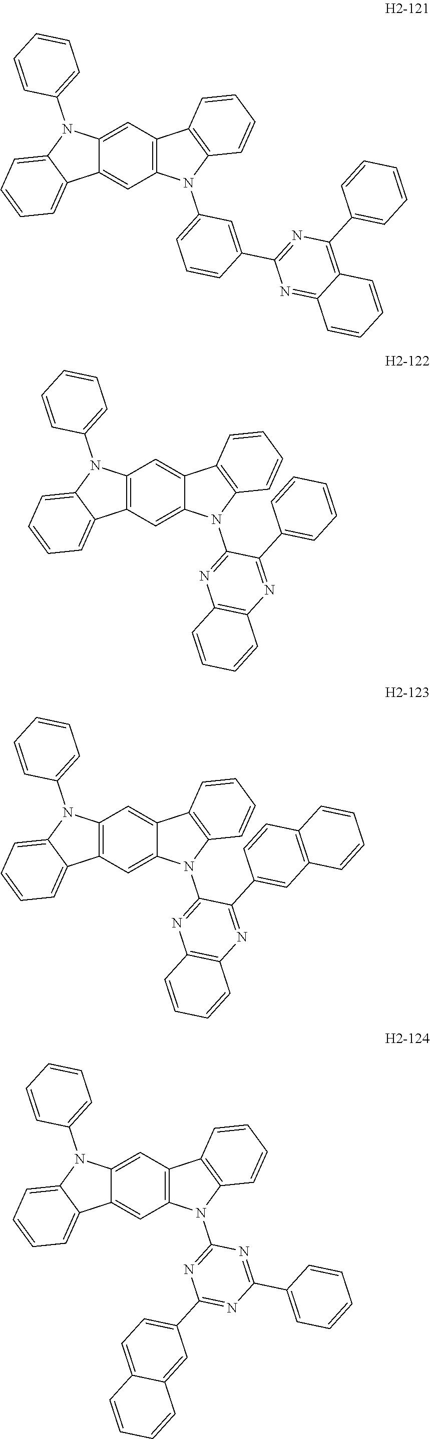 [SMILES: c1ccc(-c2nc(-c3cccc(-n4c5ccccc5c5cc6c(cc54)c4ccccc4n6-c4ccccc4)c3)nc3ccccc23)cc1 ; c1ccc(-c2nc3ccccc3nc2-n2c3ccccc3c3cc4c(cc32)c2ccccc2n4-c2ccccc2)cc1 ; c1ccc(-n2c3ccccc3c3cc4c(cc32)c2ccccc2n4-c2nc3ccccc3nc2-c2ccc3ccccc3c2)cc1 ; c1ccc(-c2nc(-c3ccc4ccccc4c3)nc(-n3c4ccccc4c4cc5c(cc43)c3ccccc3n5-c3ccccc3)n2)cc1]

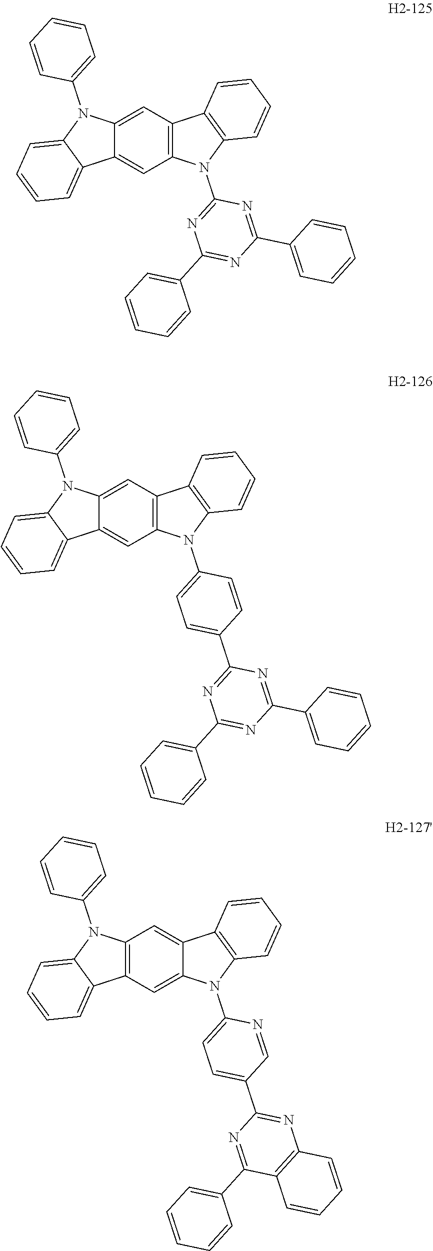 [SMILES: c1ccc(-c2nc(-c3ccccc3)nc(-n3c4ccccc4c4cc5c(cc43)c3ccccc3n5-c3ccccc3)n2)cc1 ; c1ccc(-c2nc(-c3ccccc3)nc(-c3ccc(-n4c5ccccc5c5cc6c(cc54)c4ccccc4n6-c4ccccc4)cc3)n2)cc1 ; c1ccc(-c2nc(-c3ccc(-n4c5ccccc5c5cc6c(cc54)c4ccccc4n6-c4ccccc4)nc3)nc3ccccc23)cc1]

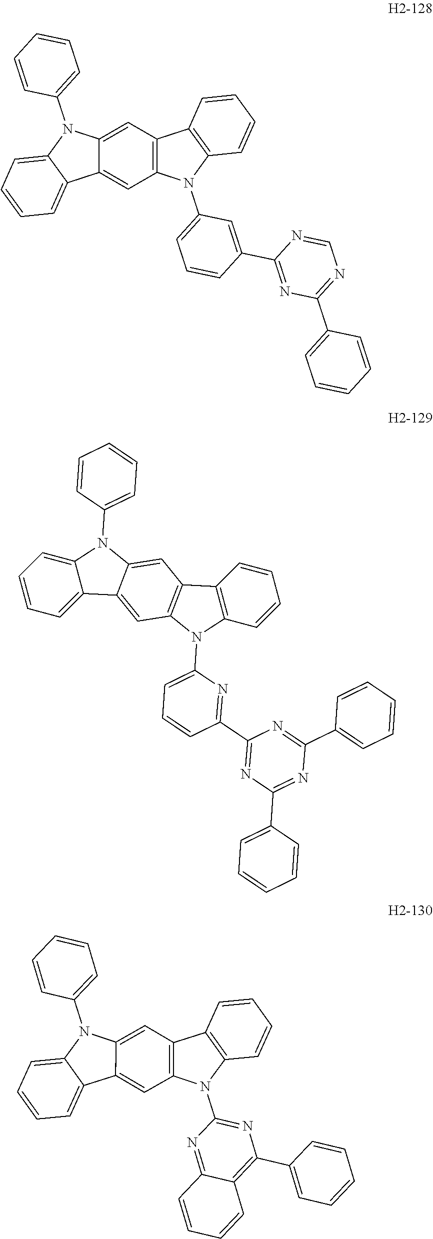 [SMILES: c1ccc(-c2ncnc(-c3cccc(-n4c5ccccc5c5cc6c(cc54)c4ccccc4n6-c4ccccc4)c3)n2)cc1 ; c1ccc(-c2nc(-c3ccccc3)nc(-c3cccc(-n4c5ccccc5c5cc6c(cc54)c4ccccc4n6-c4ccccc4)n3)n2)cc1 ; c1ccc(-c2nc(-n3c4ccccc4c4cc5c(cc43)c3ccccc3n5-c3ccccc3)nc3ccccc23)cc1]

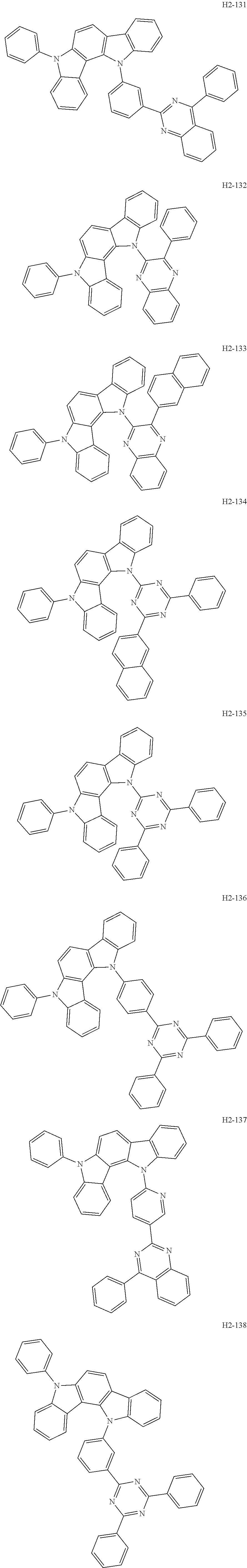

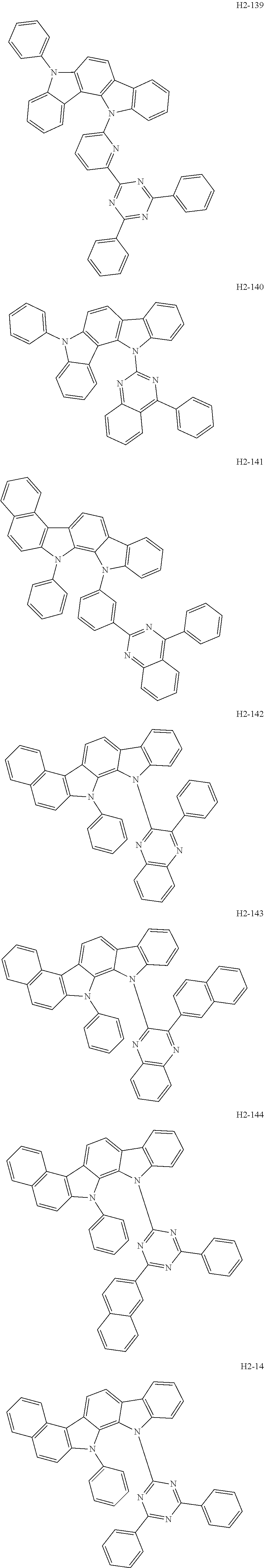

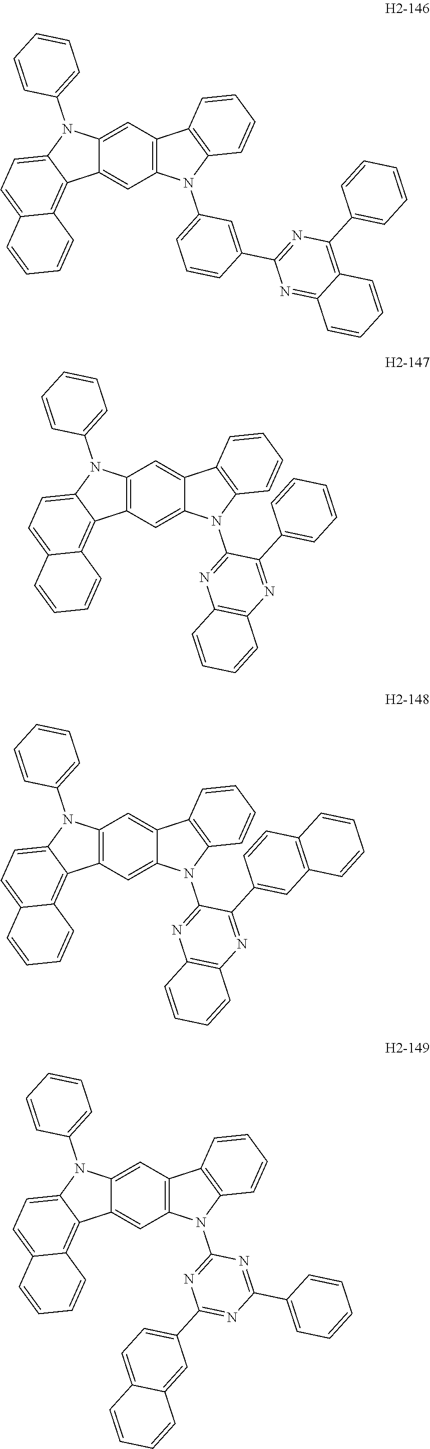 [SMILES: c1ccc(-c2nc(-c3cccc(-n4c5ccccc5c5cc6c(cc54)c4c5ccccc5ccc4n6-c4ccccc4)c3)nc3ccccc23)cc1 ; c1ccc(-c2nc3ccccc3nc2-n2c3ccccc3c3cc4c(cc32)c2c3ccccc3ccc2n4-c2ccccc2)cc1 ; c1ccc(-n2c3cc4c5ccccc5n(-c5nc6ccccc6nc5-c5ccc6ccccc6c5)c4cc3c3c4ccccc4ccc32)cc1 ; c1ccc(-c2nc(-c3ccc4ccccc4c3)nc(-n3c4ccccc4c4cc5c(cc43)c3c4ccccc4ccc3n5-c3ccccc3)n2)cc1]

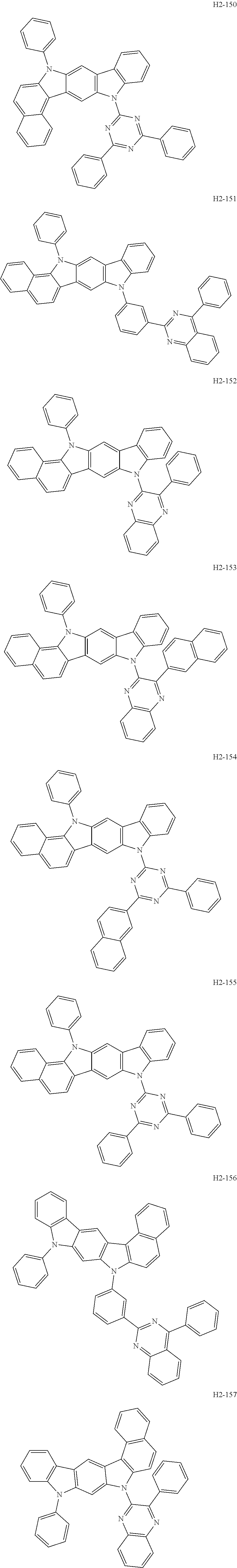

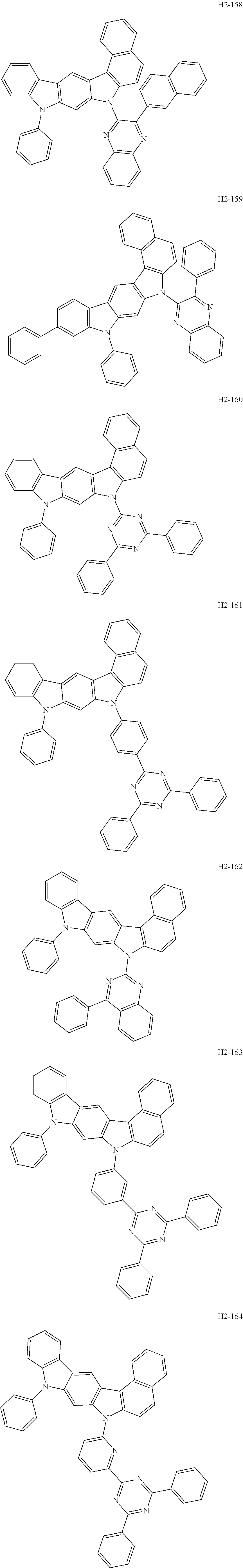

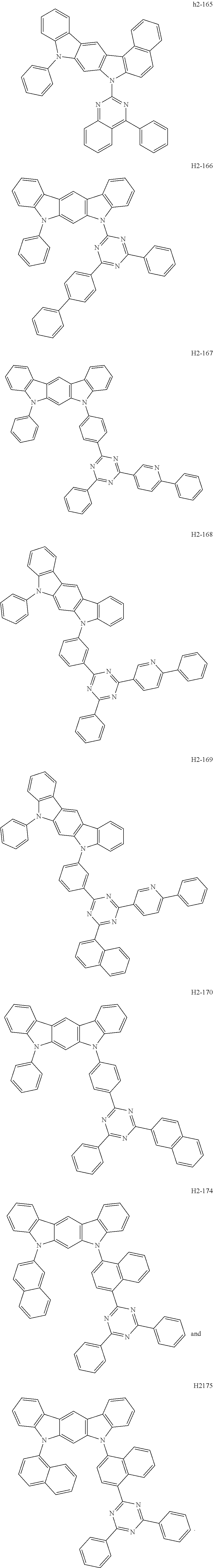

7. An organic electroluminescent device comprising an anode, a cathode, and at least one light-emitting layer formed between the anode and the cathode, wherein the light-emitting layer comprises a host and a phosphorescent dopant, and the host comprises the host materials according to claim 1.
